SEQUENCE LISTING (70 sequences)

SEQ ID NO: 1 - protein, 6 aa, synthetic construct
VTVSSH

SEQ ID NO: 2 - protein, 7 aa, synthetic construct
VTVSSHH

SEQ ID NO: 3 - protein, 8 aa, synthetic construct
VTVSSHHH

SEQ ID NO: 4 - protein, 9 aa, synthetic construct
VTVSSHHHH

SEQ ID NO: 5 - protein, 10 aa, synthetic construct
VTVSSHHHHH

SEQ ID NO: 6 - protein, 5 aa, synthetic construct
VEIKH

SEQ ID NO: 7 - protein, 6 aa, synthetic construct
VEIKHH

SEQ ID NO: 8 - protein, 7 aa, synthetic construct
VEIKHHH

SEQ ID NO: 9 - protein, 8 aa, synthetic construct
VEIKHHHH

SEQ ID NO: 10 - protein, 9 aa, synthetic construct
VEIKHHHHH

SEQ ID NO: 11 - protein, 6 aa, synthetic construct
VEIKRH

SEQ ID NO: 12 - protein, 7 aa, synthetic construct
VEIKRHH

SEQ ID NO: 13 - protein, 8 aa, synthetic construct
VEIKRHHH

SEQ ID NO: 14 - protein, 9 aa, synthetic construct
VEIKRHHHH

SEQ ID NO: 15 - protein, 10 aa, synthetic construct
VEIKRHHHHH

SEQ ID NO: 16 - protein, 7 aa, synthetic construct
VEIKRTH

SEQ ID NO: 17 - protein, 8 aa, synthetic construct
VEIKRTHH

SEQ ID NO: 18 - protein, 9 aa, synthetic construct
VEIKRTHHH

SEQ ID NO: 19 - protein, 10 aa, synthetic construct
VEIKRTHHHH

SEQ ID NO: 20 - protein, 11 aa, synthetic construct
VEIKRTHHHHH

SEQ ID NO: 21 - protein, 5 aa, Homo sapiens
  source 1..5: note = Additional organisms: Mus musculus, Macaca mulatta
VTVSS

SEQ ID NO: 22 - protein, 4 aa, Homo sapiens
  source 1..4: note = Additional organisms: Macaca mulatta, Bos taurus
VEIK

SEQ ID NO: 23 - protein, 5 aa, Homo sapiens
VEIKR

SEQ ID NO: 24 - protein, 6 aa, Homo sapiens
VEIKRT

SEQ ID NO: 25 - protein, 5 aa, synthetic construct
  source 1..5: note = up to 4 copies of H could be deleted
HHHHH

SEQ ID NO: 26 - protein, 6 aa, synthetic construct
HHHHHH

SEQ ID NO: 27 - protein, 4 aa, synthetic construct
HHHH

SEQ ID NO: 28 - protein, 5 aa, synthetic construct
HHHHH

SEQ ID NO: 29 - protein, 5 aa, Mus musculus
LTVSS

SEQ ID NO: 30 - protein, 5 aa, Mus musculus
VTVSA

SEQ ID NO: 31 - protein, 6 aa, synthetic construct
  REPEAT 6: note = H can be repeated n times, wherein n is 1, 2, 3, 4 or 5
VTVSSH

SEQ ID NO: 32 - protein, 5 aa, synthetic construct
  REPEAT 5: note = H can be repeated n times, wherein n is 1, 2, 3, 4 or 5
VEIKH

SEQ ID NO: 33 - protein, 6 aa, synthetic construct
  REPEAT 6: note = H can be repeated n times, wherein n is 1, 2, 3, 4 or 5
VEIKRH

SEQ ID NO: 34 - protein, 7 aa, synthetic construct
  REPEAT 7: note = H can be repeated n times, wherein n is 1, 2, 3, 4 or 5
VEIKRTH

SEQ ID NO: 35 - protein, 6 aa, synthetic construct
  source 1..6: note = up to 5 copies of G could be deleted
GGGGGG

SEQ ID NO: 36 - protein, 8 aa, Homo sapiens
SLSLSPGK

SEQ ID NO: 37 - protein, 8 aa, synthetic construct
TVAPTESS

SEQ ID NO: 38 - protein, 6 aa, Homo sapiens
  source 1..6: note = Additional organism: Mus musculus
TKVTVL

SEQ ID NO: 39 - protein, 6 aa, Homo sapiens
  source 1..6: note = Additional organisms: Mus musculus, Macaca mulatta
TKLTVL

SEQ ID NO: 40 - protein, 6 aa, Homo sapiens
TQLIIL

SEQ ID NO: 41 - protein, 6 aa, Homo sapiens
TELTVL

SEQ ID NO: 42 - protein, 6 aa, Homo sapiens
TQLTVL

SEQ ID NO: 43 - protein, 7 aa, Mus musculus
NGLVYAG

SEQ ID NO: 44 - protein, 7 aa, Mus musculus
NGLLYAG

SEQ ID NO: 45 - protein, 6 aa, Mus musculus
  source 1..6: note = Additional organisms: Macaca mulatta, Bos taurus
TRLTVL

SEQ ID NO: 46 - protein, 7 aa, synthetic construct
  REPEAT 7: note = H can be repeated n times, wherein n is 1, 2, 3, 4 or 5
TKVTVLH

SEQ ID NO: 47 - protein, 7 aa, synthetic construct
  REPEAT 7: note = H can be repeated n times, wherein n is 1, 2, 3, 4 or 5
TKLTVLH

SEQ ID NO: 48 - protein, 7 aa, synthetic construct
  REPEAT 7: note = H can be repeated n times, wherein n is 1, 2, 3, 4 or 5
TQLIILH

SEQ ID NO: 49 - protein, 7 aa, synthetic construct
  REPEAT 7: note = H can be repeated n times, wherein n is 1, 2, 3, 4 or 5
TELTVLH

SEQ ID NO: 50 - protein, 7 aa, synthetic construct
  REPEAT 7: note = H can be repeated n times, wherein n is 1, 2, 3, 4 or 5
TQLTVLH

SEQ ID NO: 51 - protein, 8 aa, synthetic construct
  REPEAT 8: note = H can be repeated n times, wherein n is 1, 2, 3, 4 or 5
NGLVYAGH

SEQ ID NO: 52 - protein, 8 aa, synthetic construct
  REPEAT 8: note = H can be repeated n times, wherein n is 1, 2, 3, 4 or 5
NGLLYAGH

SEQ ID NO: 53 - protein, 7 aa, synthetic construct
  REPEAT 7: note = H can be repeated n times, wherein n is 1, 2, 3, 4 or 5
TRLTVLH

SEQ ID NO: 54 - protein, 5 aa, Macaca mulatta
ITISS

SEQ ID NO: 55 - protein, 5 aa, Gallus gallus
VIVSS

SEQ ID NO: 56 - protein, 4 aa, Homo sapiens
VDIK

SEQ ID NO: 57 - protein, 4 aa, Homo sapiens
  source 1..4: note = Additional organisms: Mus musculus, Macaca mulatta
LEIK

SEQ ID NO: 58 - protein, 4 aa, Macaca mulatta
LDIK

SEQ ID NO: 59 - protein, 4 aa, Bos taurus
LEVK

SEQ ID NO: 60 - protein, 4 aa, Bos taurus
VEIN

SEQ ID NO: 61 - protein, 4 aa, Bos taurus
LEIR

SEQ ID NO: 62 - protein, 6 aa, Mus musculus
GLVYAG

SEQ ID NO: 63 - protein, 6 aa, Mus musculus
GLLYAG

SEQ ID NO: 64 - protein, 6 aa, Macaca mulatta
TQLTIV

SEQ ID NO: 65 - protein, 6 aa, Macaca mulatta
TKLTIL

SEQ ID NO: 66 - protein, 6 aa, Macaca mulatta
TRLTDI

SEQ ID NO: 67 - protein, 6 aa, Gallus gallus
  source 1..6: note = Additional organism: Bos taurus
TTLTVL

SEQ ID NO: 68 - protein, 6 aa, Bos taurus
TWVTVL

SEQ ID NO: 69 - protein, 6 aa, Bos taurus
TRVTVL

SEQ ID NO: 70 - protein, 6 aa, Bos taurus
TTVTVL

(19) United States
(12) Patent Application Publication
(10) Pub. No.: US 2025/0368756 A1
(43) Pub. Date: Dec. 4, 2025

(54) ENHANCED PROTEIN COMPOSITIONS

(71) Applicant: Janssen Biotech, Inc., Horsham, PA (US)

(72) Inventors: Jun CHEN, New Hope, PA (US); Adam ZWOLAK, Bala Cynwyd, PA (US); Partha Sarathi CHOWDHURY, Gaithersburg, MD (US); Sanjaya SINGH, Blue Bell, PA (US); Danlin YANG, Philadelphia, PA (US); Tong-Yuan YANG, Chadds Ford, PA (US)

(21) Appl. No.: 18/833,114

(22) PCT Filed: Jan. 26, 2023
(86) PCT No.: PCT/US2023/061388

§ 371 Date: Jul. 25, 2024

Related U.S. Application Data
(60) Provisional application No. 63/303,921, filed on Jan. 27, 2022.

(51) Int. Cl. C07K 16/42

(52) U.S. Cl. CPC C07K 16/42; C07K 2317/31; C07K 2317/94; C07K 2319/21

(57) ABSTRACT

The presently described invention teaches isolated polypeptides, including diagnostic, prognostic, and therapeutic polypeptides, such as, for example, polypeptides that are capable of binding to themselves or other polypeptides, said isolated polypeptides comprising an exposed carboxy-terminus (“C-terminus”) fused to a cap domain (e.g., a His cap domain, (His)6 (i.e., HHHHHH (SEQ ID NO:26)), SLSLSPGK (SEQ ID NO:36), AS, or TVAPTESS (SEQ ID NO: 37)). The presently described invention further teaches nucleic acids and/or expression vectors encoding the polypeptides; cells containing the polypeptides, nucleic acids, and/or expression vectors; and compositions comprising the polypeptides. Methods of making the polypeptides, and methods of using the polypeptides are also taught.

### 1. CROSS-REFERENCE TO RELATED APPLICATIONS

This application claims the benefit of U.S. Provisional Patent Application No. 63/303,921, filed Jan. 27, 2022, which is incorporated by reference herein in its entirety.

### 2. SEQUENCE LISTING

This application contains a computer readable Sequence Listing which has been submitted in XML file format with this application, the entire content of which is incorporated by reference herein in its entirety. The Sequence Listing XML file submitted with this application is entitled “14620-547-228_SEQ_LISTING.xml”, was created on Jan. 26, 2023 and is 67,678 bytes in size.

### 3. FIELD

The presently described invention teaches, inter alia, isolated polypeptides, including diagnostic, prognostic, and therapeutic polypeptides, such as, for example, polypeptides that are capable of binding to themselves or other polypeptides or molecules, said isolated polypeptides comprising an exposed carboxy-terminus (“C-terminus”) fused to a cap domain (e.g., a His cap domain, (His) 6 (i.e., HHHHHH (SEQ ID NO:26)), SLSLSPGK (SEQ ID NO:36), AS, or TVAPTESS (SEQ ID NO:37)). The presently described invention further teaches nucleic acids and/or expression vectors encoding the polypeptides; cells containing the polypeptides, nucleic acids, and/or expression vectors; and compositions comprising the polypeptides. Methods of making the polypeptides, and methods of using the polypeptides are also taught.

### 4. BACKGROUND

Polypeptide interactions with other molecules, including other polypeptides, are well known. (See, e.g., example Titeca, Kevin; Lemmens, Irma; Tavernier, Jan; Eyckerman, Sven (29 Jun. 2018), “Discovering cellular protein-protein interactions: Technological strategies and opportunities,” Mass Spectrometry Reviews. 38 (1): 79-111. doi: 10.1002/mas.21574. ISSN 0277-7037. PMID 29957823; and Herce H D, Deng W, Helma J, Leonhardt H, Cardoso M C (2013), “Visualization and targeted disruption of protein interactions in living cells,” Nature Communications. 4:2660). Such interactive polypeptides can be present in vitro and in vivo. (See, e.g., Jones S, Thornton JM (January 1996), “Principles of protein-protein interactions,” Proceedings of the National Academy of Sciences of the United States of America. 93 (1): 13-20. Bibcode: 1996PNAS . . . 93 . . . 13J. doi: 10.1073/pnas.93.1.13. PMC 40170. PMID 8552589). An example of a polypeptide that interacts with other molecules is a polypeptide that interacts with a protein, including antigen binding proteins, such as antibodies. (See, e.g., Curr Drug Metab. 2011 May; 12 (4): 313-328, “Characterization of Drug Interactions with Serum Proteins by Using High-Performance Affinity Chromatography,” David S. Hage, Jeanethe Anguizola, Omar Barnaby, Abby Jackson, Michelle J. Yoo, Efthimia Papastavros, Erika Pfaunmiller, Matt Sobansky, and Zenghan Tong). Proteins have been reported to bind to isolated polypeptides and in some instances elicit anti-polypeptide antibody responses, which can impact their uses, such as uses in therapy. (See, e.g., “Studies of drug interactions with glycated human serum albumin by high-performance affinity chromatography,” Ryan Matsuda, So-Hwang Kye, Jeanethe Anguizola and David S. Hage, Reviews in Analytical Chemistry).

### 5. SUMMARY

Against this backdrop, the inventors of the present invention discovered methods and compositions for the prevention or amelioration of polypeptide interactions with molecules.

In an aspect, provided herein is an isolated polypeptide comprising one or more target binding regions and an exposed C-terminus fused to a cap domain, wherein the isolated polypeptide exhibits reduced interaction with a reference relative to the interaction of the isolated polypeptide lacking the cap domain with the reference. The cap domain is heterologous to the exposed C-terminus and includes one or more amino acid residues (e.g., histidines). In some embodiments, the cap domain consists of SLSLSPGK (SEQ ID NO:36), or TVAPTESS (SEQ ID NO: 37), or a His cap domain. In some embodiments, the biological substance comprises serum. In specific embodiments, the biological substance is human serum. In some embodiments, the biological substance comprises plasma. In specific embodiments, the biological substance is human plasma. In some embodiments, the biological substance comprises serum and plasma (e.g., human serum and human plasma).

In one aspect, provided herein is an isolated polypeptide comprising one or more target binding regions and an exposed C-terminus fused to a His cap domain, wherein the isolated polypeptide exhibits reduced interaction with a reference relative to the interaction of the isolated polypeptide lacking the His cap domain with the reference. In some embodiments, the reference is a molecule present in a biological substance. In some embodiments, the biological substance comprises serum. In specific embodiments, the biological substance is human serum. In some embodiments, the biological substance comprises plasma. In specific embodiments, the biological substance is human plasma. In some embodiments, the biological substance comprises serum and plasma (e.g., human serum and human plasma).

In some embodiments, the reference comprises an anti-drug antibody. In some embodiments, the anti-drug antibody is pre-existing in serum and/or plasma of a subject. In some embodiments, the exposed C-terminus comprises an anti-drug antibody epitope.

In some embodiments, the one or more target binding regions comprise an antibody variable domain or an antigen-binding fragment thereof. In some embodiments, the one or more target binding regions comprise a light chain antibody variable domain or an antigen-binding fragment thereof. In some embodiments, the one or more target binding regions comprise a heavy antibody variable domain or an antigen-binding fragment thereof.

In some embodiments, the cap domain consists of a His cap domain, or the amino acid sequence of SLSLSPGK (SEQ ID NO:36) or TVAPTESS (SEQ ID NO:37). In some embodiments, the His cap domain consists of a single histidine residue. In some embodiments, the His cap domain consists of two histidine residues. In some embodiments, the His cap domain consists of three histidine residues. In some embodiments, the His cap domain consists of four histidine residues (SEQ ID NO:27). In some embodiments, the His cap domain consists of five histidine residues (SEQ ID NO:28).

In some embodiments, the exposed C-terminus consists of the amino acid sequence of VTVSS (SEQ ID NO:21), VEIK (SEQ ID NO:22), VEIKR (SEQ ID NO:23), or VEIKRT (SEQ ID NO: 24). In some embodiments, the exposed C-terminus consists of the amino acid sequence of TKVTVL (SEQ ID NO:38), TKLTVL (SEQ ID NO:39), TQLIIL (SEQ ID NO:40), TELTVL (SEQ ID NO:41), or TQLTVL (SEQ ID NO:42). In some embodiments, the exposed C-terminus consists of the amino acid sequence of TKLTVL (SEQ ID NO:39), TKVTVL (SEQ ID NO:38), NGLVYAG (SEQ ID NO:43), NGLLYAG (SEQ ID NO:44), or TRLTVL (SEQ ID NO:45). In some embodiments, the exposed C-terminus consists of an amino acid sequence provided in Table 1.

In some embodiments, the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO:5). In some embodiments, the exposed C-terminus fused to the His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO:9), and VEIKHHHHH (SEQ ID NO:10). In some embodiments, the exposed C-terminus fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO: 14), and VEIKRHHHHH (SEQ ID NO:15). In some embodiments, the exposed C-terminus fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO:20).

In some embodiments, the exposed C-terminus fused to the His cap domain consists of the amino acid sequence of TKVTVL (His)n (SEQ ID NO:46), TKLTVL (His)n (SEQ ID NO: 47), TQLIIL (His)n (SEQ ID NO:48), TELTVL (His)n (SEQ ID NO:49), or TQLTVL (His)n (SEQ ID NO:50), wherein n is 1, 2, 3, 4, or 5. In some embodiments, the exposed C-terminus fused to the His cap domain consists of the amino acid sequence of TKLTVL (His)n (SEQ ID NO: 47), TKVTVL (His)n (SEQ ID NO:46), NGLVYAG (His)n (SEQ ID NO:51), NGLLYAG (His)n (SEQ ID NO:52), or TRLTVL (His)n (SEQ ID NO:53), wherein n is 1, 2, 3, 4, or 5.

In some embodiments, interaction with the reference is determined by an immunoassay. In some embodiments, the interaction with the reference is assessed in vitro. In some embodiments, the interaction with the reference is assessed in vivo.

In another aspect, provided herein is an isolated polypeptide comprising one or more target binding regions and an exposed C-terminus fused to a cap domain (e.g., a His cap domain, SLSLSPGK (SEQ ID NO:36), or TVAPTESS (SEQ ID NO:37)), wherein the isolated polypeptide exhibits reduced aggregation upon incubation with a biological substance relative to the aggregation exhibited upon incubation of the isolated polypeptide lacking the cap domain (e.g., a His cap domain, SLSLSPGK (SEQ ID NO:36), or TVAPTESS (SEQ ID NO:37)) with the biological substance.

In some embodiments, incubation with the biological substance comprises incubation for about five to about fourteen days at about 37° C. In some embodiments, incubation with the biological substance comprises incubation for about seven to about fourteen days at about 40° C. In some embodiments, incubation with the biological substance comprises incubation for about two to about seven days at about 40° C. In some embodiments, incubation with the biological substance comprises incubation for about two to about five days at about 40° C. In some embodiments, incubation with the biological substance comprises incubation for about seven to about fourteen days at about 4° C. In some embodiments, incubation with the biological substance comprises incubation for about seven to about fourteen days at about 25° C. In some embodiments, incubation with the biological substance comprises incubation for about two to about seven days at about 25° C. In some embodiments, incubation with the biological substance comprises incubation for about two to about five days at about 25° C.

In some embodiments, the biological substance comprises an anti-drug antibody. In some embodiments, the biological substance comprises an anti-drug antibody pre-existing in the serum and/or plasma of a subject (e.g., a human subject). In some embodiments, the exposed C-terminus comprises an anti-drug antibody epitope. In some embodiments, the one or more target binding regions comprise an antibody variable domain or an antigen-binding fragment thereof. In some embodiments, the one or more target binding regions comprise a light chain antibody variable domain or an antigen-binding fragment thereof. In some embodiments, the one or more target binding regions comprise a heavy antibody variable domain or an antigen-binding fragment thereof.

In some embodiments, the cap domain consists of a His cap domain, or the amino acid sequence of SLSLSPGK (SEQ ID NO:36) or TVAPTESS (SEQ ID NO:37). In some embodiments, the His cap domain consists of a single histidine residue. In some embodiments, the His cap domain consists of two histidine residues. In some embodiments, the His cap domain consists of three histidine residues. In some embodiments, the His cap domain consists of four histidine residues (SEQ ID NO:27). In some embodiments, the His cap domain consists of five histidine residues (SEQ ID NO:28).

In some embodiments, the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO:5). In some embodiments, the exposed C-terminus fused to the His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO:9), and VEIKHHHHH (SEQ ID NO:10). In some embodiments, the exposed C-terminus fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15). In some embodiments, the exposed C-terminus fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO:20). In some embodiments, the exposed C-terminus fused to the His cap domain consists of the amino acid sequence of TKVTVL (His)n (SEQ ID NO:46), TKLTVL (His)n (SEQ ID NO:47), TQLIIL (His)n (SEQ ID NO:48), TELTVL (His)n (SEQ ID NO: 49), or TQLTVL (His)n (SEQ ID NO:50), wherein n is 1, 2, 3, 4, or 5. In some embodiments, the exposed C-terminus fused to the His cap domain consists of the amino acid sequence of TKLTVL (His)n (SEQ ID NO:47), TKVTVL (His)n (SEQ ID NO:46), NGLVYAG (His)n (SEQ ID NO: 51), NGLLYAG (His)n (SEQ ID NO:52), or TRLTVL (His)n (SEQ ID NO:53), wherein n is 1, 2, 3, 4, or 5.

In some embodiments, the interaction with the biological substance is assessed by size exclusion chromatography.

In another aspect, provided herein is an isolated polypeptide comprising one or more target binding regions and an exposed C-terminus fused to a cap domain (e.g., a His cap domain, SLSLSPGK (SEQ ID NO:36), or TVAPTESS (SEQ ID NO:37)), wherein the isolated polypeptide exhibits reduced self-aggregation relative to the self-aggregation exhibited by the isolated polypeptide lacking the cap domain (e.g., a His cap domain, SLSLSPGK (SEQ ID NO: 36), or TVAPTESS (SEQ ID NO:37)) under the same conditions.

In some embodiments, the exposed C-terminus comprises an anti-drug antibody epitope. In some embodiments, the one or more target binding regions comprise an antibody variable domain or an antigen-binding fragment thereof. In some embodiments, the one or more target binding regions comprise a light chain antibody variable domain or an antigen-binding fragment thereof. In some embodiments, the one or more target binding regions comprise a heavy antibody variable domain or an antigen-binding fragment thereof.

In some embodiments, the cap domain consists of a His cap domain, or the amino acid sequence of SLSLSPGK (SEQ ID NO:36) or TVAPTESS (SEQ ID NO:37). In some embodiments, the His cap domain consists of a single histidine residue. In some embodiments, the His cap domain consists of two histidine residues. In some embodiments, the His cap domain consists of three histidine residues. In some embodiments, the His cap domain consists of four histidine residues (SEQ ID NO:27). In some embodiments, the His cap domain consists of five histidine residues (SEQ ID NO:28).

In some embodiments, the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO:5). In some embodiments, the exposed C-terminus fused to the His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO:9), and VEIKHHHHH (SEQ ID NO:10). In some embodiments, the exposed C-terminus fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15). In some embodiments, the exposed C-terminus fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO:20). In some embodiments, the exposed C-terminus fused to the His cap domain consists of the amino acid sequence of TKVTVL (His)n (SEQ ID NO:46), TKLTVL (His)n (SEQ ID NO:47), TQLIIL (His)n (SEQ ID NO:48), TELTVL (His)n (SEQ ID NO: 49), or TQLTVL (His)n (SEQ ID NO:50), wherein n is 1, 2, 3, 4, or 5. In some embodiments, the exposed C-terminus fused to the His cap domain consists of the amino acid sequence of TKLTVL (His)n (SEQ ID NO:47), TKVTVL (His)n (SEQ ID NO:46), NGLVYAG (His)n (SEQ ID NO: 51), NGLLYAG (His)n (SEQ ID NO:52), or TRLTVL (His)n (SEQ ID NO:53), wherein n is 1, 2, 3, 4, or 5.

In some embodiments, self-aggregation is reduced in a high concentration liquid formulation (HCLF). In some embodiments, the HCLF concentration comprises about 50 mg/mL to about 150 mg/mL of the isolated polypeptide. In some embodiments, self-aggregation is reduced under thermal stress. In some embodiments, thermal stress comprises incubation at 40° C. for about two weeks. In some embodiments, the self-aggregation is assessed by size exclusion chromatography.

In another aspect, provided herein is an isolated protein, comprising: (a) a first polypeptide comprising a target binding region, and an exposed C-terminus fused to a cap domain (e.g., a His cap domain, SLSLSPGK (SEQ ID NO:36), or TVAPTESS (SEQ ID NO: 37)); and (b) a second polypeptide comprising a target binding region, wherein the isolated protein exhibits reduced interaction with a reference relative to the interaction of the isolated protein lacking the cap domain (e.g., a His cap domain, SLSLSPGK (SEQ ID NO:36), or TVAPTESS (SEQ ID NO:37)) with the reference. In some embodiments, the presence of the cap domain (e.g., a His cap domain, SLSLSPGK (SEQ ID NO:36), or TVAPTESS (SEQ ID NO:37)) results in reduced neutralization of the protein relative to the neutralization of the same protein lacking the cap domain (e.g., a His cap domain, SLSLSPGK (SEQ ID NO:36), or TVAPTESS (SEQ ID NO:37)). In some embodiments, the presence of the cap domain (e.g., a His cap domain, SLSLSPGK (SEQ ID NO:36), or TVAPTESS (SEQ ID NO:37)) results in improved safety of the protein relative to the safety of the same protein lacking the cap domain (e.g., a His cap domain, SLSLSPGK (SEQ ID NO:36), or TVAPTESS (SEQ ID NO:37)) with the reference. In some embodiments, the presence of the cap domain (e.g., a His cap domain, SLSLSPGK (SEQ ID NO: 36), or TVAPTESS (SEQ ID NO:37)) results in improved pharmacokinetics of the protein relative to the pharmacokinetics of the same protein lacking the cap domain (e.g., a His cap domain, SLSLSPGK (SEQ ID NO:36), or TVAPTESS (SEQ ID NO:37)) with the reference. In some embodiments, the presence of the cap domain (e.g., a His cap domain, SLSLSPGK (SEQ ID NO: 36), or TVAPTESS (SEQ ID NO:37)) results in reduced interaction between the protein and an anti-drug antibody relative to the interaction of the same protein lacking the cap domain (e.g., a His cap domain, SLSLSPGK (SEQ ID NO:36), or TVAPTESS (SEQ ID NO:37)) with the reference.

In some embodiments, the exposed C-terminus of the first polypeptide comprises an anti-drug antibody epitope. In some embodiments, the exposed C-terminus of the second polypeptide comprises an anti-drug antibody epitope. In some embodiments, the second polypeptide comprises an exposed C-terminus fused to a cap domain (e.g., a His cap domain, SLSLSPGK (SEQ ID NO:36), or TVAPTESS (SEQ ID NO:37)).

In some embodiments, the target binding region of the first and second polypeptides are capable of binding to different targets. In some embodiments, the target binding region of the first polypeptide comprises an antibody variable domain or an antigen-binding fragment thereof. In some embodiments, the target binding region of the second polypeptide comprises an antibody variable domain or an antigen-binding fragment thereof. In some embodiments, the target binding region of the first polypeptide comprises a light chain antibody variable domain or an antigen-binding fragment thereof. In some embodiments, the target binding region of the second polypeptide comprises a light chain antibody variable domain or an antigen-binding fragment thereof. In some embodiments, the target binding region of the first polypeptide comprises a heavy chain antibody variable domain or an antigen-binding fragment thereof. In some embodiments, the target binding region of the second polypeptide comprises a heavy chain antibody variable domain or an antigen-binding fragment thereof.

In some embodiments, the cap domain of the first polypeptide consists of a His cap domain, or the amino acid sequence of SLSLSPGK (SEQ ID NO:36) or TVAPTESS (SEQ ID NO: 37). In specific embodiments, the cap domain of the first polypeptide consists of a His cap domain. In some embodiments, the cap domain of the second polypeptide consists of a His cap domain, or the amino acid sequence of SLSLSPGK (SEQ ID NO:36) or TVAPTESS (SEQ ID NO: 37). In specific embodiments, the cap domain of the second polypeptide consists of a His cap domain. In some embodiments, the His cap domain of the first polypeptide consists of a single histidine residue. In some embodiments, the His cap domain of the second polypeptide consists of a single histidine residue. In some embodiments, the His cap domain of the first polypeptide consists of two histidine residues. In some embodiments, the His cap domain of the second polypeptide consists of two histidine residues. In some embodiments, the His cap domain of the first polypeptide consists of three histidine residues. In some embodiments, the His cap domain of the second polypeptide consists of three histidine residues. In some embodiments, the His cap domain of the first polypeptide consists of four histidine residues (SEQ ID NO:27). In some embodiments, the His cap domain of the second polypeptide consists of four histidine residues (SEQ ID NO:27). In some embodiments, the His cap domain of the first polypeptide consists of five histidine residues (SEQ ID NO:28). In some embodiments, the His cap domain of the second polypeptide consists of five histidine residues (SEQ ID NO:28).

In some embodiments, the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO:5). In some embodiments, the exposed C-terminus fused to the His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO:9), and VEIKHHHHH (SEQ ID NO:10). In some embodiments, the exposed C-terminus fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15). In some embodiments, the exposed C-terminus fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO:20). In some embodiments, the exposed C-terminus fused to the His cap domain consists of the amino acid sequence of TKVTVL (His)n (SEQ ID NO:46), TKLTVL (His)n (SEQ ID NO:47), TQLIIL (His)n (SEQ ID NO:48), TELTVL (His)n (SEQ ID NO: 49), or TQLTVL (His)n (SEQ ID NO:50), wherein n is 1, 2, 3, 4, or 5. In some embodiments, the exposed C-terminus fused to the His cap domain consists of the amino acid sequence of TKLTVL (His)n (SEQ ID NO:47), TKVTVL (His)n (SEQ ID NO:46), NGLVYAG (His)n (SEQ ID NO: 51), NGLLYAG (His)n (SEQ ID NO:52), or TRLTVL (His)n (SEQ ID NO:53), wherein n is 1, 2, 3, 4, or 5.

In some embodiments, the isolated protein further comprises a third polypeptide comprising a target binding region. In some embodiments, the third polypeptide comprises an exposed C-terminus fused to a cap domain (e.g., a His cap domain, SLSLSPGK (SEQ ID NO: 36), or TVAPTESS (SEQ ID NO:37)). In some embodiments, the third polypeptide is capable of binding to a different target than the first polypeptide, the second polypeptide, or both. In some embodiments, the cap domain is a His cap domain.

In some embodiments, the exposed C-terminus of the third polypeptide comprises an anti-drug antibody epitope. In some embodiments, the target binding region of the third polypeptide comprises an antibody variable domain or an antigen-binding fragment thereof. In some embodiments, the target binding region of the third polypeptide comprises a light chain antibody variable domain or an antigen-binding fragment thereof. In some embodiments, the target binding region of the third polypeptide comprises a heavy chain antibody variable domain or an antigen-binding fragment thereof.

In some embodiments, the His cap domain of the third polypeptide consists of a single histidine residue. In some embodiments, the His cap domain of the third polypeptide consists of two histidine residues. In some embodiments, the His cap domain of the third polypeptide consists of three histidine residues. In some embodiments, the His cap domain of the third polypeptide consists of four histidine residues (SEQ ID NO:27). In some embodiments, the His cap domain of the third polypeptide consists of five histidine residues (SEQ ID NO:28).

In some embodiments, the exposed C-terminus of the third polypeptide fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO:5). In some embodiments, the exposed C-terminus of the third polypeptide fused to the His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO:9), and VEIKHHHHH (SEQ ID NO:10). In some embodiments, the exposed C-terminus of the third polypeptide fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15). In some embodiments, the exposed C-terminus of the third polypeptide fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO: 19), and VEIKRTHHHHH (SEQ ID NO:20). In some embodiments, the exposed C-terminus of the third polypeptide fused to the His cap domain consists of the amino acid sequence of TKVTVL (His)n (SEQ ID NO:46), TKLTVL (His)n (SEQ ID NO:47), TQLIIL (His)n (SEQ ID NO: 48), TELTVL (His)n (SEQ ID NO:49), or TQLTVL (His)n (SEQ ID NO:50), wherein n is 1, 2, 3, 4, or 5. In some embodiments, the exposed C-terminus of the third polypeptide fused to the His cap domain consists of the amino acid sequence of TKLTVL (His)n (SEQ ID NO:47), TKVTVL (His)n (SEQ ID NO:46), NGLVYAG (His)n (SEQ ID NO:51), NGLLYAG (His)n (SEQ ID NO: 52), or TRLTVL (His)n (SEQ ID NO:53), wherein n is 1, 2, 3, 4, or 5.

In some embodiments, the isolated protein is bispecific. In some embodiments, the isolated protein is multi-specific. In some embodiments, the isolated protein is multispecific, such as, for example, trispecific. In some embodiments, the isolated protein is heterodimeric.

In another aspect, provided herein is an isolated nucleic acid sequence comprising a nucleotide sequence encoding a polypeptide provided herein, or a protein provided herein.

In another aspect, provided herein is an isolated cell expressing a polypeptide provided herein, or a protein provided herein.

In another aspect, provided herein is an expression vector comprising a nucleotide sequence encoding a polypeptide provided herein, or a protein provided herein.

In another aspect, provided herein is an isolated cell comprising a nucleotide sequence encoding a polypeptide provided herein, or a protein provided herein, or an expression vector comprising a nucleotide sequence encoding a polypeptide provided herein, or a protein provided herein.

In another aspect, provided herein is a method for producing a polypeptide provided herein, or a protein provided herein, comprising culturing the cell provided herein. In some embodiments, the method further comprises isolating the polypeptide or protein.

In a further aspect, provided herein is a bispecific or a multi-specific antibody comprising one or more target binding regions and an exposed C-terminus fused to a cap domain (e.g., a His cap domain, SLSLSPGK (SEQ ID NO:36), or TVAPTESS (SEQ ID NO:37)), wherein the antibody exhibits reduced interaction with an anti-drug antibody relative to the interaction of the antibody lacking the cap domain (e.g., a His cap domain, SLSLSPGK (SEQ ID NO: 36), or TVAPTESS (SEQ ID NO:37)) with the anti-drug antibody. In some embodiments, the anti-drug antibody is pre-existing in the serum and/or plasma of a subject (e.g., a human subject). In some embodiments, the exposed C-terminus comprises an anti-drug antibody epitope. In some embodiments, the antibody exhibits reduced interaction with an anti-drug antibody relative to the interaction of the antibody lacking the His cap domain with the anti-drug antibody in an immunoassay. In some embodiments, the one or more target binding regions comprises at least one antibody variable domain. In some embodiments, the at least one antibody variable domain comprises a heavy chain variable domain. In some embodiments, the at least one antibody variable domain comprises a light chain variable domain.

In some embodiments, the cap domain consists of a His cap domain, or the amino acid sequence of SLSLSPGK (SEQ ID NO:36) or TVAPTESS (SEQ ID NO:37). In some embodiments, the His cap domain consists of a single histidine residue. In some embodiments, the His cap domain consists of two histidine residues. In some embodiments, the His cap domain consists of three histidine residues. In some embodiments, the His cap domain consists of four histidine residues (SEQ ID NO:27). In some embodiments, the His cap domain consists of five histidine residues (SEQ ID NO:28).

In some embodiments, the exposed C-terminus fused to a His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO:5). In some embodiments, the exposed C-terminus fused to a His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO: 9), and VEIKHHHHH (SEQ ID NO:10). In some embodiments, the exposed C-terminus fused to a His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15). In some embodiments, the exposed C-terminus fused to a His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO: 20). In some embodiments, the exposed C-terminus fused to the His cap domain consists of the amino acid sequence of TKVTVL (His)n (SEQ ID NO:46), TKLTVL (His)n (SEQ ID NO: 47), TQLIIL (His)n (SEQ ID NO:48), TELTVL (His)n (SEQ ID NO:49), or TQLTVL (His)n (SEQ ID NO:50), wherein n is 1, 2, 3, 4, or 5. In some embodiments, the exposed C-terminus fused to the His cap domain consists of the amino acid sequence of TKLTVL (His)n (SEQ ID NO: 47), TKVTVL (His)n (SEQ ID NO:46), NGLVYAG (His)n (SEQ ID NO:51), NGLLYAG (His)n (SEQ ID NO:52), or TRLTVL (His)n (SEQ ID NO:53), wherein n is 1, 2, 3, 4, or 5.

In some embodiments, the multi-specific antibody comprises a trispecific antibody.

In another aspect, provided herein is a nucleic acid sequence comprising a nucleotide sequence encoding an antibody provided herein.

In another aspect, provided herein is an expression vector comprising the nucleic acid sequence comprising a nucleotide sequence encoding an antibody provided herein.

In another aspect, provided herein is an isolated cell comprising a nucleotide sequence encoding an antibody provided herein, or an expression vector comprising the nucleic acid sequence comprising a nucleotide sequence encoding an antibody provided herein.

In another aspect, provided herein is an isolated cell expressing an antibody provided herein.

In another aspect, provided herein is a method for producing an antibody provided herein, comprising culturing a cell provided herein. In some embodiments, the method further comprises isolating the antibody.

In another aspect, provided herein is a method for reducing the interaction between an isolated polypeptide and a reference, wherein the isolated polypeptide comprises one or more target binding regions and an exposed C-terminus, the method comprising fusing a cap domain (e.g., a His cap domain, SLSLSPGK (SEQ ID NO:36), or TVAPTESS (SEQ ID NO:37)) at the end of the exposed C-terminus. In some embodiments, the reference is a molecule present in a biological substance. In some embodiments, the biological substance comprises serum. In specific embodiments, the biological substance is human serum. In some embodiments, the biological substance comprises plasma. In specific embodiments, the biological substance is human plasma.

In some embodiments, the reference comprises an anti-drug antibody. In some embodiments, the reference comprises an anti-drug antibody pre-existing in the serum and/or plasma of a subject (e.g., a human subject). In some embodiments, the exposed C-terminus comprises an anti-drug antibody epitope.

In some embodiments, the one or more target binding regions comprise an antibody variable domain or an antigen-binding fragment thereof. In some embodiments, the one or more target binding regions comprise a light chain antibody variable domain or an antigen-binding fragment thereof. In some embodiments, the one or more target binding regions comprise a heavy antibody variable domain or an antigen-binding fragment thereof.

In some embodiments, the cap domain consists of a His cap domain, or the amino acid sequence of SLSLSPGK (SEQ ID NO:36) or TVAPTESS (SEQ ID NO:37). In some embodiments, the His cap domain consists of a single histidine residue. In some embodiments, the His cap domain consists of two histidine residues. In some embodiments, the His cap domain consists of three histidine residues. In some embodiments, the His cap domain consists of four histidine residues (SEQ ID NO:27). In some embodiments, the His cap domain consists of five histidine residues (SEQ ID NO:28).

In some embodiments, the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO:5). In some embodiments, the exposed C-terminus fused to the His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO:9), and VEIKHHHHH (SEQ ID NO:10). In some embodiments, the exposed C-terminus fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15). In some embodiments, the exposed C-terminus fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO:20). In some embodiments, the exposed C-terminus fused to the His cap domain consists of the amino acid sequence of TKVTVL (His)n (SEQ ID NO:46), TKLTVL (His)n (SEQ ID NO:47), TQLIIL (His)n (SEQ ID NO:48), TELTVL (His)n (SEQ ID NO: 49), or TQLTVL (His)n (SEQ ID NO:50), wherein n is 1, 2, 3, 4, or 5. In some embodiments, the exposed C-terminus fused to the His cap domain consists of the amino acid sequence of TKLTVL (His)n (SEQ ID NO:47), TKVTVL (His)n (SEQ ID NO:46), NGLVYAG (His)n (SEQ ID NO: 51), NGLLYAG (His)n (SEQ ID NO:52), or TRLTVL (His)n (SEQ ID NO:53), wherein n is 1, 2, 3, 4, or 5.

In some embodiments, the interaction with the reference is determined by an immunoassay. In some embodiments, the interaction with the reference is assessed in vitro. In some embodiments, the interaction with the reference is assessed in vivo.

In another aspect, provided herein is a method for reducing the aggregation of an isolated polypeptide in a biological substance, wherein the isolated polypeptide comprises one or more target binding regions and an exposed C-terminus, the method comprising fusing a cap domain (e.g., a His cap domain, SLSLSPGK (SEQ ID NO:36), or TVAPTESS (SEQ ID NO:37)) at the end of the exposed C-terminus. In some embodiments, the biological substance comprises serum and/or plasma. In some embodiments, the biological substance is human serum. In some embodiments, the biological substance is human plasma.

In some embodiments, incubation with the biological substance comprises incubation for about five to about fourteen days at about 37° C. In some embodiments, incubation with the biological substance comprises incubation for about two to about seven days at 37° C. In some embodiments, incubation with the biological substance comprises incubation for about two to about five days at 37° C. In some embodiments, incubation with the biological substance comprises incubation for about seven to about fourteen days at about 40° C. In some embodiments, incubation with the biological substance comprises incubation for about two to about seven days at 40° C. In some embodiments, incubation with the biological substance comprises incubation for about two to about five days at 40° C. In some embodiments, incubation with the biological substance comprises incubation for about seven to about fourteen days at about 4° C. In some embodiments, incubation with the biological substance comprises incubation for about two to about seven days at 4° C. In some embodiments, incubation with the biological substance comprises incubation for about two to about five days at 4° C. In some embodiments, incubation with the biological substance comprises incubation for about seven to about fourteen days at about 25° C. In some embodiments, incubation with the biological substance comprises incubation for about two to about seven days at 25° C. In some embodiments, incubation with the biological substance comprises incubation for about two to about five days at 25° C.

In some embodiments, the biological substance comprises an anti-drug antibody. In some embodiments, the anti-drug antibody is pre-existing in the serum and/or plasma of a subject (e.g., a human subject). In some embodiments, the exposed C-terminus comprises an anti-drug antibody epitope. In some embodiments, the one or more target binding regions comprise an antibody variable domain or an antigen-binding fragment thereof. In some embodiments, the one or more target binding regions comprise a light chain antibody variable domain or an antigen-binding fragment thereof. In some embodiments, the one or more target binding regions comprise a heavy antibody variable domain or an antigen-binding fragment thereof.

In some embodiments, the cap domain consists of a His cap domain, or the amino acid sequence of SLSLSPGK (SEQ ID NO:36) or TVAPTESS (SEQ ID NO:37). In some embodiments, the His cap domain consists of a single histidine residue. In some embodiments, the His cap domain consists of two histidine residues. In some embodiments, the His cap domain consists of three histidine residues. In some embodiments, the His cap domain consists of four histidine residues (SEQ ID NO:27). In some embodiments, the His cap domain consists of five histidine residues (SEQ ID NO:28).

In some embodiments, the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO:5). In some embodiments, the exposed C-terminus fused to the His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO:9), and VEIKHHHHH (SEQ ID NO:10). In some embodiments, the exposed C-terminus fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO: 14), and VEIKRHHHHH (SEQ ID NO:15). In some embodiments, the exposed C-terminus fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO:20). In some embodiments, the exposed C-terminus fused to the His cap domain consists of the amino acid sequence of TKVTVL (His)n (SEQ ID NO:46), TKLTVL (His)n (SEQ ID NO:47), TQLIIL (His)n (SEQ ID NO:48), TELTVL (His)n (SEQ ID NO: 49), or TQLTVL (His)n (SEQ ID NO:50), wherein n is 1, 2, 3, 4, or 5. In some embodiments, the exposed C-terminus fused to the His cap domain consists of the amino acid sequence of TKLTVL (His)n (SEQ ID NO:47), TKVTVL (His)n (SEQ ID NO:46), NGLVYAG (His)n (SEQ ID NO: 51), NGLLYAG (His)n (SEQ ID NO:52), or TRLTVL (His)n (SEQ ID NO:53), wherein n is 1, 2, 3, 4, or 5.

In some embodiments, the interaction with the biological substance is assessed by size exclusion chromatography.

In another aspect, provided herein is a method for reducing self-aggregation of an isolated polypeptide, wherein the isolated polypeptide comprises one or more target binding regions and an exposed C-terminus, the method comprising fusing a cap domain (e.g., a His cap domain, SLSLSPGK (SEQ ID NO:36), or TVAPTESS (SEQ ID NO:37)) at the end of the exposed C-terminus.

In some embodiments, the exposed C-terminus comprises an anti-drug antibody epitope. In some embodiments, the one or more target binding regions comprise an antibody variable domain or an antigen-binding fragment thereof. In some embodiments, the one or more target binding regions comprise a light chain antibody variable domain or an antigen-binding fragment thereof. In some embodiments, the one or more target binding regions comprise a heavy antibody variable domain or an antigen-binding fragment thereof.

In some embodiments, the cap domain consists of a His cap domain, or the amino acid sequence of SLSLSPGK (SEQ ID NO:36) or TVAPTESS (SEQ ID NO:37). In some embodiments, the His cap domain consists of a single histidine residue. In some embodiments, the His cap domain consists of two histidine residues. In some embodiments, the His cap domain consists of three histidine residues. In some embodiments, the His cap domain consists of four histidine residues (SEQ ID NO:27). In some embodiments, the His cap domain consists of five histidine residues (SEQ ID NO:28).

In some embodiments, the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO:5). In some embodiments, the exposed C-terminus fused to the His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO:9), and VEIKHHHHH (SEQ ID NO:10). In some embodiments, the exposed C-terminus fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15). In some embodiments, the exposed C-terminus fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO:20). In some embodiments, the exposed C-terminus fused to the His cap domain consists of the amino acid sequence of TKVTVL (His)n (SEQ ID NO:46), TKLTVL (His)n (SEQ ID NO:47), TQLIIL (His)n (SEQ ID NO:48), TELTVL (His)n (SEQ ID NO: 49), or TQLTVL (His)n (SEQ ID NO:50), wherein n is 1, 2, 3, 4, or 5. In some embodiments, the exposed C-terminus fused to the His cap domain consists of the amino acid sequence of TKLTVL (His)n (SEQ ID NO:47), TKVTVL (His)n (SEQ ID NO:46), NGLVYAG (His)n (SEQ ID NO: 51), NGLLYAG (His)n (SEQ ID NO:52), or TRLTVL (His)n (SEQ ID NO:53), wherein n is 1, 2, 3, 4, or 5.

In some embodiments, self-aggregation is reduced in a high concentration liquid formulation (HCLF). In some embodiments, the HCLF concentration comprises about 50 mg/mL to about 150 mg/mL of the isolated polypeptide. In some embodiments, self-aggregation is reduced under thermal stress. In some embodiments, thermal stress comprises incubation at 40° C. for about two weeks. In some embodiments, the self-aggregation is assessed by size exclusion chromatography.

In another aspect, provided herein is a method for reducing the interaction between an isolated protein and a reference, wherein the isolated protein comprises: (a) a first polypeptide comprising a target binding region, and an exposed C-terminus; and (b) a second polypeptide comprising a target binding region, the method comprising fusing a cap domain (e.g., a His cap domain, SLSLSPGK (SEQ ID NO:36), or TVAPTESS (SEQ ID NO:37)) at the end of the exposed C-terminus. In some embodiments, the exposed C-terminus of the first polypeptide comprises an anti-drug antibody epitope. In some embodiments, the exposed C-terminus of the second polypeptide comprises an anti-drug antibody epitope. In some embodiments, the second polypeptide comprises an exposed C-terminus and the method further comprises fusing a cap domain (e.g., a His cap domain, SLSLSPGK (SEQ ID NO:36), or TVAPTESS (SEQ ID NO:37)) to the end of the exposed C-terminus of the second polypeptide. In some embodiments, the target binding region of the first and second polypeptides are capable of binding to different targets.

In some embodiments, the target binding region of the first polypeptide comprises an antibody variable domain or an antigen-binding fragment thereof. In some embodiments, the target binding region of the second polypeptide comprises an antibody variable domain or an antigen-binding fragment thereof. In some embodiments, the target binding region of the first polypeptide comprises a light chain antibody variable domain or an antigen-binding fragment thereof. In some embodiments, the target binding region of the second polypeptide comprises a light chain antibody variable domain or an antigen-binding fragment thereof. In some embodiments, the target binding region of the first polypeptide comprises a heavy chain antibody variable domain or an antigen-binding fragment thereof. In some embodiments, the target binding region of the second polypeptide comprises a heavy chain antibody variable domain or an antigen-binding fragment thereof.

In some embodiments, the cap domain of the first polypeptide consists of a His cap domain, or the amino acid sequence of SLSLSPGK (SEQ ID NO:36) or TVAPTESS (SEQ ID NO: 37). In some embodiments, the cap domain of the second polypeptide consists of a His cap domain, or the amino acid sequence of SLSLSPGK (SEQ ID NO:36) or TVAPTESS (SEQ ID NO: 37). In some embodiments, the His cap domain of the first polypeptide consists of a single histidine residue. In some embodiments, the His cap domain of the second polypeptide consists of a single histidine residue. In some embodiments, the His cap domain of the first polypeptide consists of two histidine residues. In some embodiments, the His cap domain of the second polypeptide consists of two histidine residues. In some embodiments, the His cap domain of the first polypeptide consists of three histidine residues. In some embodiments, the His cap domain of the second polypeptide consists of three histidine residues. In some embodiments, the His cap domain of the first polypeptide consists of four histidine residues (SEQ ID NO:27). In some embodiments, the His cap domain of the second polypeptide consists of four histidine residues (SEQ ID NO:27). In some embodiments, the first polypeptide consists of five histidine residues (SEQ ID NO:28). In some embodiments, the His cap domain of the second polypeptide consists of five histidine residues (SEQ ID NO:28).

In some embodiments, the exposed C-terminus of the first polypeptide fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO:5). In some embodiments, the exposed C-terminus of the first polypeptide fused to the His cap domain consists of the amino acid sequence selected from the group consisting of: VEIKH (SEQ ID NO:6), VEIKHH (SEQ ID NO: 7), VEIKHHH (SEQ ID NO:8), VEIKHHHH (SEQ ID NO:9), and VEIKHHHHH (SEQ ID NO: 10). In some embodiments, the exposed C-terminus of the first polypeptide fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15). In some embodiments, the exposed C-terminus of the first polypeptide fused to the His cap domain consists of the amino acid sequence selected from the group consisting of: VEIKRTH (SEQ ID NO:16), VEIKRTHH (SEQ ID NO: 17), VEIKRTHHH (SEQ ID NO:18), VEIKRTHHHH (SEQ ID NO:19), and VEIKRTHHHHH (SEQ ID NO:20). In some embodiments, the exposed C-terminus of the first polypeptide fused to the His cap domain consists of the amino acid sequence of TKVTVL (His)n (SEQ ID NO:46), TKLTVL (His)n (SEQ ID NO:47), TQLIIL (His)n (SEQ ID NO:48), TELTVL (His)n (SEQ ID NO:49), or TQLTVL (His)n (SEQ ID NO:50), wherein n is 1, 2, 3, 4, or 5. In some embodiments, the exposed C-terminus of the first polypeptide fused to the His cap domain consists of the amino acid sequence of TKLTVL (His)n (SEQ ID NO:47), TKVTVL (His)n (SEQ ID NO:46), NGLVYAG (His)n (SEQ ID NO:51), NGLLYAG (His)n (SEQ ID NO: 52), or TRLTVL (His)n (SEQ ID NO:53), wherein n is 1, 2, 3, 4, or 5.

In some embodiments, the exposed C-terminus of the second polypeptide fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO:5). In some embodiments, the exposed C-terminus of the second polypeptide fused to the His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO:9), and VEIKHHHHH (SEQ ID NO:10). In some embodiments, the exposed C-terminus of the second polypeptide fused to the His cap domain consists of the amino acid sequence selected from the group consisting of: VEIKRH (SEQ ID NO:11), VEIKRHH (SEQ ID NO:12), VEIKRHHH (SEQ ID NO: 13), VEIKRHHHH (SEQ ID NO:14), and VEIKRHHHHH (SEQ ID NO:15). In some embodiments, the exposed C-terminus of the second polypeptide fused to the His cap domain consists of the amino acid sequence selected from the group consisting of: VEIKRTH (SEQ ID NO: 16), VEIKRTHH (SEQ ID NO:17), VEIKRTHHH (SEQ ID NO:18), VEIKRTHHHH (SEQ ID NO: 19), and VEIKRTHHHHH (SEQ ID NO:20). In some embodiments, the exposed C-terminus of the second polypeptide fused to the His cap domain consists of the amino acid sequence of TKVTVL (His)n (SEQ ID NO:46), TKLTVL (His)n (SEQ ID NO:47), TQLIIL (His)n (SEQ ID NO:48), TELTVL (His)n (SEQ ID NO:49), or TQLTVL (His)n (SEQ ID NO:50), wherein n is 1, 2, 3, 4, or 5. In some embodiments, the exposed C-terminus of the second polypeptide fused to the His cap domain consists of the amino acid sequence of TKLTVL (His)n (SEQ ID NO:47), TKVTVL (His)n (SEQ ID NO:46), NGLVYAG (His)n (SEQ ID NO:51), NGLLYAG (His)n (SEQ ID NO:52), or TRLTVL (His)n (SEQ ID NO:53), wherein n is 1, 2, 3, 4, or 5.

In some embodiments, the isolated protein further comprises a third polypeptide comprising a target binding region. In some embodiments, the third polypeptide comprises an exposed C-terminus and the method further comprises fusing a cap domain (e.g., a His cap domain, SLSLSPGK (SEQ ID NO:36), or TVAPTESS (SEQ ID NO:37)) to the end of the exposed C-terminus of the third polypeptide. In some embodiments, the third polypeptide is capable of binding to a different target than the first polypeptide, the second polypeptide, or both. In some embodiments, the exposed C-terminus of the third polypeptide comprises an anti-drug antibody epitope. In some embodiments, the target binding region of the third polypeptide comprises an antibody variable domain or an antigen-binding fragment thereof. In some embodiments, the target binding region of the third polypeptide comprises a light chain antibody variable domain or an antigen-binding fragment thereof. In some embodiments, the target binding region of the third polypeptide comprises a heavy chain antibody variable domain or an antigen-binding fragment thereof.

In some embodiments, the cap domain of the third polypeptide consists of a His cap domain, or the amino acid sequence of SLSLSPGK (SEQ ID NO:36) or TVAPTESS (SEQ ID NO: 37). In some embodiments, the His cap domain of the third polypeptide consists of a single histidine residue. In some embodiments, the His cap domain of the third polypeptide consists of two histidine residues. In some embodiments, the His cap domain of the third polypeptide consists of three histidine residues. In some embodiments, the His cap domain of the third polypeptide consists of four histidine residues (SEQ ID NO:27). In some embodiments, the His cap domain of the third polypeptide consists of five histidine residues (SEQ ID NO:28).

In some embodiments, the exposed C-terminus of the third polypeptide fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO:5). In some embodiments, the exposed C-terminus of the third polypeptide fused to the His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO:9), and VEIKHHHHH (SEQ ID NO:10). In some embodiments, the exposed C-terminus of the third polypeptide fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15). In some embodiments, the exposed C-terminus of the third polypeptide fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO: 19), and VEIKRTHHHHH (SEQ ID NO:20). In some embodiments, the exposed C-terminus of the third polypeptide fused to the His cap domain consists of the amino acid sequence of TKVTVL (His)n (SEQ ID NO:46), TKLTVL (His)n (SEQ ID NO:47), TQLIIL (His)n (SEQ ID NO: 48), TELTVL (His)n (SEQ ID NO:49), or TQLTVL (His)n (SEQ ID NO:50), wherein n is 1, 2, 3, 4, or 5. In some embodiments, the exposed C-terminus of the third polypeptide fused to the His cap domain consists of the amino acid sequence of TKLTVL (His)n (SEQ ID NO:47), TKVTVL (His)n (SEQ ID NO:46), NGLVYAG (His)n (SEQ ID NO:51), NGLLYAG (His)n (SEQ ID NO: 52), or TRLTVL (His)n (SEQ ID NO:53), wherein n is 1, 2, 3, 4, or 5.

In some embodiments, the isolated protein is bispecific. In some embodiments, the isolated protein is multi-specific. In some embodiments, the isolated protein is trispecific. In some embodiments, the isolated protein is heterodimeric.

In another aspect, provided herein is a capping means (e.g., a histidine capping means) for reducing interaction between an isolated polypeptide and a reference, wherein the isolated polypeptide comprises one or more target binding regions and an exposed C-terminus. In some embodiments, the one or more target binding regions comprise an antibody variable domain or antigen binding fragment thereof. In some embodiments, the reference is a molecule present in a biological substance. In some embodiments, the biological substance comprises serum and/or plasma. In some embodiments, the biological substance comprises serum (e.g., human serum). In some embodiments, the biological substance comprises plasma (e.g., human plasma). In some embodiments, the reference comprises an anti-drug antibody. In some embodiments, the reference comprises an anti-drug antibody pre-existing in the serum and/or plasma of a subject (e.g., a human subject). In some embodiments, the exposed C-terminus comprises an anti-drug antibody epitope. In some embodiments, the exposed C-terminus consists of the amino acid sequence selected from the group consisting of: VTVSS (SEQ ID NO:21), VEIK (SEQ ID NO:22), VEIKR (SEQ ID NO:23), VEIKRT (SEQ ID NO:24), TKVTVL (SEQ ID NO: 38), TKLTVL (SEQ ID NO:39), TQLIIL (SEQ ID NO:40), TELTVL (SEQ ID NO:41), and TQLTVL (SEQ ID NO:42). In some embodiments, the exposed C-terminus consists of an amino acid sequence provided in Table 1.

In another aspect, provided herein is a capping means (e.g., a histidine capping means) for reducing interaction between an isolated protein and a reference, wherein the isolated protein comprises: (a) a first polypeptide comprising a target binding region, and an exposed C-terminus; and (b) a second polypeptide comprising a target binding region. In some embodiments, the second polypeptide comprises an exposed C-terminus. In some embodiments, the target binding region of the first and second polypeptides are capable of binding to different targets. In some embodiments, the isolated protein further comprises a third polypeptide comprising a target binding region. In some embodiments, the third polypeptide comprises an exposed C-terminus. In some embodiments, the third polypeptide is capable of binding to a different target than the first polypeptide, the second polypeptide, or both. In some embodiments, the isolated protein is heterodimeric. In some embodiments, the isolated protein is bispecific or multi-specific. In specific embodiments, the multi-specific protein is trispecific. In some embodiments, the exposed C-terminus comprises an anti-drug antibody epitope. In some embodiments, the exposed C-terminus consists of the amino acid sequence selected from the group consisting of: VTVSS (SEQ ID NO:21), VEIK (SEQ ID NO:22), VEIKR (SEQ ID NO: 23), and VEIKRT (SEQ ID NO:24). In some embodiments, the reference comprises a molecule in a biological substance. In some embodiments, the biological substance comprises serum and/or plasma (e.g., human serum and/or human plasma). In some embodiments, the reference comprises an anti-drug antibody. In some embodiments, the anti-drug antibody is pre-existing in the serum and/or plasma of a subject (e.g., a human subject).

In another aspect, provided herein is a capping means (e.g., a histidine capping means) for reducing interaction between a bispecific or a multi-specific antibody and a reference, wherein the bispecific or the multi-specific antibody comprises one or more target binding regions and an exposed C-terminus. In some embodiments, the one or more target binding regions comprises at least one antibody variable domain. In some embodiments, the at least one antibody variable domain comprises a heavy chain variable domain. In some embodiments, the at least one antibody variable domain comprises a light chain variable domain. In some embodiments, the exposed C-terminus comprises an anti-drug antibody epitope. In some embodiments, the multi-specific antibody comprises a trispecific antibody. In some embodiments, the reference comprises a molecule in a biological substance. In some embodiments, the biological substance comprises serum and/or plasma (e.g., human serum and/or human plasma). In some embodiments, the reference comprises an anti-drug antibody. In some embodiments, the anti-drug antibody is pre-existing in the serum and/or plasma of a subject (e.g., a human subject).

In another aspect, provided herein is an isolated polypeptide and a capping means (e.g., a histidine capping means) for reducing interaction between the isolated polypeptide and a reference, wherein the isolated polypeptide comprises one or more target binding regions and an exposed C-terminus. In some embodiments, the one or more target binding regions comprise an antibody variable domain or antigen binding fragment thereof. In some embodiments, the reference is a molecule present in a biological substance. In some embodiments, the biological substance comprises serum and/or plasma. In some embodiments, the biological substance comprises serum. In some embodiments, the biological substance comprises plasma. In some embodiments, the reference comprises an anti-drug antibody. In some embodiments, the anti-drug antibody is pre-existing in the serum and/or plasma of the subject (e.g., a human subject). In some embodiments, the exposed C-terminus comprises an anti-drug antibody epitope. In some embodiments, the exposed C-terminus consists of the amino acid sequence selected from the group consisting of: VTVSS (SEQ ID NO:21), VEIK (SEQ ID NO:22), VEIKR (SEQ ID NO: 23), VEIKRT (SEQ ID NO:24), TKVTVL (SEQ ID NO:38), TKLTVL (SEQ ID NO:39), TQLIIL (SEQ ID NO:40), TELTVL (SEQ ID NO:41), and TQLTVL (SEQ ID NO:42). In some embodiments, the exposed C-terminus consists of an amino acid sequence provided in Table 1.

In another aspect, provided herein is an isolated protein and a capping means (e.g., a histidine capping means) for reducing interaction between the isolated protein and a reference, wherein the isolated protein comprises: (a) a first polypeptide comprising a target binding region, and an exposed C-terminus; and (b) a second polypeptide comprising a target binding region. In some embodiments, the second polypeptide comprises an exposed C-terminus. In some embodiments, the target binding region of the first and second polypeptides are capable of binding to different targets. In some embodiments, the isolated protein further comprises a third polypeptide comprising a target binding region. In some embodiments, the third polypeptide comprises an exposed C-terminus. In some embodiments, the third polypeptide is capable of binding to a different target than the first polypeptide, the second polypeptide, or both. In some embodiments, the isolated protein is heterodimeric. In some embodiments, the isolated protein is bispecific or multi-specific. In some embodiments, the multi-specific protein is trispecific. In some embodiments, the exposed C-terminus comprises an anti-drug antibody epitope. In some embodiments, the reference comprises a molecule in a biological substance. In some embodiments, the biological substance comprises serum and/or plasma. In some embodiments, the biological substance comprises serum and/or plasma (e.g., human serum and/or human plasma). In some embodiments, the reference comprises an anti-drug antibody. In some embodiments, the anti-drug antibody is pre-existing in the serum and/or plasma of a subject (e.g., a human subject).

In another aspect, provided herein is a bispecific or a multi-specific antibody and a capping means (e.g., a histidine capping means) for reducing interaction between the isolated protein and a reference, wherein the bispecific or the multi-specific antibody comprises one or more target binding regions and an exposed C-terminus. In some embodiments, the one or more target binding regions comprises at least one antibody variable domain. In some embodiments, the at least one antibody variable domain comprises a heavy chain variable domain. In some embodiments, the at least one antibody variable domain comprises a light chain variable domain. In some embodiments, the isolated protein is heterodimeric. In some embodiments, the multi-specific antibody comprises a trispecific antibody. In some embodiments, the exposed C-terminus comprises an anti-drug antibody epitope. In some embodiments, the reference comprises a molecule in a biological substance. In some embodiments, the biological substance comprises serum and/or plasma (e.g., human serum and/or human plasma). In some embodiments, the reference comprises an anti-drug antibody. In some embodiments, the anti-drug antibody is pre-existing in the serum and/or plasma of a subject (e.g., a human subject).

In one embodiment, provided herein is an isolated protein comprising a target binding region and an exposed C-terminus fused to a cap domain for use in a diagnostic assay, wherein the cap domain consists of a His cap domain, or the amino acid sequence of SLSLSPGK (SEQ ID NO: 36) or TVAPTESS (SEQ ID NO:37). In embodiments, the protein is an antibody (e.g., a bispecific or multi-specific antibody). In some embodiments, the antibody binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin. In some embodiments, the antibody binds to an antigen of a pathogen. In some embodiments, the pathogen is a virus, a bacteria, a fungus, or a parasite. In some embodiments, the protein is used in a diagnostic assay to detect the presence of a molecule in a biological substance. In some embodiments, the molecule is an antigen. In some embodiments, the biological substance comprises serum and/or plasma (e.g., human serum and/or human plasma). In some embodiments, the diagnostic assay is conducted in vitro or in vivo. In some embodiments, the exposed C-terminus comprises an anti-drug antibody epitope. In some embodiments, the exposed C-terminus consists of the amino acid sequence selected from the group consisting of: VTVSS (SEQ ID NO:21), VEIK (SEQ ID NO:22), VEIKR (SEQ ID NO:23), VEIKRT (SEQ ID NO:24), TKVTVL (SEQ ID NO:38), TKLTVL (SEQ ID NO:39), TQLIIL (SEQ ID NO:40), TELTVL (SEQ ID NO:41), and TQLTVL (SEQ ID NO:42).

In one embodiment, provided herein is an isolated therapeutic protein comprising a target binding region and an exposed C-terminus fused to a cap domain for use in therapy, wherein the cap domain consists of a His cap domain, or the amino acid sequence of SLSLSPGK (SEQ ID NO:36) or TVAPTESS (SEQ ID NO:37). In some embodiments, the protein is a cytokine or an antibody (e.g., a bispecific or multi-specific antibody). In some embodiments, the cytokine is IL-12, IL-23, IL-1β, IL-6, IL-15, IL-2, IL-5, TNF-alpha, IL-9, or IL-17. In some embodiments, the antibody binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin. In some embodiments, the antibody binds to an antigen of a pathogen. In some embodiments, the pathogen is a virus, a bacteria, a fungus, or a parasite. In some embodiments, the exposed C-terminus comprises an anti-drug antibody epitope. In some embodiments, the exposed C-terminus consists of the amino acid sequence selected from the group consisting of: VTVSS (SEQ ID NO:21), VEIK (SEQ ID NO:22), VEIKR (SEQ ID NO: 23), VEIKRT (SEQ ID NO:24), TKVTVL (SEQ ID NO:38), TKLTVL (SEQ ID NO:39), TQLIIL (SEQ ID NO:40), TELTVL (SEQ ID NO:41), and TQLTVL (SEQ ID NO:42).

In one embodiment, provided herein is an isolated protein comprising a target binding region and an exposed C-terminus fused to a cap domain, wherein the exposed C-terminus consists of the amino acid sequence VTVSS (SEQ ID NO:21), VEIK (SEQ ID NO:22), VEIKR (SEQ ID NO:23), VEIKRT (SEQ ID NO:24), TKVTVL (SEQ ID NO:38), TKLTVL (SEQ ID NO: 39), TQLIIL (SEQ ID NO:40), TELTVL (SEQ ID NO:41), or TQLTVL (SEQ ID NO:42), and wherein the cap domain consists of a His cap domain, or the amino acid sequence of SLSLSPGK (SEQ ID NO:36) or TVAPTESS (SEQ ID NO:37). In some embodiments, the protein is an antibody (e.g., a bispecific or multi-specific antibody). In some embodiments, the antibody binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin. In some embodiments, the antibody binds to an antigen of a pathogen. In some embodiments, the pathogen is a virus, a bacteria, a fungus, or a parasite.

In one embodiment, provided herein is a therapeutic bispecific or multi-specific (e.g., tri-specific) antibody comprising two or more target binding regions and an exposed C-terminus fused to a cap domain for use in therapy, wherein the cap domain is a His cap domain, or the amino acid sequence of SLSLSPGK (SEQ ID NO:36) or TVAPTESS (SEQ ID NO:37). In some embodiments, the two or more target binding regions bind to two or more antigens. In some embodiments, at least one antigen is selected from the group consisting of a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, and mesothelin. In some embodiments, the antibody binds to an antigen of a pathogen. In some embodiments, the pathogen is a virus, a bacteria, a fungus, or a parasite. In some embodiments, the exposed C-terminus comprises an anti-drug antibody epitope. In some embodiments, the exposed C-terminus consists of the amino acid sequence selected from the group consisting of: VTVSS (SEQ ID NO:21), VEIK (SEQ ID NO: 22), VEIKR (SEQ ID NO:23), VEIKRT (SEQ ID NO:24), TKVTVL (SEQ ID NO:38), TKLTVL (SEQ ID NO:39), TQLIIL (SEQ ID NO:40), TELTVL (SEQ ID NO:41), or TQLTVL (SEQ ID NO:42).

In another aspect, provided herein is a pharmaceutical composition comprising a protein (e.g., a bispecific or multi-specific antibody) comprising an exposed C-terminus and a cap domain, and a pharmaceutically acceptable excipient for use in therapy. In one embodiment, provided herein is pharmaceutical composition comprising a protein described herein, a bispecific antibody described herein, or a multi-specific (e.g., tri-specific) antibody described herein, and a pharmaceutically acceptable excipient for use in therapy.

In another aspect, provided herein is a pharmaceutical composition comprising a protein (e.g., a bispecific or multi-specific antibody) comprising an exposed C-terminus and a cap domain, and a pharmaceutically acceptable excipient for use in therapy. In another embodiment, provided herein is a pharmaceutical composition comprising a protein described herein, a bispecific antibody described herein, or a multi-specific (e.g., tri-specific) antibody described herein, and a pharmaceutically acceptable excipient for use in a diagnostic assay.

In another aspect, provided herein is use of a cap domain to reduce interaction between an anti-drug antibody and a protein, wherein the protein comprises one or more target binding regions and an exposed C-terminus. In specific embodiments, the cap domain is a His cap domain, or consists of the amino acid sequence of SLSLSPGK (SEQ ID NO:36) or TVAPTESS (SEQ ID NO:37). In some embodiments, the exposed C-terminus comprises an anti-drug antibody epitope. In some embodiments, the cap domain is a His cap domain consisting of one, two, three, four, or five histidines.

In another aspect, provided herein is use of a cap domain to reduce aggregation in a biological substance, wherein the protein comprises one or more target binding regions and an exposed C-terminus. In specific embodiments, the cap domain is a His cap domain, or consists of the amino acid sequence of SLSLSPGK (SEQ ID NO:36) or TVAPTESS (SEQ ID NO:37). In some embodiments, the biological substance comprises serum and/or plasma (e.g., human serum and/or human plasma). In some embodiments, the exposed C-terminus comprises an anti-drug antibody epitope. In some embodiments, the cap domain is a His cap domain consisting of one, two, three, four, or five histidines.

In another aspect, provided herein is use of a cap domain to reduce self-aggregation, wherein the protein comprises one or more target binding regions and an exposed C-terminus. In specific embodiments, the cap domain consists is a His cap domain, or consists of the amino acid sequence of SLSLSPGK (SEQ ID NO:36) or TVAPTESS (SEQ ID NO:37). In some embodiments, the exposed C-terminus comprises an anti-drug antibody epitope. In some embodiments, the cap domain is a His cap domain consisting of one, two, three, four, or five histidines.

In one embodiment, provided herein is an isolated protein comprising a target binding region and an exposed C-terminus fused to a cap domain, wherein the cap domain consists of His cap domain, or the amino acid sequences of HHHHHH (SEQ ID NO:26)), SLSLSPGK (SEQ ID NO: 36), or AS, TVAPTESS (SEQ ID NO:37). In some embodiments, the protein exhibits reduced interaction with an anti-drug antibody in a biological substance. In some embodiments, the biological substance comprises serum and/or plasma (e.g., human serum and/or human plasma). In some embodiments, the exposed C-terminus comprises an anti-drug antibody epitope. In some embodiments, the exposed C-terminus consists of the amino acid sequence selected from the group consisting of: VTVSS (SEQ ID NO:21), VEIK (SEQ ID NO:22), VEIKR (SEQ ID NO: 23), VEIKRT (SEQ ID NO:24), TKVTVL (SEQ ID NO:38), TKLTVL (SEQ ID NO:39), TQLIIL (SEQ ID NO:40), TELTVL (SEQ ID NO:41), or TQLTVL (SEQ ID NO:42). In some embodiments, for use in therapy or a diagnostic assay.

### 6. BRIEF DESCRIPTION OF THE DRAWINGS

The foregoing summary, as well as the following detailed description of embodiments of the present application, will be better understood when read in conjunction with the appended drawings. It should be understood, however, that the application is not limited to the precise embodiments shown in the drawings.

FIG. 1 depicts an exemplary antibody structure comprising an Fc domain and two scFvs (e.g., Fc-scFv antibody) amenable for use with the present invention (e.g., the addition of one or more cap domain(s)).

FIG. 2 depicts an exemplary antibody structure comprising an Fc domain and an scFv heterodimerized to an Fc domain (e.g., Fc-scFv×Fc antibody) amenable for use with the present invention (e.g., the addition of one or more cap domain(s)).

FIG. 3 depicts an exemplary trispecific antibody structure comprising a heavy chain and an scFv heterodimerized to an Fc domain and a scFv (e.g., HC-scFv×Fc-scFv trispecific antibody) amenable for use with the present invention (e.g., the addition of one or more cap domain(s)).

FIG. 4 depicts an exemplary antibody structure comprising a heavy chain heterodimerized with an Fc and two scFvs (e.g., HC×scFv-Fc-scFv) amenable for use with the present invention (e.g., the addition of one or more cap domain(s)).

FIG. 5 depicts an exemplary bispecific antibody structure comprising an Fc domain and two scFvs heterodimerized with an Fc and two scFvs (e.g., scFv-Fc-scFv bispecific antibody) amenable for use with the present invention (e.g., the addition of one or more cap domain(s)).

FIG. 6 depicts an exemplary antibody structure comprising a variable heavy chain domain-Fc domain fusion (e.g., Fc-VHH fusion) amenable for use with the present invention (e.g., the addition of one or more cap domain(s)).

FIG. 7 depicts an exemplary multispecific antibody structure comprising an Fc domain, an scFv, and a variable heavy chain domain heterodimerized with a heavy chain and a variable heavy chain domain (e.g., scFv-Fc-VHH×HC-VHH) amenable for use with the present invention (e.g., the addition of one or more cap domain(s)).

FIG. 8 depicts an exemplary multispecific antibody (e.g., a trispecific antibody) with a heavy chain heterodimerized with an Fc domain and two scFvs (e.g., HC×scFv-Fc-scFv) amenable for use with the present invention (e.g., the addition of one or more cap domain(s)).

FIG. 9A and FIG. 9B depict an exemplary trispecific antibody without (FIG. 9A) or with a cap domain (e.g., a His cap domain, SLSLSPGK (SEQ ID NO:36), or TVAPTESS (SEQ ID NO: 37)) at the C-terminus of an antigen-binding region of the trispecific antibody (FIG. 9B).

FIG. 10A and FIG. 10B depict exemplary trispecific antibodies with a cap domain (e.g., a His cap domain, SLSLSPGK (SEQ ID NO:36), or TVAPTESS (SEQ ID NO:37)) at the C-terminus of an antigen-binding region of the trispecific antibody.

### 7. DETAILED DESCRIPTION

The present disclosure is directed, in part, to proteins, such as therapeutic proteins (e.g., hormones, cytokines, enzymes, fusion proteins, or antibodies, or other antigen binding proteins) that are modified with a cap domain (e.g., a His cap domain, SLSLSPGK (SEQ ID NO: 36), or TVAPTESS (SEQ ID NO:37)) at an exposed C-terminus proteins. In a particular aspect, the present disclosure relates to an isolated protein comprising one or more target binding regions and an exposed C-terminus fused to a cap domain (e.g., a His cap domain, SLSLSPGK (SEQ ID NO:36), or TVAPTESS (SEQ ID NO:37)). In a specific embodiment, such a protein exhibits reduced interaction with a reference relative to the interaction of the protein lacking the cap domain (e.g., a His cap domain, SLSLSPGK (SEQ ID NO:36), or TVAPTESS (SEQ ID NO: 37)) with the reference. In another specific embodiment, such a protein exhibits reduced aggregation upon incubation with a biological substance relative to the aggregation exhibited upon incubation of the protein lacking the cap domain (e.g., a His cap domain, SLSLSPGK (SEQ ID NO: 36), or TVAPTESS (SEQ ID NO:37)) with the biological substance. In another specific embodiment, embodiment, such a protein exhibits reduced self-aggregation relative to the self-aggregation exhibited by the protein lacking the cap domain (e.g., a His cap domain, SLSLSPGK (SEQ ID NO:36), or TVAPTESS (SEQ ID NO:37)).

In a specific aspect, the present disclosure is directed to proteins, such as therapeutic proteins (e.g., hormones, cytokines, enzymes, fusion proteins, or antibodies, or other antigen binding proteins) that are modified with a cap domain (e.g., a His cap domain, SLSLSPGK (SEQ ID NO: 36), or TVAPTESS (SEQ ID NO:37)) that reduces or prevents an anti-drug antibody (ADA) response. In particular aspects, the present disclosure relates to a protein, such as a therapeutic protein, having a (His)n (SEQ ID NO:25) sequence at the end of a non-naturally occurring C-terminus or C-terminal anti-drug antibody epitope, wherein n is 1, 2, 3, 4, or 5, and the presence of the (His)n (SEQ ID NO:25) sequence reduces or prevents an ADA response.

Unless defined otherwise, all technical and scientific terms used herein have the same meaning as commonly understood to one of ordinary skill in the art to which the invention pertains. Otherwise, certain terms used herein have the meanings as set forth in the specification. All patents, published patent applications and publications cited herein are incorporated by reference as if set forth fully herein. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

As used herein, the abbreviations for the genetically encoded amino acids are conventional and are as follows:

| Amino Acid | One-Letter Symbol | Common Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

It must be noted that as used herein and in the appended claims, the singular forms “a,” “an,” and “the” include plural reference unless the context clearly dictates otherwise.

As used herein, the terms “about” and “approximately” mean within 20%, within 15%, within 10%, within 9%, within 8%, within 7%, within 6%, within 5%, within 4%, within 3%, within 2%, within 1%, or less of a given value or range.

The term “between” as used in a phrase as such “between A and B” or “between A-B” refers to a range including both A and B.

As used herein, the term “anti-drug antibody” or “ADA” refers to an antibody that is capable of interacting with a protein, such as therapeutic protein (e.g., an antibody or another antigen binding protein). In a specific embodiment, an anti-drug antibody is pre-existing in the serum of a subject (e.g., a human subject). In another specific embodiment, an anti-drug antibody is pre-existing in the plasma of a subject (e.g., a human subject). In specific embodiments, an anti-drug antibody binds to or cross-reacts with a polypeptide comprising one or more target binding regions (e.g., an antigen binding protein, such as, e.g., an antibody) or other protein, and neutralizes the polypeptide or other protein. In certain embodiments, an anti-drug antibody changes the pharmacokinetics and/or safety profile of a polypeptide comprising one or more target binding regions (e.g., an antigen binding protein, such as, e.g., an antibody) or other protein. In some embodiments, an anti-drug antibody inhibits a function of a polypeptide comprising one or more target binding regions (e.g., an antigen binding protein, such as, e.g., an antibody) or other protein (e.g., therapeutic protein). In certain embodiments, an anti-drug antibody interferes with the binding of a polypeptide comprising one or more target binding regions (e.g., an antigen binding protein, such as, e.g., an antibody) to one or more targets (e.g., one more antigens).

As used herein, the term “target binding region” refers to a plurality of contiguous or non-contiguous amino acid residues in a polypeptide that in the presence of a target can bind to the target, such as for example an antigen. In a specific embodiment, a target binding region comprises an antigen binding region.

As used herein, the term “exposed C-terminus” refers to a C-terminus of a polypeptide (e.g., an antibody, such as, e.g., a bispecific, trispecific or other multi-specific antibody), wherein the polypeptide with such C-terminus, absent a cap domain (e.g., a His cap domain, SLSLSPGK (SEQ ID NO:36), or TVAPTESS (SEQ ID NO:37)), exhibits: (1) greater interaction with a reference than the same polypeptide with the cap domain (e.g., a His cap domain, SLSLSPGK (SEQ ID NO:36), or TVAPTESS (SEQ ID NO:37)); (2) greater aggregation with a biological substance; (3) greater self-aggregation; or a combination thereof. In specific embodiments, the exposed C-terminus is present only in a non-naturally occurring polypeptide (e.g., an antibody, such as, e.g., a bispecific, trispecific or other multi-specific antibody). In specific embodiments, the exposed C-terminus is a sequence that is generally buried or otherwise not exposed in a naturally occurring polypeptide (e.g., a canonical immunoglobulin construct). In certain embodiments, the exposed C-terminus comprises a C-terminus anti-drug antibody epitope. In some embodiments, an exposed C-terminus comprises the four to six C-terminal amino acid residues of a kappa light chain, a lambda light chain, or a variable heavy chain region of any species found at, e.g., https://www.imgt.org/IMGTrepertoire/Proteins/index.php#B. In some embodiments, an exposed C-terminus consists of the four to six C-terminal amino acid residues of a kappa light chain, a lambda light chain, or a variable heavy chain region of any species found at, e.g., https://www.imgt.org/IMGTrepertoire/Proteins/index.php#B. In some embodiments, the species is human, monkey, chicken, or bovine. In some embodiments, an exposed C-terminus comprises the four to six C-terminal amino acid residues of a sequence found under IGKJ, IGLJ, or IGHJ at https://www.imgt.org/IMGTrepertoire/Proteins/index.php#B. In some embodiments, an exposed C-terminus comprises the four to six C-terminal amino acid residues of a sequence found under IGKJ, IGLJ, or IGHJ at https://www.imgt.org/IMGTrepertoire/Proteins/index.php#B. In some embodiments, an exposed C-terminus consists of a C-terminus sequence of one of those provided in Table 1. In some embodiments, an exposed C-terminus consists of the four to six C-terminal amino acid residues of a sequence found under IGKJ, IGLJ, or IGHJ at https://www.imgt.org/IMGTrepertoire/Proteins/index.php#B. In some embodiments, an exposed C-terminus consists of a C-terminus sequence of one of those provided in Table 1.

TABLE 1

| C-terminus sequence | Antibody subunit | Species |
|---|---|---|
| VTVSS (SEQ ID NO: 21) | Heavy | Human (Homo sapiens) |
| | | House mouse (Mus musculus) |
| | | Rhesus monkey (Macaca mulatta) |
| LTVSS (SEQ ID NO: 29) | Heavy | House mouse (Mus musculus) |
| VTVSA (SEQ ID NO: 30) | Heavy | House mouse (Mus musculus) |
| ITISS (SEQ ID NO: 54) | Heavy | Rhesus monkey (Macaca mulatta) |
| VIVSS (SEQ ID NO: 55) | Heavy | Chicken (Gallus gallus) |
| VEIK (SEQ ID NO: 22) | Kappa light | Human (Homo sapiens) |
| | | Rhesus monkey (Macaca mulatta) |
| | | Bovine (Bos taurus) |
| VDIK (SEQ ID NO: 56) | Kappa light | Human (Homo sapiens) |
| LEIK (SEQ ID NO: 57) | Kappa light | Human (Homo sapiens) |
| | | House mouse (Mus musculus) |
| | | Rhesus monkey (Macaca mulatta) |
| LDIK (SEQ ID NO: 58) | Kappa light | Rhesus monkey (Macaca mulatta) |
| LEVK (SEQ ID NO: 59) | Kappa light | Bovine (Bos taurus) |
| VEIN (SEQ ID NO: 60) | Kappa light | Bovine (Bos taurus) |
| LEIR (SEQ ID NO: 61) | Kappa light | Bovine (Bos taurus) |
| VEIKR (SEQ ID NO: 23) | Kappa light | Human (Homo sapiens) |
| VEIKRT (SEQ ID NO: 24) | Kappa light | Human (Homo sapiens) |
| TKLTVL (SEQ ID NO: 39) | Lambda Light | Human (Homo sapiens) |
| | | House mouse (Mus musculus) |
| | | Rhesus monkey (Macaca mulatta) |
| TKVTVL (SEQ ID | Lambda Light | Human (Homo sapiens) |
| NO: 38) | | House mouse (Mus musculus) |
| TQLIIL (SEQ ID NO: 40) | Lambda Light | Human (Homo sapiens) |
| TELTVL (SEQ ID NO: 41) | Lambda Light | Human (Homo sapiens) |
| TQLTVL (SEQ ID NO: 42) | Lambda Light | Human (Homo sapiens) |
| GLVYAG (SEQ ID | Lambda Light | House mouse (Mus musculus) |
| NO: 62) | | |
| GLLYAG (SEQ ID | Lambda Light | House mouse (Mus musculus) |
| NO: 63) | | |
| TRLTVL (SEQ ID NO: 45) | Lambda Light | House mouse (Mus musculus) |
| | | Rhesus monkey (Macaca mulatta) |
| | | Bovine (Bos taurus) |
| TQLTIV (SEQ ID NO: 64) | Lambda Light | Rhesus monkey (Macaca mulatta) |
| TKLTIL (SEQ ID NO: 65) | Lambda Light | Rhesus monkey (Macaca mulatta) |
| TRLTDI (SEQ ID NO: 66) | Lambda Light | Rhesus monkey (Macaca mulatta) |
| TTLTVL (SEQ ID NO: 67) | Lambda Light | Chicken (Gallus gallus) |
| | | Bovine (Bos taurus) |
| TWVTVL (SEQ ID | Lambda Light | Bovine (Bos taurus) |
| NO: 68) | | |
| TRVTVL (SEQ ID NO: 69) | Lambda Light | Bovine (Bos taurus) |
| TTVTVL (SEQ ID NO: 70) | Lambda Light | Bovine (Bos taurus) |

As used herein, the term “non-naturally occurring C-terminus” generally refers to a C-terminus that is not normally present as the C-terminus of a polypeptide or protein, such as a therapeutic protein or diagnostic protein (e.g., an antigen binding protein), found in nature. An example of a non-naturally occurring C-terminus of a protein is a C-terminus of a protein (e.g., an antigen binding domain) engineered or modified from its wild-type sequence. In a specific embodiment, a non-naturally occurring C-terminus of a protein is a C-terminus of a protein that is generally not naturally exposed for interaction with a reference (e.g., a biological substance or an ADA). In specific embodiments, a non-naturally occurring C-terminus is an exposed C-terminus.

As used herein, the term “at the end” when used in the context of an exposed C-terminus, refers to the last amino acid residue of the exposed C-terminus or a non-naturally occurring C-terminus or C-terminal ADA epitope. For example, in some embodiments, an exposed C-terminus terminating in the amino acid sequence VTVSS (SEQ ID NO:21), and having a His cap domain fused at the end of the C-terminus can result in an amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO: 5). Similarly, in some embodiments, an exposed C-terminus terminating in the amino acid sequence VEIK (SEQ ID NO:22), and having a His cap domain fused at the end of the C-terminus can result in an amino acid sequence selected from the group consisting of: VEIKH (SEQ ID NO:6), VEIKHH (SEQ ID NO:7), VEIKHHH (SEQ ID NO:8), VEIKHHHH (SEQ ID NO: 9), and VEIKHHHHH (SEQ ID NO:10). In other embodiments, an exposed C-terminus terminating in the amino acid sequence VEIKR (SEQ ID NO:23), and having a His cap domain fused at the end of the C-terminus can result in an amino acid sequence selected from the group consisting of: VEIKRH (SEQ ID NO:11), VEIKRHH (SEQ ID NO:12), VEIKRHHH (SEQ ID NO: 13), VEIKRHHHH (SEQ ID NO:14), and VEIKRHHHHH (SEQ ID NO:15). In further embodiments, an exposed C-terminus terminating in the amino acid sequence VEIKRT (SEQ ID NO: 24), and having a His cap domain fused at the end of the C-terminus can result in an amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO:20). In some embodiments, an exposed C-terminus terminating in the amino acid sequence TKVTVL (SEQ ID NO:38), TKLTVL (SEQ ID NO:39), TQLIIL (SEQ ID NO:40), TELTVL (SEQ ID NO:41), or TQLTVL (SEQ ID NO:42), and having a His cap domain fused at the end of the C-terminus can result in an amino acid sequence TKVTVL (His)n (SEQ ID NO:46), TKLTVL (His)n (SEQ ID NO:47), TQLIIL (His)n (SEQ ID NO: 48), TELTVL (His)n (SEQ ID NO:49), or TQLTVL (His)n (SEQ ID NO:50), wherein n is 1, 2, 3, 4, or 5. In some embodiments, an exposed C-terminus terminating in the amino acid sequence TKLTVL (SEQ ID NO:39), TKVTVL (SEQ ID NO:38), NGLVYAG (SEQ ID NO: 43), NGLLYAG (SEQ ID NO:44), or TRLTVL (SEQ ID NO:45), and having a His cap domain fused at the end of the C-terminus can result in an amino acid sequence TKLTVL (His)n (SEQ ID NO:47), TKVTVL (His)n (SEQ ID NO:46), NGLVYAG (His)n (SEQ ID NO:51), NGLLYAG (His)n (SEQ ID NO:52), or TRLTVL (His)n (SEQ ID NO:53), wherein n is 1, 2, 3, 4, or 5.

As used herein, the term “cap domain” refers to amino acid residues that are not part of the exposed C-terminus of a polypeptide (e.g., an antibody, such as, e.g., a bispecific, trispecific or other multi-specific antibody). In other words, the cap domain is heterologous to the exposed C-terminus of a polypeptide (e.g., an antibody, such as, e.g., a bispecific, trispecific or other multi-specific antibody). In a specific embodiment, the cap domain consists of (His) 6 (i.e., HHHHHH (SEQ ID NO:26)), SLSLSPGK (SEQ ID NO:36), AS, TVAPTESS (SEQ ID NO:37), or a His cap domain. In some embodiments, the cap domain does not consist of (His) 6 (i.e., HHHHHH (SEQ ID NO:26)) or AS. In specific embodiments, the cap domain consists of a His cap domain. In specific embodiments, a cap domain does not interfere with one, two, three, or more, or all of the functions of a protein as assessed by a technique described herein or known to one of skill in the art. In specific embodiments, a cap domain does not interfere with the structure of a protein as assessed by a technique described herein or known to one of skill in the art.

As used herein, the term “capping means” refers the presence or addition of an amino acid sequence consisting of (His) 6 (i.e., HHHHHH (SEQ ID NO:26)), SLSLSPGK (SEQ ID NO: 36), AS, or TVAPTESS (SEQ ID NO:37), or a His cap domain. In specific embodiments, the capping means refers to the presence or addition of an amino acid sequence consisting of SLSLSPGK (SEQ ID NO:36) or TVAPTESS (SEQ ID NO:37), or a His cap domain. In some embodiments, the capping means does not refer to the presence or addition of an amino acid sequence consisting of the amino acid sequence of (His) 6 (i.e., HHHHHH (SEQ ID NO:26)) or AS.

As used herein, the term “His cap domain” refers to one or more histidine amino acid residues that are not part of the exposed C-terminus of a polypeptide (e.g., an antibody, such as, e.g., a bispecific, trispecific or other multi-specific antibody). In other words, the His cap domain is heterologous to the exposed C-terminus of a polypeptide (e.g., an antibody, such as, e.g., a bispecific, trispecific or other multi-specific antibody). In a specific embodiment, the His cap domain consists of (His)ₙ (SEQ ID NO:25), wherein n is 1, 2, 3, 4, or 5. In another specific embodiment, the His cap domain consists of a single His. As used herein, a His cap domain does not contain (His)₆ (i.e., HHHHHH (SEQ ID NO:26)). In specific embodiments, a His cap domain does not interfere with one, two, three, or more, or all of the functions of a protein as assessed by a technique described herein or known to one of skill in the art. In specific embodiments, a His cap domain does not interfere with the structure of a protein as assessed by a technique described herein or known to one of skill in the art.

In view of the disclosure, a person of skilled in the art would understand that a “His capping means” consists of (His)ₙ (SEQ ID NO:25), wherein n is 1, 2, 3, 4, or 5. In specific embodiments, the His capping means consists of a single His. Thus, a His capping means does not contain (His)₆ (i.e., HHHHHH (SEQ ID NO:26)).

As used herein, the term “anti-drug antibody epitope” refers to a plurality of contiguous or non-contiguous amino acids that is recognized by an ADA. In certain embodiments, the anti-drug antibody epitope is a neoepitope or neoepitope-like structure in the C-terminus of a polypeptide comprising one or more target binding regions (e.g., an antibody) or other protein. In specific embodiments, the anti-drug antibody epitope is present only in a non-naturally occurring antigen binding protein (e.g., an antibody) or other protein.

As used herein, the term “reference” refers to a substance or molecule to which a polypeptide (e.g., an antibody, such as, e.g., a bispecific, trispecific or other multi-specific antibody) comprising an exposed C-terminus binds. In specific embodiments, when binding to the polypeptide, the reference contacts the exposed C-terminus. For example, in certain embodiments, the reference comprises an antigen-binding region, e.g., is an anti-drug antibody, and the reference binds an epitope of the polypeptide that comprises the exposed C-terminus.

As used herein, the term “antibody,” is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal antibodies, monovalent antibodies, multivalent antibodies, multispecific antibodies (e.g., bispecific, trispecific antibodies so long as they exhibit the desired biological activity) including—but not limited to—fusion molecules (e.g., IgG-scFv, scFv-Fc-scFv, VHH-Fc, Fc-VHH, Fc-scFv, HC-VHH, scFv-Fc-VHH, HC-scFv), single chain antibodies, and fragments thereof (e.g., domain antibodies), as described below. An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997).

Antibodies also include, but are not limited to, synthetic antibodies, nanobodies, recombinantly produced antibodies, antibodies including from Camelidae species (e.g., llama or alpaca) or their humanized variants, intrabodies, anti-idiotypic (anti-Id) antibodies, and functional fragments (e.g., antigen binding fragments) of any of the above, which refers to a portion of an antibody heavy or light chain polypeptide that retains some or all of the binding activity of the antibody from which the fragment was derived. Non-limiting examples of functional fragments (e.g., antigen binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more complementarity-determining regions (CDRs) of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

In some embodiments, the antibodies may have modifications, wherein the modifications comprise cross-linkers, glycosylation, conjugated drugs, or thio-engineered thiol-linkages.

As used herein, the term “antigen” has its ordinary meaning in the art. An “antigen” includes a structure to which an antigen binding protein (e.g., an antibody) can bind. An antigen may be a polypeptide, carbohydrate, nucleic acid, lipid, hapten, or other naturally occurring or synthetic compound. In some embodiments, the antigen is a polypeptide. In certain embodiments, an antigen is associated with a cell, for example, is present on or in a cell. In some embodiments, an antigen is associated with a cancer cell, for example, is present on or in a cancer cell. In certain embodiments, an antigen is associated with a pathogen, such as a virus, bacteria, fungus, or a parasite.

As used herein, the term “antigen binding protein” refers to a protein that binds to an antigen. An antibody is an example of an antigen binding protein. Antigen binding proteins include, but are not limited to, e.g., a single chain antibody, a nanobody, a multidomain antibody, scFv, a Fab, and a diabody.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site, such as an antigen-binding site on an antibody, and a single epitope of a target molecule, such as an antigen, is the affinity of the antibody for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and K_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

An antigen binding protein that binds to or specifically binds to an antigen can be identified, for example, by immunoassays (e.g., ELISA, radioimmunoassays, and electrochemiluminescence immunoassays), Octet®, surface plasmon resonance (e.g., BiacoreBIACore®), or other techniques known to those of skill in the art. In some embodiments, an binding protein specifically binds to an antigen when it binds to an antigen with higher affinity than to any cross-reactive antigen as determined using experimental techniques, such as radioimmunoassay (RIA) and enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of an antigen binding protein to a “non-target” protein is less than about 10% of the binding of the antigen binding protein to its particular target antigen, for example, as determined by fluorescence activated cell sorting (FACS) analysis or RIA. An antigen binding protein that binds to an antigen includes one that is capable of binding the antigen with sufficient affinity such that the antigen binding protein is useful, for example, as a therapeutic and/or diagnostic agent in targeting the antigen. In certain embodiments, an antigen binding protein that binds to an antigen has a dissociation constant (K_{D}) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antigen binding protein binds to an epitope of an antigen that is conserved among the antigen from different species.

In certain embodiments, an antigen binding protein may comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, an antigen binding protein may comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody), such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, an antigen binding protein may comprise portions of a “fully human antibody” or “human antibody,” wherein the terms are used interchangeably herein and refer to an antibody that comprises a human variable region and, for example, a human constant region. The antigen binding protein may comprise an antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1:755-68 (2006)). Also available for the preparation of human monoclonal antibodies are methods described in Cole et al., Monoclonal Antibodies and Cancer Therapy 77 (1985); Boerner et al., J. Immunol. 147 (1): 86-95 (1991); and van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6 (5): 561-66 (1995); Brüggemann and Taussing, Curr. Opin. Biotechnol. 8 (4): 455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, an antigen binding protein may comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, an antigen binding protein may comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid isomerization or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5th ed. 2001).

As used herein, the terms “Fab” and “Fab region” have their ordinary meaning in the art. Typically, the term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail in the sections below.

As used herein, the terms “variable region” and “variable domain” in the context of an antibody have their ordinary meaning in the art. Typically, the term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

As used herein, the term “heavy chain” in the context of an antibody has its ordinary meaning in the art. Typically, the term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4. The sequences of heavy chains from various species are known in the art (see, e.g., IMGT®, the international ImMunoGeneTics Information System®, imgt.org).

As used herein, the term “light chain” in the context of an antibody has its ordinary meaning in the art. Typically, the term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains. The sequences of light chains from various species are known in the art (see, e.g., IMGT®, the international ImMunoGeneTics Information System®, imgt.org). As used herein, the term “constant region” or “constant domain” in the context of an antibody has its ordinary meaning in the art. Typically, the term “constant region” or “constant domain” refers to a carboxy terminal portion of the light and heavy chain which is not directly involved in binding of the antibody to antigen but exhibits various effector function, such as interaction with the Fc receptor. Typically, the term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

As used herein, the term “framework” or “FR” in the context of an antibody has its ordinary meaning in the art. Typically, the term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

As used herein, the term “Fc region” of an antibody has its ordinary meaning in the art. Typically, the term “Fc region” is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about an 80% homology with a native sequence Fc region and/or with an Fc region of a parent polypeptide, or at least about a 90% homology therewith, for example, at least about 95% homology therewith.

In certain embodiments, homology is determined by sequence similarity. Modern protein sequence databases are very comprehensive. Widely used similarity searching programs, such as BLAST (Altschul et al. (1997); units 3.3 and 3.4), PSI-BLAST (Altschul et al., 1997), SSEARCH (Smith and Waterman (1981); Pearson (1991), unit 3.10), FASTA (Pearson and Lipman (1988) unit 3.9) and the HMMER3 (Johnson et al., 2010) programs produce accurate statistical estimates, ensuring protein sequences that share significant similarity also have similar structures.

The term “specificity” in the context of an antibody or other antibody binding protein refers to selective recognition of an antigen binding protein for a particular epitope of an antigen. Natural antibodies, for example, are monospecific. The term “multispecific” as used herein denotes that an antigen binding protein has two or more antigen-binding sites of which at least two bind different antigens. “Bispecific” as used herein denotes that an antigen binding protein has two different antigen-binding specificities. The term “monospecific” antibody as used herein denotes an antigen binding protein that has one or more binding sites each of which bind the same antigen.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide,” “nucleotide sequence,” or “nucleic acid sequence,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces an antigen binding protein of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

An “isolated” polynucleotide, nucleotide sequence, nucleic acid molecule, or nucleic acid sequence is a nucleic acid sequence, for example, an RNA sequence, DNA sequence, or a sequence containing a mixture of nucleic acids, which is substantially separated from other RNA sequences, genomic DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” polynucleotide, nucleotide sequence, nucleic acid molecule, or nucleic acid sequence, such as a cDNA molecule, can be substantially free of other cellular material, or culture medium when produced by recombinant techniques, or substantially free of chemical precursors or other chemicals when chemically synthesized. In a specific embodiment, one or more polynucleotides, nucleotide sequences, nucleic acid molecules, or nucleic acid sequences encoding an antibody or other antigen binding protein as described herein are isolated or purified. The term embraces polynucleotides, nucleotide sequences, nucleic acid molecules, and nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” polynucleotide, nucleotide sequence, nucleic acid molecule, or nucleic acid sequence encoding an antibody or antigen binding protein described herein is a nucleic acid molecule that is identified and separated from at least one contaminant nucleic acid molecule with which it is ordinarily associated in the environment in which it was produced.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “vector” refers to a substance that is used to carry or include a nucleic acid sequence, including for example, a nucleic acid encoding an antigen binding protein (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain an antigen binding protein (e.g., an antibody), for example, in isolated or purified form, together with a suitable amount of excipients.

The term “effective amount” or “therapeutically effective amount” as used herein refers to the amount of a polypeptide (e.g., an antigen binding protein, such as, e.g., antibody), agent (e.g, a therapeutic protein or other agent), or pharmaceutical composition described herein which is sufficient to result in the desired outcome.

The terms “subject” and “patient” may be used interchangeably to refer to an animal. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder. In certain embodiments, the subject is a non-human animal, such as pet or farm animal (e.g., a dog, cat, cow, pig, horse, donkey, etc.).

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Glover, ed., DNA Cloning, Volumes I and II (1985); Freshney, ed., Animal Cell Culture: Immobilized Cells and Enzymes (IRL Press, 1986); Immunochemical Methods in Cell and Molecular Biology (Academic Press, London); Scopes, Protein Purification: Principles and Practice (Springer Verlag, N.Y., 2d ed. 1987); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

In an attempt to help the reader of the application, the description has been separated in various paragraphs or sections, or, is directed to various embodiments of the application.

These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. The application contemplates use of any of the applicable components in any combination, whether or not a particular combination is expressly described.

### 7.1. Proteins

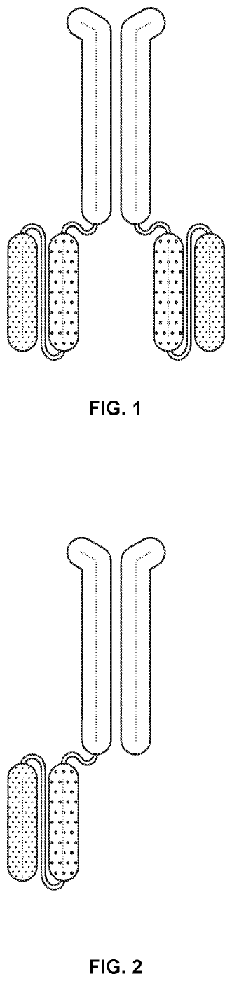

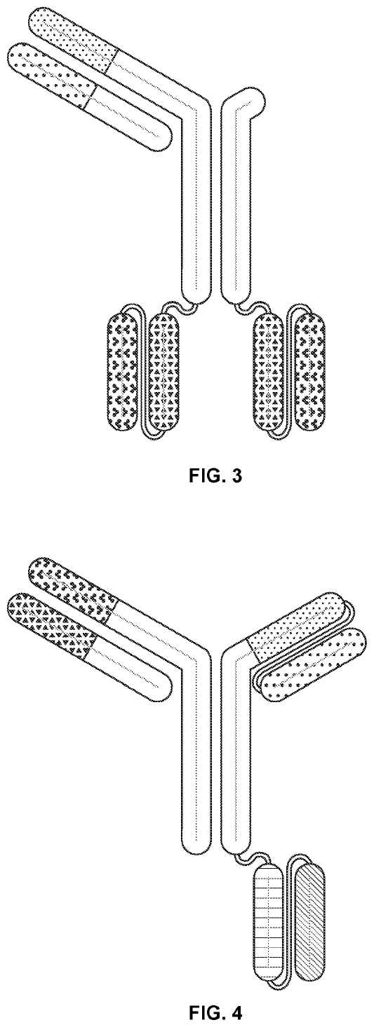

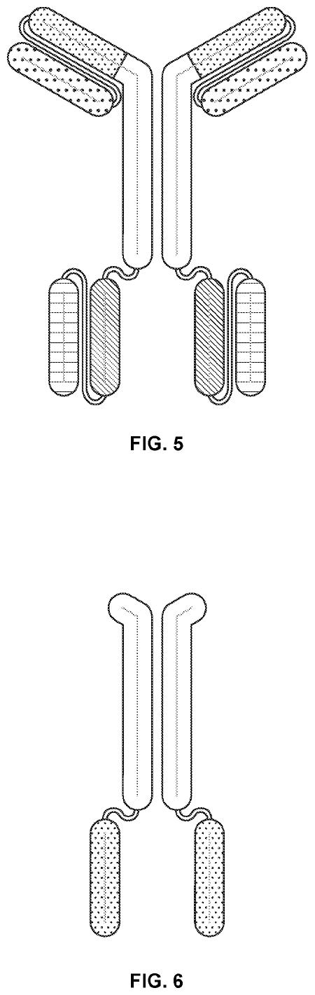

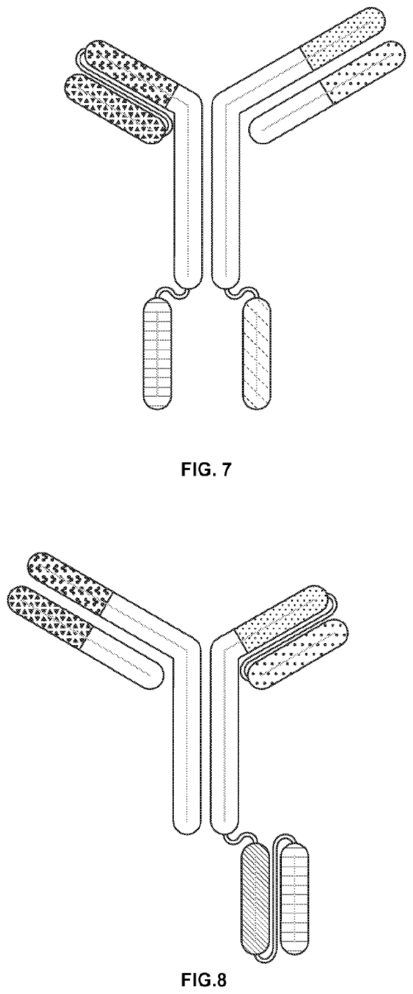

Provided herein are proteins (e.g., antibodies or other antigen binding proteins) that include an exposed C-terminus fused to a cap domain (e.g., a His cap domain, SLSLSPGK (SEQ ID NO: 36), or TVAPTESS (SEQ ID NO:37)). In specific embodiments, the proteins are therapeutic proteins. In some embodiments, the protein is an antigen binding protein. In some embodiments, the antigen binding protein is any in which there is, for example, an exposed C-terminus, e.g., the VHH or scFv/spFv on the C-terminus of the protein, such as an antibody or immunoglobulin super family domain of interest. In some embodiments, the antibody has the format as illustrated in any one of FIGS. 1-9A.

The cap domain (e.g., a His cap domain, SLSLSPGK (SEQ ID NO:36), or TVAPTESS (SEQ ID NO:37)) fused to the exposed C-terminus of a polypeptide can be achieved my any means known in the art. In some embodiments, the cap domain (e.g., a His cap domain, SLSLSPGK (SEQ ID NO:36), or TVAPTESS (SEQ ID NO:37)) is directly connected to the exposed C-terminus of a polypeptide. In some embodiments, the cap domain (e.g., a His cap domain, SLSLSPGK (SEQ ID NO:36), or TVAPTESS (SEQ ID NO:37)) is conjugated to the exposed C-terminus of a polypeptide.

In a specific aspect, provided herein is a protein comprising a cap domain, such as described in Example 8.1 and/or 8.2, infra. In specific embodiments, a capped protein provided herein has one or more of the properties of a capped tri-specific antibody described in Example 8.1 and/or 8.2, infra. In a specific embodiment, the protein is a capped tri-specific antibody described in Example 8.1 and/or 8.2, infra.

In one aspect, provided herein is a polypeptide comprising one or more target binding regions (e.g., two, three or more target binding regions) and an exposed C-terminus fused to a cap domain (e.g., a His cap domain, SLSLSPGK (SEQ ID NO:36), or TVAPTESS (SEQ ID NO: 37)). In some embodiments, the one or more target binding regions comprise an antibody variable domain (e.g., a heavy chain variable domain or a light chain variable domain) or an antigen-binding fragment thereof. In some embodiments, the cap domain consists of a His cap domain, or the amino acid sequence of SLSLSPGK (SEQ ID NO:36) or TVAPTESS (SEQ ID NO: 37). In specific embodiments, the His cap domain consists of (His)ₙ (SEQ ID NO:25), wherein n is 1, 2, 3, 4, or 5. In some embodiments, the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO:5). In some embodiments, the exposed C-terminus fused to the His cap domain consists of the amino acid sequence selected from the group consisting of: VEIKH (SEQ ID NO:6), VEIKHH (SEQ ID NO:7), VEIKHHH (SEQ ID NO: 8), VEIKHHHH (SEQ ID NO:9), and VEIKHHHHH (SEQ ID NO:10). In some embodiments, the exposed C-terminus fused to a His cap domain consists of the amino acid sequence selected from the group consisting of: VEIKRH (SEQ ID NO:11), VEIKRHH (SEQ ID NO: 12), VEIKRHHH (SEQ ID NO:13), VEIKRHHHH (SEQ ID NO:14), and VEIKRHHHHH (SEQ ID NO:15). In some embodiments, the exposed C-terminus fused to a His cap domain consists of the amino acid sequence selected from the group consisting of: VEIKRTH (SEQ ID NO: 16), VEIKRTHH (SEQ ID NO:17), VEIKRTHHH (SEQ ID NO:18), VEIKRTHHHH (SEQ ID NO: 19), and VEIKRTHHHHH (SEQ ID NO:20). In some embodiments, the exposed C-terminus fused to the His cap domain consists of the amino acid sequence of TKVTVL (His)n (SEQ ID NO:46), TKLTVL (His)n (SEQ ID NO:47), TQLIIL (His)n (SEQ ID NO:48), TELTVL (His)n (SEQ ID NO:49), or TQLTVL (His)n (SEQ ID NO:50), wherein n is 1, 2, 3, 4, or 5. In some embodiments, the exposed C-terminus fused to the His cap domain consists of the amino acid sequence of TKLTVL (His)n (SEQ ID NO:47), TKVTVL (His)n (SEQ ID NO:46), NGLVYAG (His)n (SEQ ID NO:51), NGLLYAG (His)n (SEQ ID NO:52), or TRLTVL (His)n (SEQ ID NO:53), wherein n is 1, 2, 3, 4, or 5. In a specific embodiment, the polypeptide is isolated.

In another aspect, provided herein is a protein comprising (a) a first polypeptide comprising a target binding region, and an exposed C-terminus fused to a cap domain (e.g., a His cap domain, SLSLSPGK (SEQ ID NO:36), or TVAPTESS (SEQ ID NO:37)); and (b) a second polypeptide comprising a target binding region. In some embodiments, the second polypeptide comprises an exposed C-terminus fused to a cap domain (e.g., a His cap domain, SLSLSPGK (SEQ ID NO:36), or TVAPTESS (SEQ ID NO:37)). In some embodiments, the target binding region of the first and second polypeptides are capable of binding to different targets. In certain embodiments, the protein further comprises a third polypeptide comprising a target binding region. In some embodiments, the third polypeptide comprises an exposed C-terminus fused to a cap domain (e.g., a His cap domain, SLSLSPGK (SEQ ID NO:36), or TVAPTESS (SEQ ID NO: 37)). In some embodiments, the third polypeptide is capable of binding to a different target than the first polypeptide, the second polypeptide, or both. In some embodiments, the target binding region comprises an antibody variable domain (e.g., a heavy chain variable domain or a light chain variable domain) or an antigen-binding fragment thereof. In some embodiments, the cap domain consists of a His cap domain, or the amino acid sequence of SLSLSPGK (SEQ ID NO: 36) or TVAPTESS (SEQ ID NO:37). In specific embodiments, the His cap domain consists of (His)ₙ (SEQ ID NO:25), wherein n is 1, 2, 3, 4, or 5. In some embodiments, the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO: 3), VTVSSHHHH (SEQ ID NO:4), and VTVSSHHHHH (SEQ ID NO:5). In some embodiments, the exposed C-terminus fused to the His cap domain consists of the amino acid sequence selected from the group consisting of: VEIKH (SEQ ID NO:6), VEIKHH (SEQ ID NO: 7), VEIKHHH (SEQ ID NO:8), VEIKHHHH (SEQ ID NO:9), and VEIKHHHHH (SEQ ID NO: 10). In some embodiments, the exposed C-terminus fused to a His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15). In some embodiments, the exposed C-terminus fused to a His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO:20). In some embodiments, the exposed C-terminus fused to the His cap domain consists of the amino acid sequence of TKVTVL (His)n (SEQ ID NO:46), TKLTVL (His)n (SEQ ID NO:47), TQLIIL (His)n (SEQ ID NO:48), TELTVL (His)n (SEQ ID NO:49), or TQLTVL (His)n (SEQ ID NO:50), wherein n is 1, 2, 3, 4, or 5. In some embodiments, the exposed C-terminus fused to the His cap domain consists of the amino acid sequence of TKLTVL (His)n (SEQ ID NO:47), TKVTVL (His)n (SEQ ID NO:46), NGLVYAG (His)n (SEQ ID NO:51), NGLLYAG (His)n (SEQ ID NO: 52), or TRLTVL (His)n (SEQ ID NO:53), wherein n is 1, 2, 3, 4, or 5. In some embodiments, the protein is trispecific. In some embodiments, the isolated protein is multi-specific. In a specific embodiment, the protein is isolated.

In another aspect, provided herein is a protein comprising an antigen binding region of an antibody, wherein the antigen binding region comprises an exposed C-terminus and a (His)ₙ (SEQ ID NO:25) sequence at the end of the exposed C-terminus of the antibody binding region, wherein n is 1, 2, 3, 4, or 5. In a specific embodiment, provided herein is a protein (e.g., an antigen binding protein) comprising an antibody variable domain, wherein the antibody variable domain comprises an exposed C-terminus and a (His)ₙ (SEQ ID NO:25) sequence at the end of the exposed C-terminus of the antibody variable domain, wherein n is 1, 2, 3, 4, or 5. In a specific embodiment, provided herein is a protein comprising an antibody fragment, wherein the antibody fragment comprises an exposed C-terminus and a (His)ₙ (SEQ ID NO:25) sequence at the end of the exposed C-terminus of the antibody fragment, wherein n is 1, 2, 3, 4, or 5. In some embodiments, the n of the (His)ₙ sequence is 1. In certain embodiments, the n of the (His)ₙ sequence is 2. In some embodiments, the n of the (His)ₙ sequence is 3. In certain embodiments, the n of the (His)ₙ sequence is 4 (SEQ ID NO:27). In some embodiments, the n of the (His)ₙ sequence is 5 (SEQ ID NO:28). In certain embodiments, the antibody variable domain is a heavy chain variable domain. In specific embodiments, the heavy chain variable domain is not camelized. In specific embodiments, the exposed C-terminus consists of the amino acid sequence VTVSS (SEQ ID NO:21), LTVSS (SEQ ID NO:29), or VTVSA (SEQ ID NO:30). In certain embodiments, the antibody variable domain is a light chain variable domain. In specific embodiments, the exposed C-terminus consists of the amino acid sequence VEIK (SEQ ID NO: 22), VEIKR (SEQ ID NO:23), or VEIKRT (SEQ ID NO:24). In specific embodiments, the protein is isolated.

In some embodiments, provided herein is a bispecific antibody comprising an antibody variable domain, wherein the antibody variable domain comprises an exposed C-terminus and a (His)ₙ (SEQ ID NO:25) sequence at the end of the exposed C-terminus of the antibody variable domain, wherein n is 1, 2, 3, 4, or 5. In certain embodiments, provided herein is a multi-specific antibody comprising an antibody variable domain, wherein the antibody variable domain comprises an exposed C-terminus and a (His)ₙ (SEQ ID NO:25) sequence at the end of the exposed C-terminus of the antibody variable domain, wherein n is 1, 2, 3, 4, or 5. In specific embodiments, the bispecific or multi-specific antibody is isolated. In certain embodiments, the antibody variable domain is a light chain variable domain. In other embodiments, the antibody variable domain is a heavy chain variable domain. In specific embodiments, the heavy chain variable domain is not camelized.

The C-terminal amino acid sequence of the heavy-chain immunoglobulin variable (V_{H}) domain generally has the sequence VTVSS (SEQ ID NO:21) in human, and VTVSS (SEQ ID NO: 21), LTVSS (SEQ ID NO:29), or VTVSA (SEQ ID NO:30) in mice. The C-terminal amino acid sequence of the light-chain immunoglobulin variable (V_{L}) domain generally has the sequence VEIK (SEQ ID NO:22), VEIKR (SEQ ID NO:23), or VEIKRT (SEQ ID NO:24) in humans. In the canonical immunoglobulin construct, the C-terminal amino acid of the V_{H} domain is buried by the first heavy chain constant (CH₁) domain and unexposed, and the C-terminal amino acid of the VL domain is buried by the light chain constant (C_{L}) domain and unexposed. However, antibody fragments can be engineered such that the C-terminus of either the V_{H} domain, or the V_{L} domain, or both, are no longer unexposed. This effectively results in exposure of a portion of the V_{H} domain or the V_{L} domain that would otherwise not occur naturally. Consequently, the antibody fragment with the V_{H} domain and/or the V_{L} domain that has an exposed C-terminus can trigger an ADA response.

Thus, in one aspect, provided herein is a protein (e.g., an antigen binding protein) comprising an antibody heavy chain variable domain, wherein the antibody heavy chain variable domain comprises a C-terminus amino acid sequence, wherein the last six to ten of the amino acid residues of the C-terminus amino acid sequence consists of the amino acid sequence VTVSS(H)ₙ (SEQ ID NO:31), wherein n is 1, 2, 3, 4 or 5. In some embodiments, the C-terminus amino acid sequence consists of VTVSSH (SEQ ID NO:1). In some embodiments, the C-terminus amino acid sequence consists of VTVSSHH (SEQ ID NO:2). In some embodiments, the C-terminus amino acid sequence consists of VTVSSHHH (SEQ ID NO:3). In some embodiments, the C-terminus amino acid sequence consists of VTVSSHHHH (SEQ ID NO:4). In some embodiments, the C-terminus amino acid sequence consists of VTVSSHHHHH (SEQ ID NO: 5). In specific embodiments, the protein is isolated.

In some embodiments, provided herein is a bispecific antibody comprising an antibody heavy chain variable domain, wherein the antibody heavy chain variable domain comprises a C-terminus amino acid sequence, wherein the last six to ten of the amino acid residues of the C-terminus amino acid sequence consists of the amino acid sequence VTVSS(H)n (SEQ ID NO:31), wherein n is 1, 2, 3, 4 or 5. In some embodiments, the C-terminus amino acid sequence consists of VTVSSH (SEQ ID NO:1). In some embodiments, the C-terminus amino acid sequence consists of VTVSSHH (SEQ ID NO:2). In some embodiments, the C-terminus amino acid sequence consists of VTVSSHHH (SEQ ID NO:3). In some embodiments, the C-terminus amino acid sequence consists of VTVSSHHHH (SEQ ID NO:4). In some embodiments, the C-terminus amino acid sequence consists of VTVSSHHHHH (SEQ ID NO:5). In specific embodiments, the bispecific antibody is isolated.

In certain embodiments, provided herein is a multi-specific antibody comprising an antibody heavy chain variable domain, wherein the antibody heavy chain variable domain comprises a C-terminus amino acid sequence, wherein the last six to ten of the amino acid residues of the C-terminus amino acid sequence consists of the amino acid sequence VTVSS(H)ₙ (SEQ ID NO:31), wherein n is 1, 2, 3, 4 or 5. In some embodiments, the C-terminus amino acid sequence consists of VTVSSH (SEQ ID NO:1). In some embodiments, the C-terminus amino acid sequence consists of VTVSSHH (SEQ ID NO:2). In some embodiments, the C-terminus amino acid sequence consists of VTVSSHHH (SEQ ID NO:3). In some embodiments, the C-terminus amino acid sequence consists of VTVSSHHHH (SEQ ID NO:4). In some embodiments, the C-terminus amino acid sequence consists of VTVSSHHHHH (SEQ ID NO:5). In specific embodiments, the multi-specific antibody is isolated. In certain embodiments, the multi-specific antibody is trispecific.

In another aspect, provided herein is a protein (e.g., an antigen binding protein) comprising an antibody light chain variable domain, wherein the antibody light chain variable domain comprises a C-terminus amino acid sequence, wherein the last five to eleven amino acid residues of the C-terminus amino acid sequence consists of the amino acid sequence VEIK(H)ₙ (SEQ ID NO:32), VEIKR(H)ₙ (SEQ ID NO:33), or VEIKRT(H)ₙ (SEQ ID NO:34), and wherein n is 1, 2, 3, 4 or 5. In some embodiments, the C-terminus amino acid sequence consists of VEIKH (SEQ ID NO:6). In some embodiments, the C-terminus amino acid sequence consists of VEIKHH (SEQ ID NO:7). In some embodiments, the C-terminus amino acid sequence consists of VEIKHHH (SEQ ID NO:8). In some embodiments, the C-terminus amino acid sequence consists of VEIKHHHH (SEQ ID NO:9). In some embodiments, the C-terminus amino acid sequence consists of VEIKHHHHH (SEQ ID NO:10). In some embodiments, the C-terminus amino acid sequence consists of VEIKRH (SEQ ID NO:11). In some embodiments, the C-terminus amino acid sequence consists of VEIKRHH (SEQ ID NO:12). In some embodiments, the C-terminus amino acid sequence consists of VEIKRHHH (SEQ ID NO:13). In some embodiments, the C-terminus amino acid sequence consists of VEIKRHHHH (SEQ ID NO:14). In some embodiments, the C-terminus amino acid sequence consists of VEIKRHHHHH (SEQ ID NO: 15). In some embodiments, the C-terminus amino acid sequence consists of VEIKRTH (SEQ ID NO: 16). In some embodiments, the C-terminus amino acid sequence consists of VEIKRTHH (SEQ ID NO:17). In some embodiments, the C-terminus amino acid sequence consists of VEIKRTHHH (SEQ ID NO:18). In some embodiments, the C-terminus amino acid sequence consists of VEIKRTHHHH (SEQ ID NO:19). In some embodiments, the C-terminus amino acid sequence consists of VEIKRTHHHHH (SEQ ID NO:20). In specific embodiments, the protein is isolated.

In certain embodiments, provided herein is a bispecific antibody comprising an antibody light chain variable domain, wherein the antibody light chain variable domain comprises a C-terminus amino acid sequence, wherein the last five to eleven amino acid residues of the C-terminus amino acid sequence consists of the amino acid sequence VEIK(H)ₙ (SEQ ID NO: 32), VEIKR(H)ₙ (SEQ ID NO:33), or VEIKRT(H)ₙ (SEQ ID NO:34), and wherein n is 1, 2, 3, 4 or 5. In some embodiments, the C-terminus amino acid sequence consists of VEIKH (SEQ ID NO: 6). In some embodiments, the C-terminus amino acid sequence consists of VEIKHH (SEQ ID NO:7). In some embodiments, the C-terminus amino acid sequence consists of VEIKHHH (SEQ ID NO:8). In some embodiments, the C-terminus amino acid sequence consists of VEIKHHHH (SEQ ID NO:9). In some embodiments, the C-terminus amino acid sequence consists of VEIKHHHHH (SEQ ID NO:10). In some embodiments, the C-terminus amino acid sequence consists of VEIKRH (SEQ ID NO:11). In some embodiments, the C-terminus amino acid sequence consists of VEIKRHH (SEQ ID NO:12). In some embodiments, the C-terminus amino acid sequence consists of VEIKRHHH (SEQ ID NO:13). In some embodiments, the C-terminus amino acid sequence consists of VEIKRHHHH (SEQ ID NO:14). In some embodiments, the C-terminus amino acid sequence consists of VEIKRHHHHH (SEQ ID NO: 15). In some embodiments, the C-terminus amino acid sequence consists of VEIKRTH (SEQ ID NO: 16). In some embodiments, the C-terminus amino acid sequence consists of VEIKRTHH (SEQ ID NO:17). In some embodiments, the C-terminus amino acid sequence consists of VEIKRTHHH (SEQ ID NO:18). In some embodiments, the C-terminus amino acid sequence consists of VEIKRTHHHH (SEQ ID NO:19). In some embodiments, the C-terminus amino acid sequence consists of VEIKRTHHHHH (SEQ ID NO:20). In specific embodiments, the bispecific antibody is isolated.

In some embodiments, provided herein is a multi-specific antibody (e.g., a trispecific antibody) comprising an antibody light chain variable domain, wherein the antibody light chain variable domain comprises a C-terminus amino acid sequence, wherein the last five to eleven amino acid residues of the C-terminus amino acid sequence consists of the amino acid sequence VEIK(H)ₙ (SEQ ID NO:32), VEIKR(H)ₙ (SEQ ID NO:33), or VEIKRT(H)ₙ (SEQ ID NO:34), and wherein n is 1, 2, 3, 4 or 5. In some embodiments, the C-terminus amino acid sequence consists of VEIKH (SEQ ID NO:6). In some embodiments, the C-terminus amino acid sequence consists of VEIKHH (SEQ ID NO:7). In some embodiments, the C-terminus amino acid sequence consists of VEIKHHH (SEQ ID NO:8). In some embodiments, the C-terminus amino acid sequence consists of VEIKHHHH (SEQ ID NO:9). In some embodiments, the C-terminus amino acid sequence consists of VEIKHHHHH (SEQ ID NO:10). In some embodiments, the C-terminus amino acid sequence consists of VEIKRH (SEQ ID NO:11). In some embodiments, the C-terminus amino acid sequence consists of VEIKRHH (SEQ ID NO:12). In some embodiments, the C-terminus amino acid sequence consists of VEIKRHHH (SEQ ID NO:13). In some embodiments, the C-terminus amino acid sequence consists of VEIKRHHHH (SEQ ID NO:14). In some embodiments, the C-terminus amino acid sequence consists of VEIKRHHHHH (SEQ ID NO: 15). In some embodiments, the C-terminus amino acid sequence consists of VEIKRTH (SEQ ID NO: 16). In some embodiments, the C-terminus amino acid sequence consists of VEIKRTHH (SEQ ID NO:17). In some embodiments, the C-terminus amino acid sequence consists of VEIKRTHHH (SEQ ID NO:18). In some embodiments, the C-terminus amino acid sequence consists of VEIKRTHHHH (SEQ ID NO:19). In some embodiments, the C-terminus amino acid sequence consists of VEIKRTHHHHH (SEQ ID NO:20). In specific embodiments, the multi-specific antibody is a trispecific antibody.

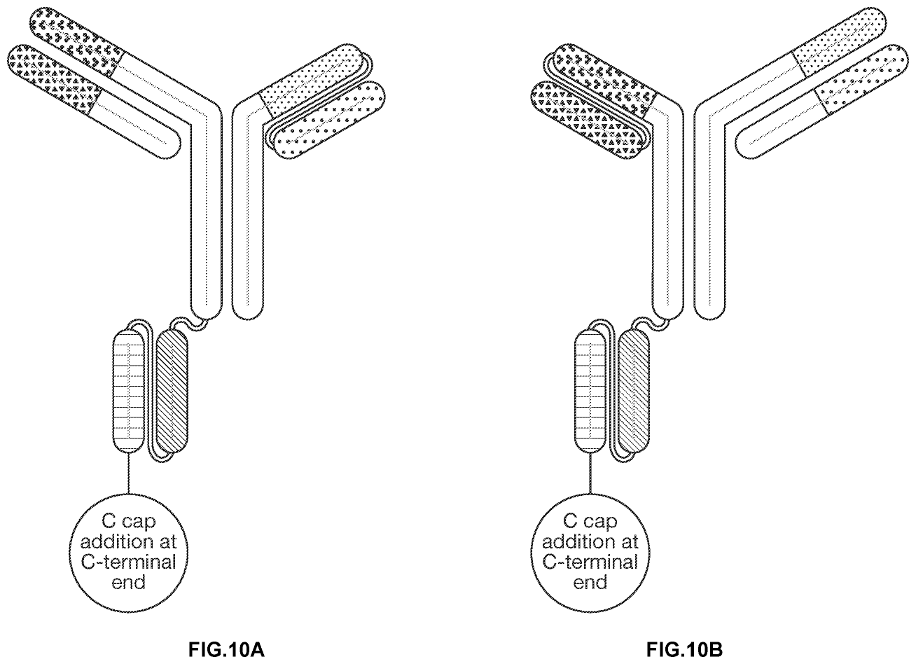

In a specific aspect, provided herein is a trispecific antibody, such as an antibody illustrated in FIGS. 10A and 10B, comprising an exposed C-terminus fused to a cap domain (e.g., a His cap domain, SLSLSPGK (SEQ ID NO:36), or TVAPTESS (SEQ ID NO:37)). For example, in some embodiments, the trispecific antibody includes an Fc-scFv fusion, and the exposed C-terminus fused to a cap domain (e.g., a His cap domain, SLSLSPGK (SEQ ID NO: 36), or TVAPTESS (SEQ ID NO:37)) is at the C-terminus of the scFv fusion. In some embodiments, the Fc-scFv fusion further comprises a second scFv with a target binding region that is different than the first sc-Fv. In some embodiments, the Fc-scFv fusion further comprises a Fab. In some embodiments, provided herein is a trispecific antibody comprising two or more exposed C-terminuses each fused to a cap domain (e.g., a His cap domain, SLSLSPGK (SEQ ID NO: 36), or TVAPTESS (SEQ ID NO:37)). In specific embodiments, the trispecific antibody includes a target binding domain that is capable of interaction with CD3 on T cells and antigens present on the surface of a cancer cell. See, e.g., Table 2 below for antigens present on cancer cells. It should be understood that the antibodies illustrated in FIGS. 10A and 10B are non-limiting and merely exemplary. In specific embodiments, the trispecific antibody is isolated.

In another aspect, provided herein is a protein (e.g., an antigen binding protein) comprising a non-naturally occurring C-terminus and a (His)ₙ (SEQ ID NO:25) sequence at the end of the non-naturally occurring C-terminus, wherein n is 1, 2, 3, 4, or 5. In a specific embodiment, provided herein is a protein comprising an antigen binding region of an antibody, wherein the antigen binding region comprises a non-naturally occurring C-terminus and a (His)ₙ (SEQ ID NO:25) sequence at the end of the non-naturally occurring C-terminus, wherein n is 1, 2, 3, 4, or 5. In a specific embodiment, provided herein is a protein comprising an antibody variable domain, wherein the antibody variable domain comprises a non-naturally occurring C-terminus and a (His)ₙ (SEQ ID NO:25) sequence at the end of the non-naturally occurring C-terminus, wherein n is 1, 2, 3, 4, or 5. In a specific embodiment, provided herein is a protein comprising an antibody fragment, wherein the antibody fragment comprises a non-naturally occurring C-terminus and a (His)ₙ (SEQ ID NO:25) sequence at the end of the non-naturally occurring C-terminus, wherein n is 1, 2, 3, 4, or 5. In some embodiments, the n of the (His)ₙ sequence is 1. In certain embodiments, the n of the (His)ₙ sequence is 2. In some embodiments, the n of the (His)ₙ sequence is 3. In certain embodiments, the n of the (His)ₙ sequence is 4 (SEQ ID NO: 27). In some embodiments, the n of the (His)ₙ sequence is 5 (SEQ ID NO:28). In specific embodiments, the protein is isolated.

In some embodiments, provided herein is a bispecific antibody comprising an antibody variable domain, wherein the antibody variable domain comprises a non-naturally occurring C-terminus and a (His)ₙ (SEQ ID NO:25) sequence at the end of the non-naturally occurring C-terminus, wherein n is 1, 2, 3, 4, or 5. In certain embodiments, provided herein is a multi-specific antibody comprising an antibody variable domain, wherein the antibody variable domain comprises a non-naturally occurring C-terminus and a (His)ₙ (SEQ ID NO:25) sequence at the end of the non-naturally occurring C-terminus, wherein n is 1, 2, 3, 4, or 5. In certain embodiments, the antibody variable domain is a light chain variable domain. In other embodiments, the antibody variable domain is a heavy chain variable domain. In specific embodiments, the heavy chain variable domain is not camelized. In specific embodiments, the bispecific or multi-specific antibody is isolated.

In certain embodiments, provided herein is a bispecific antibody comprising an antibody fragment, wherein the antibody fragment comprises a non-naturally occurring C-terminus and a (His)ₙ (SEQ ID NO:25) sequence at the end of the non-naturally occurring C-terminus, wherein n is 1, 2, 3, 4, or 5. In some embodiments, provided herein is a multi-specific antibody comprising an antibody fragment, wherein the antibody fragment comprises a non-naturally occurring C-terminus and a (His)ₙ (SEQ ID NO:25) sequence at the end of the non-naturally occurring C-terminus, wherein n is 1, 2, 3, 4, or 5. In specific embodiments, the bispecific or multi-specific antibody is isolated. In specific embodiments, the multi-specific antibody is trispecific.

In another aspect, provided herein is a protein that includes a C-terminal anti-drug antibody epitope and a (His)ₙ (SEQ ID NO:25) sequence at the end of the C-terminal anti-drug antibody epitope, wherein n is 1, 2, 3, 4, or 5. In a specific embodiment, provided herein a protein comprising a C-terminal anti-drug antibody epitope and a (His)ₙ (SEQ ID NO:25) sequence at the end of the C-terminal anti-drug antibody epitope, wherein n is 1, 2, 3, 4, or 5. In a specific embodiment, provided herein is a protein comprising an antigen binding region of an antibody, wherein the antigen binding region comprises a C-terminal anti-drug antibody epitope and a (His)ₙ (SEQ ID NO:25) sequence at the end of the C-terminal anti-drug antibody epitope, wherein n is 1, 2, 3, 4, or 5. In a specific embodiment, provided herein is a protein comprising an antibody variable region, wherein the antibody variable region comprises a C-terminal anti-drug antibody epitope and a (His)ₙ (SEQ ID NO:25) sequence at the end of the C-terminal anti-drug antibody epitope, wherein n is 1, 2, 3, 4, or 5. In a specific embodiment, provided herein is a protein comprising an antibody fragment, wherein the antibody fragment comprises a C-terminal anti-drug antibody epitope and a (His)ₙ (SEQ ID NO:25) sequence at the end of the C-terminal anti-drug antibody epitope, wherein n is 1, 2, 3, 4, or 5. In some embodiments, the n of the (His)ₙ sequence is 1. In certain embodiments, the n of the (His)ₙ sequence is 2. In some embodiments, the n of the (His)ₙ sequence is 3. In certain embodiments, the n of the (His)ₙ sequence is 4 (SEQ ID NO: 27). In some embodiments, the n of the (His)ₙ sequence is 5 (SEQ ID NO:28). In specific embodiments, the protein is isolated.

In some embodiments, provided herein is a bispecific antibody comprising an antigen binding region of an antibody, wherein the antigen binding region comprises a C-terminal anti-drug antibody epitope and a (His)ₙ (SEQ ID NO:25) sequence at the end of the C-terminal anti-drug antibody epitope, wherein n is 1, 2, 3, 4, or 5. In certain embodiments, provided herein is a multi-specific antibody (e.g., a trispecific antibody) comprising an antigen binding region of an antibody, wherein the antigen binding region comprises a C-terminal anti-drug antibody epitope and a (His)ₙ (SEQ ID NO:25) sequence at the end of the C-terminal anti-drug antibody epitope, wherein n is 1, 2, 3, 4, or 5. In specific embodiments, the bispecific or multi-specific antibody is isolated. In specific embodiments, the multi-specific antibody is trispecific.

In some embodiments, provided herein is a bispecific antibody comprising an antibody variable domain, wherein the antibody variable domain comprises a C-terminal anti-drug antibody epitope and a (His)ₙ (SEQ ID NO:25) sequence at the end of the C-terminal anti-drug antibody epitope, wherein n is 1, 2, 3, 4, or 5. In certain embodiments, provided herein is a multi-specific antibody (e.g., a trispecific antibody) comprising an antibody variable domain, wherein the antibody variable domain comprises a C-terminal anti-drug antibody epitope and a (His)ₙ (SEQ ID NO:25) sequence at the end of the C-terminal anti-drug antibody epitope, wherein n is 1, 2, 3, 4, or 5. In certain embodiments, the antibody variable domain is a light chain variable domain. In other embodiments, the antibody variable domain is a heavy chain variable domain. In specific embodiments, the heavy chain variable domain is not camelized. In specific embodiments, the bispecific or multi-specific antibody is isolated. In specific embodiments, the multi-specific antibody is trispecific.

In some embodiments, provided herein is a bispecific antibody comprising an antibody fragment, wherein the antibody fragment comprises a C-terminal anti-drug antibody epitope and a (His)ₙ (SEQ ID NO:25) sequence at the end of the C-terminal anti-drug antibody epitope, wherein n is 1, 2, 3, 4, or 5. In certain embodiments, provided herein is a multi-specific antibody (e.g., trispecific) comprising an antibody fragment, wherein the antibody fragment comprises a C-terminal anti-drug antibody epitope and a (His)ₙ (SEQ ID NO:25) sequence at the end of the C-terminal anti-drug antibody epitope, wherein n is 1, 2, 3, 4, or 5. In specific embodiments, the bispecific or multi-specific antibody is isolated. In specific embodiments, the multi-specific antibody is trispecific.

In another aspect, provided herein is a protein (e.g., a fusion protein or an antigen binding protein) that comprises an antibody fragment, wherein the antibody fragment comprises an exposed C-terminus, and a (His)ₙ (SEQ ID NO:25) sequence at the end of the C-terminus, wherein n is 1, 2, 3, 4, or 5. In some embodiments, the n of the (His)ₙ sequence is 1. In certain embodiments, the n of the (His)ₙ sequence is 2. In some embodiments, the n of the (His)ₙ sequence is 3. In certain embodiments, the n of the (His)ₙ sequence is 4 (SEQ ID NO:27). In some embodiments, the n of the (His)ₙ sequence is 5 (SEQ ID NO:28). In specific embodiments, the protein is isolated.

Without being bound by any theory, in specific embodiments, the addition of a cap domain (e.g., a His cap domain, SLSLSPGK (SEQ ID NO:36), or TVAPTESS (SEQ ID NO:37)) or (His)ₙ sequence to the end of an exposed C-terminus, the end of a non-naturally occurring C-terminus, or the end of a C-terminal anti-drug antibody epitope of a polypeptide or protein, alters the exposed C-terminus, the non-naturally occurring C-terminus, or the C-terminal anti-drug antibody epitope so that the polypeptide or protein no longer interacts with a reference, or the interaction with the reference is reduced. In some embodiments, the reference is a molecule present in a biological substance. Accordingly, in some embodiments, the protein having an exposed C-terminus fused to a cap domain (e.g., a His cap domain, SLSLSPGK (SEQ ID NO: 36), or TVAPTESS (SEQ ID NO:37)) exhibits reduced interaction with a molecule present in a biological substance. In some embodiments, the biological substance comprises serum and/or plasma (e.g., human serum and/or human plasma). In specific embodiments, the biological substance comprises serum. In specific embodiments, the biological substance comprises plasma. In specific embodiments, the interaction is determined by an aggregation assay described herein (such as an assay described in Section 7.9.1) or known to one of skill in the art.

In some embodiments, the addition of a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in a reduction in the interaction of the polypeptide or the protein with a reference relative to the interaction of the same reference with the same polypeptide or the same protein, respectively, lacking the cap domain. In some embodiments, the reduction is at least 10%, at least 20%, at least 30%, at least 40%, or at least 50%. In some embodiments, the reduction is at least 60%, at least 70%, at least 80%, at least 90%, or at least 95%. In specific embodiments, the interaction is determined by an immunoassay described herein (such as an assay described in Section 7.9.1) or known to one of skill in the art.

In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in a reduction in the interaction of the polypeptide or the protein with a reference relative to the interaction of the same reference with the same polypeptide or the same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 10% reduction in the interaction of the polypeptide or the protein with a reference, relative to the interaction of the same reference with the same polypeptide or the same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 20% reduction in the interaction of the polypeptide or the protein with a reference, relative to the interaction of the same reference with the same polypeptide or the same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 30% reduction in the interaction of the polypeptide or the protein with a reference, relative to the interaction of the same reference with the same polypeptide or the same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 40% reduction in the interaction of the polypeptide or the protein with a reference, relative to the interaction of the same reference with the same polypeptide or the same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 50% reduction in the interaction of the polypeptide or the protein with a reference, relative to the interaction of the same reference with the same polypeptide or the same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 60% reduction in the interaction of the polypeptide or the protein with a reference, relative to the interaction of the same reference with the same polypeptide or the same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 70% reduction in the interaction of the polypeptide or the protein with a reference, relative to the interaction of the same reference with the same polypeptide or the same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 80% reduction in the interaction of the polypeptide or the protein with a reference, relative to the interaction of the same reference with the same polypeptide or the same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 90% reduction in the interaction of the polypeptide or the protein with a reference, relative to the interaction of the same reference with the same polypeptide or the same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 95% reduction in the interaction of the polypeptide or the protein with a reference, relative to the interaction of the same reference with the same polypeptide or the same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or protein (e.g., an antibody such as e.g., a bispecific antibody, or a multispecific antibody (e.g., trispecific antibody)) eliminates the interaction of the polypeptide or the protein with a reference. In specific embodiments, the interaction is determined by an immunoassay described herein (such as an assay described in Section 7.9.1) or known to one of skill in the art.

In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in a reduction in the interaction of the polypeptide or the protein with a reference relative to the interaction of the same reference with the same polypeptide or the same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 10% reduction in the interaction of the polypeptide or the protein with a reference, relative to the interaction of the same reference with the same polypeptide or the same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 20% reduction in the interaction of the polypeptide or the protein with a reference, relative to the interaction of the same reference with the same polypeptide or the same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 30% reduction in the interaction of the polypeptide or the protein with a reference, relative to the interaction of the same reference with the same polypeptide or the same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 40% reduction in the interaction of the polypeptide or the protein with a reference, relative to the interaction of the same reference with the same polypeptide or the same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 50% reduction in the interaction of the polypeptide or the protein with a reference, relative to the interaction of the same reference with the same polypeptide or the same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 60% reduction in the interaction of the polypeptide or the protein with a reference, relative to the interaction of the same reference with the same polypeptide or the same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 70% reduction in the interaction of the polypeptide or the protein with a reference, relative to the interaction of the same reference with the same polypeptide or the same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 80% reduction in the interaction of the polypeptide or the protein with a reference, relative to the interaction of the same reference with the same polypeptide or the same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 90% reduction in the interaction of the polypeptide or the protein with a reference, relative to the interaction of the same reference with the same polypeptide or the same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 95% reduction in the interaction of the polypeptide or the protein with a reference, relative to the interaction of the same reference with the same polypeptide or the same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or protein (e.g., an antibody such as e.g., a bispecific antibody, or a multispecific antibody (e.g., trispecific antibody)) eliminates the interaction of the polypeptide or the protein with a reference. In specific embodiments, the interaction is determined by an immunoassay described herein (such as an assay described in Section 7.9.1) or known to one of skill in the art.

In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in a reduction in the interaction of the polypeptide or the protein with a reference relative to the interaction of the same reference with the same polypeptide or the same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 10% reduction in the interaction of the polypeptide or the protein with a reference, relative to the interaction of the same reference with the same polypeptide or the same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 20% reduction in the interaction of the polypeptide or the protein with a reference, relative to the interaction of the same reference with the same polypeptide or the same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 30% reduction in the interaction of the polypeptide or the protein with a reference, relative to the interaction of the same reference with the same polypeptide or the same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 40% reduction in the interaction of the polypeptide or the protein with a reference, relative to the interaction of the same reference with the same polypeptide or the same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 50% reduction in the interaction of the polypeptide or the protein with a reference, relative to the interaction of the same reference with the same polypeptide or the same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 60% reduction in the interaction of the polypeptide or the protein with a reference, relative to the interaction of the same reference with the same polypeptide or the same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 70% reduction in the interaction of the polypeptide or the protein with a reference, relative to the interaction of the same reference with the same polypeptide or the same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 80% reduction in the interaction of the polypeptide or the protein with a reference, relative to the interaction of the same reference with the same polypeptide or the same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 90% reduction in the interaction of the polypeptide or the protein with a reference, relative to the interaction of the same reference with the same polypeptide or the same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 95% reduction in the interaction of the polypeptide or the protein with a reference, relative to the interaction of the same reference with the same polypeptide or the same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or protein (e.g., an antibody such as e.g., a bispecific antibody, or a multispecific antibody (e.g., trispecific antibody)) eliminates the interaction of the polypeptide or the protein with a reference. In specific embodiments, the interaction is (such as an assay described in Section 7.9.1) or known to one of skill in the art.

In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in a reduction in the interaction of the polypeptide or the protein with a reference relative to the interaction of the same reference with the same polypeptide or the same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 10% reduction in the interaction of the polypeptide or the protein with a reference, relative to the interaction of the same reference with the same polypeptide or the same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 20% reduction in the interaction of the polypeptide or the protein with a reference, relative to the interaction of the same reference with the same polypeptide or the same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 30% reduction in the interaction of the polypeptide or the protein with a reference, relative to the interaction of the same reference with the same polypeptide or the same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 40% reduction in the interaction of the polypeptide or the protein with a reference, relative to the interaction of the same reference with the same polypeptide or the same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 50% reduction in the interaction of the polypeptide or the protein with a reference, relative to the interaction of the same reference with the same polypeptide or the same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 60% reduction in the interaction of the polypeptide or the protein with a reference, relative to the interaction of the same reference with the same polypeptide or the same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 70% reduction in the interaction of the polypeptide or the protein with a reference, relative to the interaction of the same reference with the same polypeptide or the same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 80% reduction in the interaction of the polypeptide or the protein with a reference, relative to the interaction of the same reference with the same polypeptide or the same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 90% reduction in the interaction of the polypeptide or the protein with a reference, relative to the interaction of the same reference with the same polypeptide or the same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 95% reduction in the interaction of the polypeptide or the protein with a reference, relative to the interaction of the same reference with the same polypeptide or the same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or protein (e.g., an antibody such as e.g., a bispecific antibody, or a multispecific antibody (e.g., trispecific antibody)) eliminates the interaction of the polypeptide or the protein with a reference. In specific embodiments, the interaction is (such as an assay described in Section 7.9.1) or known to one of skill in the art.

In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in a reduction in the interaction of the polypeptide or the protein with a reference relative to the interaction of the same reference with the same polypeptide or the same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 10% reduction in the interaction of the polypeptide or the protein with a reference, relative to the interaction of the same reference with the same polypeptide or the same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 20% reduction in the interaction of the polypeptide or the protein with a reference, relative to the interaction of the same reference with the same polypeptide or the same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 30% reduction in the interaction of the polypeptide or the protein with a reference, relative to the interaction of the same reference with the same polypeptide or the same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 40% reduction in the interaction of the polypeptide or the protein with a reference, relative to the interaction of the same reference with the same polypeptide or the same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 50% reduction in the interaction of the polypeptide or the protein with a reference, relative to the interaction of the same reference with the same polypeptide or the same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 60% reduction in the interaction of the polypeptide or the protein with a reference, relative to the interaction of the same reference with the same polypeptide or the same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 70% reduction in the interaction of the polypeptide or the protein with a reference, relative to the interaction of the same reference with the same polypeptide or the same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 80% reduction in the interaction of the polypeptide or the protein with a reference, relative to the interaction of the same reference with the same polypeptide or the same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 90% reduction in the interaction of the polypeptide or the protein with a reference, relative to the interaction of the same reference with the same polypeptide or the same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or protein (e.g., an antibody, such as, e.g., a bispecific antibody, or a multispecific antibody (e.g., trispecific antibody)) results in at least about a 95% reduction in the interaction of the polypeptide or the protein with a reference, relative to the interaction of the same reference with the same polypeptide or the same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or protein (e.g., an antibody such as e.g., a bispecific antibody, or a multispecific antibody (e.g., trispecific antibody)) eliminates the interaction of the polypeptide or the protein with a reference. In specific embodiments, the interaction is (such as an assay described in Section 7.9.1) or known to one of skill in the art.

In some embodiments, the reference comprises the reference is a molecule present in a biological substance. In some embodiments, the biological substance comprises serum. In some embodiments, the biological substance comprises plasma. In some embodiments, the biological substance comprises serum and/or plasma (e.g., human serum and/or human plasma). In some embodiments, the reference comprises an anti-drug antibody. In some embodiments, the anti-drug antibody is pre-existing in the serum and/or plasma of a subject (e.g., a human subject).

In some embodiments, the addition of a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a protein (e.g., an antigen binding protein) results in a reduction in the interaction of the protein with an ADA relative to the interaction of the same protein lacking the cap domain with the ADA. In some embodiments, the reduction is at least 10%, at least 20%, at least 30%, at least 40%, or at least 50%. In some embodiments, the reduction is at least 60%, at least 70%, at least 80%, at least 90%, or at least 95%.

In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in a reduction in the interaction of the protein with an ADA relative to the interaction of the same protein lacking the (His)ₙ (SEQ ID NO:25) sequence with the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 10% reduction in the interaction of the protein with an ADA, relative to the interaction of the same protein lacking the (His)ₙ (SEQ ID NO:25) sequence with the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 20% reduction in the interaction of the protein with an ADA, relative to the interaction of the same protein lacking the (His)ₙ (SEQ ID NO:25) sequence with the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 30% reduction in the interaction of the protein with an ADA, relative to the interaction of the same protein lacking the (His)ₙ (SEQ ID NO:25) sequence with the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 40% reduction in the interaction of the protein with an ADA, relative to the interaction of the same protein lacking the (His)ₙ (SEQ ID NO:25) sequence with the ADA. In some embodiments, the addition of a (His)ₙ sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 50% reduction in the interaction of the protein with an ADA, relative to the interaction of the same protein lacking the (His)ₙ (SEQ ID NO:25) sequence with the ADA. In some embodiments, the addition of a (His)ₙ (SEQ ID NO:25) sequence at the end of the exposed C-terminus, the end of the non-naturally occurring C-terminus, or the end of the C-terminal anti-drug antibody epitope of a therapeutic protein (e.g., an antigen binding protein), wherein n is 1, 2, 3, 4, or 5, results in at least about a 60% reduction in the interaction of the protein with an ADA, relative to the interaction of the same protein lacking the (His)ₙ (SEQ ID NO: 25) sequence with the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 70% reduction in the interaction of the protein with an ADA, relative to the interaction of the same protein lacking the (His)ₙ (SEQ ID NO: 25) sequence with the ADA. In some embodiments, the addition of a (His)ₙ (SEQ ID NO: 25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about an 80% reduction in the interaction of the protein with an ADA, relative to the interaction of the same protein lacking the (His)ₙ (SEQ ID NO:25) sequence with the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 90% reduction in the interaction of the protein with an ADA, relative to the interaction of the same protein lacking the (His)ₙ (SEQ ID NO:25) sequence with the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 95% reduction in the interaction of the protein with an ADA, relative to the interaction of the same protein lacking the (His)ₙ (SEQ ID NO:25) sequence with the ADA. Various assays known to one of skill in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), electrochemiluminescence (ECL)-based assay or other immunoassay described herein or known to one of skill in the art.

In specific embodiments, the addition of a single histidine at the end of an exposed C-terminus, the end of a non-naturally occurring C-terminus, or the end of a C-terminal anti-drug antibody epitope of a protein (e.g., an antigen binding protein) results in a reduction in the interaction of the protein with an ADA relative to the interaction of the same protein lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antigen binding protein) results in at least about a 10% reduction in the interaction of the protein with an ADA, relative to the interaction of the same protein lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antigen binding protein) results in at least about a 20% reduction in the interaction of the protein with an ADA, relative to the interaction of the same protein lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antigen binding protein) results in at least about a 30% reduction in the interaction of the protein with an ADA, relative to the interaction of the same protein lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antigen binding protein) results in at least about a 40% reduction in the interaction of the protein with an ADA, relative to the interaction of the same protein lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antigen binding protein) results in at least about a 50% reduction in the interaction of the protein with an ADA, relative to the interaction of the same protein lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antigen binding protein) results in at least about a 60% reduction in the interaction of the protein with an ADA, relative to the interaction of the same protein lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antigen binding protein) results in at least about a 70% reduction in the interaction of the protein with an ADA, relative to the interaction of the same protein lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antigen binding protein) results in at least about an 80% reduction in the interaction of the protein with an ADA, relative to the interaction of the same protein lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antigen binding protein) results in at least about a 90% reduction in the interaction of the protein with an ADA, relative to the interaction of the same protein lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antigen binding protein) results in at least about a 95% reduction in the interaction of the protein with an ADA, relative to the interaction of the same protein lacking the single histidine with the ADA. In particular embodiments, the addition of a single histidine at the end of an exposed C-terminus, the end of a non-naturally occurring C-terminus, or the end of a C-terminal anti-drug antibody epitope of a protein (e.g., an antigen binding protein) eliminates interaction of the protein with an ADA. Various assays known to one of skill in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In specific embodiments, the addition of two histidines at the end of an exposed C-terminus, the end of a non-naturally occurring C-terminus, or the end of a C-terminal anti-drug antibody epitope of a protein (e.g., an antigen binding protein) results in a reduction in the interaction of the protein with an ADA relative to the interaction of the same protein lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 10% reduction in the interaction of the protein with an ADA, relative to the interaction of the same protein lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 20% reduction in the interaction of the protein with an ADA, relative to the interaction of the same protein lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 30% reduction in the interaction of the protein with an ADA, relative to the interaction of the same protein lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 40% reduction in the interaction of the protein with an ADA, relative to the interaction of the same protein lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 50% reduction in the interaction of the protein with an ADA, relative to the interaction of the same protein lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 60% reduction in the interaction of the protein with an ADA, relative to the interaction of the same protein lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 70% reduction in the interaction of the protein with an ADA, relative to the interaction of the same protein lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about an 80% reduction in the interaction of the protein with an ADA, relative to the interaction of the same protein lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 90% reduction in the interaction of the protein with an ADA, relative to the interaction of the same protein lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 95% reduction in the interaction of the protein with an ADA, relative to the interaction of the same protein lacking the two histidines with the ADA. In particular embodiments, the addition of two histidines at the end of an exposed C-terminus, the end of a non-naturally occurring C-terminus, or the end of a C-terminal anti-drug antibody epitope of a protein (e.g., an antigen binding protein) eliminates interaction of the protein with an ADA. Various assays known to one of skill in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In specific embodiments, the addition of three histidines at the end of an exposed C-terminus, the end of a non-naturally occurring C-terminus, or the end of a C-terminal anti-drug antibody epitope of a protein (e.g., an antigen binding protein) results in a reduction in the interaction of the protein with an ADA relative to the interaction of the same protein lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 10% reduction in the interaction of the protein with an ADA, relative to the interaction of the same protein lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 20% reduction in the interaction of the protein with an ADA, relative to the interaction of the same protein lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 30% reduction in the interaction of the protein with an ADA, relative to the interaction of the same protein lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 40% reduction in the interaction of the protein with an ADA, relative to the interaction of the same protein lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 50% reduction in the interaction of the protein with an ADA, relative to the interaction of the same protein lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 60% reduction in the interaction of the protein with an ADA, relative to the interaction of the same protein lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 70% reduction in the interaction of the protein with an ADA, relative to the interaction of the same protein lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about an 80% reduction in the interaction of the protein with an ADA, relative to the interaction of the same protein lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 90% reduction in the interaction of the protein with an ADA, relative to the interaction of the same protein lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 95% reduction in the interaction of the protein with an ADA, relative to the interaction of the same protein lacking the three histidines with the ADA. In particular embodiments, the addition of three histidines at the end of an exposed C-terminus, the end of a non-naturally occurring C-terminus, or the end of a C-terminal anti-drug antibody epitope of a protein (e.g., an antigen binding protein) eliminates interaction of the protein with an ADA. Various assays known to one of skill in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In specific embodiments, the addition of four histidines at the end of an exposed C-terminus, the end of a non-naturally occurring C-terminus, or the end of a C-terminal anti-drug antibody epitope of a protein (e.g., an antigen binding protein) results in a reduction in the interaction of the protein with an ADA relative to the interaction of the same protein lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 10% reduction in the interaction of the protein with an ADA, relative to the interaction of the same protein lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 20% reduction in the interaction of the protein with an ADA, relative to the interaction of the same protein lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 30% reduction in the interaction of the protein with an ADA, relative to the interaction of the same protein lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 40% reduction in the interaction of the protein with an ADA, relative to the interaction of the same protein lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 50% reduction in the interaction of the protein with an ADA, relative to the interaction of the same protein lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 60% reduction in the interaction of the protein with an ADA, relative to the interaction of the same protein lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 70% reduction in the interaction of the protein with an ADA, relative to the interaction of the same protein lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about an 80% reduction in the interaction of the protein with an ADA, relative to the interaction of the same protein lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 90% reduction in the interaction of the protein with an ADA, relative to the interaction of the same protein lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 95% reduction in the interaction of the protein with an ADA, relative to the interaction of the same protein lacking the four histidines with the ADA. In particular embodiments, the addition of four histidines at the end of an exposed C-terminus, the end of a non-naturally occurring C-terminus, or the end of a C-terminal anti-drug antibody epitope of a protein (e.g., an antigen binding protein) eliminates interaction of the protein with an ADA. Various assays known to one of skill in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In specific embodiments, the addition of five histidines at the end of an exposed C-terminus, the end of a non-naturally occurring C-terminus, or the end of a C-terminal anti-drug antibody epitope of a protein (e.g., an antigen binding protein) results in a reduction in the interaction of the protein with an ADA relative to the interaction of the same protein lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 10% reduction in the interaction of the protein with an ADA, relative to the interaction of the same protein lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 20% reduction in the interaction of the protein with an ADA, relative to the interaction of the same protein lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 30% reduction in the interaction of the protein with an ADA, relative to the interaction of the same protein lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 40% reduction in the interaction of the protein with an ADA, relative to the interaction of the same protein lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 50% reduction in the interaction of the protein with an ADA, relative to the interaction of the same protein lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 60% reduction in the interaction of the protein with an ADA, relative to the interaction of the same protein lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 70% reduction in the interaction of the protein with an ADA, relative to the interaction of the same protein lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about an 80% reduction in the interaction of the protein with an ADA, relative to the interaction of the same protein lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 90% reduction in the interaction of the protein with an ADA, relative to the interaction of the same protein lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 95% reduction in the interaction of the protein with an ADA, relative to the interaction of the same protein lacking the five histidines with the ADA. In particular embodiments, the addition of five histidines at the end of an exposed C-terminus, the end of a non-naturally occurring C-terminus, or the end of a C-terminal anti-drug antibody epitope of a protein (e.g., an antigen binding protein) eliminates interaction of the protein with an ADA. Various assays known to one of skill in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in reduced aggregation upon incubation with a biological substance relative to the aggregation exhibited upon incubation of the same polypeptide or same protein, respectively, lacking the cap domain (e.g., a His cap domain, SLSLSPGK (SEQ ID NO:36), or TVAPTESS (SEQ ID NO:37)) with the biological substance. In some embodiments, the addition a cap domain (e.g., a His cap domain, SLSLSPGK (SEQ ID NO: 36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 10% reduction in aggregation upon incubation with a biological substance relative to the aggregation exhibited upon incubation of the same polypeptide or same protein, respectively, lacking the cap domain (e.g., a His cap domain, SLSLSPGK (SEQ ID NO:36), or TVAPTESS (SEQ ID NO:37)) with the biological substance. In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 20% reduction in aggregation upon incubation with a biological substance relative to the aggregation exhibited upon incubation of the same polypeptide or same protein, respectively, lacking the cap domain (e.g., a His cap domain, SLSLSPGK (SEQ ID NO:36), or TVAPTESS (SEQ ID NO:37)) with the biological substance. In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 30% reduction in aggregation upon incubation with a biological substance relative to the aggregation exhibited upon incubation of the same polypeptide or same protein, respectively, lacking the cap domain (e.g., a His cap domain, SLSLSPGK (SEQ ID NO:36), or TVAPTESS (SEQ ID NO:37)) with the biological substance. In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 40% reduction in aggregation upon incubation with a biological substance relative to the aggregation exhibited upon incubation of the same polypeptide or same protein, respectively, lacking the cap domain (e.g., a His cap domain, SLSLSPGK (SEQ ID NO:36), or TVAPTESS (SEQ ID NO:37)) with the biological substance. In some embodiments, the addition a cap domain (e.g., a His cap domain, SLSLSPGK (SEQ ID NO:36), or TVAPTESS (SEQ ID NO: 37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 50% reduction in aggregation upon incubation with a biological substance relative to the aggregation exhibited upon incubation of the same polypeptide or same protein, respectively, lacking the cap domain (e.g., a His cap domain, SLSLSPGK (SEQ ID NO:36), or TVAPTESS (SEQ ID NO:37)) with the biological substance. In some embodiments, the addition a cap domain (e.g., a His cap domain, SLSLSPGK (SEQ ID NO: 36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 60% reduction in aggregation upon incubation with a biological substance relative to the aggregation exhibited upon incubation of the same polypeptide or same protein, respectively, lacking the cap domain (e.g., a His cap domain, SLSLSPGK (SEQ ID NO:36), or TVAPTESS (SEQ ID NO:37)) with the biological substance. In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 70% reduction in aggregation upon incubation with a biological substance relative to the aggregation exhibited upon incubation of the same polypeptide or same protein, respectively, lacking the cap domain (e.g., a His cap domain, SLSLSPGK (SEQ ID NO:36), or TVAPTESS (SEQ ID NO:37)) with the biological substance. In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 80% reduction in aggregation upon incubation with a biological substance relative to the aggregation exhibited upon incubation of the same polypeptide or same protein, respectively, lacking the cap domain (e.g., a His cap domain, SLSLSPGK (SEQ ID NO:36), or TVAPTESS (SEQ ID NO:37)) with the biological substance. In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 90% reduction in aggregation upon incubation with a biological substance relative to the aggregation exhibited upon incubation of the same polypeptide or same protein, respectively, lacking the cap domain (e.g., a His cap domain, SLSLSPGK (SEQ ID NO:36), or TVAPTESS (SEQ ID NO:37)) with the biological substance. In some embodiments, the addition a cap domain (e.g., a His cap domain, SLSLSPGK (SEQ ID NO:36), or TVAPTESS (SEQ ID NO: 37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 95% reduction in aggregation upon incubation with a biological substance relative to the aggregation exhibited upon incubation of the same polypeptide or same protein, respectively, lacking the cap domain (e.g., a His cap domain, SLSLSPGK (SEQ ID NO:36), or TVAPTESS (SEQ ID NO:37)) with the biological substance. In specific embodiments, the aggregation is determined by an aggregation assay described herein (such as an assay described in Section 7.9.7) or known to one of skill in the art.

In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in reduced aggregation upon incubation with a biological substance relative to the aggregation exhibited upon incubation of the same polypeptide or same protein, respectively, lacking the one histidine with the biological substance. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 10% reduction in aggregation upon incubation with a biological substance relative to the aggregation exhibited upon incubation of the same polypeptide or same protein, respectively, lacking the one histidine with the biological substance. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 20% reduction in aggregation upon incubation with a biological substance relative to the aggregation exhibited upon incubation of the same polypeptide or same protein, respectively, lacking the one histidine with the biological substance. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 30% reduction in aggregation upon incubation with a biological substance relative to the aggregation exhibited upon incubation of the same polypeptide or same protein, respectively, lacking the one histidine with the biological substance. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 40% reduction in aggregation upon incubation with a biological substance relative to the aggregation exhibited upon incubation of the same polypeptide or same protein, respectively, lacking the one histidine with the biological substance. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 50% reduction in aggregation upon incubation with a biological substance relative to the aggregation exhibited upon incubation of the same polypeptide or same protein, respectively, lacking the one histidine with the biological substance. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 60% reduction in aggregation upon incubation with a biological substance relative to the aggregation exhibited upon incubation of the same polypeptide or same protein, respectively, lacking the one histidine with the biological substance. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 70% reduction in aggregation upon incubation with a biological substance relative to the aggregation exhibited upon incubation of the same polypeptide or same protein, respectively, lacking the one histidine with the biological substance. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 80% reduction in aggregation upon incubation with a biological substance relative to the aggregation exhibited upon incubation of the same polypeptide or same protein, respectively, lacking the one histidine with the biological substance. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 90% reduction in aggregation upon incubation with a biological substance relative to the aggregation exhibited upon incubation of the same polypeptide or same protein, respectively, lacking the one histidine with the biological substance. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 95% reduction in aggregation upon incubation with a biological substance relative to the aggregation exhibited upon incubation of the same polypeptide or same protein, respectively, lacking the one histidine with the biological substance. In specific embodiments, the aggregation is determined by an aggregation assay described herein (such as an assay described in Section 7.9.7) or known to one of skill in the art. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in reduced aggregation upon incubation with a biological substance relative to the aggregation exhibited upon incubation of the same polypeptide or same protein, respectively, lacking the two histidines with the biological substance. In specific embodiments, the aggregation is determined by an aggregation assay described herein (such as an assay described in Section 7.9.7) or known to one of skill in the art.

In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 10% reduction in aggregation upon incubation with a biological substance relative to the aggregation exhibited upon incubation of the same polypeptide or same protein, respectively, lacking the two histidines with the biological substance. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 20% reduction in aggregation upon incubation with a biological substance relative to the aggregation exhibited upon incubation of the same polypeptide or same protein, respectively, lacking the two histidines with the biological substance. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 30% reduction in aggregation upon incubation with a biological substance relative to the aggregation exhibited upon incubation of the same polypeptide or same protein, respectively, lacking the two histidines with the biological substance. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 40% reduction in aggregation upon incubation with a biological substance relative to the aggregation exhibited upon incubation of the same polypeptide or same protein, respectively, lacking the two histidines with the biological substance. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 50% reduction in aggregation upon incubation with a biological substance relative to the aggregation exhibited upon incubation of the same polypeptide or same protein, respectively, lacking the two histidines with the biological substance. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 60% reduction in aggregation upon incubation with a biological substance relative to the aggregation exhibited upon incubation of the same polypeptide or same protein, respectively, lacking the two histidines with the biological substance. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 70% reduction in aggregation upon incubation with a biological substance relative to the aggregation exhibited upon incubation of the same polypeptide or same protein, respectively, lacking the two histidines with the biological substance. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 80% reduction in aggregation upon incubation with a biological substance relative to the aggregation exhibited upon incubation of the same polypeptide or same protein, respectively, lacking the two histidines with the biological substance. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 90% reduction in aggregation upon incubation with a biological substance relative to the aggregation exhibited upon incubation of the same polypeptide or same protein, respectively, lacking the two histidines with the biological substance. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 95% reduction in aggregation upon incubation with a biological substance relative to the aggregation exhibited upon incubation of the same polypeptide or same protein, respectively, lacking the two histidines with the biological substance. In specific embodiments, the aggregation is determined by an aggregation assay described herein (such as an assay described in Section 7.9.7) or known to one of skill in the art.

In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in reduced aggregation upon incubation with a biological substance relative to the aggregation exhibited upon incubation of the same polypeptide or same protein, respectively, lacking the three histidines with the biological substance. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 10% reduction in aggregation upon incubation with a biological substance relative to the aggregation exhibited upon incubation of the same polypeptide or same protein, respectively, lacking the three histidines with the biological substance. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 20% reduction in aggregation upon incubation with a biological substance relative to the aggregation exhibited upon incubation of the same polypeptide or same protein, respectively, lacking the three histidines with the biological substance. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 30% reduction in aggregation upon incubation with a biological substance relative to the aggregation exhibited upon incubation of the same polypeptide or same protein, respectively, lacking the three histidines with the biological substance. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 40% reduction in aggregation upon incubation with a biological substance relative to the aggregation exhibited upon incubation of the same polypeptide or same protein, respectively, lacking the three histidines with the biological substance. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 50% reduction in aggregation upon incubation with a biological substance relative to the aggregation exhibited upon incubation of the same polypeptide or same protein, respectively, lacking the three histidines with the biological substance. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 60% reduction in aggregation upon incubation with a biological substance relative to the aggregation exhibited upon incubation of the same polypeptide or same protein, respectively, lacking the three histidines with the biological substance. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 70% reduction in aggregation upon incubation with a biological substance relative to the aggregation exhibited upon incubation of the same polypeptide or same protein, respectively, lacking the three histidines with the biological substance. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 80% reduction in aggregation upon incubation with a biological substance relative to the aggregation exhibited upon incubation of the same polypeptide or same protein, respectively, lacking the three histidines with the biological substance. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 90% reduction in aggregation upon incubation with a biological substance relative to the aggregation exhibited upon incubation of the same polypeptide or same protein, respectively, lacking the three histidines with the biological substance. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 95% reduction in aggregation upon incubation with a biological substance relative to the aggregation exhibited upon incubation of the same polypeptide or same protein, respectively, lacking the three histidines with the biological substance. In specific embodiments, the aggregation is determined by an aggregation assay described herein (such as an assay described in Section 7.9.7) or known to one of skill in the art.

In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in reduced aggregation upon incubation with a biological substance relative to the aggregation exhibited upon incubation of the same polypeptide or same protein, respectively, lacking the four histidines with the biological substance. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 10% reduction in aggregation upon incubation with a biological substance relative to the aggregation exhibited upon incubation of the same polypeptide or same protein, respectively, lacking the four histidines with the biological substance. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 20% reduction in aggregation upon incubation with a biological substance relative to the aggregation exhibited upon incubation of the same polypeptide or same protein, respectively, lacking the four histidines with the biological substance. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 30% reduction in aggregation upon incubation with a biological substance relative to the aggregation exhibited upon incubation of the same polypeptide or same protein, respectively, lacking the four histidines with the biological substance. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 40% reduction in aggregation upon incubation with a biological substance relative to the aggregation exhibited upon incubation of the same polypeptide or same protein, respectively, lacking the four histidines with the biological substance. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 50% reduction in aggregation upon incubation with a biological substance relative to the aggregation exhibited upon incubation of the same polypeptide or same protein, respectively, lacking the four histidines with the biological substance. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 60% reduction in aggregation upon incubation with a biological substance relative to the aggregation exhibited upon incubation of the same polypeptide or same protein, respectively, lacking the four histidines with the biological substance. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 70% reduction in aggregation upon incubation with a biological substance relative to the aggregation exhibited upon incubation of the same polypeptide or same protein, respectively, lacking the four histidines with the biological substance. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 80% reduction in aggregation upon incubation with a biological substance relative to the aggregation exhibited upon incubation of the same polypeptide or same protein, respectively, lacking the four histidines with the biological substance. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 90% reduction in aggregation upon incubation with a biological substance relative to the aggregation exhibited upon incubation of the same polypeptide or same protein, respectively, lacking the four histidines with the biological substance. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 95% reduction in aggregation upon incubation with a biological substance relative to the aggregation exhibited upon incubation of the same polypeptide or same protein, respectively, lacking the four histidines with the biological substance. In specific embodiments, the aggregation is determined by an aggregation assay described herein (such as an assay described in Section 7.9.7) or known to one of skill in the art.

In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in reduced aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 10% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 20% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 30% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 40% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 50% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 60% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 70% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 80% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 90% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 95% reduction in aggregation upon incubation with a biological substance relative to the aggregation exhibited upon incubation of the same polypeptide or same protein, respectively, lacking the five histidines with the biological substance. In specific embodiments, the self-aggregation is determined by an aggregation assay described herein (such as an assay described in Section 7.9.7) or known to one of skill in the art.

In specific embodiments, the addition of four histidines at the end of an exposed C-terminus, the end of a non-naturally occurring C-terminus, or the end of a C-terminal anti-drug antibody epitope of a polypeptide, a protein (e.g., an antibody), a bispecific antibody, or a multispecific antibody (e.g., trispecific antibody) results in a reduction of self-aggregation of the polypeptide, the protein (e.g., an antibody), the bispecific antibody, or the multispecific antibody relative to the self-aggregation of the same polypeptide, the same protein (e.g., an antibody), the same bispecific antibody, or the same multispecific antibody (e.g., trispecific antibody), respectively, lacking the four histidines. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 10% reduction in the self-aggregation of the polypeptide, the protein (e.g., an antibody), the bispecific antibody, or the multispecific antibody (e.g., trispecific antibody), relative to the self-aggregation of the same protein (e.g., an antibody), the same bispecific antibody, or the same multispecific antibody (e.g., trispecific antibody), respectively, lacking the four histidines. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 20% reduction in the self-aggregation of the polypeptide, the protein (e.g., an antibody), the bispecific antibody, or the multispecific antibody (e.g., trispecific antibody), relative to the self-aggregation of the same protein (e.g., an antibody), the same bispecific antibody, or the same multispecific antibody (e.g., trispecific antibody), respectively, lacking the four histidines. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 30% reduction in the self-aggregation of the polypeptide, the protein (e.g., an antibody), the bispecific antibody, or the multispecific antibody (e.g., trispecific antibody), relative to the self-aggregation of the same protein (e.g., an antibody), the same bispecific antibody, or the same multispecific antibody (e.g., trispecific antibody), respectively, lacking the four histidines. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 40% reduction in the self-aggregation of the polypeptide, the protein (e.g., an antibody), the bispecific antibody, or the multispecific antibody (e.g., trispecific antibody), relative to the self-aggregation of the same protein (e.g., an antibody), the same bispecific antibody, or the same multispecific antibody (e.g., trispecific antibody), respectively, lacking the four histidines. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 50% reduction in the self-aggregation of the polypeptide, the protein (e.g., an antibody), the bispecific antibody, or the multispecific antibody (e.g., trispecific antibody), relative to the self-aggregation of the same protein (e.g., an antibody), the same bispecific antibody, or the same multispecific antibody (e.g., trispecific antibody), respectively, lacking the four histidines. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 60% reduction in the self-aggregation of the polypeptide, the protein (e.g., an antibody), the bispecific antibody, or the multispecific antibody (e.g., trispecific antibody), relative to the self-aggregation of the same protein (e.g., an antibody), the same bispecific antibody, or the same multispecific antibody (e.g., trispecific antibody), respectively, lacking the four histidines. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 70% reduction in the self-aggregation of the polypeptide, the protein (e.g., an antibody), the bispecific antibody, or the multispecific antibody (e.g., trispecific antibody), relative to the self-aggregation of the same protein (e.g., an antibody), the same bispecific antibody, or the same multispecific antibody (e.g., trispecific antibody), respectively, lacking the four histidines. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 80% reduction in the self-aggregation of the polypeptide, the protein (e.g., an antibody), the bispecific antibody, or the multispecific antibody (e.g., trispecific antibody), relative to the self-aggregation of the same protein (e.g., an antibody), the same bispecific antibody, or the same multispecific antibody (e.g., trispecific antibody), respectively, lacking the four histidines. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 90% reduction in the self-aggregation of the polypeptide, the protein (e.g., an antibody), the bispecific antibody, or the multispecific antibody (e.g., trispecific antibody), relative to the self-aggregation of the same protein (e.g., an antibody), the same bispecific antibody, or the same multispecific antibody (e.g., trispecific antibody), respectively, lacking the four histidines. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 95% reduction in the self-aggregation of the polypeptide, the protein (e.g., an antibody), the bispecific antibody, or the multispecific antibody (e.g., trispecific antibody), relative to the self-aggregation of the same protein (e.g., an antibody), the same bispecific antibody, or the same multispecific antibody (e.g., trispecific antibody), respectively, lacking the four histidines. In particular embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) eliminates aggregation of the polypeptide, the protein (e.g., an antibody), the bispecific antibody, or the multispecific antibody. In specific embodiments, the self-aggregation is determined by an aggregation assay described herein (such as an assay described in Section 7.9.7) or known to one of skill in the art.

In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in reduced aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 10% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 20% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 30% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 40% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 50% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 60% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 70% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 80% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 90% reduction in aggregation upon incubation with a biological substance relative to the aggregation exhibited upon incubation of the same polypeptide or same protein, respectively, lacking the five histidines with the biological substance. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 95% reduction in aggregation upon incubation with a biological substance relative to the aggregation exhibited upon incubation of the same polypeptide or same protein, respectively, lacking the five histidines with the biological substance. In specific embodiments, the aggregation is determined by an aggregation assay described herein (such as an assay described in Section 7.9.7) or known to one of skill in the art.

In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in reduced self-aggregation relative to the self-aggregation exhibited by the same polypeptide or same protein, respectively, lacking the cap domain (e.g., a His cap domain, SLSLSPGK (SEQ ID NO:36), or TVAPTESS (SEQ ID NO: 37)). In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 10% reduction in self-aggregation relative to the self-aggregation exhibited by the same polypeptide or same protein, respectively, lacking the cap domain (e.g., a His cap domain, SLSLSPGK (SEQ ID NO: 36), or TVAPTESS (SEQ ID NO:37)). In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 20% reduction in self-aggregation relative to the self-aggregation exhibited by the same polypeptide or same protein, respectively, lacking the cap domain (e.g., a His cap domain, SLSLSPGK (SEQ ID NO:36), or TVAPTESS (SEQ ID NO:37)). In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 30% reduction in self-aggregation relative to the self-aggregation exhibited by the same polypeptide or same protein, respectively, lacking the cap domain (e.g., a His cap domain, SLSLSPGK (SEQ ID NO:36), or TVAPTESS (SEQ ID NO: 37)). In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 40% reduction in self-aggregation relative to the self-aggregation exhibited by the same polypeptide or same protein, respectively, lacking the cap domain (e.g., a His cap domain, SLSLSPGK (SEQ ID NO: 36), or TVAPTESS (SEQ ID NO:37)). In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 50% reduction in self-aggregation relative to the self-aggregation exhibited by the same polypeptide or same protein, respectively, lacking the cap domain (e.g., a His cap domain, SLSLSPGK (SEQ ID NO:36), or TVAPTESS (SEQ ID NO:37)). In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 60% reduction in self-aggregation relative to the self-aggregation exhibited by the same polypeptide or same protein, respectively, lacking the cap domain (e.g., a His cap domain, SLSLSPGK (SEQ ID NO:36), or TVAPTESS (SEQ ID NO: 37)). In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 70% reduction in self-aggregation relative to the self-aggregation exhibited by the same polypeptide or same protein, respectively, lacking the cap domain (e.g., a His cap domain, SLSLSPGK (SEQ ID NO: 36), or TVAPTESS (SEQ ID NO:37)). In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 80% reduction in self-aggregation relative to the self-aggregation exhibited by the same polypeptide or same protein, respectively, lacking the cap domain (e.g., a His cap domain, SLSLSPGK (SEQ ID NO:36), or TVAPTESS (SEQ ID NO:37)). In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 90% reduction in self-aggregation relative to the self-aggregation exhibited by the same polypeptide or same protein, respectively, lacking the cap domain (e.g., a His cap domain, SLSLSPGK (SEQ ID NO:36), or TVAPTESS (SEQ ID NO: 37)). In some embodiments, the addition a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in at least about a 95% reduction in self-aggregation relative to the self-aggregation exhibited by the same polypeptide or same protein, respectively, lacking the cap domain (e.g., a His cap domain, SLSLSPGK (SEQ ID NO: 36), or TVAPTESS (SEQ ID NO:37)). In specific embodiments, the self-aggregation is determined by an aggregation assay described herein (such as an assay described in Section 7.9.7) or known to one of skill in the art.

In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in reduced self-aggregation relative to the self-aggregation exhibited by the same polypeptide or same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 10% reduction in self-aggregation relative to the self-aggregation exhibited by the same polypeptide or same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 20% reduction in self-aggregation relative to the self-aggregation exhibited by the same polypeptide or same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 30% reduction in self-aggregation relative to the self-aggregation exhibited by the same polypeptide or same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 40% reduction in self-aggregation relative to the self-aggregation exhibited by the same polypeptide or same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 50% reduction in self-aggregation relative to the self-aggregation exhibited by the same polypeptide or same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 60% reduction in self-aggregation relative to the self-aggregation exhibited by the same polypeptide or same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 70% reduction in self-aggregation relative to the self-aggregation exhibited by the same polypeptide or same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 80% reduction in self-aggregation relative to the self-aggregation exhibited by the same polypeptide or same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 90% reduction in self-aggregation relative to the self-aggregation exhibited by the same polypeptide or same protein, respectively, lacking the one histidine. In some embodiments, the addition of one histidine at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 95% reduction in self-aggregation relative to the self-aggregation exhibited by the same polypeptide or same protein, respectively, lacking the one histidine. In specific embodiments, the self-aggregation is determined by an aggregation assay described herein (such as an assay described in Section 7.9.7) or known to one of skill in the art.

In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in reduced self-aggregation relative to the self-aggregation exhibited by the same polypeptide or same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 10% reduction in self-aggregation relative to the self-aggregation exhibited by the same polypeptide or same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 20% reduction in self-aggregation relative to the self-aggregation exhibited by the same polypeptide or same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 30% reduction in self-aggregation relative to the self-aggregation exhibited by the same polypeptide or same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 40% reduction in self-aggregation relative to the self-aggregation exhibited by the same polypeptide or same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 50% reduction in self-aggregation relative to the self-aggregation exhibited by the same polypeptide or same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 60% reduction in self-aggregation relative to the self-aggregation exhibited by the same polypeptide or same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 70% reduction in self-aggregation relative to the self-aggregation exhibited by the same polypeptide or same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 80% reduction in self-aggregation relative to the self-aggregation exhibited by the same polypeptide or same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 90% reduction in self-aggregation relative to the self-aggregation exhibited by the same polypeptide or same protein, respectively, lacking the two histidines. In some embodiments, the addition of two histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 95% reduction in self-aggregation relative to the self-aggregation exhibited by the same polypeptide or same protein, respectively, lacking the two histidines. In specific embodiments, the self-aggregation is determined by an aggregation assay described herein (such as an assay described in Section 7.9.7) or known to one of skill in the art.

In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in reduced self-aggregation relative to the self-aggregation exhibited by the same polypeptide or same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 10% reduction in self-aggregation relative to the self-aggregation exhibited by the same polypeptide or same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 20% reduction in self-aggregation relative to the self-aggregation exhibited by the same polypeptide or same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 30% reduction in self-aggregation relative to the self-aggregation exhibited by the same polypeptide or same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 40% reduction in self-aggregation relative to the self-aggregation exhibited by the same polypeptide or same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 50% reduction in self-aggregation relative to the self-aggregation exhibited by the same polypeptide or same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 60% reduction in self-aggregation relative to the self-aggregation exhibited by the same polypeptide or same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 70% reduction in self-aggregation relative to the self-aggregation exhibited by the same polypeptide or same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 80% reduction in self-aggregation relative to the self-aggregation exhibited by the same polypeptide or same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 90% reduction in self-aggregation relative to the self-aggregation exhibited by the same polypeptide or same protein, respectively, lacking the three histidines. In some embodiments, the addition of three histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 95% reduction in self-aggregation relative to the self-aggregation exhibited by the same polypeptide or same protein, respectively, lacking the three histidines. In specific embodiments, the self-aggregation is determined by an aggregation assay described herein (such as an assay described in Section 7.9.7) or known to one of skill in the art.

In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in reduced self-aggregation relative to the self-aggregation exhibited by the same polypeptide or same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 10% reduction in self-aggregation relative to the self-aggregation exhibited by the same polypeptide or same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 20% reduction in self-aggregation relative to the self-aggregation exhibited by the same polypeptide or same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 30% reduction in self-aggregation relative to the self-aggregation exhibited by the same polypeptide or same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 40% reduction in self-aggregation relative to the self-aggregation exhibited by the same polypeptide or same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 50% reduction in self-aggregation relative to the self-aggregation exhibited by the same polypeptide or same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 60% reduction in self-aggregation relative to the self-aggregation exhibited by the same polypeptide or same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 70% reduction in self-aggregation relative to the self-aggregation exhibited by the same polypeptide or same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 80% reduction in self-aggregation relative to the self-aggregation exhibited by the same polypeptide or same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 90% reduction in self-aggregation relative to the self-aggregation exhibited by the same polypeptide or same protein, respectively, lacking the four histidines. In some embodiments, the addition of four histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 95% reduction in self-aggregation relative to the self-aggregation exhibited by the same polypeptide or same protein, respectively, lacking the four histidines. In specific embodiments, the self-aggregation is determined by an aggregation assay described herein (such as an assay described in Section 7.9.7) or known to one of skill in the art.

In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in reduced self-aggregation relative to the self-aggregation exhibited by the same polypeptide or same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 10% reduction in self-aggregation relative to the self-aggregation exhibited by the same polypeptide or same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 20% reduction in self-aggregation relative to the self-aggregation exhibited by the same polypeptide or same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 30% reduction in self-aggregation relative to the self-aggregation exhibited by the same polypeptide or same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 40% reduction in self-aggregation relative to the self-aggregation exhibited by the same polypeptide or same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 50% reduction in self-aggregation relative to the self-aggregation exhibited by the same polypeptide or same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 60% reduction in self-aggregation relative to the self-aggregation exhibited by the same polypeptide or same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 70% reduction in self-aggregation relative to the self-aggregation exhibited by the same polypeptide or same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 80% reduction in self-aggregation relative to the self-aggregation exhibited by the same polypeptide or same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 90% reduction in self-aggregation relative to the self-aggregation exhibited by the same polypeptide or same protein, respectively, lacking the five histidines. In some embodiments, the addition of five histidines at the end of an exposed C-terminus, the end of a non-naturally occurring C-terminus, or the end of a C-terminal anti-drug antibody epitope of a polypeptide or a protein (e.g., an antibody, such as, e.g., a bispecific antibody, or a multispecific antibody (e.g., trispecific antibody)) results in about a 95% reduction in self-aggregation relative to the self-aggregation exhibited by the same polypeptide or same protein, respectively, lacking the five histidines. In specific embodiments, the self-aggregation is determined by an aggregation assay described herein (such as an assay described in Section 7.9.7) or known to one of skill in the art.

In specific embodiments, the addition of a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a protein (e.g., an antigen binding protein) results in a reduction in the neutralization of the protein by an ADA relative to the neutralization of the same protein lacking the cap domain by the ADA. In some embodiments, the reduction is at least 10%, at least 20%, at least 30%, at least 40%, or at least 50%. In some embodiments, the reduction is at least 60%, at least 70%, at least 80%, at least 90%, or at least 95%.

In specific embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in a reduction in the neutralization of the protein by an ADA relative to the neutralization of the same protein lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 10% reduction in the neutralization of the protein by an ADA, relative to the neutralization of the same protein lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 20% reduction in the neutralization of the protein by an ADA, relative to the neutralization of the same protein lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 30% reduction in the neutralization of the protein by an ADA, relative to the neutralization of the same protein lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 40% reduction in the neutralization of the protein by an ADA, relative to the neutralization of the same protein lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 50% reduction in the neutralization of the protein by an ADA, relative to the neutralization of the same protein lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 60% reduction in the neutralization of the protein by an ADA, relative to the neutralization of the same protein lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 70% reduction in the neutralization of the protein by an ADA, relative to the neutralization of the same protein lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about an 80% reduction in the neutralization of the protein by an ADA, relative to the neutralization of the same protein lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 90% reduction in the neutralization of the protein by an ADA, relative to the neutralization of the same protein lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 95% reduction in the neutralization of the protein by an ADA, relative to the neutralization of the same protein lacking the (His)ₙ (SEQ ID NO:25) sequence by the ADA. In specific embodiments, the neutralization of a protein by an ADA is determined by neutralization assay described herein (such as an assay described in Section 7.9.4) or known to one of skill in the art.

In specific embodiments, the addition of one histidine at the end of an exposed C-terminus, the end of a non-naturally occurring C-terminus, or the end of a C-terminal anti-drug antibody epitope of a protein (e.g., an antigen binding protein) results in a reduction in the neutralization of the protein by an ADA relative to the neutralization of the same protein lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antigen binding protein) results in at least about a 10% reduction in the neutralization of the protein by an ADA, relative to the neutralization of the same protein lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antigen binding protein) results in at least about a 20% reduction in the neutralization of the protein by an ADA, relative to the neutralization of the same protein lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antigen binding protein) results in at least about a 30% reduction in the neutralization of the protein by an ADA, relative to the neutralization of the same protein lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antigen binding protein) results in at least about a 40% reduction in the neutralization of the protein by an ADA, relative to the neutralization of the same protein lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antigen binding protein) results in at least about a 50% reduction in the neutralization of the protein by an ADA, relative to the neutralization of the same protein lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antigen binding protein) results in at least about a 60% reduction in the neutralization of the protein by an ADA, relative to the neutralization of the same protein lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antigen binding protein) results in at least about a 70% reduction in the neutralization of the protein by an ADA, relative to the neutralization of the same protein lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antigen binding protein) results in at least about an 80% reduction in the neutralization of the protein by an ADA, relative to the neutralization of the same protein lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antigen binding protein) results in at least about a 90% reduction in the neutralization of the protein by an ADA, relative to the neutralization of the same protein lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antigen binding protein) results in at least about a 95% reduction in the neutralization of the protein by an ADA, relative to the neutralization of the same protein lacking the one histidine by the ADA. In particular embodiments, the addition of one histidine at the end of an exposed C-terminus, the end of a non-naturally occurring C-terminus, or the end of a C-terminal anti-drug antibody epitope of a protein (e.g., an antigen binding protein) eliminates neutralization of the protein by an ADA. In specific embodiments, the neutralization of a protein by an ADA is determined by neutralization assay described herein (such as an assay described in Section 7.9.4) or known to one of skill in the art.

In specific embodiments, the addition of two histidines at the end of an exposed C-terminus, the end of a non-naturally occurring C-terminus, or the end of a C-terminal anti-drug antibody epitope of a protein (e.g., an antigen binding protein) results in a reduction in the neutralization of the protein by an ADA relative to the neutralization of the same protein lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 10% reduction in the neutralization of the protein by an ADA, relative to the neutralization of the same protein lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 20% reduction in the neutralization of the protein by an ADA, relative to the neutralization of the same protein lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 30% reduction in the neutralization of the protein by an ADA, relative to the neutralization of the same protein lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 40% reduction in the neutralization of the protein by an ADA, relative to the neutralization of the same protein lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 50% reduction in the neutralization of the protein by an ADA, relative to the neutralization of the same protein lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 60% reduction in the neutralization of the protein by an ADA, relative to the neutralization of the same protein lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 70% reduction in the neutralization of the protein by an ADA, relative to the neutralization of the same protein lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about an 80% reduction in the neutralization of the protein by an ADA, relative to the neutralization of the same protein lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 90% reduction in the neutralization of the protein by an ADA, relative to the neutralization of the same protein lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antigen binding protein) results in at least about a 95% reduction in the neutralization of the protein by an ADA, relative to the neutralization of the same protein lacking the two histidines by the ADA. In particular embodiments, the addition of two histidines at the end of an exposed C-terminus, the end of a non-naturally occurring C-terminus, or the end of a C-terminal anti-drug antibody epitope of a protein (e.g., an antigen binding protein) eliminates neutralization of the protein by an ADA. In specific embodiments, the neutralization of a protein by an ADA is determined by neutralization assay described herein (such as an assay described in Section 7.9.4) or known to one of skill in the art.

In specific embodiments, the addition of three histidines at the end of an exposed C-terminus, the end of a non-naturally occurring C-terminus, or the end of a C-terminal anti-drug antibody epitope of a protein (e.g., an antigen binding protein) results in a reduction in the neutralization of the protein by an ADA relative to the neutralization of the same protein lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 10% reduction in the neutralization of the protein by an ADA, relative to the neutralization of the same protein lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 20% reduction in the neutralization of the protein by an ADA, relative to the neutralization of the same protein lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 30% reduction in the neutralization of the protein by an ADA, relative to the neutralization of the same protein lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 40% reduction in the neutralization of the protein by an ADA, relative to the neutralization of the same protein lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 50% reduction in the neutralization of the protein by an ADA, relative to the neutralization of the same protein lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 60% reduction in the neutralization of the protein by an ADA, relative to the neutralization of the same protein lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 70% reduction in the neutralization of the protein by an ADA, relative to the neutralization of the same protein lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about an 80% reduction in the neutralization of the protein by an ADA, relative to the neutralization of the same protein lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 90% reduction in the neutralization of the protein by an ADA, relative to the neutralization of the same protein lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antigen binding protein) results in at least about a 95% reduction in the neutralization of the protein by an ADA, relative to the neutralization of the same protein lacking the three histidines by the ADA. In particular embodiments, the addition of three histidines at the end of an exposed C-terminus, the end of a non-naturally occurring C-terminus, or the end of a C-terminal anti-drug antibody epitope of a protein (e.g., an antigen binding protein) eliminates neutralization of the protein by an ADA. In specific embodiments, the neutralization of a protein by an ADA is determined by neutralization assay described herein (such as an assay described in Section 7.9.4) or known to one of skill in the art.

In specific embodiments, the addition of four histidines at the end of an exposed C-terminus, the end of a non-naturally occurring C-terminus, or the end of a C-terminal anti-drug antibody epitope of a protein (e.g., an antigen binding protein) results in a reduction in the neutralization of the protein by an ADA relative to the neutralization of the same protein lacking the four histidines by the ADA. In some embodiments, the addition of four histidines at the end of an exposed C-terminus, the end of a non-naturally occurring C-terminus, or the end of the C-terminal anti-drug antibody epitope of a protein (e.g., an antigen binding protein) results in at least about a 10% reduction in the neutralization of the protein by an ADA, relative to the neutralization of the same protein lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 20% reduction in the neutralization of the protein by an ADA, relative to the neutralization of the same protein lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 30% reduction in the neutralization of the protein by an ADA, relative to the neutralization of the same protein lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 40% reduction in the neutralization of the protein by an ADA, relative to the neutralization of the same protein lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 50% reduction in the neutralization of the protein by an ADA, relative to the neutralization of the same protein lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 60% reduction in the neutralization of the protein by an ADA, relative to the neutralization of the same protein lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 70% reduction in the neutralization of the protein by an ADA, relative to the neutralization of the same protein lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about an 80% reduction in the neutralization of the protein by an ADA, relative to the neutralization of the same protein lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 90% reduction in the neutralization of the protein by an ADA, relative to the neutralization of the same protein lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antigen binding protein) results in at least about a 95% reduction in the neutralization of the protein by an ADA, relative to the neutralization of the same protein lacking the four histidines by the ADA. In particular embodiments, the addition of four histidines at the end of an exposed C-terminus, the end of a non-naturally occurring C-terminus, or the end of a C-terminal anti-drug antibody epitope of a protein (e.g., an antigen binding protein) eliminates neutralization of the protein by an ADA. In specific embodiments, the neutralization of a protein by an ADA is determined by neutralization assay described herein (such as an assay described in Section 7.9.4) or known to one of skill in the art.

In specific embodiments, the addition of five histidines at the end of an exposed C-terminus, the end of a non-naturally occurring C-terminus, or the end of a C-terminal anti-drug antibody epitope of a protein (e.g., an antigen binding protein) results in a reduction in the neutralization of the protein by an ADA relative to the neutralization of the same protein lacking the five histidines by the ADA. In some embodiments, the addition of five histidines at the end of an exposed C-terminus, the end of a non-naturally occurring C-terminus, or the end of the C-terminal anti-drug antibody epitope of a protein (e.g., an antigen binding protein) results in at least about a 10% reduction in the neutralization of the protein by an ADA, relative to the neutralization of the same protein lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 20% reduction in the neutralization of the protein by an ADA, relative to the neutralization of the same protein lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 30% reduction in the neutralization of the protein by an ADA, relative to the neutralization of the same protein lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 40% reduction in the neutralization of the protein by an ADA, relative to the neutralization of the same protein lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 50% reduction in the neutralization of the protein by an ADA, relative to the neutralization of the same protein lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 60% reduction in the neutralization of the protein by an ADA, relative to the neutralization of the same protein lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 70% reduction in the neutralization of the protein by an ADA, relative to the neutralization of the same protein lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about an 80% reduction in the neutralization of the protein by an ADA, relative to the neutralization of the same protein lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 90% reduction in the neutralization of the protein by an ADA, relative to the neutralization of the same protein lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antigen binding protein) results in at least about a 95% reduction in the neutralization of the protein by an ADA, relative to the neutralization of the same protein lacking the five histidines by the ADA. In particular embodiments, the addition of five histidines at the end of an exposed C-terminus, the end of a non-naturally occurring C-terminus, or the end of a C-terminal anti-drug antibody epitope of a protein (e.g., an antigen binding protein) eliminates neutralization of the protein by an ADA. In specific embodiments, the neutralization of a protein by an ADA is determined by neutralization assay described herein (such as an assay described in Section 7.9.4) or known to one of skill in the art.

In specific embodiments, the addition of a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a protein (e.g., an antigen binding protein) results in an improvement in the pharmacokinetics of the protein relative to the pharmacokinetics of the same protein lacking the cap domain. In some embodiments, the improvement is at least 10%, at least 20%, at least 30%, at least 40%, or at least 50% improvement. In some embodiments, the improvement is at least 60%, at least 70%, at least 80%, at least 90%, or at least 95% improvement.

In specific embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in an improvement in the pharmacokinetics of the protein relative to the pharmacokinetics of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 10% improvement in the pharmacokinetics of the protein, relative to the pharmacokinetics of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 20% improvement in the pharmacokinetics of the protein, relative to the pharmacokinetics of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 30% improvement in the pharmacokinetics of the protein, relative to the pharmacokinetics of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 40% improvement in the pharmacokinetics of the protein, relative to the pharmacokinetics of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 50% improvement in the pharmacokinetics of the protein, relative to the pharmacokinetics of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 60% improvement in the pharmacokinetics of the protein, relative to the pharmacokinetics of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 70% improvement in the pharmacokinetics of the protein, relative to the pharmacokinetics of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about an 80% improvement in the pharmacokinetics of the protein, relative to the pharmacokinetics of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 90% improvement in the pharmacokinetics of the protein, relative to the pharmacokinetics of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 95% improvement in the pharmacokinetics of the protein, relative to the pharmacokinetics of the same protein lacking the (His)ₙ (SEQ ID NO:25) sequence. In specific embodiments, the pharmacokinetics of a protein is determined by pharmacokinetic assays described herein (such as an assay described in Section 7.9.2) or known to one of skill in the art.

In specific embodiments, the addition of a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a protein (e.g., an antigen binding protein) results in an improvement in the safety of the protein relative to the safety of the same protein lacking the cap domain. In some embodiments, the improvement is at least 10%, at least 20%, at least 30%, at least 40%, or at least 50% improvement. In some embodiments, the improvement is at least 60%, at least 70%, at least 80%, at least 90%, or at least 95% improvement.

In specific embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in an improvement in the safety of the protein relative to the safety of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 10% improvement in the safety of the protein, relative to the safety of the same protein lacking the (His)ₙ (SEQ ID NO:25) sequence. In some embodiments, the addition of a (His)ₙ (SEQ ID NO: 25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 20% improvement in the safety of the protein, relative to the safety of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 30% improvement in the safety of the protein, relative to the safety of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 40% improvement in the safety of the protein, relative to the safety of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 50% improvement in the safety of the protein, relative to the safety of the same protein lacking the (His)ₙ (SEQ ID NO:25) sequence. In some embodiments, the addition of a (His)ₙ (SEQ ID NO: 25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 60% improvement in the safety of the protein, relative to the safety of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 70% improvement in the safety of the protein, relative to the safety of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about an 80% improvement in the safety of the protein, relative to the safety of the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 90% improvement in the safety of the protein, relative to the safety of the same protein lacking the (His)ₙ (SEQ ID NO:25) sequence. In some embodiments, the addition of a (His)ₙ (SEQ ID NO: 25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 95% improvement in the safety of the protein, relative to the safety of the same protein lacking the (His)ₙ (SEQ ID NO:25) sequence. In specific embodiments, the safety of a protein is determined by safety assays described herein (such as an assay described in Section 7.9.3) or known to one of skill in the art.

In specific embodiments, the addition of a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a protein (e.g., an antigen binding protein results in decreased affinity for an ADA, relative to the same protein lacking the cap domain. In some embodiments, the decrease is at least 10%, at least 20%, at least 30%, at least 40%, or at least 50%. In some embodiments, the decrease is at least 60%, at least 70%, at least 80%, at least 90%, or at least 95%.

In specific embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in decreased affinity for an ADA, relative to the same protein lacking the (His)ₙ (SEQ ID NO:25) sequence. In some embodiments, the addition of a (His)ₙ (SEQ ID NO: 25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 2-fold decrease in affinity for an ADA, relative to the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 5-fold decrease in affinity for an ADA, relative to the same protein lacking the (His)ₙ (SEQ ID NO:25) sequence. In specific embodiments, the pharmacokinetics of a protein is determined by pharmacokinetic assays described herein (such as an assay described in Section 7.9.2) or known to one of skill in the art. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 10-fold decrease in affinity for an ADA, relative to the same protein lacking the (His)ₙ (SEQ ID NO:25) sequence.

In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 20-fold decrease in affinity for an ADA, relative to the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 50-fold decrease in affinity for an ADA, relative to the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 100-fold decrease in affinity for an ADA, relative to the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in at least about a 500-fold decrease in affinity for an ADA, relative to the same protein lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antigen binding protein), wherein n is 1, 2, 3, 4, or 5, results in greater than 500-fold decrease in affinity for an ADA, relative to the same protein lacking the (His)ₙ (SEQ ID NO:25) sequence. In specific embodiments, affinity of a protein for an ADA is determined by measuring interaction between the protein and an ADA (such as in assay described in Section 7.9.1) or known to one of skill in the art.

In a specific embodiment, the addition of a cap domain (e.g., a His cap domain, SLSLSPGK (SEQ ID NO:36), or TVAPTESS (SEQ ID NO:37)) to an exposed C-terminus, a non-naturally occurring C-terminus, or a C-terminal anti-drug epitope of a protein, does not interfere with one, two, three, or more, or all of the functions of the protein as assessed by a technique described herein or known to one of skill in the art. In a specific embodiment, the addition of a (His)ₙ (SEQ ID NO:25) sequence to an exposed C-terminus, a non-naturally occurring C-terminus, or a C-terminal anti-drug epitope of a protein, wherein n is 1, 2, 3, 4, or 5, does not interfere with one, two, three, or more, or all of the functions of the protein as assessed by a technique described herein or known to one of skill in the art. For example, the addition of a (His)ₙ (SEQ ID NO:25) sequence to an exposed C-terminus, a non-naturally occurring C-terminus, or a C-terminal anti-drug epitope of an antibody or other antigen binding protein, wherein n is 1, 2, 3, 4, or 5, does not reduce or inhibit the binding of the antibody or other antigen binding protein to its target antigen. In specific embodiments, the addition of a cap domain (e.g., a His cap domain, SLSLSPGK (SEQ ID NO:36), or TVAPTESS (SEQ ID NO:37)) to an exposed C-terminus, a non-naturally occurring C-terminus, or a C-terminal anti-drug epitope of antibody does not interfere with the structure of the protein as assessed by a technique described herein (such as an assay described in Section 7.9.5) or known to one of skill in the art. In specific embodiments, the addition of a (His)ₙ (SEQ ID NO:25) sequence to an exposed C-terminus, a non-naturally occurring C-terminus, or a C-terminal anti-drug epitope of, wherein n is 1, 2, 3, 4, or 5, does not interfere with the structure of the protein as assessed by a technique described herein (such as an assay described in Section 7.9.5) or known to one of skill in the art. In specific embodiments, a protein described herein (e.g., a therapeutic protein) comprises an exposed C-terminus. In specific embodiments, a protein described herein (e.g., a therapeutic protein) comprises a non-naturally occurring C-terminus. In specific embodiments, a protein described herein (e.g., a therapeutic protein) comprises a C-terminal anti-drug antibody epitope.

In specific embodiments, a protein described herein (e.g., a therapeutic protein) comprises an exposed C-terminus and cap domain (e.g., a His cap domain, SLSLSPGK (SEQ ID NO: 36), or TVAPTESS (SEQ ID NO:37)). In specific embodiments, a protein described herein (e.g., a therapeutic protein) comprises a non-naturally occurring C-terminus and a (His)ₙ (SEQ ID NO: 25) sequence at the end of the non-naturally occurring C-terminus, wherein n is 1, 2, 3, 4, or 5. In specific embodiments, a protein described herein (e.g., a therapeutic protein) comprises a C-terminal anti-drug antibody epitope and a (His)ₙ (SEQ ID NO:25) sequence at the end of the C-terminal anti-drug antibody epitope, wherein n is 1, 2, 3, 4, or 5.

In specific embodiments, a protein described herein comprising an exposed C-terminus fused to a cap domain (e.g., a His cap domain, SLSLSPGK (SEQ ID NO:36), or TVAPTESS (SEQ ID NO:37)) exhibits reduced interaction with a reference relative to the interaction exhibited by the same protein lacking the cap domain (e.g., a His cap domain, SLSLSPGK (SEQ ID NO:36), or TVAPTESS (SEQ ID NO:37)) under the same conditions, as determined by the same assay. In certain embodiments, the interaction between the protein and reference is reduced by at least 10%, at least 15%, at least 20%, or at least 25%. In some embodiments, the interaction between the protein and reference is reduced by at least 30%, at least 35%, at least 40%, or at least 45%. In certain embodiments, the interaction between the protein and reference is reduced by at least 50%, at least 65%, at least 70%, or at least 85%. In certain embodiments, the interaction between the protein and reference is reduced by at least 90%, at least 95%, or at least 98%.

In some embodiments, the reference comprises the reference is a molecule present in a biological substance. In some embodiments, the biological substance comprises serum and/or plasma (e.g., human serum and/or human plasma). In some embodiments, the biological substance comprises serum (e.g., human serum). In some embodiments, the biological substance comprises plasma (e.g., human plasma). In some embodiments, the reference comprises an anti-drug antibody. In some embodiments, the anti-drug antibody is pre-existing in the serum and/or plasma of a subject (e.g., human subject).

In specific embodiments, a protein described herein comprising a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope exhibits a reduction in the interaction of the protein with an ADA relative to the interaction of the same protein lacking the cap domain with the ADA under the same conditions, as determined by the same assay. In specific embodiments, a protein described herein comprising a (His)ₙ (SEQ ID NO:25) sequence at the end of an exposed C-terminus, the end of a non-naturally occurring C-terminus, or the end of a C-terminal anti-drug antibody epitope, wherein n is 1, 2, 3, 4, or 5, exhibits a reduction in the interaction of the protein with an ADA relative to the interaction of the same protein lacking the (His)ₙ (SEQ ID NO:25) sequence with the ADA under the same conditions, as determined by the same assay. In certain embodiments, the interaction between the protein and ADA is reduced by at least 10%, at least 15%, at least 20%, or at least 25%. In some embodiments, the interaction between the protein and ADA is reduced by at least 30%, at least 35%, at least 40%, at least 45%, or at least 50%. In certain embodiments, the interaction between the protein and ADA is reduced by at least 55%, at least 60%, at least 65%, at least 70%, or at least 75%. In some embodiments, the interaction between the protein and ADA is reduced by at least 80%, at least 85%, at least 90%, at least 95%, or at least 98%.

In specific embodiments, a protein described herein comprising a cap domain ((e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope exhibits a reduction in the neutralization of the protein by an ADA relative to the neutralization of the same protein lacking the cap domain by the ADA under the same conditions, as determined by the same assay. In specific embodiments, a protein described herein comprising a (His)ₙ (SEQ ID NO:25) sequence at the end of an exposed C-terminus, the end of a non-naturally occurring C-terminus, or the end of a C-terminal anti-drug antibody epitope, wherein n is 1, 2, 3, 4, or 5, exhibits a reduction in the neutralization of the protein by an ADA relative to the neutralization of the same protein lacking the (His)ₙ (SEQ ID NO:25) sequence by the ADA under the same conditions, as determined by the same assay. In certain embodiments, the neutralization of the protein by the ADA is reduced by at least 10%, at least 15%, at least 20%, or at least 25%. In some embodiments, the neutralization of the protein by the ADA is reduced by at least 30%, at least 35%, at least 40%, at least 45%, or at least 50%. In certain embodiments, the neutralization of the protein by the ADA is reduced by at least 55%, at least 60%, at least 65%, at least 70%, or at least 75%. In some embodiments, the neutralization of the protein by the ADA is reduced by at least 80%, at least 85%, at least 90%, at least 95%, or at least 98%.

In specific embodiments, a protein described herein comprising a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope exhibits an improvement of the pharmacokinetics of the protein relative to the pharmacokinetics of the same protein lacking the cap domain under the same conditions, as determined by the same assay. In specific embodiments, a protein described herein comprising a (His)ₙ (SEQ ID NO:25) sequence at the end of an exposed C-terminus, the end of a non-naturally occurring C-terminus, or the end of a C-terminal anti-drug antibody epitope, wherein n is 1, 2, 3, 4, or 5, exhibits an improvement of the pharmacokinetics of the protein relative to the pharmacokinetics of the same protein lacking the (His)ₙ (SEQ ID NO:25) sequence under the same conditions, as determined by the same assay. In certain embodiments, the pharmacokinetics of the protein is improved by at least 10%, at least 15%, at least 20%, or at least 25%. In some embodiments, the pharmacokinetics of the protein is improved by at least 30%, at least 35%, at least 40%, at least 45%, or at least 50%. In certain embodiments, the pharmacokinetics of the protein is improved by at least 55%, at least 60%, at least 65%, at least 70%, or at least 75%. In some embodiments, the pharmacokinetics of the protein is improved by at least 80%, at least 85%, at least 90%, at least 95%, or at least 98%.

In specific embodiments, a protein described herein comprising cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope exhibits an improvement of the safety of the protein relative to the safety of the same protein lacking the cap domain under the same conditions, as determined by the same assay. In specific embodiments, a protein described herein comprising a (His)ₙ (SEQ ID NO: 25) sequence at the end of an exposed C-terminus, the end of a non-naturally occurring C-terminus, or the end of a C-terminal anti-drug antibody epitope, wherein n is 1, 2, 3, 4, or 5, exhibits an improvement of the safety of the protein relative to the safety of the same protein lacking the (His)ₙ (SEQ ID NO:25) sequence under the same conditions, as determined by the same assay. In certain embodiments, the safety of the protein is improved by at least 10%, at least 15%, at least 20%, or at least 25%. In some embodiments, the safety of the protein is improved by at least 30%, at least 35%, at least 40%, at least 45%, or at least 50%. In certain embodiments, the safety of the protein is improved by at least 55%, at least 60%, at least 65%, at least 70%, or at least 75%. In some embodiments, the safety of the protein is improved by at least 80%, at least 85%, at least 90%, at least 95%, or at least 98%.

In specific embodiments, a protein described herein comprising a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope exhibits reduced aggregation relative to the same protein lacking the cap domain under the same conditions, as determined by the same assay. In specific embodiments, a protein described herein comprising a (His)ₙ (SEQ ID NO:25) sequence at the end of an exposed C-terminus, the end of a non-naturally occurring C-terminus, or the end of a C-terminal anti-drug antibody epitope, wherein n is 1, 2, 3, 4, or 5, exhibits reduced aggregation relative to the same protein lacking the (His)ₙ (SEQ ID NO:25) sequence under the same conditions, as determined by the same assay. In certain embodiments, aggregation is reduced by at least 10%, at least 15%, at least 20%, or at least 25%. In some embodiments, aggregation is reduced by at least 30%, at least 35%, at least 40%, at least 45%, or at least 50%. In certain embodiments, aggregation is reduced by at least 55%, at least 60%, at least 65%, at least 70%, or at least 75%. In some embodiments, aggregation is reduced by at least 80%, at least 85%, at least 90%, at least 95%, or at least 98%. In specific embodiments, the aggregation is self-aggregation. In some embodiments, the aggregation is reduced in a high concentration liquid formulation (HCLF). In specific embodiments, the HCLF is about 50 mg/mL to about 150 mg/mL. In some embodiments, the aggregation is reduced in a biological substance (e.g., serum and/or plasma). In some embodiments, aggregation is determined under thermal stress (e.g., incubation at about 40° C.). In some embodiments, aggregation is determined under physiological stress (e.g., incubation at about 37° C.). In some embodiments, aggregation is determined by incubation at about 40° C. for about two weeks.

As provided herein, in some embodiments, the addition of a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or protein does not alter the stability of the polypeptide or the protein. In some embodiments, the addition of a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a polypeptide or protein does not alter the thermal stability of the polypeptide or the protein. In specific embodiments, the stability of the polypeptide or the protein is determined by a protein stability assay (such as in assay described in Section 7.9.6) or known to one of skill in the art.

In certain embodiments, a protein described herein is a cytokine. In some embodiments, the cytokine is IL-12, IL-23, IL-1β, IL-6, IL-15, IL-2, TNF-alpha, IL-9, or IL-17. In some embodiments, a protein described herein is an enzyme. In certain embodiments, a protein described herein is a hormone. In some embodiments, a protein described herein comprises an antigen binding protein. In certain embodiments, a protein described herein comprises an antibody. In specific embodiments, a protein described herein is an antibody. In some embodiments, a protein described herein comprises an antigen binding region of an antibody. In certain embodiments, a protein described herein comprises an antigen binding region of an antibody. In some embodiments, a protein described herein comprises an antibody fragment. The antibody fragment may be a functional antibody fragment. In some embodiments, a protein described herein is a fusion protein. The fusion protein may comprise an antigen binding region of antibody, such as, e.g., a variable heavy domain, a variable light domain, a heavy chain, or a light chain. The fusion protein may comprise an antibody fragment and the antibody fragment may be a functional fragment. In specific embodiments, a protein described herein is isolated. In certain embodiments, a protein described herein is recombinantly produced. In specific embodiments, a protein described herein is a therapeutic protein.

In some embodiments, a protein described herein comprises an antibody, antibody binding region, or an antibody fragment recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90, about 100, about 150, about 200, about 250, about 300, about 350, about 400, about 450 or about 500 amino acids, or over 500 amino acids) to generate fusion proteins, as well as uses thereof. In certain embodiments, provided herein are fusion proteins comprising an antigen binding fragment of the antibody (e.g., CDR1, CDR2, and/or CDR3) and a heterologous protein. In certain embodiments, a protein described herein comprises an antibody, antibody binding region, or an antibody fragment is linked to a heterologous protein via a linker. The linker may be any linker known to one of skill in the art so long as the linker does not interfere with the function and/or structure of the antibody, antibody binding region or antibody fragment, and/or heterologous protein. For example, the linker may be a glycine linker ((Gly)ₙ (SEQ ID NO: 35), wherein n is 1, 2, 3, 4, 5, 6 or more) or a glycine-serine linker. The linker may be a flexible linker. In certain embodiments, an antibody, antibody binding region, or an antibody fragment is genetically conjugated to a therapeutic molecule, with a hinge region linking the antibody to the therapeutic molecule. In certain embodiments, the antibody is bispecific or multispecific. In certain embodiments, the multi-specific antibody is trispecific.

Antibody fragments include antibody functional fragments that retain the ability to bind to an antigen. Exemplary functional fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab and an additional portion of the heavy chain through the hinge region); F(ab′)2 (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of, e.g., 10-25 amino acids); a disulfide-linked Fv, or dsFv (e.g., the variable, antigen-binding determinative region of a single light and heavy chain of an antibody linked together by a disulfide bond); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); a triabody (e.g., a trimerized scFv, formed in a manner similar to a diabody, but in which three antigen-binding domains are created in a single complex; the three antigen binding domains may be directed towards the same or different epitopes); and a tetrabody (e.g., a tetramerized scFv, formed in a manner similar to a diabody, but in which four antigen-binding domains are created in a single complex; the four antigen binding domains may be directed towards the same or different epitopes).

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. For example, Fab, Fv, and scFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., 1992, Bio/Technology 10:163-67). According to another approach, F(ab′)₂ fragments can be isolated directly from recombinant host cell culture. Fab and F(ab′)₂ fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of a scFv (See, e.g., Borrebaeck ed., supra). The antibody fragment may also be a “linear antibody,” for example, as described in the references cited above. Such linear antibodies may be monospecific or multi-specific, such as bispecific.

In specific embodiments, an antibody fragment comprises a single-chain fragment variable (scFv) or a single domain antibody. In some embodiments, an antibody fragment comprises a single-chain fragment variable (scFv). In some embodiments, an antibody fragment comprises a single domain antibody. In certain embodiments, an antibody fragment consists of a single-chain fragment variable (scFv). In certain embodiments, an antibody fragment consists of a single domain antibody. In some embodiments, the scFv has been chemically modified. In some embodiments, the scFv has thio-engineered thiol-linkages.

In some embodiments, a protein described herein comprises the structure of one depicted in any one of FIGS. 1-9A. In some embodiments, a protein described herein (e.g., an antibody or other antigen binding protein) is chemically modified, for example, by glycosylation, acetylation, pegylation, phosphorylation, amidation, derivatization by known protecting/blocking groups, proteolytic cleavage, linkage to a cellular ligand or other protein, or conjugation to one or more immunoglobulin domains (e.g., Fc or a portion of an Fc). Any of numerous chemical modifications may be carried out by known techniques, including, but not limited to, specific chemical cleavage, acetylation, formulation, metabolic synthesis of tunicamycin, etc. Additionally, the protein may contain one or more non-classical amino acids.

As provided herein, in some aspects a protein described herein (e.g., an antibody or other antigen binding protein) may bind to a tumor antigen. In some embodiments, a protein described herein is a bispecific or multispecific antibody (e.g., trispecific antibody) that binds to two, three, or more of tumor antigens. In specific embodiments, a protein described herein is a trispecific antibody that binds to three different tumor antigens. Tumor antigens include proteins that are produced by tumor cells that can elicit an immune response, particularly T-cell mediated immune responses. Exemplary tumor antigens include, but not limited to, a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, and mesothelin.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). A TSA is unique to tumor cells and does not occur on other cells in the body. A TAA is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA include: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

In other aspects, a protein described herein (e.g., an antibody or other antigen binding protein) may bind to non-tumor antigen, such as a cytokine (e.g., IL-12/23, IL-1β, IL-6), a cytokine receptor (e.g., IL-6R), a pathogen (e.g., a virus, a bacteria, a fungus or a parasite), or any other molecule capable of inducing or promoting a disease or disorder. In some embodiments, an antibody that binds to the same target as an antibody listed in Table 2 below is suitable for use in the present disclosure.

Various commercially available antibodies are provided in Table 2 below, and are suitable for use in the present disclosure, either as an intact antibody, as an antibody fragment, or as a portion of a multidimeric antibody (e.g., a fusion protein).

An antibody that binds to an antigen of an organism provided in Table 3 is suitable for use in the present disclosure. For example, the antigen of an organism may be one provided in Table 3.

TABLE 2
Commercially available antibodies

| Ab | Brand name | Company | Target | Indication |
|---|---|---|---|---|
| Muromonab- | Orthoclone | Centocor Ortho | CD3 | Kidney transplant |
| CD3 | OKT3 | Biotech Products LP. | | rejection |
| Abciximab | Reopro | Centocor Inc./Eli | GPIIb/IIIa | Prevention of blood |
| | | Lilly/Janssen Biotech | | clots in angioplasty |
| | | Inc. | | |
| Rituximab | MabThera, | Biogen Inc./Roche, F. | CD20 | Non-Hodgkin lymphoma |
| | Rituxan | Hoffmann-La Roche | | |
| | | Ltd./Genentech Inc. | | |
| Palivizumab | Synagis | MedImmune/AbbVie | RSV | Prevention of respiratory |
| | | Inc. | | syncytial virus infection |
| Infliximab | Remicade | Janssen Biotech Inc. | TNFα | Crohn's disease |
| Trastuzumab | Herceptin | Roche, F. Hoffmann- | HER2 | Breast cancer |
| | | La Roche, Ltd./ | | |
| | | Genentech Inc. | | |
| Alemtuzumab | Campath, | Berlex Inc./Genzyme | CD52 | Chronic myeloid leukemia |
| | Lemtrada | Corp./Millennium | | |
| | | Pharmaceuticals Inc. | | |
| Adalimumab | Humira | AbbVie Inc. | TNFα | Rheumatoid arthritis |
| Ibritumomab | Zevalin | Biogen Inc./Schering | CD20 | Non-Hodgkin lymphoma |
| tiuxetan | | AG/Spectrum | | |
| | | Pharmaceuticals Inc. | | |
| Omalizumab | Xolair | Roche, F. Hoffmann- | IgE | Asthma |
| | | La Roche, Ltd./ | | |
| | | Genentech Inc./ | | |
| | | Novartis | | |
| | | Pharmaceuticals | | |
| | | Corp./Tanox Inc. | | |
| Cetuximab | Erbitux | Bristol-Myers | EGFR | Colorectal cancer |
| | | Squibb/Merck & Co. | | |
| | | Inc./Eli Lilly/ImClone | | |
| | | Systems Inc. | | |
| Bevacizumab | Avastin | Roche, F. Hoffmann- | VEGF-A | Colorectal cancer |
| | | La Roche, Ltd./ | | |
| | | Genentech Inc. | | |
| Natalizumab | Tysabri | Biogen Inc./Elan | ITGA4 | Multiple sclerosis |
| | | Pharmaceuticals | | |
| | | International, Ltd. | | |
| Panitumumab | Vectibix | Amgen | EGFR | Colorectal cancer |
| Ranibizumab | Lucentis | Roche, F. Hoffmann- | VEGF-A | Macular degeneration |
| | | La Roche Ltd./ | | |
| | | Genentech Inc./ | | |
| | | Novartis | | |
| | | Pharmaceuticals Corp. | | |
| Eculizumab | Soliris | Alexion | C5 | Paroxysmal nocturnal |
| | | Pharmaceuticals Inc. | | hemoglobinuria |
| Certolizumab | Cimzia | Celltech, UCB. | TNFα | Crohn's disease |
| pegol | | | | |
| Ustekinumab | Stelara | Medarex/Centocor | IL-12/23 | Psoriasis |
| | | Ortho Biotech Inc./ | | |
| | | Janssen Biotech Inc. | | |
| Canakinumab | Ilaris | Novartis | IL-1β | Muckle-Wells syndrome |
| | | Pharmaceuticals Corp. | | |
| Golimumab | Simponi | Centocor Ortho | TNFα | Rheumatoid and psoriatic |
| | | Biotech Inc./Janssen | | arthritis, ankylosing |
| | | Biotech Inc. | | spondylitis |
| Ofatumumab | Arzerra | Genmab A/S/ | CD20 | Chronic lymphocytic |
| | | GlaxoSmithKline/ | | leukemia |
| | | Novartis. | | |
| Tocilizumab | RoActemra, | Chugai Pharmaceutical | IL-6R | Rheumatoid arthritis |
| | Actemra | Co., Ltd./Roche, F. | | |
| | | Hoffmann-La Roche. | | |
| | | Ltd./Genentech Inc. | | |
| Denosumab | Xgeva, | Amgen | RANKL | Bone loss |
| | Prolia | | | |
| Belimumab | Benlysta | GlaxoSmithKline/ | BLyS | Systemic lupus |
| | | Human Genome | | erythematosus |
| | | Sciences Inc. | | |
| Ipilimumab | Yervoy | Bristol-Myers | CTLA-4 | Metastatic melanoma |
| | | Squibb/Medarex | | |
| Brentuximab | Adcetris | Seattle genetics | CD30 | Hodgkin lymphoma, |
| vedotin | | Inc./Takeda | | systemic anaplastic |
| | | Pharmaceutical Co., | | large cell lymphoma |
| | | Ltd. | | |
| Pertuzumab | Perjeta | Roche, F. Hoffmann- | HER2 | Breast Cancer |
| | | La Roche, Ltd./ | | |
| | | Genentech Inc. | | |
| Trastuzumab | Kadcyla | Roche, F. Hoffmann- | HER2 | Breast cancer |
| emtansine | | La Roche Ltd./ | | |
| | | Genentech Inc./ | | |
| | | ImmunoGen Inc. | | |
| Raxibacumab | Abthrax | GlaxoSmithKline/ | B. | Anthrax infection |
| | | Human Genome | anthrasis | |
| | | Sciences Inc. (HGSI) | PA | |
| Obinutuzumab | Gazyva, | Biogen Inc./Roche, F. | CD20 | Chronic lymphocytic |
| | Gazyvaro | Hoffmann-La Roche, | | leukemia |
| | | Ltd./Genentech Inc. | | |
| Siltuximab | Sylvant | Centocor Inc./Janssen | IL-6 | Castleman disease |
| | | Biotech Inc./Janssen- | | |
| | | Cilag International NV | | |
| Ramucirumab | Cyramza | Eli Lilly/ImClone | VEGFR2 | Gastric cancer |
| | | Systems Inc. | | |
| Vedolizumab | Entyvio | Genentech | α4β7 | Ulcerative colitis, |
| | | Inc./Millennium | integrin | Crohn disease |
| | | Pharmaceuticals | | |
| | | Inc./Takeda | | |
| | | Pharmaceuticals | | |
| | | U.S.A. Inc. | | |
| Blinatumomab | Blincyto | Amgen | CD19, CD3 | Acute lymphoblastic |
| | | | | leukemia |
| Nivolumab | Opdivo | Bristol-Myers Squibb/ | PD-1 | Melanoma, non-small cell |
| | | Ono Pharmaceutical | | lung cancer |
| | | Co., Ltd. | | |
| Pembrolizumab | Keytruda | Merck & Co. Inc. | PD-1 | Melanoma |
| Idarucizumab | Praxbind | Boehringer Ingelheim | Dabigatran | Reversal of dabigatran- |
| | | Pharmaceuticals | | induced anticoagulation |
| Necitumumab | Portrazza | Eli Lilly/ImClone | EGFR | Non-small cell lung |
| | | Systems Inc. | | cancer |
| Dinutuximab | Unituxin | United Therapeutics | GD2 | Neuroblastoma |
| | | Corporation | | |
| Secukinumab | Cosentyx | Novartis | IL-17α | Psoriasis |
| | | Pharmaceuticals Corp. | | |
| Mepolizumab | Nucala | Centocor Inc./ | IL-5 | Severe eosinophilic |
| | | GlaxoSmithKline | | asthma |
| Alirocumab | Praluent | Regeneron | PCSK9 | High cholesterol |
| | | Pharmaceuticals | | |
| | | Inc./Sanofi. | | |
| Evolocumab | Repatha | Amgen/Amgen | PCSK9 | High cholesterol |
| | | Astellas BioPharma | | |
| | | K. K. | | |
| Daratumumab | Darzalex | Genmab A/S/Janssen | CD38 | Multiple myeloma |
| | | Biotech Inc. | | |
| Elotuzumab | Empliciti | Bristol-Myers | SLAMF7 | Multiple myeloma |
| | | Squibb/AbbVie Inc. | | |
| Ixekizumab | Taltz | Eli Lilly | IL-17α | Psoriasis |
| Reslizumab | Cinqaero, | Celltech, UCB/ | IL-5 | Asthma |
| | Cinqair | Schering-Plough/ | | |
| | | Teva Pharmaceutical | | |
| | | Industries, Ltd. | | |
| Olaratumab | Lartruvo | Eli Lilly/ImClone | PDGFRα | Soft tissue sarcoma |
| | | Systems Inc. | | |
| Bezlotoxumab | Zinplava | Merck & Co. Inc. | Clostridium | Prevention of |
| | | | difficile | Clostridium difficile |
| | | | enterotoxin B | infection recurrence |
| Atezolizumab | Tecentriq | Roche, F. Hoffmann- | PD-L1 | Bladder cancer |
| | | La Roche, Ltd./ | | |
| | | Genentech Inc. | | |
| Obiltoxaximab | Anthim | Elusys Therapeutics | B. | Prevention of |
| | | Inc. | anthrasis | inhalational anthrax |
| | | | PA | |
| Inotuzumab | Besponsa | Wyeth | CD22 | Acute lymphoblastic |
| ozogamicin | | Pharmaceuticals/Pfizer. | | leukemia |
| Brodalumab | Siliq, | MedImmune/Amgen/ | IL-17R | Plaque psoriasis |
| | Lumicef | Kyowa Hakko Kirin/ | | |
| | | AstraZeneca/Valeant | | |
| | | Pharmaceuticals | | |
| | | International Inc. | | |
| Guselkumab | Tremfya | MorphoSys/Janssen | IL-23 p19 | Plaque psoriasis |
| | | Biotech Inc. | | |
| Dupilumab | Dupixent | Regeneron | IL-4Rα | Atopic dermatitis |
| | | Pharmaceuticals | | |
| | | Inc./Sanofi | | |
| Sarilumab | Kevzara | Regeneron | IL-6R | Rheumatoid arthritis |
| | | Pharmaceuticals | | |
| | | Inc./Sanofi | | |
| Avelumab | Bavencio | Merck Serono | PD-L1 | Merkel cell carcinoma |
| | | International | | |
| | | S. A./Pfizer | | |
| Ocrelizumab | Ocrevus | Biogen Inc./Roche, F. | CD20 | Multiple sclerosis |
| | | Hoffmann-La Roche, | | |
| | | Ltd./Genentech | | |
| | | Inc./SIGMA-TAU | | |
| | | Industrie | | |
| | | Farmaceutiche Riunite | | |
| | | S.p.A. | | |
| Emicizumab | Hemlibra | Chugai Pharmaceutical | Factor IXa, | Hemophilia A |
| | | Co., Ltd./Roche, F. | X | |
| | | Hoffmann-La Roche, | | |
| | | Ltd. | | |
| Benralizumab | Fasenra | MedImmune/Kyowa | IL-5Rα | Asthma |
| | | Hakko | | |
| | | Kirin/AstraZeneca | | |
| Gemtuzumab | Mylotarg | Pfizer | CD33 | Acute myeloid leukemia |
| ozogamicin | | | | |
| Durvalumab | Imfinzi | MedImmune/AstraZeneca | PD-L1 | Bladder cancer |
| Burosumab | Crysvita | Kyowa Hakko | FGF23 | X-linked |
| | | Kirin/Ultragenyx | | hypophosphatemia |
| | | Pharmaceutical Inc. | | |
| Lanadelumab | Takhzyro | Dyax Corp. | Plasma | Hereditary angioedema |
| | | | kallikrein | attacks |
| Mogamulizumab | Poteligeo | Kyowa Hakko Kirin | CCR4 | Mycosis fungoides or |
| | | | | Sézary syndrome |
| Erenumab | Aimovig | Novartis | CGRPR | Migraine prevention |
| Galcanezumab | Emgality | Eli Lilly | CGRP | Migraine prevention |
| Tildrakizumab | Ilumya | Merck & Co. Inc./Sun | IL-23 p19 | Plaque psoriasis |
| | | Pharmaceutical | | |
| | | Industries, Ltd. | | |
| Cemiplimab | Libtayo | Regeneron | PD-1 | Cutaneous squamous cell |
| | | Pharmaceuticals Inc. | | carcinoma |
| Emapalumab | Gamifant | NovImmmune | IFNγ | Primary hemophagocytic |
| | | | | lymphohistiocytosis |
| Fremanezumab | Ajovy | Teva Pharmaceutical | CGRP | Migraine prevention |
| | | Industries, Ltd. | | |
| Ibalizumab | Trogarzo | Taimed Biologics | CD4 | HIV infection |
| | | Inc./Theratechnologies | | |
| | | Inc. | | |
| Moxetumomab | Lumoxiti | MedImmune/AstraZeneca | CD22 | Hairy cell leukemia |
| pasudodox | | | | |
| Ravulizumab | Ultomiris | Alexion | C5 | Paroxysmal nocturnal |
| | | Pharmaceuticals Inc. | | bemoglobinuria |
| Caplacizumab | Cablivi | Ablynx | von | Acquired thrombotic |
| | | | Willebrand | thrombocytopenia purpura |
| | | | factor | |
| Romosozumab | Evenity | Amgen/UCB | Sclerostin | Osteoporosis in |
| | | | | postmenopausal women at |
| | | | | increased risk of fracture |
| Risankizumab | Skyrizi | Boehringer Ingelheim | IL-23 p19 | Plaque psoriasis |
| | | Pharmaceuticals/ | | |
| | | AbbVie Inc. | | |
| Polatuzumab | Polivy | Roche, F. Hoffmann- | CD79β | Diffuse large B-cell |
| vedotin | | La Roche, Ltd. | | lymphoma |
| Brolucizumab | Beovu | Novartis | VEGF-A | Macular degeneration |
| | | Pharmaceuticals Corp. | | |
| Crizanlizumab | Adakveo | Novartis | P-selectin | Sickle cell disease |
| | | Pharmaceuticals Corp. | | |

TABLE 3

| Organism | Exemplary Antigens |
|---|---|
| Legionella Pneumophila | |
| Salmonella | O or R antigen |
| Staphylococcus | poly-N-succinyl β-1-6 |
| | glucosamine (PNSG), or |
| | Protein A |
| Ureaplasma urealyticum | |
| Aeromonas | |
| Arthrobacter Globiformis | |
| Bacillus (e.g., B. thermophilus, B. halodurans. | |
| B. alcalophilus). | |
| Bordetella Pertussis | |
| Borrelia (e.g., B. burgdorferi sensu stricto, B. garinii, | |
| B. bavariensis, B. afzelii and B. spielmanii) | |
| Brucella Abortus | |
| Campylobacter Jejuni | |
| Candida Albicans | |
| Chlamydia Trachomatis | |
| Chlamydophila Pneumoniae | |
| Clostridium tetani | |
| Corynebacterium Diphtheriae | |
| Escherichia coli | LPS (O antigens) an PS |
| | (K antigens) |
| Haemophilus influenzae | |
| Helicobacter pylori | |
| Leptospira biflexa | |
| Listeria monocytogenes | |
| Mycobacterium Tuberculosis | |
| Mycoplasma Pneumoniae | |
| Streptococcus Pneumoniae | M and T antigens |
| Streptomyces Avidinii | |
| Treponema Pallidum | |
| Yersinia Enterocolitica | |
| Adenovirus | |
| ASFV | |
| Chikungunya Virus | |
| Coronavirus | |
| Coxsackie Virus | |
| Crimean-Congo Hemorrhagic Fever Virus | |
| Cytomegalovirus | |
| Dengue Virus | |
| Ebola Virus | |
| Echovirus | |
| Enterovirus | |
| Epstein-Barr Virus | |
| Hepatitis A virus (HAV) | Surface glycoprotein |
| Hepatitis B virus (HBV) | Hepatitis surface antigen |
| Human cytomegalovirus (HCMV) | gB or gH |
| Hepatitis C virus (HCV) | S protein or E2 protein |
| Hepatitis Delta virus (HDV) | |
| Hepatitis E virus (HEV) | |
| Human immunodeficiency virus (HIV) (e.g., type 1 or 2) | Gp120 or Nef |
| Human papillomavirus (HPV) | E6 or E7 |
| Herpes simplex virus (HSV) | gB, gD, and gH/gL |
| Human T-lymphotropic virus (HTLV) (e.g., type 1 or 2) | Tax protein |
| Influenza A Virus (e.g., group 1 or 2) | Hemagglutinin or |
| | neuraminidase |
| Influenza B Virus | Hemagglutinin or |
| | neuraminidase |
| Japanese Encephalitis | Envelope protein |
| Leukemia Virus | |
| Marburg Virus | Surface glycoprotein |
| Measles Virus | H, F, M, NP, or P |
| Metapneumovirus | |
| Molluscum Contagiosum | |
| Mumps Virus | |
| Nipah Virus | |
| Norovirus | |
| Orf Virus | |
| Parainfluenza Virus | |
| Parvovirus | |
| Poliovirus | |
| Rabies Virus | |
| Respiratory Syncytial Virus | F or G protein |
| Rhinovirus | |
| Rift Valley Fever Virus | |
| Poliovirus | |
| Rotavirus | |
| Rubella Virus | |
| Severe acute respiratory syndrome coronavirus 1 | Spike protein or |
| (SARS-CoV-1) | nucleoprotein |
| Severe acute respiratory syndrome coronavirus 2 | Spike protein or |
| (SARS-CoV-2) | nucleoprotein |
| Simian (Macaque) Immunodeficiency Virus | |
| TBEV | |
| Tobacco Etch Virus | |
| Varicella Zoster Virus | |
| Variola | |
| Venezuelan Equine Encephalitis Virus | |
| West Nile Virus | |
| Yellow Fever Virus | |
| Zika Virus | |
| Aspergillus | |
| Saccharomyces (e.g., Saccharomyces cerevisiae) | |
| Leishmania | |
| Plasmodium Falciparum | |
| Plasmodium Vivax | |
| Plasmodium | |
| Toxoplasma Gondii | |
| Caenorhabditis Elegans | |
| Echinococcus Granulosus | |
| Malaria | |
| Schistosoma Japonicum | |
| Trichomonas Vaginalis | |
| Trypanosoma cruzi (T. cruzi) | |

### 7.2. Polynucleotides

In one aspect, provided herein is a nucleic acid sequence comprising a nucleotide sequence encoding a protein described herein. The nucleic acid sequences of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the nucleic acid sequence is in the form of cDNA. In some embodiments, the nucleic acid sequence is a synthetic polynucleotide.

In another aspect, provided herein are vectors comprising the nucleic acid sequences described herein. In an embodiment, the nucleic acid sequences can be incorporated into a recombinant expression vector. The present disclosure provides recombinant expression vectors comprising any of the nucleic acid sequences of the disclosure. In a specific embodiment, a “recombinant expression vector” refers to genetically-modified oligonucleotide or polynucleotide construct that permits the expression of an mRNA, protein, polypeptide, or peptide by a host cell (e.g., bacterium, plant, fungus, or an animal cell), when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole; however, parts of the vectors can be naturally-occurring. The described recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. The non-naturally occurring or altered nucleotides or internucleotide linkages do not hinder the transcription or replication of the vector.

In an embodiment, the recombinant expression vector of the disclosure can be any suitable recombinant expression vector, and can be used to transform or transfect any suitable host (e.g., bacterium, plant, fungus, or animal). Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, La Jolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Examples of plant expression vectors include pBI01, pBI01.2, pBI121, pBI101.3, and pBIN19 (Clontech). Examples of animal expression vectors include pEUK-Cl, pMAM, and pMAMneo (Clontech). The recombinant expression vector may be a viral vector, e.g., a retroviral vector, e.g., a gamma retroviral vector.

In an embodiment, the recombinant expression vectors are prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, SV40, 2μ plasmid, λ, bovine papilloma virus, and the like.

The recombinant expression vector may comprise regulatory sequences, such as transcription and translation initiation and termination codons, which are specific to the type of host (e.g., bacterium, plant, fungus, or animal) into which the vector is to be introduced, as appropriate, and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the described expression vectors include, for instance, neomycin/G418 resistance genes, histidinol×resistance genes, histidinol resistance genes, tetracycline resistance genes, and ampicillin resistance genes.

The recombinant expression vector can comprise a native or normative promoter operably linked to the nucleotide sequence of the disclosure. The selection of promoters, e.g., strong, weak, tissue-specific, inducible and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an RSV promoter, an SV40 promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

In certain embodiments, a nucleic acid sequence is isolated. In certain embodiments, a nucleic acid sequence is substantially pure.

Also provided are cells comprising the nucleic acid sequences described herein. The cell may be any cell that contains a heterologous nucleic acid sequence. The heterologous nucleic acid sequence can be a vector (e.g., an expression vector). For example, a cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as cells for vector replication and/or expression. Additionally, bacterial cells such as E. coli LE392 could be used as cells for phage viruses. Eukaryotic cells that can be used as cells include, but are not limited to yeast (e.g., YPH499, YPH500 and YPH501), insects and mammals. Examples of mammalian eukaryotic cells for replication and/or expression of a vector include, but are not limited to, HeLa, NIH3T3, Jurkat, 293, COS, Saos, PC12, SP2/0 (American Type Culture Collection (ATCC), Manassas, VA, CRL-1581), NS0 (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATCC CRL-TIB-196). Other useful cell lines include those derived from Chinese Hamster Ovary (CHO) cells such as CHO-K1SV (Lonza Biologics, Walkersville, MD), CHO-K1 (ATCC CRL-61) or DG44. A cell transfected or transformed with a nucleic acid sequence includes progeny or potential progeny of such a cell. Progeny of such a cell may not be identical to the parent cell transfected or transformed with the nucleic acid sequence due to mutations or environmental influences that may occur in succeeding generations or integration of the nucleic acid sequence into the cell genome.

### 7.3. Methods for Producing Protein

In one aspect, provided herein are methods for producing a polypeptide or protein described herein (e.g., an antibody or another antigen binding protein), comprising culturing a cell(s) transfected or transformed with a nucleic acid sequence comprising a nucleotide sequence encoding the polypeptide or protein. The cell may be any cell described herein (e.g., in this section or Section 7.2). In specific embodiments, the methods further comprise isolating the protein from the cell(s).

In another aspect, provided herein are methods for producing a protein described herein (e.g., an antibody or another antigen binding protein), comprising culturing cells transformed or transfected with a vector containing a nucleic acid sequence encoding the protein. The cell may be any cell described herein (e.g., in this section or Section 7.2) or known in the art. In specific embodiments, the methods further comprise isolated the protein from the cell(s).

Polynucleotide sequences encoding polypeptide components of a protein (e.g., an antibody or other antigen binding protein) of the present disclosure can be obtained using standard recombinant techniques. Desired polynucleotide sequences may be isolated and sequenced from protein (e.g., antibody) producing cells such as hybridomas cells or B cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in cells. Many vectors that are available and known in the art can be used for the purpose of the present disclosure. Selection of an appropriate vector will depend mainly on the size of the nucleic acid sequence to be inserted into the vector and the particular cell to be transformed with the vector.

Cells suitable for expressing polypeptides or proteins (e.g., antibodies) of the present disclosure include prokaryotes such as Archaebacteria and Eubacteria, including Gram-negative or Gram-positive organisms, eukaryotic microbes such as filamentous fungi or yeast, invertebrate cells such as insect or plant cells, and vertebrate cells such as mammalian cell lines. Cells are transformed with the above-described expression vectors and cultured in conventional nutrient media modified as appropriate for inducing promoters, selecting transformants, or amplifying the genes encoding the desired sequences. A polypeptide or protein (e.g., an antibody) produced by the cells may be isolated or purified using standard purification methods as known in the art.

Methods for antibody production including vector construction, expression, and purification are further described in Plückthun et al., Antibody Engineering: Producing antibodies in Escherichia coli: From PCR to fermentation 203-52 (McCafferty et al. eds., 1996); Kwong and Rader, E. coli Expression and Purification of Fab Antibody Fragments, in Current Protocols in Protein Science (2009); Tachibana and Takekoshi, Production of Antibody Fab Fragments in Escherichia coli, in Antibody Expression and Production (Al-Rubeai ed., 2011); and Therapeutic Monoclonal Antibodies: From Bench to Clinic (An ed., 2009).

It is, of course, contemplated that alternative methods, which are well known in the art, may be employed to prepare a protein described herein (e.g., an antibody or another antigen binding protein). For instance, the appropriate amino acid sequence, or portions thereof, may be produced by direct peptide synthesis using solid-phase techniques (see, e.g., Stewart et al., Solid-Phase Peptide Synthesis (1969); and Merrifield, J. Am. Chem. Soc. 85:2149-54 (1963)). In vitro protein synthesis may be performed using manual techniques or by automation. Various portions of the protein (e.g., antibody or another antigen binding protein) may be chemically synthesized separately and combined using chemical or enzymatic methods to produce the desired protein.

### 7.3.1. Recombinant Production in Prokaryotic Cells

Sequences encoding a polypeptide or protein described herein may be inserted into a recombinant vector capable of replicating and expressing heterologous polynucleotides in prokaryotic hosts. Many vectors that are available and known in the art can be used for the purpose of the present disclosure. Selection of an appropriate vector will depend mainly on the size of the nucleic acid sequence to be inserted into the vector and the particular host cell to be transformed with the vector. Each vector contains various components, depending on its function (amplification or expression of heterologous polynucleotide, or both) and its compatibility with the particular cell in which it resides. The vector components generally include, but are not limited to, an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the cell are used. The vector ordinarily carries a replication site, as well as marking sequences which are capable of providing phenotypic selection in transformed cells. For example, E. coli is typically transformed using pBR322, a plasmid derived from an E. coli species. Examples of pBR322 derivatives used for expression of particular polypeptides or proteins (e.g., antibodies) are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector which can be used to transform susceptible host cells such as E. coli LE392.

The expression vector of the present application may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential cells are well known. The selected promoter can be operably linked to cistron DNA encoding a polypeptide or protein described herein (e.g., an antibody) by removing the promoter from the source DNA via restriction enzyme digestion and inserting the isolated promoter sequence into the vector of the present application. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleic acid sequences have been published, thereby enabling a skilled worker operably to ligate them to cistrons encoding the target peptide (Siebenlist et al. Cell 20:269 (1980)) using linkers or adaptors to supply any required restriction sites.

In one aspect, each cistron within the recombinant vector comprises a secretion signal sequence component that directs translocation of the expressed polypeptides across a membrane.

In general, the signal sequence may be a component of the vector, or it may be a part of the target polypeptide DNA that is inserted into the vector. The signal sequence selected should be one that is recognized and processed (i.e. cleaved by a signal peptidase) by the cell. For prokaryotic cells that do not recognize and process the signal sequences native to the heterologous polypeptides, the signal sequence can be substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP.

In some embodiments, the production of the polypeptides or proteins (e.g., antibodies) according to the present disclosure can occur in the cytoplasm of the cell, and therefore does not require the presence of secretion signal sequences within each cistron. Certain strains (e.g., the E. coli trxB⁻ strains) provide cytoplasm conditions that are favorable for disulfide bond formation, thereby permitting proper folding and assembly of expressed polypeptide or protein subunits.

Prokaryotic cells suitable for expressing the polypeptides or proteins (e.g., antibodies) of the present disclosure include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In one embodiment, E. coli cells are used as hosts. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A (nmpc-fepE) degP41 kan^{R} (U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well-known plasmids such as pBR322, pBR325, pACYC177, or pKN410 are used to supply the replicon.

Typically, the host cell should secrete minimal amounts of proteolytic enzymes, and additional protease inhibitors may desirably be incorporated in the cell culture.

Cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides or proteins (e.g., antibodies) of the present application are grown in media known in the art and suitable for culture of the selected cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol. The prokaryotic host cells are cultured at suitable temperatures and pHs.

If an inducible promoter is used in the expression vector of the present application, polypeptide or protein expression is induced under conditions suitable for the activation of the promoter. In one aspect of the present application, PhoA promoters are used for controlling transcription of the polypeptides. Accordingly, the transformed cells are cultured in a phosphate-limiting medium for induction. In some embodiments, the phosphate-limiting medium is the completely radical alkaline phosphatase (C.R.A.P) medium (see, e.g., Simmons et al., J. Immunol. Methods 263:133-147 (2002)). A variety of other inducers may be used, according to the vector construct employed, as is known in the art.

The expressed polypeptides or proteins (e.g., antibodies) of the present disclosure are secreted into and recovered from the periplasm of the cells. Polypeptide or protein recovery typically involves disrupting the microorganism, generally by such means as osmotic shock, sonication or lysis. Once cells are disrupted, cell debris or whole cells may be removed by centrifugation or filtration. The polypeptides or proteins may be further purified, for example, by affinity resin chromatography. Alternatively, polypeptides or proteins can be transported into the culture media and isolated therein. Cells may be removed from the culture and the culture supernatant being filtered and concentrated for further purification of the polypeptides or proteins produced. The expressed polypeptides or proteins can be further isolated and identified using commonly known methods such as polyacrylamide gel electrophoresis (PAGE) and Western blot assay.

Alternatively, polypeptides or protein production is conducted in large quantity by a fermentation process. Various large-scale fed-batch fermentation procedures are available for production of recombinant polypeptides or proteins. To improve the production yield and quality of the polypeptides or proteins (e.g., antibodies) of the present disclosure, various fermentation conditions can be modified. For example, the chaperone proteins have been demonstrated to facilitate the proper folding and solubility of heterologous proteins produced in bacterial cells. Chen et al. J Bio Chem 274:19601-19605 (1999); U.S. Pat. Nos. 6,083,715; 6,027,888; Bothmann and Pluckthun, J. Biol. Chem. 275:17100-17105 (2000); Ramm and Pluckthun, J. Biol. Chem. 275:17106-17113 (2000); Arie et al., Mol. Microbiol. 39:199-210 (2001).

To minimize proteolysis of expressed heterologous proteins (especially those that are proteolytically sensitive), certain host strains deficient for proteolytic enzymes can be used for the present invention, as described in, for example, U.S. Pat. Nos. 5,264,365; 5,508,192; Hara et al., Microbial Drug Resistance, 2:63-72 (1996). E. coli strains deficient for proteolytic enzymes and transformed with plasmids overexpressing one or more chaperone proteins may be used as cells in the expression system encoding the proteins (e.g., antibodies) of the present application.

The polypeptides or proteins (e.g., antibodies) produced herein can be further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75. Protein A immobilized on a solid phase for example can be used in some embodiments for immunoaffinity purification of binding molecules of the present disclosure. In some embodiments, the solid phase to which Protein A is immobilized is a column comprising a glass or silica surface. In some embodiments, the solid phase to which Protein A is immobilized is a controlled pore glass column or a silicic acid column. In some embodiments, the column has been coated with a reagent, such as glycerol, in an attempt to prevent nonspecific adherence of contaminants. The solid phase is then washed to remove contaminants non-specifically bound to the solid phase. Finally, the polypeptide or protein (e.g., antibody) of interest is recovered from the solid phase by elution.

### 7.3.2. Recombinant Production in Eukaryotic Cells

For eukaryotic expression, the vector components generally include, but are not limited to, one or more of the following, a signal sequence, an origin of replication, one or more marker genes, and enhancer element, a promoter, and a transcription termination sequence.

A vector for use in a eukaryotic host may also include an insert that encodes a signal sequence or other polypeptide having a specific cleavage site at the N-terminus of the mature protein or polypeptide. The heterologous signal sequence selected preferably is one that is recognized and processed (i.e., cleaved by a signal peptidase) by the cell. In mammalian cell expression, mammalian signal sequences as well as viral secretory leaders, for example, the herpes simplex gD signal, are available. The DNA for such precursor region can be ligated in reading frame to DNA encoding the antibodies of the present application.

Generally, the origin of replication component is not needed for mammalian expression vectors (the SV40 origin may typically be used only because it contains the early promoter).

Expression and cloning vectors may contain a selection gene, also termed a selectable marker. Selection genes may encode proteins that confer resistance to antibiotics or other toxins, e.g., ampicillin, neomycin, methotrexate, or tetracycline; complement auxotrophic deficiencies; or supply critical nutrients not available from complex media.

One example of a selection scheme utilizes a drug to arrest growth of a cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up nucleic acid encoding the antibodies of the present application. For example, cells transformed with the DHFR selection gene are first identified by culturing all of the transformants in a culture medium that contains methotrexate (Mtx), a competitive antagonist of DHFR. An exemplary appropriate cell when wild-type DHFR is employed is the Chinese hamster ovary (CHO) cell line deficient in DHFR activity. Alternatively, cells (particularly wild-type hosts that contain endogenous DHFR) transformed or co-transformed with the polypeptide encoding-DNA sequences, wild-type DHFR protein, and another selectable marker such as aminoglycoside 3′-phosphotransferase (APH) can be selected by cell growth in medium containing a selection agent for the selectable marker such as an aminoglycosidic antibiotic.

Expression and cloning vectors usually contain a promoter that is recognized by the host organism and is operably linked to the nucleic acid encoding the desired polypeptide sequences. Eukaryotic genes have an AT-rich region located approximately 25 to 30 based upstream from the site where transcription is initiated. Another sequence found 70 to 80 bases upstream from the start of the transcription of many genes may be included. The 3′ end of most eukaryotic may be the signal for addition of the poly A tail to the 3′ end of the coding sequence. All of these sequences may be inserted into eukaryotic expression vectors.

Polypeptide transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding the polypeptides or proteins (e.g., antibodies) of the present disclosure by higher eukaryotes is often increased by inserting an enhancer sequence into the vector. Many enhancer sequences are now known from mammalian genes (globin, elastase, albumin, α-fetoprotein, and insulin). Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide encoding sequence, but is preferably located at a site 5′ from the promoter.

Expression vectors used in eukaryotic host cells (yeast, fungi, insect, plant, animal, human, or nucleated cells from other multicellular organisms) also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA. One useful transcription termination component is the bovine growth hormone polyadenylation region.

Suitable cells for cloning or expressing the DNA in the vectors herein include higher eukaryote cells described herein, including vertebrate host cells. Propagation of vertebrate cells in culture (tissue culture) has become a routine procedure. Examples of useful mammalian cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Cells can be transformed with the above-described expression or cloning vectors for polypeptide or protein (e.g., antibody) production and cultured in conventional nutrient media modified as appropriate for inducing promoters, selecting transformants, or amplifying the genes encoding the desired sequences.

The cells used to produce the polypeptides or proteins (e.g., antibodies) of the present application may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the polypeptides or proteins (e.g., antibodies) can be produced intracellularly, in the periplasmic space, or directly secreted into the medium. If the polypeptide or protein (e.g., antibody) is produced intracellularly, as a first step, the particulate debris, either cells or lysed fragments, are removed, for example, by centrifugation or ultrafiltration. Where the polypeptide or protein (e.g., antibody) is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The polypeptide or protein composition prepared from the cells can be purified using, for example, hydroxylapatite chromatography, gel electrophoresis, dialysis, and affinity chromatography, with affinity chromatography being the preferred purification technique. The matrix to which the affinity ligand is attached is most often agarose, but other matrices are available. Mechanically stable matrices such as controlled pore glass or poly (styrene-divinyl)benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the polypeptide or protein (e.g., antibody) to be recovered. Following any preliminary purification step(s), the mixture comprising the polypeptide or protein (e.g, antibody) of interest and contaminants may be subjected to low pH hydrophobic interaction chromatography.

### 7.4. Compositions

In one aspect, the present disclosure provides pharmaceutical compositions comprising at least one polypeptide or protein (e.g., an antibody or another antigen binding protein) of the present disclosure. In some embodiments, a pharmaceutical composition comprises an effective amount of a polypeptide or protein (e.g., an antibody or another antigen binding protein) provided herein and a pharmaceutically acceptable excipient. The therapeutically effective amount of a polypeptide or protein (e.g., an antibody) may be the dose approved the FDA or another regulatory agency, or known to one of skill in the art. For example, the therapeutically effective amount may be the amount of a protein in Table 2, above, which has been approved by the FDA or another regulatory agency.

Pharmaceutical compositions comprising a polypeptide or protein (e.g., an antibody or another antigen binding protein) may be prepared for storage by mixing the polypeptide or protein having the desired degree of purity with optional physiologically acceptable excipients (see, e.g., Remington, Remington's Pharmaceutical Sciences (18th ed. 1980)) in the form of aqueous solutions or lyophilized or other dried forms.

A polypeptide or protein (e.g., an antibody or another antigen binding protein) of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

A polypeptide or protein (e.g., an antibody or another antigen binding protein) described herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with a polypeptide or protein (e.g., an antibody or another antigen binding protein) described herein, including, but not limited to, encapsulation in liposomes, microparticles, microcapsules, recombinant cells capable of expressing the antibody or antibody fragment, receptor-mediated endocytosis (see, e.g., Wu and Wu, 1987, J. Biol. Chem. 262:4429-32), construction of a nucleic acid sequence as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody or antibody fragment as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more polypeptides or proteins (e.g., antibodies or other antigen binding proteins) described herein (see, e.g., U.S. Pat. No. 4,526,938, International Patent Application Publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### 7.5. Methods for Reducing the Interaction Between a Polypeptide or a Protein and a Reference

Provided herein are methods for reducing interaction reducing the interaction between a polypeptide and a reference, wherein the polypeptide comprises one or more target binding regions (e.g., two, three, or more target binding regions) and an exposed C-terminus, the method comprising fusing a cap domain (e.g., a His cap domain, SLSLSPGK (SEQ ID NO:36), or TVAPTESS (SEQ ID NO:37)) at the end of the exposed C-terminus. In specific embodiments, the reference is a molecule present in a biological substance. In some embodiments, the biological substance comprises serum and/or plasma (e.g., human serum and/or human plasma). In some embodiments, the biological substance comprises serum (e.g., human serum). In some embodiments, the biological substance comprises plasma (e.g., human plasma). In specific embodiments, the reference comprises an anti-drug antibody. In some embodiments, the anti-drug antibody is pre-existing in serum and/or plasma of a subject (e.g., a human subject). In some embodiments, the one or more target binding regions comprise an antibody variable domain or an antigen binding fragment thereof. In specific embodiments, the exposed C-terminus comprises an anti-drug antibody epitope. In some embodiments, the cap domain is a His cap domain. In some embodiments, the exposed C-terminus fused to a His cap domain consists of the amino acid sequence selected from the group consisting of: VTVSSH (SEQ ID NO:1), VTVSSHH (SEQ ID NO: 2), VTVSSHHH (SEQ ID NO:3), VTVSSHHHH (SEQ ID NO:4), and VTVSSHHHHH (SEQ ID NO:5). In other embodiments, the exposed C-terminus fused to a His cap domain consists of the amino acid sequence selected from the group consisting of: VEIKH (SEQ ID NO: 6), VEIKHH (SEQ ID NO:7), VEIKHHH (SEQ ID NO:8), VEIKHHHH (SEQ ID NO:9), and VEIKHHHHH (SEQ ID NO:10). In yet other embodiments, the exposed C-terminus fused to a His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15). In still further embodiments, the exposed C-terminus fused to a His cap domain consists of the amino acid sequence selected from the group consisting of: VEIKRTH (SEQ ID NO:16), VEIKRTHH (SEQ ID NO: 17), VEIKRTHHH (SEQ ID NO:18), VEIKRTHHHH (SEQ ID NO:19), and VEIKRTHHHHH (SEQ ID NO:20). In some embodiments, the exposed C-terminus fused to the His cap domain consists of the amino acid sequence of TKVTVL (His)n (SEQ ID NO:46), TKLTVL (His)n (SEQ ID NO:47), TQLIIL (His)n (SEQ ID NO:48), TELTVL (His)n (SEQ ID NO: 49), or TQLTVL (His)n (SEQ ID NO:50), wherein n is 1, 2, 3, 4, or 5. In some embodiments, the exposed C-terminus fused to the His cap domain consists of the amino acid sequence of TKLTVL (His)n (SEQ ID NO:47), TKVTVL (His)n (SEQ ID NO:46), NGLVYAG (His)n (SEQ ID NO: 51), NGLLYAG (His)n (SEQ ID NO:52), or TRLTVL (His)n (SEQ ID NO:53), wherein n is 1, 2, 3, 4, or 5.

In specific embodiments, interaction is determined by an immunoassay.

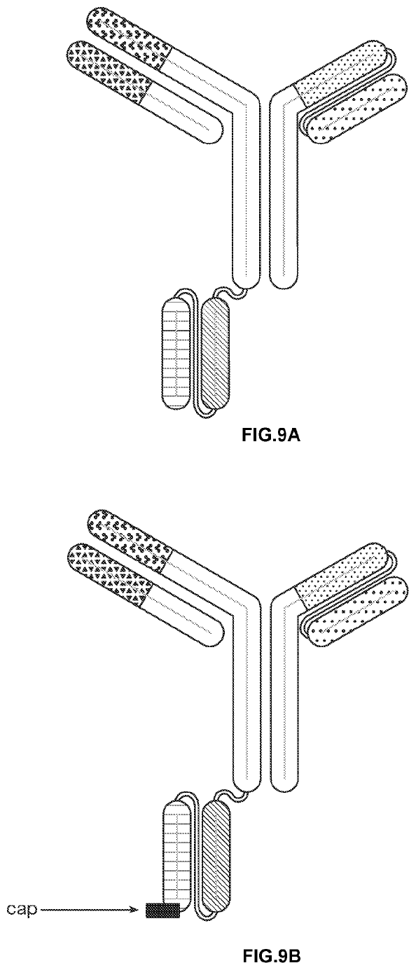

Various techniques for adding a cap domain (e.g., a His cap domain, SLSLSPGK (SEQ ID NO:36), or TVAPTESS (SEQ ID NO:37)) are known in the art, such as insertion of a nucleotide sequence encoding the cap domain at an exposed C-terminus. Conventional molecular biology techniques may be used to add a cap domain. In specific embodiments, a cap domain is added to at the end of an exposed C-terminus of an antigen binding domain (e.g., an scFv, a heavy chain, a heavy chain variable domain, a light chain, or a light chain variable domain). See, e.g., FIGS. 9B, 10A and 10B for an example of where a cap domain may be added.

In another aspect, provided herein are methods for reducing the interaction between a polypeptide and a reference, wherein the polypeptide comprises one or more target binding regions (e.g., two, three, or more target binding regions) and an exposed C-terminus, the method comprising fusing a cap domain (e.g., a His cap domain, SLSLSPGK (SEQ ID NO:36), or TVAPTESS (SEQ ID NO:37)) at the end of the exposed C-terminus. In some embodiments, the one or more target binding regions comprise an antibody variable domain or antigen binding fragment thereof. In another aspect, provided herein are methods for reducing the interaction between a protein and a reference, wherein the protein includes (a) a first polypeptide comprising a target binding region, and an exposed C-terminus; and (b) a second polypeptide comprising a target binding region, the method comprising fusing a cap domain (e.g., a His cap domain, SLSLSPGK (SEQ ID NO:36), or TVAPTESS (SEQ ID NO:37)) at the end of the exposed C-terminus. In some embodiments, the second polypeptide comprises an exposed C-terminus. In some embodiments, the target binding region of the first and second polypeptides are capable of binding to different targets. In certain embodiments, the protein further comprises a third polypeptide comprising a target binding region. In some embodiments, the third polypeptide comprises an exposed C-terminus. In some embodiments, the third polypeptide is capable of binding to a different target than the first polypeptide, the second polypeptide, or both. In some embodiments, the protein is heterodimeric. In some embodiments, the protein is bispecific. In some embodiments, the protein is trispecific. In some embodiments, the protein is multi-specific. In another aspect, provided herein are methods for reducing the interaction between a bispecific or multi-specific antibody and a reference, wherein the antibody comprises one or more target binding regions and an exposed C-terminus, the method comprising fusing a cap domain (e.g., a His cap domain, SLSLSPGK (SEQ ID NO:36), or TVAPTESS (SEQ ID NO:37)) at the end of the exposed C-terminus. In some embodiments, the cap domain is a His cap domain. In specific embodiments, the polypeptide, protein, antibody is isolated. In specific embodiments, the interaction is determined by an assay described herein (such as an assay described in Section 7.9.1) or known to one of skill in the art.

In some embodiments, the addition of a cap domain (e.g., a His cap domain, SLSLSPGK (SEQ ID NO:36), or TVAPTESS (SEQ ID NO:37)) at the end of an exposed C-terminus of a polypeptide or a protein (e.g., an antibody, such as, e.g., a bispecific antibody, or a multispecific antibody (e.g., trispecific antibody)) results in a reduction in the interaction of the polypeptide or the protein with a reference relative to the interaction of the same reference with the same polypeptide or the same protein, respectively, lacking the cap domain. In some embodiments, the addition of one histidine at the end of an exposed C-terminus of a polypeptide or a protein (e.g., an antibody, such as, e.g., a bispecific antibody, or a multispecific antibody (e.g., trispecific antibody)) results in a reduction in the interaction of the polypeptide or the protein with a reference relative to the interaction of the same reference with the same polypeptide or the same protein, respectively, lacking the one histidine. In some embodiments, the addition of two histidines at the end of an exposed C-terminus of a polypeptide or a protein (e.g., an antibody, such as, e.g., a bispecific antibody, or a multispecific antibody (e.g., trispecific antibody)) results in a reduction in the interaction of the polypeptide or the protein with a reference relative to the interaction of the same reference with the same polypeptide or the same protein, respectively, lacking the two histidines. In some embodiments, the addition of three histidines at the end of an exposed C-terminus of a polypeptide or a protein (e.g., an antibody, such as, e.g., a bispecific antibody, or a multispecific antibody (e.g., trispecific antibody)) results in a reduction in the interaction of the polypeptide or the protein with a reference relative to the interaction of the same reference with the same polypeptide or the same protein, respectively, lacking the three histidines. In some embodiments, the addition of four histidines at the end of an exposed C-terminus of a polypeptide or a protein (e.g., an antibody, such as, e.g., a bispecific antibody, or a multispecific antibody (e.g., trispecific antibody)) results in a reduction in the interaction of the polypeptide or the protein with a reference relative to the interaction of the same reference with the same polypeptide or the same protein, respectively, lacking the four histidines. In some embodiments, the addition of five histidines at the end of an exposed C-terminus of a polypeptide or a protein (e.g., an antibody, such as, e.g., a bispecific antibody, or a multispecific antibody (e.g., trispecific antibody)) results in a reduction in the interaction of the polypeptide or the protein with a reference relative to the interaction of the same reference with the same polypeptide or the same protein, respectively, lacking the five histidines. In certain embodiments, the interaction is reduced by at least 10%, at least 15%, at least 20%, or at least 25%. In some embodiments, the interaction is reduced by at least 30%, at least 35%, at least 40%, or at least 45%. In some embodiments, the interaction is reduced by at least 50%, at least 55%, at least 60%, or at least 65%. In some embodiments, the interaction is reduced by at least 70%, at least 75%, at least 80%, or at least 85%. In some embodiments, the interaction is reduced by at least 90%, at least 95%, or at least 98%. In specific embodiments, the interaction between the polypeptide or protein is assessed in an assay described herein or known to one of skill in the art.

Also provided herein are methods for reducing the aggregation of a polypeptide in a biological substance, wherein the polypeptide comprises one or more target binding regions and an exposed C-terminus, the method comprising fusing a cap domain (e.g., a His cap domain, SLSLSPGK (SEQ ID NO:36), or TVAPTESS (SEQ ID NO:37)) at the end of the exposed C-terminus. In some embodiments, the biological substance comprises serum and/or plasma (e.g., human serum and/or plasma). In specific embodiments, the biological substance is human serum. In specific embodiments, the biological substance is human plasma. In some embodiments, the polypeptide in the biological substance is incubated at a certain temperature for a certain period of time before aggregation is assessed. In some embodiments, incubation comprises about seven days at about 37° C. in human serum and/or human plasma. In some embodiments, incubation comprises about two to about seven days at about 37° C. in human serum and/or human plasma. In some embodiments, incubation comprises about two to about five days at about 37° C. in human serum and/or human plasma. In some embodiments, the one or more target binding regions comprise an antibody variable domain or an antigen binding fragment thereof. In specific embodiments, the exposed C-terminus comprises an anti-drug antibody epitope. In some embodiments, the exposed C-terminus fused to a His cap domain consists of the amino acid sequence selected from the group consisting of: VTVSSH (SEQ ID NO:1), VTVSSHH (SEQ ID NO: 2), VTVSSHHH (SEQ ID NO:3), VTVSSHHHH (SEQ ID NO:4), and VTVSSHHHHH (SEQ ID NO:5). In other embodiments, the exposed C-terminus fused to a His cap domain consists of the amino acid sequence selected from the group consisting of: VEIKH (SEQ ID NO: 6), VEIKHH (SEQ ID NO:7), VEIKHHH (SEQ ID NO:8), VEIKHHHH (SEQ ID NO:9), and VEIKHHHHH (SEQ ID NO:10). In yet other embodiments, the exposed C-terminus fused to a His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15). In still further embodiments, the exposed C-terminus fused to a His cap domain consists of the amino acid sequence selected from the group consisting of: VEIKRTH (SEQ ID NO:16), VEIKRTHH (SEQ ID NO: 17), VEIKRTHHH (SEQ ID NO:18), VEIKRTHHHH (SEQ ID NO:19), and VEIKRTHHHHH (SEQ ID NO:20). In some embodiments, the exposed C-terminus fused to the His cap domain consists of the amino acid sequence of TKVTVL (His)n (SEQ ID NO:46), TKLTVL (His)n (SEQ ID NO:47), TQLIIL (His)n (SEQ ID NO:48), TELTVL (His)n (SEQ ID NO: 49), or TQLTVL (His)n (SEQ ID NO:50), wherein n is 1, 2, 3, 4, or 5. In some embodiments, the exposed C-terminus fused to the His cap domain consists of the amino acid sequence of TKLTVL (His)n (SEQ ID NO:47), TKVTVL (His)n (SEQ ID NO:46), NGLVYAG (His)n (SEQ ID NO: 51), NGLLYAG (His)n (SEQ ID NO:52), or TRLTVL (His)n (SEQ ID NO:53), wherein n is 1, 2, 3, 4, or 5.

In another aspect, provided herein are methods for reducing the aggregation of a polypeptide in a biological substance, wherein the polypeptide comprises one or more target binding regions (e.g., two, three, or more target binding regions) and an exposed C-terminus, the method comprising fusing a cap domain (e.g., a His cap domain, SLSLSPGK (SEQ ID NO:36), or TVAPTESS (SEQ ID NO:37)) at the end of the exposed C-terminus. In a further aspect, provided herein are methods for reducing aggregation of a protein, wherein the protein includes (a) a first polypeptide comprising a target binding region and an exposed C-terminus; and (b) a second polypeptide comprising a target binding region, the method comprising fusing a cap domain (e.g., a His cap domain, SLSLSPGK (SEQ ID NO:36), or TVAPTESS (SEQ ID NO:37)) at the end of the exposed C-terminus. In some embodiments, the second polypeptide comprises an exposed C-terminus. In some embodiments, the target binding region of the first and second polypeptides are capable of binding to different targets. In certain embodiments, the protein further comprises a third polypeptide comprising a target binding region. In some embodiments, the third polypeptide comprises an exposed C-terminus. In some embodiments, the third polypeptide is capable of binding to a different target than the first polypeptide, the second polypeptide, or both. In some embodiments, the one or more target binding regions comprise an antibody variable domain or antigen binding fragment thereof. In some embodiments, the protein is heterodimeric. In some embodiments, the protein is bispecific. In some embodiments, the protein is trispecific. In some embodiments, the protein is multi-specific. In another aspect, provided herein are methods for reducing aggregation of a bispecific or multi-specific antibody, wherein the antibody comprises one or more target binding regions and an exposed C-terminus, the method comprising fusing a cap domain (e.g., a His cap domain, SLSLSPGK (SEQ ID NO: 36), or TVAPTESS (SEQ ID NO:37)) at the end of the exposed C-terminus. In some embodiments, the cap domain is a His cap domain. In specific embodiments, the polypeptide, protein, or antibody is isolated. In specific embodiments, the aggregation is determined by an aggregation assay described herein (such as an assay described in Section 7.9.7) or known to one of skill in the art.

In some embodiments, the addition of a cap domain (e.g., a His cap domain, SLSLSPGK (SEQ ID NO:36), or TVAPTESS (SEQ ID NO:37)) at the end of an exposed C-terminus of a polypeptide or a protein (e.g., an antibody, such as, e.g., a bispecific antibody, or a multispecific antibody (e.g., trispecific antibody)) results in a reduction in the aggregation of the polypeptide or the protein in a biological substance relative to the aggregation of the same reference with the same polypeptide or the same protein, respectively, lacking the cap domain in the biological substance. In some embodiments, the addition of one histidine at the end of an exposed C-terminus of a polypeptide or a protein (e.g., an antibody, such as, e.g., a bispecific antibody, or a multispecific antibody (e.g., trispecific antibody)) results in a reduction in the aggregation of the polypeptide or the protein in a biological substance relative to the aggregation of the same reference with the same polypeptide or the same protein, respectively, lacking the one histidine in the biological substance. In some embodiments, the addition of two histidines at the end of an exposed C-terminus of a polypeptide or a protein (e.g., an antibody, such as, e.g., a bispecific antibody, or a multispecific antibody (e.g., trispecific antibody)) results in a reduction in the aggregation of the polypeptide or the protein in a biological substance relative to the aggregation of the same reference with the same polypeptide or the same protein, respectively, lacking the two histidines in the biological substance. In some embodiments, the addition of three histidines at the end of an exposed C-terminus of a polypeptide or a protein (e.g., an antibody, such as, e.g., a bispecific antibody, or a multispecific antibody (e.g., trispecific antibody)) results in a reduction in the aggregation of the polypeptide or the protein in a biological substance relative to the aggregation of the same reference with the same polypeptide or the same protein, respectively, lacking the three histidines in the biological substance. In some embodiments, the addition of four histidines at the end of an exposed C-terminus of a polypeptide or a protein (e.g., an antibody, such as, e.g., a bispecific antibody, or a multispecific antibody (e.g., trispecific antibody)) results in a reduction in the aggregation of the polypeptide or the protein in a biological substance relative to the aggregation of the same reference with the same polypeptide or the same protein, respectively, lacking the four histidines in the biological substance. In some embodiments, the addition of five histidines at the end of an exposed C-terminus of a polypeptide or a protein (e.g., an antibody, such as, e.g., a bispecific antibody, or a multispecific antibody (e.g., trispecific antibody)) results in a reduction in the aggregation of the polypeptide or the protein in a biological substance relative to the aggregation of the same reference with the same polypeptide or the same protein, respectively, lacking the five histidines in the biological substance. In certain embodiments, the aggregation is reduced by at least 10%, at least 15%, at least 20%, or at least 25%. In some embodiments, the aggregation is reduced by at least 30%, at least 35%, at least 40%, or at least 45%. In some embodiments, the aggregation is reduced by at least 50%, at least 55%, at least 60%, or at least 65%. In some embodiments, the aggregation is reduced by at least 70%, at least 75%, at least 80%, or at least 85%. In some embodiments, the aggregation is reduced by at least 90%, at least 95%, or at least 98%. In specific embodiments, the aggregation is assessed in an aggregation assay described herein or known to one of skill in the art.

The present disclosure also provides methods for reducing self-aggregation of a polypeptide, wherein the polypeptide comprises one or more target binding regions and an exposed C-terminus, the method comprising fusing a cap domain (e.g., a His cap domain, SLSLSPGK (SEQ ID NO:36), or TVAPTESS (SEQ ID NO:37)) at the end of the exposed C-terminus. In some embodiments, self-aggregation is reduced in a high concentration liquid formulation (HCLF). In specific embodiments, the HCLF concentration is about 50 mg/mL to about 150 mg/mL. In some embodiments, self-aggregation is reduced under physiological stress. In specific embodiments, thermal stress comprises incubation at 37° C. for about two weeks. In some embodiments, aggregation is reduced under thermal stress. In specific embodiments, thermal stress comprises incubation at 40° C. for about two weeks. In some embodiments, self-aggregation is reduced upon incubation for about two to about five days at about 37° C. In some embodiments, self-aggregation is reduced upon incubation for about two to about seven days at about 37° C. In specific embodiments, self-aggregation is reduced upon incubation for about five to about fourteen days at about 37° C. In some embodiments, self-aggregation is reduced upon incubation for about two to about seven days at about 40° C. In some embodiments, self-aggregation is reduced upon incubation for about two to about five days at about 40° C. In specific embodiments, self-aggregation is reduced upon incubation for about seven to about fourteen days at about 40° C. In specific embodiments, self-aggregation is reduced upon incubation for about fourteen days at about 4° C. In some embodiments, self-aggregation is reduced upon incubation for about two to about seven days at about 4° C. In some embodiments, self-aggregation is reduced upon incubation for about two to about five days at about 4° C. In specific embodiments, self-aggregation is reduced upon incubation for about five to about fourteen days at about 25° C. In some embodiments, self-aggregation is reduced upon incubation for about two to about seven days at about 25° C. In some embodiments, self-aggregation is reduced upon incubation for about two to about five days at about 25° C. In some embodiments, the incubation is performed in a biological substance (e.g., human serum and/or plasma). In some embodiments, the one or more target binding regions comprise an antibody variable domain or an antigen binding fragment thereof. In specific embodiments, the exposed C-terminus comprises an anti-drug antibody epitope. In some embodiments, the cap domain is a His cap domain. In some embodiments, the exposed C-terminus fused to a His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO:5). In other embodiments, the exposed C-terminus fused to a His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO: 9), and VEIKHHHHH (SEQ ID NO:10). In yet other embodiments, the exposed C-terminus fused to a His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15). In still further embodiments, the exposed C-terminus fused to a His cap domain consists of the amino acid sequence selected from the group consisting of: VEIKRTH (SEQ ID NO:16), VEIKRTHH (SEQ ID NO: 17), VEIKRTHHH (SEQ ID NO:18), VEIKRTHHHH (SEQ ID NO:19), and VEIKRTHHHHH (SEQ ID NO:20). In some embodiments, the exposed C-terminus fused to the His cap domain consists of the amino acid sequence of TKVTVL (His)n (SEQ ID NO:46), TKLTVL (His)n (SEQ ID NO:47), TQLIIL (His)n (SEQ ID NO:48), TELTVL (His)n (SEQ ID NO: 49), or TQLTVL (His)n (SEQ ID NO:50), wherein n is 1, 2, 3, 4, or 5. In some embodiments, the exposed C-terminus fused to the His cap domain consists of the amino acid sequence of TKLTVL (His)n (SEQ ID NO:47), TKVTVL (His)n (SEQ ID NO:46), NGLVYAG (His)n (SEQ ID NO: 51), NGLLYAG (His)n (SEQ ID NO:52), or TRLTVL (His)n (SEQ ID NO:53), wherein n is 1, 2, 3, 4, or 5.

In another aspect provided herein are methods for reducing self-aggregation of a polypeptide, wherein the polypeptide comprises one or more target binding regions and an exposed C-terminus, the method comprising fusing a cap domain (e.g., a His cap domain, SLSLSPGK (SEQ ID NO:36), or TVAPTESS (SEQ ID NO:37)) at the end of the exposed C-terminus. In a further aspect, provided herein are methods for reducing self-aggregation of a protein, wherein the protein includes (a) a first polypeptide comprising a target binding region, and an exposed C-terminus; and (b) a second polypeptide comprising a target binding region, the method comprising fusing a cap domain (e.g., a His cap domain, SLSLSPGK (SEQ ID NO:36), or TVAPTESS (SEQ ID NO:37)) at the end of the exposed C-terminus. In some embodiments, the second polypeptide comprises an exposed C-terminus. In some embodiments, the target binding region of the first and second polypeptides are capable of binding to different targets. In certain embodiments, the protein further comprises a third polypeptide comprising a target binding region. In some embodiments, the third polypeptide comprises an exposed C-terminus. In some embodiments, the third polypeptide is capable of binding to a different target than the first polypeptide, the second polypeptide, or both. In some embodiments, the one or more target binding regions comprise an antibody variable domain or antigen binding fragment thereof. In some embodiments, the protein is heterodimeric. In some embodiments, the protein is bispecific. In some embodiments, the protein is trispecific. In some embodiments, the protein is multi-specific. In specific embodiments, the polypeptide or protein is isolated. In specific embodiments, the self-aggregation is determined by an aggregation assay described herein (such as an assay described in Section 7.9.7) or known to one of skill in the art.

In some embodiments, the addition of a cap domain (e.g., a His cap domain, SLSLSPGK (SEQ ID NO:36), or TVAPTESS (SEQ ID NO:37)) at the end of an exposed C-terminus of a polypeptide or a protein (e.g., an antibody, such as, e.g., a bispecific antibody, or a multispecific antibody (e.g., trispecific antibody)) results in a reduction in self-aggregation of the polypeptide or the protein relative to the self-aggregation of the same reference with the same polypeptide or the same protein, respectively, lacking the cap domain. In some embodiments, the addition of one histidine at the end of an exposed C-terminus of a polypeptide or a protein (e.g., an antibody, such as, e.g., a bispecific antibody, or a multispecific antibody (e.g., trispecific antibody)) results in a reduction in self-aggregation of the polypeptide or the protein relative to the self-aggregation of the same reference with the same polypeptide or the same protein, respectively, lacking the one histidine. In some embodiments, the addition of two histidines at the end of an exposed C-terminus of a polypeptide or a protein (e.g., an antibody, such as, e.g., a bispecific antibody, or a multispecific antibody (e.g., trispecific antibody)) results in a reduction in self-aggregation of the polypeptide or the protein relative to the self-aggregation of the same reference with the same polypeptide or the same protein, respectively, lacking the two histidines. In some embodiments, the addition of three histidines at the end of an exposed C-terminus of a polypeptide or a protein (e.g., an antibody, such as, e.g., a bispecific antibody, or a multispecific antibody (e.g., trispecific antibody)) results in a reduction in self-aggregation of the polypeptide or the protein relative to the self-aggregation of the same reference with the same polypeptide or the same protein, respectively, lacking the three histidines. In some embodiments, the addition of four histidines at the end of an exposed C-terminus of a polypeptide or a protein (e.g., an antibody, such as, e.g., a bispecific antibody, or a multispecific antibody (e.g., trispecific antibody)) results in a reduction in self-aggregation of the polypeptide or the protein relative to the self-aggregation of the same reference with the same polypeptide or the same protein, respectively, lacking the four histidines. In some embodiments, the addition of five histidines at the end of an exposed C-terminus of a polypeptide or a protein (e.g., an antibody, such as, e.g., a bispecific antibody, or a multispecific antibody (e.g., trispecific antibody)) results in a reduction in self-aggregation of the polypeptide or the protein relative to the self-aggregation of the same reference with the same polypeptide or the same protein, respectively, lacking the five histidines. In certain embodiments, the self-aggregation is reduced by at least 10%, at least 15%, at least 20%, or at least 25%. In some embodiments, the self-aggregation is reduced by at least 30%, at least 35%, at least 40%, or at least 45%. In some embodiments, the self-aggregation is reduced by at least 50%, at least 55%, at least 60%, or at least 65%. In some embodiments, the self-aggregation is reduced by at least 70%, at least 75%, at least 80%, or at least 85%. In some embodiments, the self-aggregation is reduced by at least 90%, at least 95%, or at least 98%. In specific embodiments, the self-aggregation is assessed in an aggregation assay described herein or known to one of skill in the art.

In a specific aspect, provided herein are methods for reducing the interaction between a protein (e.g., an antibody) and an anti-drug antibody, wherein the protein comprises an exposed C-terminus, the method comprising adding a cap domain (e.g., a His cap domain, SLSLSPGK (SEQ ID NO:36), or TVAPTESS (SEQ ID NO:37)) at the end of the exposed C-terminus, wherein n is 1, 2, 3, 4 or 5, and wherein the protein exhibits reduced interaction with an anti-drug antibody relative to that of the protein lacking the cap domain. In a specific aspect, provided herein are methods for reducing the interaction between a protein (e.g., an antibody) and an anti-drug antibody, wherein the protein comprises an exposed C-terminus, the method comprising adding a (His)ₙ (SEQ ID NO:25) sequence at the end of the exposed C-terminus, wherein n is 1, 2, 3, 4 or 5, and wherein the protein exhibits reduced interaction with an anti-drug antibody relative to that of the protein lacking the (His)ₙ (SEQ ID NO:25) sequence. In some embodiments, the anti-drug antibody is pre-existing in serum and/or plasma of a subject (e.g., a human subject). Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In a specific embodiment, provided herein is a method for reducing the interaction between a protein and an anti-drug antibody, wherein the protein comprises an antigen binding region of an antibody, and wherein the antigen binding region comprises an exposed C-terminus, the method comprising adding a (His)ₙ (SEQ ID NO:25) sequence at the end of the exposed C-terminus, wherein n is 1, 2, 3, 4 or 5, and wherein the protein exhibits reduced interaction with an anti-drug antibody relative to that of the protein lacking the (His)ₙ (SEQ ID NO:25) sequence. In another specific embodiment, provided herein is a method for reducing the interaction between a protein and an anti-drug antibody, wherein the protein comprises an antibody variable domain, and wherein the antibody variable domain comprises an exposed C-terminus, the method comprising adding a (His)ₙ (SEQ ID NO:25) sequence at the end of the exposed C-terminus, wherein n is 1, 2, 3, 4 or 5, and wherein the protein exhibits reduced interaction with an anti-drug antibody relative to that of the protein lacking the (His)ₙ (SEQ ID NO:25) sequence. In another specific embodiment, provided herein is a method for reducing the interaction between a protein and an anti-drug antibody, wherein the protein comprises an antibody fragment, and wherein the antibody fragment comprises an exposed C-terminus, the method comprising adding a (His)ₙ (SEQ ID NO:25) sequence at the end of the exposed C-terminus, wherein n is 1, 2, 3, 4 or 5, and wherein the protein exhibits reduced interaction with an anti-drug antibody relative to that of the protein lacking the (His)ₙ (SEQ ID NO:25) sequence. Various assays known to one of skill in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In a specific embodiment, provided herein is a method for reducing the interaction between a bispecific antibody and an anti-drug antibody, wherein the bispecific antibody comprises an antigen binding region of an antibody, and wherein the antigen binding region comprises an exposed C-terminus, the method comprising adding a (His)ₙ (SEQ ID NO:25) sequence at the end of the exposed C-terminus, wherein n is 1, 2, 3, 4 or 5, and wherein the bispecific antibody exhibits reduced interaction with an anti-drug antibody relative to that of the bispecific antibody lacking the (His)ₙ (SEQ ID NO:25) sequence. In another specific embodiment, provided herein is a method for reducing the interaction between a bispecific antibody and an anti-drug antibody, wherein the bispecific antibody comprises an antibody variable domain, and wherein the antibody variable domain comprises an exposed C-terminus, the method comprising adding a (His)ₙ (SEQ ID NO:25) sequence at the end of the exposed C-terminus, wherein n is 1, 2, 3, 4 or 5, and wherein the bispecific antibody exhibits reduced interaction with an anti-drug antibody relative to that of the bispecific antibody lacking the (His)ₙ (SEQ ID NO:25) sequence. In another specific embodiment, provided herein is a method for reducing the interaction between a bispecific antibody and an anti-drug antibody, wherein the bispecific antibody comprises an antibody fragment, and wherein the antibody fragment comprises an exposed C-terminus, the method comprising adding a (His)ₙ (SEQ ID NO:25) sequence at the end of the exposed C-terminus, wherein n is 1, 2, 3, 4 or 5, and wherein the bispecific antibody exhibits reduced interaction with an anti-drug antibody relative to that of the bispecific antibody lacking the (His)ₙ (SEQ ID NO:25) sequence. Various assays known to one of skill in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In a specific embodiment, provided herein is a method for reducing the interaction between a multi-specific antibody and an anti-drug antibody, wherein the multi-specific antibody comprises an antigen binding region of an antibody, and wherein the antigen binding region comprises an exposed C-terminus, the method comprising adding a (His)ₙ (SEQ ID NO:25) sequence at the end of the exposed C-terminus, wherein n is 1, 2, 3, 4 or 5, and wherein the multi-specific antibody exhibits reduced interaction with an anti-drug antibody relative to that of the multi-specific antibody lacking the (His)ₙ (SEQ ID NO:25) sequence. In another specific embodiment, provided herein is a method for reducing the interaction between a multi-specific antibody and an anti-drug antibody, wherein the multi-specific antibody comprises an antibody variable domain, and wherein the antibody variable domain comprises an exposed C-terminus, the method comprising adding a (His)ₙ (SEQ ID NO:25) sequence at the end of the exposed C-terminus, wherein n is 1, 2, 3, 4 or 5, and wherein the multi-specific antibody exhibits reduced interaction with an anti-drug antibody relative to that of the multi-specific antibody lacking the (His)ₙ (SEQ ID NO:25) sequence. In another specific embodiment, provided herein is a method for reducing the interaction between a multi-specific antibody and an anti-drug antibody, wherein the multi-specific antibody comprises an antibody fragment, and wherein the antibody fragment comprises an exposed C-terminus, the method comprising adding a (His)ₙ (SEQ ID NO:25) sequence at the end of the exposed C-terminus, wherein n is 1, 2, 3, 4 or 5, and wherein the multi-specific antibody exhibits reduced interaction with an anti-drug antibody relative to that of the multi-specific antibody lacking the (His)ₙ (SEQ ID NO:25) sequence. Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In another aspect, provided herein are methods for reducing the interaction between a protein (e.g., an antibody) and an anti-drug antibody, wherein the protein comprises a non-naturally occurring C-terminus, the method comprising adding a cap domain (e.g., a His cap domain, SLSLSPGK (SEQ ID NO:36), or TVAPTESS (SEQ ID NO:37)) at the end of the non-naturally occurring C-terminus, wherein the protein exhibits reduced interaction with an anti-drug antibody relative to that of the protein lacking the cap domain. Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In another aspect, provided herein are methods for reducing the interaction between a protein (e.g., an antibody) and an anti-drug antibody, wherein the protein comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits reduced interaction with an anti-drug antibody relative to that of the protein lacking the (His)ₙ (SEQ ID NO:25) sequence. In a specific embodiment, provided herein is a method for reducing the interaction between a protein and an anti-drug antibody, wherein the protein comprises an antigen binding region of an antibody, and wherein the antigen binding region comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits reduced interaction with an anti-drug antibody relative to that of the protein lacking the (His)ₙ (SEQ ID NO:25) sequence. In another specific embodiment, provided herein is a method for reducing the interaction between a protein and an anti-drug antibody, wherein the protein comprises an antibody variable domain, and wherein the antibody variable domain comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits reduced interaction with an anti-drug antibody relative to that of the protein lacking the (His)ₙ (SEQ ID NO:25) sequence. In another specific embodiment, provided herein is a method for reducing the interaction between a protein and an anti-drug antibody, wherein the protein comprises an antibody fragment, and wherein the antibody fragment comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits reduced interaction with an anti-drug antibody relative to that of the protein lacking the (His)ₙ (SEQ ID NO:25) sequence. Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In a specific embodiment, provided herein is a method for reducing the interaction between a bispecific antibody and an anti-drug antibody, wherein the bispecific antibody comprises an antigen binding region of an antibody, and wherein the antigen binding region comprises a non-naturally occurring C-terminus, the method comprising adding a (His)ₙ (SEQ ID NO: 25) sequence at the end of the non-naturally occurring C-terminus, wherein n is 1, 2, 3, 4 or 5, and wherein the bispecific antibody exhibits reduced with an anti-drug antibody relative to that of the bispecific antibody lacking the (His)ₙ (SEQ ID interaction NO: 25) sequence. In another specific embodiment, provided herein is a method for reducing the interaction between a bispecific antibody and an anti-drug antibody, wherein the bispecific antibody comprises an antibody variable domain, and wherein the antibody variable domain comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the bispecific antibody exhibits reduced interaction with an anti-drug antibody relative to that of the bispecific antibody lacking the (His)ₙ (SEQ ID NO:25) sequence. In another specific embodiment, provided herein is a method for reducing the interaction between a bispecific antibody and an anti-drug antibody, wherein the bispecific antibody comprises an antibody fragment, and wherein the antibody fragment comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the bispecific antibody exhibits reduced interaction with an anti-drug antibody relative to that of the bispecific antibody lacking the (His)ₙ (SEQ ID NO:25) sequence. Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In a specific embodiment, provided herein is a method for reducing the interaction between a multi-specific antibody and an anti-drug antibody, wherein the multi-specific antibody comprises an antigen binding region of an antibody, and wherein the antigen binding region comprises a non-naturally occurring C-terminus, the method comprising adding a (His)ₙ (SEQ ID NO: 25) sequence at the end of the non-naturally occurring C-terminus, wherein n is 1, 2, 3, 4 or 5, and wherein the multi-specific antibody exhibits reduced interaction with an anti-drug antibody relative to that of the multi-specific antibody lacking the (His)ₙ (SEQ ID NO:25) sequence. In another specific embodiment, provided herein is a method for reducing the interaction between a multi-specific antibody and an anti-drug antibody, wherein the multi-specific antibody comprises an antibody variable region, and wherein the antibody variable region comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the multi-specific antibody exhibits reduced interaction with an anti-drug antibody relative to that of the multi-specific antibody lacking the (His)ₙ (SEQ ID NO:25) sequence. In another specific embodiment, provided herein is a method for reducing the interaction between a multi-specific antibody and an anti-drug antibody, wherein the multi-specific antibody comprises an antibody fragment, and wherein the antibody fragment comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the multi-specific antibody exhibits reduced interaction with an anti-drug antibody relative to that of the multi-specific antibody lacking the (His)ₙ (SEQ ID NO:25) sequence. Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In another aspect, provided herein are methods for reducing the interaction between a protein (e.g., an antibody) and an anti-drug antibody, wherein the protein comprises a C-terminal anti-drug epitope, the method comprising adding a cap domain (e.g., a His cap domain, SLSLSPGK (SEQ ID NO:36), or TVAPTESS (SEQ ID NO:37)) at the end of the C-terminal anti-drug epitope, wherein the protein exhibits reduced interaction with an anti-drug antibody relative to that of the protein lacking the cap domain. Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In another aspect, provided herein are methods for reducing the interaction between a protein (e.g., an antibody) and an anti-drug antibody, wherein the protein comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the protein exhibits reduced interaction with an anti-drug antibody relative to that of the protein lacking the (His)ₙ (SEQ ID NO:25) sequence. In a specific embodiment, provided herein is a method for reducing the interaction between a protein and an anti-drug antibody, wherein the protein comprises an antigen binding region of an antibody, and wherein the antigen binding region comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the protein exhibits reduced interaction with an anti-drug antibody relative to that of the protein lacking the (His)ₙ (SEQ ID NO:25) sequence. In another specific embodiment, provided herein is a method for reducing the interaction between a protein and an anti-drug antibody, wherein the protein comprises an antibody variable region, and wherein the antibody variable region comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the protein exhibits reduced interaction with an anti-drug antibody relative to that of the protein lacking the (His)ₙ (SEQ ID NO:25) sequence. In another specific embodiment, provided herein is a method for reducing the interaction between a protein and an anti-drug antibody, wherein the protein comprises an antibody fragment, and wherein the antibody fragment comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the protein exhibits reduced interaction with an anti-drug antibody relative to that of the protein lacking the (His)ₙ (SEQ ID NO:25) sequence. Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In a specific embodiment, provided herein is a method for reducing the interaction between a bispecific antibody and an anti-drug antibody, wherein the bispecific antibody comprises an antigen binding region of an antibody, and wherein the antigen binding region comprises a C-terminal anti-drug epitope, the method comprising adding a (His)ₙ (SEQ ID NO: 25) sequence at the end of the C-terminal anti-drug epitope, wherein n is 1, 2, 3, 4 or 5, and wherein the bispecific antibody exhibits reduced interaction with an anti-drug antibody relative to that of the bispecific antibody lacking the (His)ₙ (SEQ ID NO:25) sequence. In another specific embodiment, provided herein is a method for reducing the interaction between a bispecific antibody and an anti-drug antibody, wherein the bispecific antibody comprises an antibody variable domain antibody, and wherein the antibody variable domain comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the bispecific antibody exhibits reduced interaction with an anti-drug antibody relative to that of the bispecific antibody lacking the (His)ₙ (SEQ ID NO:25) sequence. In another specific embodiment, provided herein is a method for reducing the interaction between a bispecific antibody and an anti-drug antibody, wherein the bispecific antibody comprises an antibody fragment, and wherein the antibody fragment comprises a C-terminal anti-drug epitope, the method comprising adding a (His)ₙ (SEQ ID NO: 25) sequence at the end of the C-terminal anti-drug epitope, wherein n is 1, 2, 3, 4 or 5, and wherein the bispecific antibody exhibits reduced interaction with an anti-drug antibody relative to that of the bispecific antibody lacking the (His)ₙ (SEQ ID NO:25) sequence. Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In a specific embodiment, provided herein is a method for reducing the interaction between a multi-specific antibody and an anti-drug antibody, wherein the multi-specific antibody comprises an antigen binding region of an antibody, and wherein the antigen binding region comprises a C-terminal anti-drug epitope, the method comprising adding a (His)ₙ (SEQ ID NO: 25) sequence at the end of the C-terminal anti-drug epitope, wherein n is 1, 2, 3, 4 or 5, and wherein the multi-specific antibody exhibits reduced interaction with an anti-drug antibody relative to that of the multi-specific antibody lacking the (His)ₙ (SEQ ID NO:25) sequence. In some embodiments, the multi-specific antibody is a trispecific antibody. In another specific embodiment, provided herein is a method for reducing the interaction between a multi-specific antibody and an anti-drug antibody, wherein the multi-specific antibody comprises an antibody variable domain antibody, and wherein the antibody variable domain comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the multi-specific antibody exhibits reduced interaction with an anti-drug antibody relative to that of the multi-specific antibody lacking the (His)ₙ (SEQ ID NO:25) sequence. In another specific embodiment, provided herein is a method for reducing the interaction between a multi-specific antibody and an anti-drug antibody, wherein the multi-specific antibody comprises an antibody fragment, and wherein the antibody fragment comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the multi-specific antibody exhibits reduced interaction with an anti-drug antibody relative to that of the multi-specific antibody lacking the (His)ₙ (SEQ ID NO: 25) sequence. Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In specific embodiments, the addition of a cap domain (e.g., a His cap domain, SLSLSPGK (SEQ ID NO:36), or TVAPTESS (SEQ ID NO:37)) at the end of an exposed C-terminus, the end of a non-naturally occurring C-terminus, or the end of a C-terminal anti-drug antibody epitope of a protein (e.g., an antibody), a bispecific antibody, or a multispecific antibody results in a reduction in the interaction of the protein (e.g., an antibody), the bispecific antibody, or the multispecific antibody with an ADA relative to the interaction of the same protein (e.g., an antibody), the same bispecific antibody, or the same multispecific antibody, respectively, lacking the cap domain with the ADA. In some embodiments, the reduction is at least 10%, at least 20%, at least 30%, at least 40%, or at least 50%. In some embodiments, the reduction is at least 60%, at least 70%, at least 80%, at least 90%, or at least 95%.

In specific embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in a reduction in the interaction of the protein (e.g., an antibody), the bispecific antibody, or the multispecific antibody with an ADA relative to the interaction of the same protein (e.g., an antibody), the same bispecific antibody, or the same multispecific antibody, respectively, lacking the (His)ₙ (SEQ ID NO:25) sequence with the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about a 10% reduction in the interaction of the protein (e.g., an antibody), the bispecific antibody, or the multispecific antibody with an ADA, relative to the interaction of the same protein (e.g., an antibody), the same bispecific antibody, or the same multispecific antibody, respectively, lacking the (His)ₙ (SEQ ID NO:25) sequence with the ADA. In some embodiments, the addition of a (His)ₙ (SEQ ID NO: 25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about a 20% reduction in the interaction of the protein (e.g., an antibody), the bispecific antibody, or the multispecific antibody with an ADA, relative to the same protein (e.g., an antibody), the same bispecific antibody, or the same multispecific antibody, respectively, lacking the (His)ₙ (SEQ ID NO:25) sequence with the ADA. In some embodiments, the addition of a (His)ₙ (SEQ ID NO: 25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about a 30% reduction in the interaction of the protein with an ADA, relative to the interaction of the same protein (e.g., an antibody), the same bispecific antibody, or the same multispecific antibody, respectively, lacking the (His)ₙ (SEQ ID NO:25) sequence with the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about a 40% reduction in the interaction of the protein (e.g., an antibody), the bispecific antibody, or the multispecific antibody with an ADA, relative to the interaction of the same protein (e.g., an antibody), the same bispecific antibody, or the same multispecific antibody, respectively, lacking the (His)ₙ (SEQ ID NO:25) sequence with the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 50% reduction in the interaction of the protein (e.g., an antibody), the bispecific antibody, or the multispecific antibody with an ADA, relative to the interaction of the same protein (e.g., an antibody), the same bispecific antibody, or the same multispecific antibody (e.g., trispecific antibody), respectively, lacking the (His)ₙ (SEQ ID NO:25) sequence with the ADA. In some embodiments, the addition of a (His)ₙ (SEQ ID NO:25) sequence at the end of an exposed C-terminus, the end of a non-naturally occurring C-terminus, or the end of A C-terminal anti-drug antibody epitope of a therapeutic protein (e.g., an antigen binding protein), wherein n is 1, 2, 3, 4, or 5, results in at least about a 60% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody, respectively, lacking the (His)ₙ (SEQ ID NO:25) sequence with the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 70% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody, respectively, lacking the (His)ₙ (SEQ ID NO:25) sequence with the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about an 80% reduction in the interaction of the protein (e.g., an antibody), the bispecific antibody, or the multispecific (e.g., trispecific antibody) antibody with an ADA, relative to the interaction of the same protein (e.g., an antibody), the same bispecific antibody, or the same multispecific antibody (e.g., trispecific antibody), respectively, lacking the (His)ₙ (SEQ ID NO:25) sequence with the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 90% reduction in the interaction of the protein (e.g., an antibody), the bispecific antibody, or the multispecific (e.g., trispecific antibody) antibody with an ADA, relative to the interaction of the same protein (e.g., an antibody), the same bispecific antibody, or the same multispecific antibody (e.g., trispecific antibody), respectively, lacking the (His)ₙ (SEQ ID NO:25) sequence with the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 95% reduction in the interaction of the protein (e.g., an antibody), the bispecific antibody, or the multispecific antibody with an ADA, relative to the interaction of the same protein (e.g., an antibody), the same bispecific antibody, or the same multispecific antibody (e.g., trispecific antibody), respectively, lacking the (His)ₙ (SEQ ID NO:25) sequence with the ADA. Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In specific embodiments, the addition of a single histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in a reduction in the interaction of the protein (e.g., an antibody), the bispecific antibody, or the multispecific antibody with an ADA relative to the interaction of the same protein (e.g., an antibody), the same bispecific antibody, or the same multispecific antibody (e.g., trispecific antibody), respectively, lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 10% reduction in the interaction of the protein (e.g., an antibody), the bispecific antibody, or the multispecific antibody with an ADA, relative to the interaction of the same protein (e.g., an antibody), the same bispecific antibody (e.g., trispecific antibody), or the same multispecific antibody, respectively, lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 20% reduction in the interaction of the protein (e.g., an antibody), the bispecific antibody, or the multispecific antibody with an ADA, relative to the interaction of the same protein (e.g., an antibody), the same bispecific antibody, or the same multispecific antibody (e.g., trispecific antibody), respectively, lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 30% reduction in the interaction of the protein (e.g., an antibody), the bispecific antibody, or the multispecific antibody with an ADA, relative to the interaction of the same protein (e.g., an antibody), the same bispecific antibody, or the same multispecific antibody (e.g., trispecific antibody), respectively, lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 40% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody, respectively, lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antibody), a bispecific antibody, or a multispecific antibody results in at least about a 50% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody, respectively, lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 60% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody, (e.g., trispecific antibody) respectively, lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 70% reduction in the interaction of the protein (e.g., an antibody), the bispecific antibody, or the multispecific antibody with an ADA, relative to the interaction of the same protein (e.g., an antibody), the same bispecific antibody, or the same multispecific antibody, respectively, lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about an 80% reduction in the interaction of the protein (e.g., an antibody), the bispecific antibody, or the multispecific antibody with an ADA, relative to the interaction of the same protein (e.g., an antibody), the same bispecific antibody, or the same multispecific antibody (e.g., trispecific antibody), respectively, lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 90% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the single histidine with the ADA. In some embodiments, the addition of a single histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 95% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody, respectively, lacking the single histidine with the ADA. In particular embodiments, the addition of a single histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) eliminates interaction of the protein (e.g., an antibody), the bispecific antibody, or the multispecific antibody (e.g., trispecific antibody) with an ADA. Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In specific embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in a reduction in the interaction of the protein (e.g., an antibody), the bispecific antibody, or the multispecific antibody (e.g., trispecific antibody) with an ADA relative to the interaction of the same protein (e.g., an antibody), the same bispecific antibody, or the same multispecific antibody (e.g., trispecific antibody), respectively, lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 10% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 20% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 30% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 40% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 50% reduction in the interaction of the protein (e.g., an antibody), the bispecific antibody, or the multispecific antibody with an ADA, relative to the interaction of the same protein (e.g., an antibody), the same bispecific antibody, or the same multispecific antibody (e.g., trispecific antibody), respectively, lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 60% reduction in the interaction of the protein (e.g., an antibody), the bispecific antibody, or the multispecific antibody with an ADA, relative to the interaction of the same protein (e.g., an antibody), the same bispecific antibody, or the same multispecific antibody (e.g., trispecific antibody), respectively, lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 70% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about an 80% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody, respectively, lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 90% reduction in the interaction of the protein (e.g., an antibody), the bispecific antibody, or the multispecific antibody (e.g., trispecific antibody) with an ADA, relative to the interaction the same protein (e.g., an antibody), the same bispecific antibody, or the same multispecific antibody (e.g., trispecific antibody), respectively, lacking the two histidines with the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 95% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the two histidines with the ADA. In particular embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) eliminates interaction of the protein (e.g., an antibody), the bispecific antibody, or the multispecific antibody (e.g., trispecific antibody) with an ADA. Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In specific embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in a reduction in the interaction of the protein (e.g., an antibody), the bispecific antibody, or the multispecific antibody (e.g., trispecific antibody) with an ADA relative to the interaction of the same protein (e.g., an antibody), the same bispecific antibody, or the same multispecific antibody (e.g., trispecific antibody), respectively, lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 10% reduction in the interaction of the protein (e.g., an antibody), the bispecific antibody, or the multispecific antibody with an ADA, relative to the interaction of the same protein (e.g., an antibody), the same bispecific antibody, or the same multispecific antibody (e.g., trispecific antibody), respectively, lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 20% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 30% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 40% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 50% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 60% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 70% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about an 80% reduction in the interaction of the protein (e.g., an antibody), the bispecific antibody, or the multispecific antibody with an ADA, relative to the interaction of the same protein (e.g., an antibody), the same bispecific antibody, or the same multispecific antibody (e.g., trispecific antibody), respectively, lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 90% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the three histidines with the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 95% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the three histidines with the ADA. In particular embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) eliminates interaction of the protein (e.g., an antibody), the bispecific antibody, or the multispecific antibody (e.g., trispecific antibody) with an ADA. Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In specific embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody results in a reduction in the interaction of the protein (e.g., an antibody), the bispecific antibody, or the multispecific antibody (e.g., trispecific antibody) with an ADA relative to the interaction of the same protein (e.g., an antibody), the same bispecific antibody, or the same multispecific antibody (e.g., trispecific antibody), respectively, lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 10% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 20% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 30% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 40% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody, respectively, lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 50% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 60% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 70% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about an 80% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 90% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the four histidines with the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 95% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the four histidines with the ADA. In particular embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) eliminates interaction of the protein (e.g., an antibody), the bispecific antibody, or the multispecific antibody (e.g., trispecific antibody) with an ADA. Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In specific embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in a reduction in the interaction of the protein (e.g., an antibody), the bispecific antibody, or the multispecific antibody (e.g., trispecific antibody) with an ADA relative to the interaction of the same protein (e.g., an antibody), the same bispecific antibody, or the same multispecific antibody (e.g., trispecific antibody), respectively, lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 10% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 20% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 30% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 40% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 50% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 60% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 70% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about an 80% reduction in the interaction of the protein (e.g., an antibody), the bispecific antibody, or the multispecific antibody with an ADA, relative to the interaction of the same protein (e.g., an antibody), the same bispecific antibody, or the same multispecific antibody (e.g., trispecific antibody), respectively, lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 90% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody, respectively, lacking the five histidines with the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 95% reduction in the interaction of the protein (e.g., an antibody), the bispecific antibody, or the multispecific antibody (e.g., trispecific antibody) with an ADA, relative to the interaction of the same protein (e.g., an antibody), the same bispecific antibody, or the same multispecific antibody (e.g., trispecific antibody), respectively, lacking the five histidines with the ADA. In particular embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) eliminates interaction of the protein (e.g., an antibody), the bispecific antibody, or the multispecific antibody (e.g., trispecific antibody) with an ADA. Various assays known in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In specific embodiments, the addition of a cap domain (e.g., a His cap domain, SLSLSPGK (SEQ ID NO: 36), or TVAPTESS (SEQ ID NO:37)) at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in decreased affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the cap domain. In some embodiments, the decrease is at least 2-fold, at least 5-fold, at least 10-fold, at least 50-fold, or at least 100-fold. In some embodiments, the decrease is 2- to 10-fold, 5- to 20-fold, 10- to 50-fold, or 50- to 100-fold.

In specific embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in decreased affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 2-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO: 25) sequence at the end of an exposed C-terminus, the end of the non-naturally occurring C-terminus, or the end of the C-terminal anti-drug antibody epitope of a protein (e.g., an antibody), a bispecific antibody, or a multispecific antibody (e.g., trispecific antibody), wherein n is 1, 2, 3, 4, or 5, results in at least about a 5-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of the non-naturally occurring C-terminus, or the end of the C-terminal anti-drug antibody epitope of a protein (e.g., an antibody), a bispecific antibody, or a multispecific antibody (e.g., trispecific antibody), wherein n is 1, 2, 3, 4, or 5, results in at least about a 10-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody, respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of the C-terminal anti-drug antibody epitope of a protein (e.g., an antibody), a bispecific antibody, or a multispecific antibody, wherein n is 1, 2, 3, 4, or 5, results in at least about a 20-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 50-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 100-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 500-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in greater than 500-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO: 25) sequence. In specific embodiments, affinity of a protein for an ADA is determined by measuring interaction between the protein and an ADA (such as in assay described in Section 7.9.1) or known to one of skill in the art.

In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in about 2-fold to about 500-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in about 2-fold to about 100-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in about 2-fold to about 50-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in about 2-fold to about 20-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO: 25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in about 2-fold to about 10-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about 2-fold to about 5-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in about 5-fold to about 100-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in about 5-fold to about 50-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO: 25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in about 5-fold to about 20-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about 5-fold to about 10-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in about 10-fold to about 100-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in about 10-fold to about 50-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO: 25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in about 10-fold to about 20-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in about 50-fold to about 100-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in about 100-fold to about 500-fold decrease in affinity of the protein (e.g., an antibody), the bispecific antibody, or the multispecific antibody (e.g., trispecific antibody) for an ADA, relative to the same protein (e.g., an antibody), the same bispecific antibody, or the same multispecific antibody (e.g., trispecific antibody), respectively, lacking the (His)ₙ (SEQ ID NO:25) sequence. In specific embodiments, affinity of a protein for an ADA is determined by measuring interaction between the protein and an ADA (such as in assay described in Section 7.9.1) or known to one of skill in the art.

In another aspect, provided herein are methods for reducing the neutralization of a protein (e.g., an antibody) by an anti-drug antibody, wherein the protein comprises an exposed C-terminus, the method comprising adding a cap domain (e.g., a His cap domain, SLSLSPGK (SEQ ID NO:36), or TVAPTESS (SEQ ID NO:37)) at the end of the exposed C-terminus, wherein the protein exhibits reduced neutralization by an anti-drug antibody relative to that of the protein lacking the cap domain. In some embodiments, the reduction is at least 10%, at least 20%, at least 30%, at least 40%, or at least 50%. In some embodiments, the reduction is at least 60%, at least 70%, at least 80%, at least 90%, or at least 95%. In specific embodiments, the neutralization of a protein by an ADA is determined by neutralization assay described herein (such as an assay described in Section 7.9.4) or known to one of skill in the art.

In another aspect, provided herein are methods for reducing the neutralization of a protein (e.g., an antibody) by an anti-drug antibody, wherein the protein comprises an exposed C-terminus, the method comprising adding a (His)ₙ (SEQ ID NO:25) sequence at the end of the exposed C-terminus, wherein n is 1, 2, 3, 4 or 5, and wherein the protein exhibits reduced neutralization by an anti-drug antibody relative to that of the protein lacking the (His)ₙ (SEQ ID NO: 25) sequence. In a specific embodiment, provided herein is a method for reducing the neutralization of a protein by an anti-drug antibody, wherein the protein comprises an antigen binding region of an antibody, and wherein the antigen binding region comprises an exposed C-terminus, the method comprising adding a (His)ₙ (SEQ ID NO:25) sequence at the end of the exposed C-terminus, wherein n is 1, 2, 3, 4 or 5, and wherein the protein exhibits reduced neutralization by an anti-drug antibody relative to that of the protein lacking the (His)ₙ (SEQ ID NO: 25) sequence. In another specific embodiment, provided herein is a method for reducing the neutralization of a protein by an anti-drug antibody, wherein the protein comprises an antibody variable domain, and wherein the antibody variable domain comprises an exposed C-terminus, the method comprising adding a (His)ₙ (SEQ ID NO:25) sequence at the end of the exposed C-terminus, wherein n is 1, 2, 3, 4 or 5, and wherein the protein exhibits reduced neutralization by an anti-drug antibody relative to that of the protein lacking the (His)ₙ (SEQ ID NO:25) sequence. In another specific embodiment, provided herein is a method for reducing the neutralization of a protein by an anti-drug antibody, wherein the protein comprises an antibody fragment, and wherein the antibody fragment comprises an exposed C-terminus, the method comprising adding a (His)ₙ (SEQ ID NO:25) sequence at the end of the exposed C-terminus, wherein n is 1, 2, 3, 4 or 5, and wherein the protein exhibits reduced neutralization by an anti-drug antibody relative to that of the protein lacking the (His)ₙ (SEQ ID NO:25) sequence. In specific embodiments, the neutralization of a protein by an ADA is determined by neutralization assay described herein (such as an assay described in Section 7.9.4) or known to one of skill in the art.

In a specific embodiment, provided herein is a method for reducing the neutralization of a bispecific antibody by an anti-drug antibody, wherein the bispecific antibody comprises an antigen binding region of an antibody, and wherein the antigen binding region comprises an exposed C-terminus, the method comprising adding a (His)ₙ (SEQ ID NO:25) sequence at the end of the exposed C-terminus, wherein n is 1, 2, 3, 4 or 5, and wherein the bispecific antibody exhibits reduced neutralization by an anti-drug antibody relative to that of the bispecific antibody lacking the (His)ₙ (SEQ ID NO:25) sequence. In another specific embodiment, provided herein is a method for reducing the neutralization of a bispecific antibody by an anti-drug antibody, wherein the bispecific antibody comprises an antibody variable domain, and wherein the antibody variable domain comprises an exposed C-terminus, the method comprising adding a (His)ₙ (SEQ ID NO:25) sequence at the end of the exposed C-terminus, wherein n is 1, 2, 3, 4 or 5, and wherein the bispecific antibody exhibits reduced neutralization by an anti-drug antibody relative to that of the bispecific antibody lacking the (His)ₙ (SEQ ID NO:25) sequence. In another specific embodiment, provided herein is a method for reducing the neutralization of a bispecific antibody by an anti-drug antibody, wherein the bispecific antibody comprises an antibody fragment, and wherein the antibody fragment comprises an exposed C-terminus, the method comprising adding a (His)ₙ (SEQ ID NO:25) sequence at the end of the exposed C-terminus, wherein n is 1, 2, 3, 4 or 5, and wherein the bispecific antibody exhibits reduced neutralization by an anti-drug antibody relative to that of the bispecific antibody lacking the (His)ₙ (SEQ ID NO: 25) sequence. In specific embodiments, the neutralization of a protein by an ADA is determined by neutralization assay described herein (such as an assay described in Section 7.9.4) or known to one of skill in the art.

In a specific embodiment, provided herein is a method for reducing the neutralization of a multi-specific antibody (e.g., trispecific antibody) by an anti-drug antibody, wherein the multi-specific antibody comprises an antigen binding region of an antibody, and wherein the antigen binding region comprises an exposed C-terminus, the method comprising adding a (His)ₙ (SEQ ID NO:25) sequence at the end of the exposed C-terminus, wherein n is 1, 2, 3, 4 or 5, and wherein the multi-specific antibody exhibits reduced neutralization by an anti-drug antibody relative to that of the multi-specific antibody lacking the (His)ₙ (SEQ ID NO:25) sequence. In another specific embodiment, provided herein is a method for reducing the neutralization of a multi-specific antibody by an anti-drug antibody, wherein the multi-specific antibody comprises an antibody variable domain, and wherein the antibody variable domain comprises an exposed C-terminus, the method comprising adding a (His)ₙ (SEQ ID NO:25) sequence at the end of the exposed C-terminus, wherein n is 1, 2, 3, 4 or 5, and wherein the multi-specific antibody exhibits reduced neutralization by an anti-drug antibody relative to that of the multi-specific antibody lacking the (His)ₙ (SEQ ID NO:25) sequence. In another specific embodiment, provided herein is a method for reducing the neutralization of a multi-specific antibody by an anti-drug antibody, wherein the multi-specific antibody comprises an antibody fragment, and wherein the antibody fragment comprises an exposed C-terminus, the method comprising adding a (His)ₙ (SEQ ID NO: 25) sequence at the end of the exposed C-terminus, wherein n is 1, 2, 3, 4 or 5, and wherein the multi-specific antibody exhibits reduced neutralization by an anti-drug antibody relative to that of the multi-specific antibody lacking the (His)ₙ (SEQ ID NO:25) sequence. In specific embodiments, the neutralization of a protein by an ADA is determined by neutralization assay described herein (such as an assay described in Section 7.9.4) or known to one of skill in the art.

In another aspect, provided herein are methods for reducing the neutralization of a protein (e.g., an antibody) by an anti-drug antibody, wherein the protein comprises a non-naturally occurring C-terminus, the method comprising adding a cap domain (e.g., a His cap domain, SLSLSPGK (SEQ ID NO:36), or TVAPTESS (SEQ ID NO:37)) at the end of the non-naturally occurring C-terminus, wherein the protein exhibits reduced neutralization by an anti-drug antibody relative to that of the protein lacking the cap domain. In some embodiments, the reduction is at least 10%, at least 20%, at least 30%, at least 40%, or at least 50%. In some embodiments, the reduction is at least 60%, at least 70%, at least 80%, at least 90%, or at least 95%. In specific embodiments, the neutralization of a protein by an ADA is determined by neutralization assay described herein (such as an assay described in Section 7.9.4) or known to one of skill in the art.

In another aspect, provided herein are methods for reducing the neutralization of a protein (e.g., an antibody) by an anti-drug antibody, wherein the protein comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits reduced neutralization by an anti-drug antibody relative to that of the protein lacking the (His)ₙ (SEQ ID NO:25) sequence. In a specific embodiment, provided herein is a method for reducing the neutralization of a protein by an anti-drug antibody, wherein the protein comprises an antigen binding region of an antibody, and wherein the antigen binding region comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits reduced neutralization by an anti-drug antibody relative to that of the protein lacking the (His)ₙ (SEQ ID NO:25) sequence. In another specific embodiment, provided herein is a method for reducing the neutralization of a protein by an anti-drug antibody, wherein the protein comprises an antibody variable domain, and wherein the antibody variable domain comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits reduced neutralization by an anti-drug antibody relative to that of the protein lacking the (His)ₙ (SEQ ID NO:25) sequence. In another specific embodiment, provided herein is a method for reducing the neutralization of a protein by an anti-drug antibody, wherein the protein comprises an antibody fragment, and wherein the antibody fragment comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits reduced neutralization by an anti-drug antibody relative to that of the protein lacking the (His)ₙ (SEQ ID NO: 25) sequence. In specific embodiments, the neutralization of a protein by an ADA is determined by neutralization assay described herein (such as an assay described in Section 7.9.4) or known to one of skill in the art.

In a specific embodiment, provided herein is a method for reducing the neutralization of a bispecific antibody by an anti-drug antibody, wherein the bispecific antibody comprises an antigen binding region of an antibody, and wherein the antigen binding region comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the bispecific antibody exhibits reduced neutralization by an anti-drug antibody relative to that of the bispecific antibody lacking the (His)ₙ (SEQ ID NO:25) sequence. In another specific embodiment, provided herein is a method for reducing the neutralization of a bispecific antibody by an anti-drug antibody, wherein the bispecific antibody comprises an antibody variable domain, and wherein the antibody variable domain comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the bispecific antibody exhibits reduced neutralization by an anti-drug antibody relative to that of the bispecific antibody lacking the (His)ₙ (SEQ ID NO:25) sequence. In another specific embodiment, provided herein is a method for reducing the neutralization of a bispecific antibody by an anti-drug antibody, wherein the bispecific antibody comprises an antibody fragment, and wherein the antibody fragment comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the bispecific antibody exhibits reduced neutralization by an anti-drug antibody relative to that of the bispecific antibody lacking the (His)ₙ (SEQ ID NO: 25) sequence. In specific embodiments, the neutralization of a protein by an ADA is determined by neutralization assay described herein (such as an assay described in Section 7.9.4) or known to one of skill in the art.

In a specific embodiment, provided herein is a method for reducing the neutralization of a multi-specific antibody (e.g., trispecific antibody) by an anti-drug antibody, wherein the multi-specific antibody comprises an antigen binding region of an antibody, and wherein the antigen binding region comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the multi-specific antibody exhibits reduced neutralization by an anti-drug antibody relative to that of the multi-specific antibody lacking the (His)ₙ (SEQ ID NO:25) sequence. In another specific embodiment, provided herein is a method for reducing the neutralization of a multi-specific antibody by an anti-drug antibody, wherein the multi-specific antibody comprises an antibody variable domain, and wherein the antibody variable domain comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the multi-specific antibody exhibits reduced neutralization by an anti-drug antibody relative to that of the multi-specific antibody lacking the (His)ₙ (SEQ ID NO: 25) sequence. In another specific embodiment, provided herein is a method for reducing the neutralization of a multi-specific antibody by an anti-drug antibody, wherein the multi-specific antibody comprises an antibody fragment, and wherein the antibody fragment comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the multi-specific antibody exhibits reduced neutralization by an anti-drug antibody relative to that of the multi-specific antibody lacking the (His)ₙ (SEQ ID NO:25) sequence. In specific embodiments, the neutralization of a protein by an ADA is determined by neutralization assay described herein (such as an assay described in Section 7.9.4) or known to one of skill in the art.

In another aspect, provided herein are methods for reducing the neutralization of a protein (e.g., an antibody) by an anti-drug antibody, wherein the protein comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the protein exhibits reduced neutralization by an anti-drug antibody relative to that of the protein lacking the (His)ₙ (SEQ ID NO:25) sequence. In a specific embodiment, provided herein is a method for reducing the neutralization of a protein by an anti-drug antibody, wherein the protein comprises an antigen binding region of an antibody, and wherein the antigen binding region comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the protein exhibits reduced neutralization by an anti-drug antibody relative to that of the protein lacking the (His)ₙ (SEQ ID NO:25) sequence. In another specific embodiment, provided herein is a method for reducing the neutralization of a protein by an anti-drug antibody, wherein the protein comprises an antibody variable domain, and wherein the antibody variable domain comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the protein exhibits reduced neutralization by an anti-drug antibody relative to that of the protein lacking the (His)ₙ (SEQ ID NO:25) sequence. In another specific embodiment, provided herein is a method for reducing the neutralization of a protein by an anti-drug antibody, wherein the protein comprises an antibody fragment, and wherein the antibody fragment comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the protein exhibits reduced neutralization by an anti-drug antibody relative to that of the protein lacking the (His)ₙ (SEQ ID NO: 25) sequence. In specific embodiments, the neutralization of a protein by an ADA is determined by neutralization assay described herein (such as an assay described in Section 7.9.4) or known to one of skill in the art.

In a specific embodiment, provided herein is a method for reducing the neutralization of a bispecific antibody by an anti-drug antibody, wherein the bispecific antibody comprises an antigen binding region of an antibody, and wherein the antigen binding region comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the bispecific antibody exhibits reduced neutralization by an anti-drug antibody relative to that of the bispecific antibody lacking the (His)ₙ (SEQ ID NO:25) sequence. In another specific embodiment, provided herein is a method for reducing the neutralization of a bispecific antibody by an anti-drug antibody, wherein the bispecific antibody comprises an antibody variable domain, and wherein the antibody variable domain comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the bispecific antibody exhibits reduced neutralization by an anti-drug antibody relative to that of the bispecific antibody lacking the (His)ₙ (SEQ ID NO:25) sequence. In another specific embodiment, provided herein is a method for reducing the neutralization of a bispecific antibody by an anti-drug antibody, wherein the bispecific antibody comprises an antibody fragment, and wherein the antibody fragment comprises a C-terminal anti-drug epitope, the method comprising adding a (His)ₙ (SEQ ID NO: 25) sequence at the end of the C-terminal anti-drug epitope, wherein n is 1, 2, 3, 4 or 5, and wherein the bispecific antibody exhibits reduced neutralization by an anti-drug antibody relative to that of the bispecific antibody lacking the (His)ₙ (SEQ ID NO:25) sequence. In specific embodiments, the neutralization of a protein by an ADA is determined by neutralization assay described herein (such as an assay described in Section 7.9.4) or known to one of skill in the art.

In a specific embodiment, provided herein is a method for reducing the neutralization of a multi-specific antibody (e.g., trispecific antibody) by an anti-drug antibody, wherein the multi-specific antibody comprises an antigen binding region of an antibody, and wherein the antigen binding region comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the multi-specific antibody exhibits reduced neutralization by an anti-drug antibody relative to that of the multi-specific antibody lacking the (His)ₙ (SEQ ID NO:25) sequence. In another specific embodiment, provided herein is a method for reducing the neutralization of a multi-specific antibody by an anti-drug antibody, wherein the multi-specific antibody comprises an antibody variable domain, and wherein the antibody variable domain comprises a C-terminal anti-drug epitope, the method comprising adding a (His)ₙ (SEQ ID NO: 25) sequence at the end of the C-terminal anti-drug epitope, wherein n is 1, 2, 3, 4 or 5, and wherein the multi-specific antibody exhibits reduced neutralization by an anti-drug antibody relative to that of the multi-specific antibody lacking the (His)ₙ (SEQ ID NO:25) sequence. In another specific embodiment, provided herein is a method for reducing the neutralization of a multi-specific antibody by an anti-drug antibody, wherein the multi-specific antibody comprises an antibody fragment, and wherein the antibody fragment comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the multi-specific antibody exhibits reduced neutralization by an anti-drug antibody relative to that of the multi-specific antibody lacking the (His)ₙ (SEQ ID NO:25) sequence. In specific embodiments, the neutralization of a protein by an ADA is determined by neutralization assay described herein (such as an assay described in Section 7.9.4) or known to one of skill in the art.

In specific embodiments, the addition of a cap domain at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in a reduction in the neutralization of the protein (e.g., an antibody), the bispecific antibody, or the multispecific antibody (e.g., trispecific antibody) by an ADA relative to the neutralization of the same protein (e.g., an antibody), the same bispecific antibody, or the same multispecific antibody (e.g., trispecific antibody), respectively, lacking the cap domain by the ADA. In some embodiments, the reduction is at least 10%, at least 20%, at least 30%, at least 40%, or at least 50%. In some embodiments, the reduction is at least 60%, at least 70%, at least 80%, at least 90%, or at least 95%. In specific embodiments, the neutralization of a protein by an ADA is determined by neutralization assay described herein (such as an assay described in Section 7.9.4) or known to one of skill in the art.

In specific embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in a reduction in the neutralization of the protein (e.g., an antibody), the bispecific antibody, or the multispecific antibody (e.g., trispecific antibody) by an ADA relative to the neutralization of the same protein (e.g., an antibody), the same bispecific antibody, or the same multispecific antibody (e.g., trispecific antibody), respectively, lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 10% reduction in the neutralization of the protein (e.g., an antibody), the bispecific antibody, or the multispecific antibody by an ADA, relative to the neutralization of the same protein (e.g., an antibody), the same bispecific antibody, or the same multispecific antibody, respectively, lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 20% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 30% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about a 40% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 50% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody, respectively, lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 60% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 70% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the (His)ₙ (SEQ ID NO: 25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about an 80% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody, respectively, lacking the (His)ₙ (SEQ ID NO:25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 90% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the (His)ₙ (SEQ ID NO: 25) sequence by the ADA. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific (e.g., trispecific antibody) antibody, wherein n is 1, 2, 3, 4, or 5, results in at least about a 95% reduction in the neutralization of the protein (e.g., an antibody), the bispecific antibody, or the multispecific antibody by an ADA, relative to the neutralization of the same protein (e.g., an antibody), the same bispecific antibody, or the same multispecific antibody (e.g., trispecific antibody), respectively, lacking the (His)ₙ (SEQ ID NO:25) sequence by the ADA. In specific embodiments, the neutralization of a protein by an ADA is determined by neutralization assay described herein (such as an assay described in Section 7.9.4) or known to one of skill in the art.

In specific embodiments, the addition of one histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in a reduction in the neutralization of the protein (e.g., an antibody), the bispecific antibody, or the multispecific antibody by an ADA relative to the neutralization of the same protein (e.g., an antibody), the same bispecific antibody, or the same multispecific antibody (e.g., trispecific antibody), respectively, lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 10% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 20% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 30% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 40% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 50% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 60% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 70% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about an 80% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 90% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the one histidine by the ADA. In some embodiments, the addition of one histidine at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 95% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the one histidine by the ADA. In particular embodiments, the addition of one histidine at the end of an exposed C-terminus, the end of a non-naturally occurring C-terminus, or the end of a C-terminal anti-drug antibody epitope of a protein (e.g., an antibody), a bispecific antibody, or a multispecific antibody eliminates neutralization of the protein (e.g., an antibody), the bispecific antibody, or the multispecific antibody (e.g., trispecific antibody) by an ADA. In specific embodiments, the neutralization of a protein by an ADA is determined by neutralization assay described herein (such as an assay described in Section 7.9.4) or known to one of skill in the art.

In specific embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in a reduction in the neutralization of the protein (e.g., an antibody), the bispecific antibody, or the multispecific antibody (e.g., trispecific antibody) by an ADA relative to the neutralization of the same protein (e.g., an antibody), the same bispecific antibody, or the same multispecific antibody (e.g., trispecific antibody), respectively, lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 10% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody, respectively, lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 20% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 30% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 40% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 50% reduction in the neutralization of the protein (e.g., an antibody), the bispecific antibody, or the multispecific antibody (e.g., trispecific antibody) by an ADA, relative to the neutralization the same protein (e.g., an antibody), the same bispecific antibody, or the same multispecific antibody (e.g., trispecific antibody), respectively, lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 60% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 70% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about an 80% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 90% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the two histidines by the ADA. In some embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 95% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the two histidines by the ADA. In particular embodiments, the addition of two histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) eliminates neutralization of the protein (e.g., an antibody), the bispecific antibody, or the multispecific antibody by an ADA. In specific embodiments, the neutralization of a protein by an ADA is determined by neutralization assay described herein (such as an assay described in Section 7.9.4) or known to one of skill in the art.

In specific embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in a reduction in the neutralization of the protein (e.g., an antibody), the bispecific antibody, or the multispecific antibody (e.g., trispecific antibody) by an ADA relative to the neutralization the same protein (e.g., an antibody), the same bispecific antibody, or the same multispecific antibody (e.g., trispecific antibody), respectively, lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 10% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 20% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 30% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 40% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 50% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 60% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 70% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about an 80% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 90% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the three histidines by the ADA. In some embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 95% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the three histidines by the ADA. In particular embodiments, the addition of three histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) eliminates neutralization of the protein (e.g., an antibody), the bispecific antibody, or the multispecific antibody (e.g., trispecific antibody) by an ADA. In specific embodiments, the neutralization of a protein by an ADA is determined by neutralization assay described herein (such as an assay described in Section 7.9.4) or known to one of skill in the art.

In specific embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in a reduction in the neutralization of the protein (e.g., an antibody), the bispecific antibody, or the multispecific antibody (e.g., trispecific antibody) by an ADA relative to the neutralization of the same protein (e.g., an antibody), the same bispecific antibody, or the same multispecific antibody (e.g., trispecific antibody), respectively, lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 10% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 20% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 30% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 40% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 50% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 60% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 70% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about an 80% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 90% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the four histidines by the ADA. In some embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 95% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody, respectively, lacking the four histidines by the ADA. In particular embodiments, the addition of four histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) eliminates neutralization of the protein (e.g., an antibody), the bispecific antibody, or the multispecific antibody (e.g., trispecific antibody) by an ADA. In specific embodiments, the neutralization of a protein by an ADA is determined by neutralization assay described herein (such as an assay described in Section 7.9.4) or known to one of skill in the art.

In specific embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in a reduction in the neutralization of the protein (e.g., an antibody), the bispecific antibody, or the multispecific antibody by an ADA relative to the neutralization of the same protein (e.g., an antibody), the same bispecific antibody, or the same multispecific antibody (e.g., trispecific antibody), respectively, lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 10% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 20% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 30% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 40% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 50% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 60% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 70% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about an 80% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 90% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the five histidines by the ADA. In some embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) results in at least about a 95% reduction in the neutralization of the protein (e.g., an antibody), the bispecific antibody, or the multispecific antibody (e.g., trispecific antibody) by an ADA, relative to the neutralization of the same protein (e.g., an antibody), the same bispecific antibody, or the same multispecific antibody (e.g., trispecific antibody), respectively, lacking the five histidines by the ADA. In particular embodiments, the addition of five histidines at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody) eliminates neutralization of the protein (e.g., an antibody), the bispecific antibody, or the multispecific antibody (e.g., trispecific antibody) by an ADA. In specific embodiments, the neutralization of a protein by an ADA is determined by neutralization assay described herein (such as an assay described in Section 7.9.4) or known to one of skill in the art.

In another aspect, provided herein are methods for improving the pharmacokinetics of a protein (e.g., an antibody), wherein the protein comprises an exposed C-terminus, the method comprising adding a cap domain (e.g., a His cap domain, SLSLSPGK (SEQ ID NO:36), or TVAPTESS (SEQ ID NO:37)) at the end of the exposed C-terminus, wherein the protein exhibits improved pharmacokinetics relative to that of the protein lacking the cap domain. In some embodiments, the improvement is at least 10%, at least 20%, at least 30%, at least 40%, or at least 50% improvement. In some embodiments, the improvement is at least 60%, at least 70%, at least 80%, at least 90%, or at least 95% improvement.

In another aspect, provided herein are methods for improving the pharmacokinetics of a protein (e.g., an antibody), wherein the protein comprises an exposed C-terminus, the method comprising adding a (His)ₙ (SEQ ID NO:25) sequence at the end of the exposed C-terminus, wherein n is 1, 2, 3, 4 or 5, and wherein the protein exhibits improved pharmacokinetics relative to that of the protein lacking the (His)ₙ (SEQ ID NO:25) sequence. In a specific embodiment, provided herein is a method for improving the pharmacokinetics of a protein (e.g., an antibody), wherein the protein comprises an antigen binding region of an antibody, and wherein the antigen binding region comprises an exposed C-terminus, the method comprising adding a (His)ₙ (SEQ ID NO: 25) sequence at the end of the exposed C-terminus, wherein n is 1, 2, 3, 4 or 5, and wherein the protein exhibits improved pharmacokinetics relative to that of the protein lacking the (His)ₙ (SEQ ID NO:25) sequence. In another specific embodiment, provided herein is a method for improving the pharmacokinetics of a protein (e.g., an antibody), wherein the protein comprises an antibody variable domain, and wherein the antibody variable domain comprises an exposed C-terminus, the method comprising adding a (His)ₙ (SEQ ID NO:25) sequence at the end of the exposed C-terminus, wherein n is 1, 2, 3, 4 or 5, and wherein the protein exhibits improved pharmacokinetics relative to that of the protein lacking the (His)ₙ (SEQ ID NO:25) sequence. In another specific embodiment, provided herein is a method for improving the pharmacokinetics of a protein (e.g., an antibody), wherein the protein comprises an antibody fragment, and wherein the antibody fragment comprises an exposed C-terminus, the method comprising adding a (His)ₙ (SEQ ID NO:25) sequence at the end of the exposed C-terminus, wherein n is 1, 2, 3, 4 or 5, and wherein the protein exhibits improved pharmacokinetics relative to that of the protein lacking the (His)ₙ (SEQ ID NO:25) sequence. In specific embodiments, the pharmacokinetics of a protein is determined by pharmacokinetic assays described herein (such as an assay described in Section 7.9.2) or known to one of skill in the art.

In a specific embodiment, provided herein is a method for improving the pharmacokinetics of a bispecific antibody, wherein the bispecific antibody comprises an antigen binding region of an antibody, and wherein the antigen binding region comprises an exposed C-terminus, the method comprising adding a (His)ₙ (SEQ ID NO:25) sequence at the end of the exposed C-terminus, wherein n is 1, 2, 3, 4 or 5, and wherein the bispecific antibody exhibits improved pharmacokinetics relative to that of the bispecific antibody lacking the (His)ₙ (SEQ ID NO: 25) sequence. In another specific embodiment, provided herein is a method for improving the pharmacokinetics of a bispecific antibody, wherein the bispecific antibody comprises an antibody variable domain, and wherein the variable domain comprises an exposed C-terminus, the method comprising adding a (His)ₙ (SEQ ID NO:25) sequence at the end of the exposed C-terminus, wherein n is 1, 2, 3, 4 or 5, and wherein the bispecific antibody exhibits improved pharmacokinetics relative to that of the bispecific antibody lacking the (His)ₙ (SEQ ID NO:25) sequence. In another specific embodiment, provided herein is a method for improving the pharmacokinetics of a bispecific antibody, wherein the bispecific antibody comprises an antibody fragment, and wherein the antibody fragment comprises an exposed C-terminus, the method comprising adding a (His)ₙ (SEQ ID NO:25) sequence at the end of the exposed C-terminus, wherein n is 1, 2, 3, 4 or 5, and wherein the bispecific antibody exhibits improved pharmacokinetics relative to that of the bispecific antibody lacking the (His)ₙ (SEQ ID NO:25) sequence. In specific embodiments, the pharmacokinetics of a protein is determined by pharmacokinetic assays described herein (such as an assay described in Section 7.9.2) or known to one of skill in the art.

In a specific embodiment, provided herein is a method for improving the pharmacokinetics of a multi-specific antibody (e.g., trispecific antibody), wherein the multi-specific antibody comprises an antigen binding region of an antibody, and wherein the antigen binding region comprises an exposed C-terminus, the method comprising adding a (His)ₙ (SEQ ID NO: 25) sequence at the end of the exposed C-terminus, wherein n is 1, 2, 3, 4 or 5, and wherein the multi-specific antibody exhibits improved pharmacokinetics relative to that of the multi-specific antibody lacking the (His)ₙ (SEQ ID NO:25) sequence. In another specific embodiment, provided herein is a method for improving the pharmacokinetics of a multi-specific antibody (e.g., trispecific antibody), wherein the multi-specific antibody comprises an antibody variable domain, and wherein the antibody variable domain comprises an exposed C-terminus, the method comprising adding a (His)ₙ (SEQ ID NO:25) sequence at the end of the exposed C-terminus, wherein n is 1, 2, 3, 4 or 5, and wherein the multi-specific antibody exhibits improved pharmacokinetics relative to that of the multi-specific antibody lacking the (His)ₙ (SEQ ID NO: 25) sequence. In another specific embodiment, provided herein is a method for improving the pharmacokinetics of a multi-specific antibody (e.g., trispecific antibody), wherein the multi-specific antibody comprises an antibody fragment, and wherein the antibody fragment comprises an exposed C-terminus, the method comprising adding a (His)ₙ (SEQ ID NO:25) sequence at the end of the exposed C-terminus, wherein n is 1, 2, 3, 4 or 5, and wherein the multi-specific antibody exhibits improved pharmacokinetics relative to that of the multi-specific antibody lacking the (His)ₙ (SEQ ID NO:25) sequence. In specific embodiments, the pharmacokinetics of a protein is determined by pharmacokinetic assays described herein (such as an assay described in Section 7.9.2) or known to one of skill in the art.

In another aspect, provided herein are methods for improving the pharmacokinetics of a protein, wherein the protein comprises a non-naturally occurring C-terminus, the method comprising adding a cap domain at the end of the non-naturally occurring C-terminus, wherein n is 1, 2, 3, 4 or 5, and wherein the protein exhibits improved pharmacokinetics relative to that of the protein lacking the cap domain. In some embodiments, the improvement is at least 10%, at least 20%, at least 30%, at least 40%, or at least 50% improvement. In some embodiments, the improvement is at least 60%, at least 70%, at least 80%, at least 90%, or at least 95% improvement.

In another aspect, provided herein are methods for improving the pharmacokinetics of a protein, wherein the protein comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits improved pharmacokinetics relative to that of the protein lacking the (His)ₙ (SEQ ID NO:25) sequence. In a specific embodiment, provided herein is a method for improving the pharmacokinetics of a protein, wherein the protein comprises an antigen binding region of an antibody, and wherein the antigen binding region comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits improved pharmacokinetics relative to that of the protein lacking the (His)ₙ (SEQ ID NO:25) sequence. In another specific embodiment, provided herein is a method for improving the pharmacokinetics of a protein, wherein the protein comprises an antibody variable domain, and wherein the antibody variable domain comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits improved pharmacokinetics relative to that of the protein lacking the (His)ₙ (SEQ ID NO:25) sequence. In another specific embodiment, provided herein is a method for improving the pharmacokinetics of a protein, wherein the protein comprises an antibody fragment, and wherein the antibody fragment comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits improved pharmacokinetics relative to that of the protein lacking the (His)ₙ (SEQ ID NO: 25) sequence. In specific embodiments, the pharmacokinetics of a protein is determined by pharmacokinetic assays described herein (such as an assay described in Section 7.9.2) or known to one of skill in the art.

In a specific embodiment, provided herein is a method for improving the pharmacokinetics of a bispecific antibody, wherein the bispecific antibody comprises an antigen binding region of an antibody, and wherein the antigen binding region comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the bispecific antibody exhibits improved pharmacokinetics relative to that of the bispecific antibody lacking the (His)ₙ (SEQ ID NO:25) sequence. In another specific embodiment, provided herein is a method for improving the pharmacokinetics of a bispecific antibody, wherein the bispecific antibody comprises an antibody variable domain, and wherein the antibody variable domain comprises a non-naturally occurring C-terminus, the method comprising adding a (His)ₙ (SEQ ID NO: 25) sequence at the end of the non-naturally occurring C-terminus, wherein n is 1, 2, 3, 4 or 5, and wherein the bispecific antibody exhibits improved pharmacokinetics relative to that of the bispecific antibody lacking the (His)ₙ (SEQ ID NO:25) sequence. In another specific embodiment, provided herein is a method for improving the pharmacokinetics of a bispecific antibody, wherein the bispecific antibody comprises an antibody fragment, and wherein the antibody fragment comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the bispecific antibody exhibits improved pharmacokinetics by an anti-drug antibody relative to that of the bispecific antibody lacking the (His)ₙ (SEQ ID NO:25) sequence. In specific embodiments, the pharmacokinetics of a protein is determined by pharmacokinetic assays described herein (such as an assay described in Section 7.9.2) or known to one of skill in the art.

In a specific embodiment, provided herein is a method for improving the pharmacokinetics of a multi-specific antibody (e.g., trispecific antibody), wherein the multi-specific antibody comprises an antigen binding region of an antibody, and wherein the antigen binding region comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the multi-specific antibody exhibits improved pharmacokinetics relative to that of the multi-specific antibody lacking the (His)ₙ (SEQ ID NO:25) sequence. In another specific embodiment, provided herein is a method for improving the pharmacokinetics of a multi-specific antibody, wherein the multi-specific antibody comprises an antibody variable domain, and wherein the antibody variable domain comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the multi-specific antibody exhibits improved pharmacokinetics relative to that of the multi-specific antibody lacking the (His)ₙ (SEQ ID NO:25) sequence. In another specific embodiment, provided herein is a method for improving the pharmacokinetics of a multi-specific antibody, wherein the multi-specific antibody comprises an antibody fragment, and wherein the antibody fragment comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the multi-specific antibody exhibits improved pharmacokinetics relative to that of the multi-specific antibody lacking the (His)ₙ (SEQ ID NO:25) sequence. In specific embodiments, the pharmacokinetics of a protein is determined by pharmacokinetic assays described herein (such as an assay described in Section 7.9.2) or known to one of skill in the art.

In another aspect, provided herein are methods for improving the pharmacokinetics of a protein (e.g., an antibody), wherein the protein comprises a C-terminal anti-drug epitope, the method comprising adding a cap domain (e.g., a His cap domain, SLSLSPGK (SEQ ID NO:36), or TVAPTESS (SEQ ID NO:37)) at the end of the C-terminal anti-drug epitope, wherein the protein exhibits improved pharmacokinetics relative to that of the protein lacking the cap domain. In some embodiments, the improvement is at least 10%, at least 20%, at least 30%, at least 40%, or at least 50% improvement. In some embodiments, the improvement is at least 60%, at least 70%, at least 80%, at least 90%, or at least 95% improvement.

In another aspect, provided herein are methods for improving the pharmacokinetics of a protein (e.g., an antibody), wherein the protein comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the protein exhibits improved pharmacokinetics relative to that of the protein lacking the (His)ₙ (SEQ ID NO:25) sequence. In a specific embodiment, provided herein is a method for improving the pharmacokinetics of a protein (e.g., an antibody), wherein the protein comprises an antigen binding region of an antibody, and wherein the antigen binding region comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the protein exhibits improved pharmacokinetics relative to that of the protein lacking the (His)ₙ (SEQ ID NO:25) sequence. In another specific embodiment, provided herein is a method for improving the pharmacokinetics of a protein (e.g., an antibody), wherein the protein comprises an antibody variable domain, and wherein the antibody variable domain comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the protein exhibits improved pharmacokinetics relative to that of the protein lacking the (His)ₙ (SEQ ID NO:25) sequence. In another specific embodiment, provided herein is a method for improving the pharmacokinetics of a protein (e.g., an antibody), wherein the protein comprises an antibody fragment, and wherein the antibody fragment comprises a C-terminal anti-drug epitope, the method comprising adding a (His)ₙ (SEQ ID NO: 25) sequence at the end of the C-terminal anti-drug epitope, wherein n is 1, 2, 3, 4 or 5, and wherein the protein exhibits improved pharmacokinetics relative to that of the protein lacking the (His)ₙ (SEQ ID NO:25) sequence. In specific embodiments, the pharmacokinetics of a protein is determined by pharmacokinetic assays described herein (such as an assay described in Section 7.9.2) or known to one of skill in the art.

In a specific embodiment, provided herein is a method for improving the pharmacokinetics of a bispecific antibody, wherein the bispecific antibody comprises an antigen binding region of an antibody, and wherein the antigen binding region comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the bispecific antibody exhibits improved neutralization relative to that of the bispecific antibody lacking the (His)ₙ (SEQ ID NO:25) sequence. In another specific embodiment, provided herein is a method for improving the pharmacokinetics of a bispecific antibody, wherein the bispecific antibody comprises an antibody variable domain, and wherein the antibody variable domain comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the bispecific antibody exhibits improved pharmacokinetics relative to that of the bispecific antibody lacking the (His)ₙ (SEQ ID NO:25) sequence. In another specific embodiment, provided herein is a for improving the pharmacokinetics of a bispecific antibody, wherein the bispecific antibody comprises an antibody fragment, and wherein the antibody fragment comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the bispecific antibody exhibits improved pharmacokinetics relative to that of the bispecific antibody lacking the (His)ₙ (SEQ ID NO:25) sequence. In specific embodiments, the pharmacokinetics of the bispecific antibody is determined by pharmacokinetic assays described herein (such as an assay described in Section 7.9.2) or known to one of skill in the art.

In a specific embodiment, provided herein is a method for improving the pharmacokinetics of a multi-specific antibody (e.g., trispecific antibody), wherein the multi-specific antibody comprises an antigen binding region of an antibody, and wherein the antigen binding region comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the multi-specific antibody exhibits improved pharmacokinetics relative to that of the multi-specific antibody lacking the (His)ₙ (SEQ ID NO:25) sequence. In another specific embodiment, provided herein is a method for improving the pharmacokinetics of a multi-specific antibody, wherein the multi-specific antibody comprises an antibody variable domain, and wherein the antibody variable domain comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the multi-specific antibody exhibits improved pharmacokinetics relative to that of the multi-specific antibody lacking the (His)ₙ (SEQ ID NO: 25) sequence. In another specific embodiment, provided herein is a method for improving the pharmacokinetics of a multi-specific antibody, wherein the multi-specific antibody comprises an antibody fragment, and wherein the antibody fragment comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the multi-specific antibody exhibits improved pharmacokinetics relative to that of the multi-specific antibody lacking the (His)ₙ (SEQ ID NO:25) sequence. In specific embodiments, the pharmacokinetics of a multi-specific antibody is determined by pharmacokinetic assays described herein (such as an assay described in Section 7.9.2) or known to one of skill in the art.

In specific embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in an improvement in the pharmacokinetics of the protein (e.g., an antibody), the bispecific antibody, or the multispecific antibody (e.g., trispecific antibody) relative to the pharmacokinetics of the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 10% improvement in the pharmacokinetics of the protein (e.g., an antibody), the bispecific antibody, or the multispecific antibody (e.g., trispecific antibody), relative to the pharmacokinetics of the same protein (e.g., an antibody), the same bispecific antibody, or the same multispecific antibody, respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 20% improvement in the pharmacokinetics of the protein (e.g., an antibody), the bispecific antibody, or the multispecific antibody (e.g., trispecific antibody), relative to the pharmacokinetics of the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 30% improvement in the pharmacokinetics of the protein (e.g., an antibody), the bispecific antibody, or the multispecific antibody (e.g., trispecific antibody), relative to the pharmacokinetics of the same protein (e.g., an antibody), the same bispecific antibody, or the same multispecific antibody (e.g., trispecific antibody), respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, (e.g., trispecific antibody) wherein n is 1, 2, 3, 4, or 5, results in at least about a 40% improvement in the pharmacokinetics of the protein (e.g., an antibody), the bispecific antibody, or the multispecific antibody (e.g., trispecific antibody), relative to the pharmacokinetics of the same protein (e.g., an antibody), the same bispecific antibody, or the same multispecific antibody, respectively, lacking the (His)ₙ (SEQ ID NO:25) sequence. In some embodiments, the addition of a (His)ₙ (SEQ ID NO: 25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about a 50% improvement in the pharmacokinetics of the protein (e.g., an antibody), the bispecific antibody, or the multispecific antibody (e.g., trispecific antibody), relative to the pharmacokinetics of the same protein (e.g., an antibody), the same bispecific antibody, or the same multispecific antibody (e.g., trispecific antibody), respectively, lacking the (His)ₙ (SEQ ID NO: 25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 60% improvement in the pharmacokinetics of the protein (e.g., an antibody), the bispecific antibody, or the multispecific antibody (e.g., trispecific antibody), relative to the pharmacokinetics of the same protein (e.g., an antibody), the same bispecific antibody, or the same multispecific antibody (e.g., trispecific antibody), respectively, lacking the (His)ₙ (SEQ ID NO: 25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 70% improvement in the pharmacokinetics of the protein (e.g., an antibody), the bispecific antibody, or the multispecific antibody (e.g., trispecific antibody), relative to the pharmacokinetics of the same protein (e.g., an antibody), the same bispecific antibody, or the same multispecific antibody (e.g., trispecific antibody), respectively, lacking the (His)ₙ (SEQ ID NO: 25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about an 80% improvement in the pharmacokinetics of the protein (e.g., an antibody), the bispecific antibody, or the multispecific antibody (e.g., trispecific antibody), relative to the pharmacokinetics of the same protein (e.g., an antibody), the same bispecific antibody, or the same multispecific antibody (e.g., trispecific antibody), respectively, lacking the (His)ₙ (SEQ ID NO: 25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 90% improvement in the pharmacokinetics of the protein (e.g., an antibody), the bispecific antibody, or the multispecific antibody (e.g., trispecific antibody), relative to the pharmacokinetics of the same protein (e.g., an antibody), the same bispecific antibody, or the same multispecific antibody, respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody (e.g., trispecific antibody), wherein n is 1, 2, 3, 4, or 5, results in at least about a 95% improvement in the pharmacokinetics of the protein (e.g., an antibody), the bispecific antibody, or the multispecific antibody (e.g., trispecific antibody), relative to the pharmacokinetics of the same protein (e.g., an antibody), the same bispecific antibody, or the same multispecific antibody, respectively, lacking the (His)ₙ (SEQ ID NO:25) sequence. In specific embodiments, the pharmacokinetics of a protein is determined by pharmacokinetic assays described herein (such as an assay described in Section 7.9.2) or known to one of skill in the art.

In another aspect, provided herein are methods for improving the safety of a protein (e.g., an antibody), wherein the protein comprises an exposed C-terminus, the method comprising adding a cap domain (e.g., a His cap domain, SLSLSPGK (SEQ ID NO:36), or TVAPTESS (SEQ ID NO:37)) at the end of the exposed C-terminus, wherein the protein exhibits improved safety relative to that of the protein lacking the cap domain. In some embodiments, the improvement is at least 10%, at least 20%, at least 30%, at least 40%, or at least 50% improvement. In some embodiments, the improvement is at least 60%, at least 70%, at least 80%, at least 90%, or at least 95% improvement.

In another aspect, provided herein are methods for improving the safety of a protein (e.g., an antibody), wherein the protein comprises an exposed C-terminus, the method comprising adding a (His)ₙ (SEQ ID NO:25) sequence at the end of the exposed C-terminus, wherein n is 1, 2, 3, 4 or 5, and wherein the protein exhibits improved safety relative to that of the protein lacking the (His)ₙ (SEQ ID NO:25) sequence. In a specific embodiment, provided herein is a method for improving the safety of a protein (e.g., an antibody), wherein the protein comprises an antigen binding region of an antibody, and wherein the antigen binding region comprises an exposed C-terminus, the method comprising adding a (His)ₙ (SEQ ID NO:25) sequence at the end of the exposed C-terminus, wherein n is 1, 2, 3, 4 or 5, and wherein the protein exhibits improved safety relative to that of the protein lacking the (His)ₙ (SEQ ID NO:25) sequence. In another specific embodiment, provided herein is a method for improving the safety of a protein (e.g., an antibody), wherein the protein comprises an antibody variable domain, and wherein the antibody variable domain comprises an exposed C-terminus, the method comprising adding a (His)ₙ (SEQ ID NO:25) sequence at the end of the exposed C-terminus, wherein n is 1, 2, 3, 4 or 5, and wherein the protein exhibits improved safety relative to that of the protein lacking the (His)ₙ (SEQ ID NO:25) sequence. In another specific embodiment, provided herein is a method for improving the safety of a protein (e.g., an antibody), wherein the protein comprises an antibody fragment, and wherein the antibody fragment comprises an exposed C-terminus, the method comprising adding a (His)ₙ (SEQ ID NO:25) sequence at the end of the exposed C-terminus, wherein n is 1, 2, 3, 4 or 5, and wherein the protein exhibits improved safety relative to that of the protein lacking the (His)ₙ (SEQ ID NO:25) sequence. In specific embodiments, the safety of a protein is determined by safety assays described herein (such as an assay described in Section 7.9.3) or known to one of skill in the art.

In a specific embodiment, provided herein is a method for improving the safety of a bispecific antibody, wherein the bispecific antibody comprises an antigen binding region of an antibody, and wherein the antigen binding region comprises an exposed C-terminus, the method comprising adding a (His)ₙ (SEQ ID NO:25) sequence at the end of the exposed C-terminus, wherein n is 1, 2, 3, 4 or 5, and wherein the bispecific antibody exhibits improved safety relative to that of the bispecific antibody lacking the (His)ₙ (SEQ ID NO:25) sequence. In another specific embodiment, provided herein is a method for improving the safety of a bispecific antibody, wherein the bispecific antibody comprises an antibody variable domain, and wherein the antibody variable domain comprises an exposed C-terminus, the method comprising adding a (His)ₙ (SEQ ID NO:25) sequence at the end of the exposed C-terminus, wherein n is 1, 2, 3, 4 or 5, and wherein the bispecific antibody exhibits improved safety relative to that of the bispecific antibody lacking the (His)ₙ (SEQ ID NO:25) sequence. In another specific embodiment, provided herein is a method for improving the safety of a bispecific antibody, wherein the bispecific antibody comprises an antibody fragment, and wherein the antibody fragment comprises an exposed C-terminus, the method comprising adding a (His)ₙ (SEQ ID NO:25) sequence at the end of the exposed C-terminus, wherein n is 1, 2, 3, 4 or 5, and wherein the bispecific antibody exhibits improved safety relative to that of the bispecific antibody lacking the (His)ₙ (SEQ ID NO: 25) sequence. In specific embodiments, the safety of a bispecific antibody is determined by safety assays described herein (such as an assay described in Section 7.9.3) or known to one of skill in the art.

In a specific embodiment, provided herein is a method for improving the safety of a multi-specific antibody (e.g., trispecific antibody), wherein the multi-specific antibody comprises an antigen binding region of an antibody, and wherein the antigen binding region comprises an exposed C-terminus, the method comprising adding a (His)ₙ (SEQ ID NO:25) sequence at the end of the exposed C-terminus, wherein n is 1, 2, 3, 4 or 5, and wherein the multi-specific antibody exhibits improved safety relative to that of the multi-specific antibody lacking the (His)ₙ (SEQ ID NO:25) sequence. In another specific embodiment, provided herein is a method for improving the safety of a multi-specific antibody, wherein the multi-specific antibody comprises an antibody variable domain, and wherein the antibody variable domain comprises an exposed C-terminus, the method comprising adding a (His)ₙ (SEQ ID NO:25) sequence at the end of the exposed C-terminus, wherein n is 1, 2, 3, 4 or 5, and wherein the multi-specific antibody exhibits improved safety relative to that of the multi-specific antibody lacking the (His)ₙ (SEQ ID NO:25) sequence. In another specific embodiment, provided herein is a method for improving the safety of a multi-specific antibody, wherein the multi-specific antibody comprises an antibody fragment, and wherein the antibody fragment comprises an exposed C-terminus, the method comprising adding a (His)ₙ (SEQ ID NO:25) sequence at the end of the exposed C-terminus, wherein n is 1, 2, 3, 4 or 5, and wherein the multi-specific antibody exhibits improved safety relative to that of the multi-specific antibody lacking the (His)ₙ (SEQ ID NO:25) sequence. In specific embodiments, the safety of a multi-specific antibody is determined by safety assays described herein (such as an assay described in Section 7.9.3) or known to one of skill in the art.

In another aspect, provided herein are methods for improving the safety of a protein, wherein the protein comprises a non-naturally occurring C-terminus, the method comprising adding a cap domain (e.g., a His cap domain, SLSLSPGK (SEQ ID NO:36), or TVAPTESS (SEQ ID NO:37)) at the end of the non-naturally occurring C-terminus, wherein the protein exhibits improved safety relative to that of the protein lacking the cap domain. In some embodiments, the improvement is at least 10%, at least 20%, at least 30%, at least 40%, or at least 50% improvement. In some embodiments, the improvement is at least 60%, at least 70%, at least 80%, at least 90%, or at least 95% improvement.

In another aspect, provided herein are methods for improving the safety of a protein, wherein the protein comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits improved safety relative to that of the protein lacking the (His)ₙ (SEQ ID NO:25) sequence. In a specific embodiment, provided herein is a method for improving the safety of a protein, wherein the protein comprises an antigen binding region of an antibody, and wherein the antigen binding region comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits improved safety relative to that of the protein lacking the (His)ₙ (SEQ ID NO:25) sequence. In another specific embodiment, provided herein is a method for improving the safety of a protein, wherein the protein comprises an antibody variable domain, and wherein the antibody variable domain comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits improved safety relative to that of the protein lacking the (His)ₙ (SEQ ID NO:25) sequence. In another specific embodiment, provided herein is a method for improving the safety of a protein, wherein the protein comprises an antibody fragment, and wherein the antibody fragment comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the protein exhibits improved safety relative to that of the protein lacking the (His)ₙ (SEQ ID NO:25) sequence. In specific embodiments, the safety of a protein is determined by safety assays described herein (such as an assay described in Section 7.9.3) or known to one of skill in the art.

In a specific embodiment, provided herein is a method for improving the safety of a bispecific antibody, wherein the bispecific antibody comprises an antigen binding region of an antibody, and wherein the antigen binding region comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the bispecific antibody exhibits improved safety relative to that of the bispecific antibody lacking the (His)ₙ (SEQ ID NO:25) sequence. In another specific embodiment, provided herein is a method for improving the safety of a bispecific antibody, wherein the bispecific antibody comprises an antibody variable domain, and wherein the antibody variable domain comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the bispecific antibody exhibits improved safety relative to that of the bispecific antibody lacking the (His)ₙ (SEQ ID NO:25) sequence. In another specific embodiment, provided herein is a method for improving the safety of a bispecific antibody, wherein the bispecific antibody comprises an antibody fragment, and wherein the antibody fragment comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the bispecific antibody exhibits improved safety by an anti-drug antibody relative to that of the bispecific antibody lacking the (His)ₙ (SEQ ID NO:25) sequence. In specific embodiments, the safety of a bispecific antibody is determined by safety assays described herein (such as an assay described in Section 7.9.3) or known to one of skill in the art.

In a specific embodiment, provided herein is a method for improving the safety of a multi-specific antibody, wherein the multi-specific antibody comprises an antigen binding region of an antibody, and wherein the antigen binding region comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the multi-specific antibody exhibits improved safety relative to that of the multi-specific antibody lacking the (His)ₙ (SEQ ID NO:25) sequence. In another specific embodiment, provided herein is a method for improving the safety of a multi-specific antibody, wherein the multi-specific antibody comprises an antibody variable domain, and wherein the antibody variable domain comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the multi-specific antibody exhibits improved safety relative to that of the multi-specific antibody lacking the (His)ₙ (SEQ ID NO:25) sequence. In another specific embodiment, provided herein is a method for improving the safety of a multi-specific antibody, wherein the multi-specific antibody comprises an antibody fragment, and wherein the antibody fragment comprises a non-naturally occurring C-terminus, the method comprising adding a (His)ₙ (SEQ ID NO:25) sequence at the end of the non-naturally occurring C-terminus, wherein n is 1, 2, 3, 4 or 5, and wherein the multi-specific antibody exhibits improved safety relative to that of the multi-specific antibody lacking the (His)ₙ (SEQ ID NO:25) sequence. In specific embodiments, the safety of a multi-specific antibody is determined by safety assays described herein (such as an assay described in Section 7.9.3) or known to one of skill in the art.

In another aspect, provided herein are methods for improving the safety of a protein (e.g., an antibody), wherein the protein comprises a C-terminal anti-drug epitope, the method comprising adding a cap domain (e.g., a His cap domain, SLSLSPGK (SEQ ID NO:36), or TVAPTESS (SEQ ID NO:37)) at the end of the C-terminal anti-drug epitope, wherein the protein exhibits improved safety relative to that of the protein lacking the cap domain. In some embodiments, the improvement is at least 10%, at least 20%, at least 30%, at least 40%, or at least 50% improvement. In some embodiments, the improvement is at least 60%, at least 70%, at least 80%, at least 90%, or at least 95% improvement.

In another aspect, provided herein are methods for improving the safety of a protein (e.g., an antibody), wherein the protein comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the protein exhibits improved safety relative to that of the protein lacking the (His)ₙ (SEQ ID NO:25) sequence. In a specific embodiment, provided herein is a method for improving the safety of a protein (e.g., an antibody), wherein the protein comprises an antigen binding region of an antibody, and wherein the antigen binding region comprises a C-terminal anti-drug epitope, the method comprising adding a (His)ₙ (SEQ ID NO: 25) sequence at the end of the C-terminal anti-drug epitope, wherein n is 1, 2, 3, 4 or 5, and wherein the protein exhibits improved safety relative to that of the protein lacking the (His)ₙ (SEQ ID NO:25) sequence. In another specific embodiment, provided herein is a method for improving the safety of a protein (e.g., an antibody), wherein the protein comprises an antibody variable domain, and wherein the antibody variable domain comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the protein exhibits improved safety relative to that of the protein lacking the (His)ₙ (SEQ ID NO:25) sequence. In another specific embodiment, provided herein is a method for improving the safety of a protein (e.g., an antibody), wherein the protein comprises an antibody fragment, and wherein the antibody fragment comprises a C-terminal anti-drug epitope, the method comprising adding a (His)ₙ (SEQ ID NO: 25) sequence at the end of the C-terminal anti-drug epitope, wherein n is 1, 2, 3, 4 or 5, and wherein the protein exhibits improved safety relative to that of the protein lacking the (His)ₙ (SEQ ID NO:25) sequence. In specific embodiments, the safety of a protein is determined by safety assays described herein (such as an assay described in Section 7.9.3) or known to one of skill in the art.

In a specific embodiment, provided herein is a method for improving the safety of a bispecific antibody, wherein the bispecific antibody comprises an antigen binding region of an antibody, and wherein the antigen binding region comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the bispecific antibody exhibits improved neutralization relative to that of the bispecific antibody lacking the (His)ₙ (SEQ ID NO:25) sequence. In another specific embodiment, provided herein is a method for improving the safety of a bispecific antibody, wherein the bispecific antibody comprises an antibody variable domain, and wherein the antibody variable domain comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the bispecific antibody exhibits improved safety relative to that of the bispecific antibody lacking the (His)ₙ (SEQ ID NO:25) sequence. In another specific embodiment, provided herein is a for improving the safety of a bispecific antibody, wherein the bispecific antibody comprises an antibody fragment, and wherein the antibody fragment comprises a C-terminal anti-drug epitope, the method comprising adding a (His)ₙ (SEQ ID NO: 25) sequence at the end of the C-terminal anti-drug epitope, wherein n is 1, 2, 3, 4 or 5, and wherein the bispecific antibody exhibits improved safety relative to that of the bispecific antibody lacking the (His)ₙ (SEQ ID NO:25) sequence. In specific embodiments, the safety of a protein is determined by safety assays described herein (such as an assay described in Section 7.9.3) or known to one of skill in the art.

In a specific embodiment, provided herein is a method for improving the safety of a multi-specific antibody, wherein the multi-specific antibody comprises an antigen binding region of an antibody, and wherein the antigen binding region comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the multi-specific antibody exhibits improved safety relative to that of the multi-specific antibody lacking the (His)ₙ (SEQ ID NO:25) sequence. In another specific embodiment, provided herein is a method for improving the safety of a multi-specific antibody, wherein the multi-specific antibody comprises an antibody variable domain, and wherein the antibody variable domain comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the multi-specific antibody exhibits improved safety relative to that of the multi-specific antibody lacking the (His)ₙ (SEQ ID NO:25) sequence. In another specific embodiment, provided herein is a method for improving the safety of a multi-specific antibody, wherein the multi-specific antibody comprises an antibody fragment, and wherein the antibody fragment comprises a C-terminal anti-drug epitope, the method comprising adding a (His)ₙ (SEQ ID NO:25) sequence at the end of the C-terminal anti-drug epitope, wherein n is 1, 2, 3, 4 or 5, and wherein the multi-specific antibody exhibits improved safety relative to that of the multi-specific antibody lacking the (His)ₙ (SEQ ID NO:25) sequence. In specific embodiments, the safety of a protein is determined by safety assays described herein (such as an assay described in Section 7.9.3) or known to one of skill in the art.

In specific embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in an improvement in the safety of the protein (e.g., an antibody), the bispecific antibody, or the multispecific antibody relative to the safety of the same protein (e.g., an antibody), the same bispecific antibody, or the same multispecific antibody, respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about a 10% improvement in the safety of the protein (e.g., an antibody), the bispecific antibody, or the multispecific antibody, relative to the safety of the same protein (e.g., an antibody), the same bispecific antibody, or the same multispecific antibody, respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about a 20% improvement in the safety of the protein (e.g., an antibody), the bispecific antibody, or the multispecific antibody, relative to the safety of the same protein (e.g., an antibody), the same bispecific antibody, or the same multispecific antibody, respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about a 30% improvement in the safety of the protein (e.g., an antibody), the bispecific antibody, or the multispecific antibody, relative to the safety of the same protein (e.g., an antibody), the same bispecific antibody, or the same multispecific antibody, respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about a 40% improvement in the safety of the protein (e.g., an antibody), the bispecific antibody, or the multispecific antibody, relative to the safety of the same protein (e.g., an antibody), the same bispecific antibody, or the same multispecific antibody, respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about a 50% improvement in the safety of the protein (e.g., an antibody), the bispecific antibody, or the multispecific antibody, relative to the safety of the same protein (e.g., an antibody), the same bispecific antibody, or the same multispecific antibody, respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about a 60% improvement in the safety of the protein (e.g., an antibody), the bispecific antibody, or the multispecific antibody, relative to the safety of the same protein (e.g., an antibody), the same bispecific antibody, or the same multispecific antibody, respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about a 70% improvement in the safety of the protein (e.g., an antibody), the bispecific antibody, or the multispecific antibody, relative to the safety of the same protein (e.g., an antibody), the same bispecific antibody, or the same multispecific antibody, respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about an 80% improvement in the safety of the protein (e.g., an antibody), the bispecific antibody, or the multispecific antibody, relative to the safety of the same protein (e.g., an antibody), the same bispecific antibody, or the same multispecific antibody, respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about a 90% improvement in the safety of the protein (e.g., an antibody), the bispecific antibody, or the multispecific antibody, relative to the safety of the same protein (e.g., an antibody), the same bispecific antibody, or the same multispecific antibody, respectively, lacking the (His)ₙ (SEQ ID NO:25) sequence. In some embodiments, the addition of a (His)ₙ (SEQ ID NO:25) sequence at the end of an exposed C-terminus, the end of a non-naturally occurring C-terminus, or the end of a C-terminal anti-drug antibody epitope of a protein (e.g., an antibody), a bispecific antibody, or a multispecific antibody, wherein n is 1, 2, 3, 4, or 5, results in at least about a 95% improvement in the safety of the protein (e.g., an antibody), the bispecific antibody, or the multispecific antibody, relative to the safety of the same protein (e.g., an antibody), the same bispecific antibody, or the same multispecific antibody, respectively, lacking the (His)ₙ (SEQ ID NO:25) sequence. In specific embodiments, the safety of a protein is determined by safety assays described herein (such as an assay described in Section 7.9.3) or known to one of skill in the art.

In another aspect, provided herein are methods for reducing the interaction between a protein and an anti-drug antibody, wherein the protein comprises an antibody heavy chain variable domain and the antibody heavy chain variable domain comprises an exposed C-terminus amino sequence, and wherein the last amino acid residues of the exposed C-terminus amino acid sequence consist of the amino acid sequence VTVSS (SEQ ID NO:21), VEIK (SEQ ID NO:22), VEIKR (SEQ ID NO:23), VEIKRT (SEQ ID NO:24), TKVTVL (SEQ ID NO:38), TKLTVL (SEQ ID NO:39), TQLIIL (SEQ ID NO:40), TELTVL (SEQ ID NO:41), or TQLTVL (SEQ ID NO: 42), the method comprising adding a cap domain (e.g., a His cap domain, SLSLSPGK (SEQ ID NO: 36), or TVAPTESS (SEQ ID NO:37)) after the last amino acid of the amino acid sequence, wherein the addition of the cap domain results in reduced interaction between the anti-drug antibody and protein relative to the interaction between the anti-drug antibody and the protein lacking the cap domain. In some embodiments, the reduction is at least 10%, at least 20%, at least 30%, at least 40%, or at least 50%. In some embodiments, the reduction is at least 60%, at least 70%, at least 80%, at least 90%, or at least 95%. In specific embodiments, the interaction between a protein and an ADA is determined by an ELISA, surface plasmon resonance assay (e.g., BIACORE), or other immunoassay described herein or known to one of skill in the art.

In another aspect, provided herein are methods for reducing the interaction between a protein and an anti-drug antibody, wherein the protein comprises an antibody heavy chain variable domain and the antibody heavy chain variable domain comprises an exposed C-terminus amino sequence, and wherein the last five amino acid residues of the exposed C-terminus amino acid sequence consist of the amino acid sequence VTVSS (SEQ ID NO:21), the method comprising adding a (His)ₙ (SEQ ID NO:25) sequence after the last S of the amino acid sequence VTVSS (SEQ ID NO:21), wherein n is 1, 2, 3, 4 or 5, and wherein the addition of the (His)ₙ (SEQ ID NO:25) sequence results in reduced interaction between the anti-drug antibody and protein relative to the interaction between the anti-drug antibody and the protein lacking the (His)ₙ (SEQ ID NO:25) sequence. In a specific embodiment, provided herein is a method for reducing the interaction between a bispecific antibody and an anti-drug antibody, wherein the bispecific antibody comprises an antibody heavy chain variable domain and the antibody heavy chain variable domain comprises an exposed C-terminus amino sequence, and wherein the last five amino acid residues of the exposed C-terminus amino acid sequence consist of the amino acid sequence VTVSS (SEQ ID NO:21), the method comprising adding a (His)ₙ (SEQ ID NO: 25) sequence after the last S of the amino acid sequence VTVSS (SEQ ID NO:21), wherein n is 1, 2, 3, 4 or 5, and wherein the addition of the (His)ₙ (SEQ ID NO:25) sequence results in reduced interaction between the anti-drug antibody and bispecific antibody relative to the interaction between the anti-drug antibody and the bispecific antibody lacking the (His)ₙ (SEQ ID NO: 25) sequence. In another specific embodiment, provided herein is a method for reducing the interaction between a multi-specific antibody and an anti-drug antibody, wherein the multi-specific antibody comprises an antibody heavy chain variable domain and the antibody heavy chain variable domain comprises an exposed C-terminus amino sequence, and wherein the last five amino acid residues of the exposed C-terminus amino acid sequence consist of the amino acid sequence VTVSS (SEQ ID NO:21), the method comprising adding a (His)ₙ (SEQ ID NO: 25) sequence after the last S of the amino acid sequence VTVSS (SEQ ID NO:21), wherein n is 1, 2, 3, 4 or 5, and wherein the addition of the (His)ₙ (SEQ ID NO:25) sequence results in reduced interaction between the anti-drug antibody and multi-specific relative to the interaction between the anti-drug antibody and the multi-specific lacking the (His)ₙ (SEQ ID NO:25) sequence. Various assays known to one of skill in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In another aspect, provided herein are methods for reducing the neutralization of a protein by an anti-drug antibody, wherein the protein comprises an antibody heavy chain variable domain and the antibody heavy chain variable domain comprises an exposed C-terminus amino sequence, and wherein the last amino acid residues of the exposed C-terminus amino acid sequence consist of the amino acid sequence VTVSS (SEQ ID NO:21), VEIK (SEQ ID NO:22), VEIKR (SEQ ID NO:23), VEIKRT (SEQ ID NO:24), TKVTVL (SEQ ID NO:38), TKLTVL (SEQ ID NO:39), TQLIIL (SEQ ID NO:40), TELTVL (SEQ ID NO:41), or TQLTVL (SEQ ID NO: 42), the method comprising adding a cap domain (e.g., a His cap domain, SLSLSPGK (SEQ ID NO: 36), or TVAPTESS (SEQ ID NO:37)) after the last amino acid of the amino acid sequence, wherein the addition of the cap domain results in reduced neutralization of the protein by the anti-drug antibody relative to the neutralization of the protein lacking the cap domain by the anti-drug antibody. In some embodiments, the reduction is at least 10%, at least 20%, at least 30%, at least 40%, or at least 50%. In some embodiments, the reduction is at least 60%, at least 70%, at least 80%, at least 90%, or at least 95%.

In another aspect, provided herein are methods for reducing the neutralization of a protein by an anti-drug antibody, wherein the protein comprises an antibody heavy chain variable domain and the antibody heavy chain variable domain comprises an exposed C-terminus amino sequence, and wherein the last five amino acid residues of the exposed C-terminus amino acid sequence consist of the amino acid sequence VTVSS (SEQ ID NO:21), the method comprising adding a (His)ₙ (SEQ ID NO:25) sequence after the last S of the amino acid sequence VTVSS (SEQ ID NO:21), wherein n is 1, 2, 3, 4 or 5, and wherein the addition of the (His)ₙ (SEQ ID NO: 25) sequence results in reduced neutralization of the protein by the anti-drug antibody relative to the neutralization of the protein lacking the (His)ₙ (SEQ ID NO:25) sequence by the anti-drug antibody. In a specific embodiment, provided herein is a method for reducing the neutralization of a bispecific antibody by an anti-drug antibody, wherein the bispecific antibody comprises an antibody heavy chain variable domain and the antibody heavy chain variable domain comprises an exposed C-terminus amino sequence, and wherein the last five amino acid residues of the exposed C-terminus amino acid sequence consist of the amino acid sequence VTVSS (SEQ ID NO:21), the method comprising adding a (His)ₙ (SEQ ID NO:25) sequence after the last S of the amino acid sequence VTVSS (SEQ ID NO:21), wherein n is 1, 2, 3, 4 or 5, and wherein the addition of the (His)ₙ (SEQ ID NO:25) sequence results in reduced neutralization of the bispecific antibody by the anti-drug antibody relative to the neutralization of the bispecific antibody lacking the (His)ₙ (SEQ ID NO:25) sequence by the anti-drug antibody. In another specific embodiment, provided herein is a method for reducing the neutralization of a multi-specific antibody by an anti-drug antibody, wherein the multi-specific antibody comprises an antibody heavy chain variable domain and the antibody heavy chain variable domain comprises an exposed C-terminus amino sequence, and wherein the last five amino acid residues of the exposed C-terminus amino acid sequence consist of the amino acid sequence VTVSS (SEQ ID NO:21), the method comprising adding a (His)ₙ (SEQ ID NO:25) sequence after the last S of the amino acid sequence VTVSS (SEQ ID NO:21), wherein n is 1, 2, 3, 4 or 5, and wherein the addition of the (His)ₙ (SEQ ID NO:25) sequence results in reduced neutralization of the multi-specific antibody by the anti-drug antibody relative to the neutralization of the multi-specific antibody lacking the (His)ₙ (SEQ ID NO:25) sequence by the anti-drug antibody. In specific embodiments, the neutralization of a protein by an ADA is determined by neutralization assay described herein (such as an assay described in Section 7.9.4) or known to one of skill in the art.

In another aspect, provided herein are methods for improving the pharmacokinetics of a protein, wherein the protein comprises an antibody heavy chain variable domain and the antibody heavy chain variable domain comprises an exposed C-terminus amino sequence, and wherein the last amino acid residues of the exposed C-terminus amino acid sequence consist of the amino acid sequence VTVSS (SEQ ID NO:21), VEIK (SEQ ID NO:22), VEIKR (SEQ ID NO: 23), VEIKRT (SEQ ID NO:24), TKVTVL (SEQ ID NO:38), TKLTVL (SEQ ID NO:39), TQLIIL (SEQ ID NO:40), TELTVL (SEQ ID NO:41), or TQLTVL (SEQ ID NO:42), the method comprising adding a cap domain (e.g., a His cap domain, SLSLSPGK (SEQ ID NO:36), or TVAPTESS (SEQ ID NO:37)) after the last amino acid of the amino acid sequence, wherein the addition of cap domain results in improved pharmacokinetics of the protein relative to the pharmacokinetics of the protein lacking the cap domain. In some embodiments, the improvement is at least 10%, at least 20%, at least 30%, at least 40%, or at least 50% improvement. In some embodiments, the improvement is at least 60%, at least 70%, at least 80%, at least 90%, or at least 95% improvement.

In another aspect, provided herein are methods for improving the pharmacokinetics of a protein, wherein the protein comprises an antibody heavy chain variable domain and the antibody heavy chain variable domain comprises an exposed C-terminus amino sequence, and wherein the last five amino acid residues of the exposed C-terminus amino acid sequence consist of the amino acid sequence VTVSS (SEQ ID NO:21), the method comprising adding a (His)ₙ (SEQ ID NO:25) sequence after the last S of the amino acid sequence VTVSS (SEQ ID NO:21), wherein n is 1, 2, 3, 4 or 5, and wherein the addition of the (His)ₙ (SEQ ID NO:25) sequence results in improved pharmacokinetics of the protein relative to the pharmacokinetics of the protein lacking the (His)ₙ (SEQ ID NO:25) sequence. In a specific embodiment, provided herein is a method for improving the pharmacokinetics of a bispecific antibody, wherein the bispecific antibody comprises an antibody heavy chain variable domain and the antibody heavy chain variable domain comprises an exposed C-terminus amino sequence, and wherein the last five amino acid residues of the exposed C-terminus amino acid sequence consist of the amino acid sequence VTVSS (SEQ ID NO:21), the method comprising adding a (His)ₙ (SEQ ID NO:25) sequence after the last S of the amino acid sequence VTVSS (SEQ ID NO:21), wherein n is 1, 2, 3, 4 or 5, and wherein the addition of the (His)ₙ (SEQ ID NO:25) sequence results in improved pharmacokinetics of the bispecific antibody relative to the pharmacokinetics of the bispecific antibody lacking the (His)ₙ (SEQ ID NO:25) sequence. In another specific embodiment, provided herein is a method for improving the pharmacokinetics of a multi-specific antibody, wherein the multi-specific antibody comprises an antibody heavy chain variable domain and the antibody heavy chain variable domain comprises an exposed C-terminus amino sequence, and wherein the last five amino acid residues of the exposed C-terminus amino acid sequence consist of the amino acid sequence VTVSS (SEQ ID NO:21), the method comprising adding a (His)ₙ (SEQ ID NO: 25) sequence after the last S of the amino acid sequence VTVSS (SEQ ID NO:21), wherein n is 1, 2, 3, 4 or 5, and wherein the addition of the (His)ₙ (SEQ ID NO:25) sequence results in improved pharmacokinetics of the multi-specific antibody relative to the pharmacokinetics of the multi-specific antibody lacking the (His)ₙ (SEQ ID NO:25) sequence. In specific embodiments, the pharmacokinetics of a protein is determined by pharmacokinetic assays described herein (such as an assay described in Section 7.9.2) or known to one of skill in the art.

In another aspect, provided herein are methods for improving the safety of a protein, wherein the protein comprises an antibody heavy chain variable domain and the antibody heavy chain variable domain comprises an exposed C-terminus amino sequence, and wherein the last amino acid residues of the exposed C-terminus amino acid sequence consist of the amino acid sequence VTVSS (SEQ ID NO:21), VEIK (SEQ ID NO:22), VEIKR (SEQ ID NO:23), VEIKRT (SEQ ID NO:24), TKVTVL (SEQ ID NO:38), TKLTVL (SEQ ID NO:39), TQLIIL (SEQ ID NO: 40), TELTVL (SEQ ID NO:41), or TQLTVL (SEQ ID NO:42), the method comprising adding a cap domain (e.g., a His cap domain, SLSLSPGK (SEQ ID NO:36), or TVAPTESS (SEQ ID NO:37)) after the last amino acid of the amino acid sequence, wherein the addition of the cap domain results in improved safety of the protein relative to the safety of the protein lacking the cap domain. In some embodiments, the improvement is at least 10%, at least 20%, at least 30%, at least 40%, or at least 50% improvement. In some embodiments, the improvement is at least 60%, at least 70%, at least 80%, at least 90%, or at least 95% improvement.

In another aspect, provided herein are methods for improving the safety of a protein, wherein the protein comprises an antibody heavy chain variable domain and the antibody heavy chain variable domain comprises an exposed C-terminus amino sequence, and wherein the last five amino acid residues of the exposed C-terminus amino acid sequence consist of the amino acid sequence VTVSS (SEQ ID NO:21), the method comprising adding a (His)ₙ (SEQ ID NO: 25) sequence after the last S of the amino acid sequence VTVSS (SEQ ID NO:21), wherein n is 1, 2, 3, 4 or 5, and wherein the addition of the (His)ₙ (SEQ ID NO:25) sequence results in improved safety of the protein relative to the safety of the protein lacking the (His)ₙ (SEQ ID NO: 25) sequence. In a specific embodiment, provided herein is a method for improving the safety of a bispecific antibody, wherein the bispecific antibody comprises an antibody heavy chain variable domain and the antibody heavy chain variable domain comprises an exposed C-terminus amino sequence, and wherein the last five amino acid residues of the exposed C-terminus amino acid sequence consist of the amino acid sequence VTVSS (SEQ ID NO:21), the method comprising adding a (His)ₙ (SEQ ID NO:25) sequence after the last S of the amino acid sequence VTVSS (SEQ ID NO:21), wherein n is 1, 2, 3, 4 or 5, and wherein the addition of the (His)ₙ (SEQ ID NO:25) sequence results in improved safety of the bispecific antibody relative to the safety of the bispecific antibody lacking the (His)ₙ (SEQ ID NO:25) sequence. In another specific embodiment, provided herein is a method for improving the safety of a multi-specific antibody, wherein the multi-specific antibody comprises an antibody heavy chain variable domain and the antibody heavy chain variable domain comprises an exposed C-terminus amino sequence, and wherein the last five amino acid residues of the exposed C-terminus amino acid sequence consist of the amino acid sequence VTVSS (SEQ ID NO:21), the method comprising adding a (His)ₙ (SEQ ID NO:25) sequence after the last S of the amino acid sequence VTVSS (SEQ ID NO:21), wherein n is 1, 2, 3, 4 or 5, and wherein the addition of the (His)ₙ (SEQ ID NO: 25) sequence results in improved safety of the multi-specific antibody relative to the safety of the multi-specific antibody lacking the (His)ₙ (SEQ ID NO:25) sequence. In specific embodiments, the safety of a protein is determined by safety assays described herein (such as an assay described in Section 7.9.3) or known to one of skill in the art.

In another aspect, provided herein are methods for reducing the interaction between a protein and an anti-drug antibody, wherein the protein comprises an antibody light chain variable domain and the antibody light chain variable domain comprises an exposed C-terminus amino sequence, and wherein the last four to six amino acid residues of the exposed C-terminus amino acid sequence consist of the amino acid sequence VEIK (SEQ ID NO:22), VEIKR (SEQ ID NO: 23), or VEIKRT (SEQ ID NO:24), the method comprising adding a (His)ₙ (SEQ ID NO:25) sequence after the last amino acid residue of the C-terminus of the VEIK (SEQ ID NO:22), VEIKR (SEQ ID NO:23), or VEIKRT (SEQ ID NO:24) amino acid sequence, wherein n is 1, 2, 3, 4 or 5, and wherein the addition of the (His)ₙ (SEQ ID NO:25) sequence results in reduced interaction between the anti-drug antibody and protein relative to the interaction between the anti-drug antibody and the protein lacking the (His)ₙ (SEQ ID NO:25) sequence. In a specific embodiment, provided herein is a method for reducing the interaction between a bispecific antibody and an anti-drug antibody, wherein the protein comprises an antibody light chain variable domain and the antibody light chain variable domain comprises an exposed C-terminus amino sequence, and wherein the last four to six amino acid residues of the exposed C-terminus amino acid sequence consist of the amino acid sequence VEIK, VEIKR, or VEIKRT, the method comprising adding a (His)ₙ (SEQ ID NO:25) sequence after the last amino acid residue of the C-terminus of the VEIK (SEQ ID NO:22), VEIKR (SEQ ID NO:23), or VEIKRT (SEQ ID NO:24) amino acid sequence, wherein n is 1, 2, 3, 4 or 5, and wherein the addition of the (His)ₙ (SEQ ID NO: 25) sequence results in reduced interaction between the anti-drug antibody and bispecific antibody relative to the interaction between the anti-drug antibody and the bispecific antibody lacking the (His)ₙ (SEQ ID NO:25) sequence. In another specific embodiment, provided herein is a method for reducing the interaction between a multi-specific antibody and an anti-drug antibody, wherein the protein comprises an antibody light chain variable domain and the antibody light chain variable domain comprises an exposed C-terminus amino sequence, and wherein the last four to six amino acid residues of the exposed C-terminus amino acid sequence consist of the amino acid sequence VEIK (SEQ ID NO:22), VEIKR (SEQ ID NO:23), or VEIKRT (SEQ ID NO:24), the method comprising adding a (His)ₙ (SEQ ID NO:25) sequence after the last amino acid residue of the C-terminus of the VEIK (SEQ ID NO:22), VEIKR (SEQ ID NO: 23), or VEIKRT (SEQ ID NO:24) amino acid sequence, wherein n is 1, 2, 3, 4 or 5, and wherein the addition of the (His)ₙ (SEQ ID NO:25) sequence results in reduced interaction between the anti-drug antibody and multi-specific antibody relative to the interaction between the anti-drug antibody and the multi-specific antibody lacking the (His)ₙ (SEQ ID NO:25) sequence. Various assays known to one of skill in the art or described herein can be used to measure and/or detect interaction of the protein with an ADA, such as an assay described in Section 7.9.1. In specific embodiments, the interaction between a protein and an ADA is determined by an ELISA, surface plasmon resonance assay (e.g., BIACORE), or other immunoassay described herein or known to one of skill in the art.

In another aspect, provided herein are methods for reducing the neutralization of a protein by an anti-drug antibody, wherein the protein comprises an antibody light chain variable domain and the antibody light chain variable domain comprises an exposed C-terminus amino sequence, and wherein the last four to six amino acid residues of the exposed C-terminus amino acid sequence consist of the amino acid sequence VEIK (SEQ ID NO:22), VEIKR (SEQ ID NO: 23), or VEIKRT (SEQ ID NO:24), the method comprising adding a (His)ₙ (SEQ ID NO:25) sequence after the last amino acid residue of the C-terminus of the VEIK (SEQ ID NO:22), VEIKR (SEQ ID NO:23), or VEIKRT (SEQ ID NO:24) amino acid sequence, wherein n is 1, 2, 3, 4 or 5, and wherein the addition of the (His)ₙ (SEQ ID NO:25) sequence results in reduced neutralization of the protein by the anti-drug antibody relative to the neutralization of the protein lacking the (His)ₙ (SEQ ID NO:25) sequence by the anti-drug antibody. In a specific embodiment, provided herein is a method for reducing the neutralization of a bispecific antibody by an anti-drug antibody, wherein the protein comprises an antibody light chain variable domain and the antibody light chain variable domain comprises an exposed C-terminus amino sequence, and wherein the last four to six amino acid residues of the exposed C-terminus amino acid sequence consist of the amino acid sequence VEIK (SEQ ID NO:22), VEIKR (SEQ ID NO:23), or VEIKRT (SEQ ID NO:24), the method comprising adding a (His)ₙ (SEQ ID NO:25) sequence after the last amino acid residue of the C-terminus of the VEIK (SEQ ID NO:22), VEIKR (SEQ ID NO: 23), or VEIKRT (SEQ ID NO:24) amino acid sequence, wherein n is 1, 2, 3, 4 or 5, and wherein the addition of the (His)ₙ (SEQ ID NO:25) sequence results in reduced neutralization of the bispecific antibody by the anti-drug antibody relative to the neutralization of the bispecific antibody lacking the (His)ₙ (SEQ ID NO:25) sequence by the anti-drug antibody. In another specific embodiment, provided herein is a method for reducing the neutralization of a multi-specific antibody by an anti-drug antibody, wherein the protein comprises an antibody light chain variable domain and the antibody light chain variable domain comprises an exposed C-terminus amino sequence, and wherein the last four to six amino acid residues of the exposed C-terminus amino acid sequence consist of the amino acid sequence VEIK (SEQ ID NO:22), VEIKR (SEQ ID NO: 23), or VEIKRT (SEQ ID NO:24), the method comprising adding a (His)ₙ (SEQ ID NO: 25) sequence after the last amino acid residue of the C-terminus of the VEIK (SEQ ID NO: 22), VEIKR (SEQ ID NO:23), or VEIKRT (SEQ ID NO:24) amino acid sequence, wherein n is 1, 2, 3, 4 or 5, and wherein the addition of the (His)ₙ (SEQ ID NO:25) sequence results in reduced neutralization of the multi-specific antibody by the anti-drug antibody relative to the neutralization of the multi-specific antibody lacking the (His)ₙ (SEQ ID NO:25) sequence by the anti-drug antibody. In specific embodiments, the neutralization of a protein by an ADA is determined by neutralization assay described herein (such as an assay described in Section 7.9.4) or known to one of skill in the art.

In another aspect, provided herein are methods for improving the pharmacokinetics of a protein, wherein the protein comprises an antibody light chain variable domain and the antibody light chain variable domain comprises an exposed C-terminus amino sequence, and wherein the last four to six amino acid residues of the exposed C-terminus amino acid sequence consist of the amino acid sequence VEIK (SEQ ID NO:22), VEIKR (SEQ ID NO:23), or VEIKRT (SEQ ID NO:24), the method comprising adding a (His)ₙ (SEQ ID NO:25) sequence after the last amino acid residue of the C-terminus of the VEIK (SEQ ID NO:22), VEIKR (SEQ ID NO: 23), or VEIKRT (SEQ ID NO:24) amino acid sequence, wherein n is 1, 2, 3, 4 or 5, and wherein the addition of the (His)ₙ (SEQ ID NO:25) sequence results in improved pharmacokinetics of the protein relative to the pharmacokinetics of the protein lacking the (His)ₙ (SEQ ID NO:25) sequence. In a specific embodiment, provided herein is a method for improving the pharmacokinetics of a bispecific antibody, wherein the protein comprises an antibody light chain variable domain and the antibody light chain variable domain comprises an exposed C-terminus amino sequence, and wherein the last four to six amino acid residues of the exposed C-terminus amino acid sequence consist of the amino acid sequence VEIK (SEQ ID NO:22), VEIKR (SEQ ID NO:23), or VEIKRT (SEQ ID NO:24), the method comprising adding a (His)ₙ (SEQ ID NO:25) sequence after the last amino acid residue of the C-terminus of the VEIK (SEQ ID NO: 22), VEIKR (SEQ ID NO:23), or VEIKRT (SEQ ID NO:24) amino acid sequence, wherein n is 1, 2, 3, 4 or 5, and wherein the addition of the (His)ₙ (SEQ ID NO:25) sequence results in improved pharmacokinetics of the bispecific antibody relative to the pharmacokinetics of the bispecific antibody lacking the (His)ₙ (SEQ ID NO:25) sequence. In another specific embodiment, provided herein is a method for improving the pharmacokinetics of a multi-specific antibody, wherein the protein comprises an antibody light chain variable domain and the antibody light chain variable domain comprises an exposed C-terminus amino sequence, and wherein the last four to six amino acid residues of the exposed C-terminus amino acid sequence consist of the amino acid sequence VEIK (SEQ ID NO:22), VEIKR (SEQ ID NO:23), or VEIKRT (SEQ ID NO:24), the method comprising adding a (His)ₙ (SEQ ID NO:25) sequence after the last amino acid residue of the C-terminus of the VEIK (SEQ ID NO:22), VEIKR (SEQ ID NO: 23), or VEIKRT (SEQ ID NO:24) amino acid sequence, wherein n is 1, 2, 3, 4 or 5, and wherein the addition of the (His)ₙ (SEQ ID NO:25) sequence results in improved pharmacokinetics of the multi-specific antibody relative to the pharmacokinetics of the multi-specific antibody lacking the (His)ₙ (SEQ ID NO:25) sequence. In specific embodiments, the pharmacokinetics of a protein is determined by pharmacokinetic assays described herein (such as an assay described in Section 7.9.2) or known to one of skill in the art.

In another aspect, provided herein are methods for improving the safety of a protein, wherein the protein comprises an antibody light chain variable domain and the antibody light chain variable domain comprises an exposed C-terminus amino sequence, and wherein the last four to six amino acid residues of the exposed C-terminus amino acid sequence consist of the amino acid sequence VEIK (SEQ ID NO:22), VEIKR (SEQ ID NO:23), or VEIKRT (SEQ ID NO: 24), the method comprising adding a (His)ₙ (SEQ ID NO:25) sequence after the last amino acid residue of the C-terminus of the VEIK (SEQ ID NO:22), VEIKR (SEQ ID NO:23), or VEIKRT (SEQ ID NO:24) amino acid sequence, wherein n is 1, 2, 3, 4 or 5, and wherein the addition of the (His)ₙ (SEQ ID NO:25) sequence results in improved safety of the protein relative to the safety of the protein lacking the (His)ₙ (SEQ ID NO:25) sequence. In a specific embodiment, provided herein is a method for improving the safety of a bispecific antibody, wherein the protein comprises an antibody light chain variable domain and the antibody light chain variable domain comprises an exposed C-terminus amino sequence, and wherein the last four to six amino acid residues of the exposed C-terminus amino acid sequence consist of the amino acid sequence VEIK (SEQ ID NO:22), VEIKR (SEQ ID NO:23), or VEIKRT (SEQ ID NO: 24), the method comprising adding a (His)ₙ (SEQ ID NO:25) sequence after the last amino acid residue of the C-terminus of the VEIK (SEQ ID NO:22), VEIKR (SEQ ID NO:23), or VEIKRT (SEQ ID NO:24) amino acid sequence, wherein n is 1, 2, 3, 4 or 5, and wherein the addition of the (His)ₙ (SEQ ID NO:25) sequence results in improved safety of the bispecific antibody relative to the safety of the bispecific antibody lacking the (His)ₙ (SEQ ID NO:25) sequence. In another specific embodiment, provided herein is a method for improving the safety of a multi-specific antibody, wherein the protein comprises an antibody light chain variable domain and the antibody light chain variable domain comprises an exposed C-terminus amino sequence, and wherein the last four to six amino acid residues of the exposed C-terminus amino acid sequence consist of the amino acid sequence VEIK (SEQ ID NO:22), VEIKR (SEQ ID NO: 23), or VEIKRT (SEQ ID NO:24), the method comprising adding a (His)ₙ (SEQ ID NO:25) sequence after the last amino acid residue of the C-terminus of the VEIK (SEQ ID NO:22), VEIKR (SEQ ID NO:23), or VEIKRT (SEQ ID NO:24) amino acid sequence, wherein n is 1, 2, 3, 4 or 5, and wherein the addition of the (His)ₙ (SEQ ID NO:25) sequence results in improved safety of the multi-specific antibody relative to the safety of the multi-specific antibody lacking the (His)ₙ (SEQ ID NO:25) sequence. In specific embodiments, the safety of a protein is determined by safety assays described herein (such as an assay described in Section 7.9.3) or known to one of skill in the art.

### 7.6. Capping Means

In one aspect, provided herein is a capping means (e.g., a histidine capping means) for reducing interaction between a reference and an isolated polypeptide that includes one or more target binding regions and an exposed C-terminus, or an isolated protein that includes (a) a first polypeptide comprising a target binding region, and an exposed C-terminus; and (b) a second polypeptide comprising a target binding region. In some embodiments, the one or more target binding regions comprise an antibody variable domain or antigen binding fragment thereof. In some embodiments, the target binding region of the first and second polypeptides are capable of binding to different targets. In certain embodiments, the isolated protein further comprises a third polypeptide comprising a target binding region. In some embodiments, the third polypeptide is capable of binding to a different target than the first polypeptide, the second polypeptide, or both. In some embodiments, the isolated protein is heterodimeric. In some embodiments, the isolated protein is bispecific. In some embodiments, the isolated protein is trispecific. In some embodiments, the isolated protein is multi-specific. In some embodiments, the reference is a molecule present in a biological substance. In some embodiments, the biological substance comprises serum and/or plasma (e.g., human serum and/or human plasma). In specific embodiments, the biological substance comprises serum (e.g., human serum). In specific embodiments, the biological substance comprises plasma (e.g., human plasma). In some embodiments, the reference comprises an ADA. In some embodiments, the ADA is pre-existing in the serum and/or plasma of a subject (e.g., a human subject).

Also provided herein is a capping means (e.g., a histidine capping means) for reducing aggregation of an isolated polypeptide that includes one or more target binding regions and an exposed C-terminus, or an isolated protein that includes (a) a first polypeptide comprising a target binding region, and an exposed C-terminus; and (b) a second polypeptide comprising a target binding region. In some embodiments, provided herein is a capping means (e.g., a histidine capping means) for reducing aggregation of a polypeptide that includes one or more target binding regions and an exposed C-terminus, or an isolated protein that includes (a) a first polypeptide comprising a target binding region, and an exposed C-terminus; and (b) a second polypeptide comprising a target binding region. In some embodiments, the one or more target binding regions comprise an antibody variable domain or antigen binding fragment thereof. In some embodiments, the target binding region of the first and second polypeptides are capable of binding to different targets. In some embodiments, the second polypeptide comprises an exposed C-terminus. In certain embodiments, the isolated protein further comprises a third polypeptide comprising a target binding region. In some embodiments, the third polypeptide comprises an exposed C-terminus. In some embodiments, the first polypeptide, the second polypeptide, and the third polypeptide each comprise an exposed C-terminus. In some embodiments, the third polypeptide is capable of binding to a different target than the first polypeptide, the second polypeptide, or both. In some embodiments, the isolated protein is heterodimeric. In some embodiments, the isolated protein is bispecific. In some embodiments, the isolated protein is trispecific. In some embodiments, the isolated protein is multi-specific. In some embodiments, the polypeptide is in a biological substance. biological substance is serum and/or plasma (e.g., human serum and/or human plasma). In specific embodiments, the biological substance is human plasma. In specific embodiments, the biological substance is human serum. In some embodiments, the polypeptide in the biological substance is incubated at a certain temperature for a certain period of time. In some embodiments, incubation comprises about five to about fourteen days at about 37° C. in a biological substance (e.g., human serum and/or human plasma). In some embodiments, incubation comprises about two to about seven days at about 37° C. in a biological substance (e.g., human serum and/or human plasma). In some embodiments, incubation comprises about two to about five days at about 37° C. in a biological substance (e.g., human serum and/or human plasma). In some embodiments, incubation comprises about seven to about fourteen days at about 40° C. in a biological substance (e.g., human serum and/or human plasma). In some embodiments, incubation comprises about two to about seven days at about 40° C. in a biological substance (e.g., human serum and/or human plasma). In some embodiments, incubation comprises about two to about five days at about 40° C. in a biological substance (e.g., human serum and/or human plasma). In some embodiments, incubation comprises about seven to about fourteen days at about 4° C. in a biological substance (e.g., human serum and/or human plasma). In some embodiments, incubation comprises about two to about seven days at about 4° C. in a biological substance (e.g., human serum and/or human plasma). In some embodiments, incubation comprises about two to about five days at about 4° C. in a biological substance (e.g., human serum and/or human plasma). In some embodiments, incubation comprises about five to about fourteen days at about 25° C. in a biological substance (e.g., human serum and/or human plasma). In some embodiments, incubation comprises about two to about seven days at about 25° C. in a biological substance (e.g., human serum and/or human plasma). In some embodiments, incubation comprises about two to about five days at about 25° C. in a biological substance (e.g., human serum and/or human plasma).

In another aspect, provided herein is a capping means (e.g., a histidine capping means) for reducing self-aggregation of an isolated polypeptide that includes one or more target binding regions and an exposed C-terminus, or an isolated protein that includes (a) a first polypeptide comprising a target binding region, and an exposed C-terminus; and (b) a second polypeptide comprising a target binding region. In some embodiments, the one or more target binding regions comprise an antibody variable domain or antigen binding fragment thereof. In some embodiments, the target binding region of the first and second polypeptides are capable of binding to different targets. In certain embodiments, the isolated protein further comprises a third polypeptide comprising a target binding region. In some embodiments, the third polypeptide is capable of binding to a different target than the first polypeptide, the second polypeptide, or both. In some embodiments, the isolated protein is heterodimeric. In some embodiments, the isolated protein is bispecific. In some embodiments, the isolated protein is trispecific. In some embodiments, the isolated protein is multi-specific. In some embodiments, the aggregation is reduced in a high concentration liquid formulation (HCLF). In specific embodiments, the HCLF is about 50 mg/mL to about 150 mg/mL. In some embodiments, the aggregation is reduced in a biological substance (e.g., serum). In some embodiments, aggregation is determined under thermal stress (e.g., incubation at about 40° C.). In some embodiments, aggregation is determined under physiological stress (e.g., incubation at about 37° C.). In some embodiments, aggregation is determined by incubation at about 40° C. for about two weeks.

In a specific aspect, provided herein is a capping means (e.g., a histidine capping means) for reducing interaction between a protein and an anti-drug antibody, wherein the protein comprises an exposed C-terminus. In a specific embodiment, provided herein is a capping means (e.g., a histidine capping means) for reducing interaction between a protein and an anti-drug antibody, wherein the protein comprises an antigen binding region of an antibody, and wherein the antigen binding region comprises an exposed C-terminus. In another specific embodiment, provided herein is a capping means (e.g., a histidine capping means) for reducing interaction between a protein and an anti-drug antibody, wherein the protein comprises an antibody variable domain, and wherein the antibody variable domain comprises an exposed C-terminus. In a specific embodiment, provided herein is a capping means (e.g., a histidine capping means) for reducing interaction between a protein and an anti-drug antibody, wherein the protein comprises an antibody fragment, and wherein the antibody fragment comprises an exposed C-terminus. In certain embodiments, the protein is an antibody or an antibody fragment. In some embodiments, the antibody or antibody fragment comprises is a scFv or single domain antibody. In a specific embodiment, provided herein is a capping means (e.g., a histidine capping means) for reducing interaction between a bispecific antibody and an anti-drug antibody, wherein the bispecific antibody comprises an antigen binding region of an antibody, and wherein the antigen binding region comprises an exposed C-terminus. In another specific embodiment, provided herein is a capping means (e.g., a histidine capping means) for reducing interaction between a bispecific antibody and an anti-drug antibody, wherein the bispecific antibody comprises an antibody variable domain, and wherein the antibody variable domain comprises an exposed C-terminus. In a specific embodiment, provided herein is a capping means (e.g., a histidine capping means) for reducing interaction between a bispecific antibody and an anti-drug antibody, wherein the bispecific antibody comprises an antibody fragment, and wherein the antibody fragment comprises an exposed C-terminus. In some embodiments, the antibody fragment comprises is a scFv or single domain antibody. In certain embodiments, the antibody variable domain is a light chain variable domain. In other embodiments, the antibody variable domain is heavy chain variable domain. In some embodiments, the anti-drug antibody is pre-existing in the serum and/or plasma of a subject (e.g., a human subject).

In a specific embodiment, provided herein is a capping means (e.g., a histidine capping means) for reducing interaction between a multi-specific antibody and an anti-drug antibody, wherein the multi-specific antibody comprises an antigen binding region of an antibody, and wherein the antigen binding region comprises an exposed C-terminus. In another specific embodiment, provided herein is a capping means (e.g., a histidine capping means) for reducing interaction between a multi-specific antibody and an anti-drug antibody, wherein the multi-specific antibody comprises an antibody variable domain, and wherein the antibody variable domain comprises an exposed C-terminus. In a specific embodiment, provided herein is a capping means (e.g., a histidine capping means) for reducing interaction between a multi-specific antibody and an anti-drug antibody, wherein the multi-specific antibody comprises an antibody fragment, and wherein the antibody fragment comprises an exposed C-terminus. In some embodiments, the antibody fragment comprises is a scFv or single domain antibody. In certain embodiments, the antibody variable domain is a light chain variable domain. In other embodiments, the antibody variable domain is heavy chain variable domain. In some embodiments, the anti-drug antibody is pre-existing in the serum and/or plasma of a subject (e.g., a human subject).

In another aspect, provided herein is a capping means (e.g., a histidine capping means) for reducing interaction between a protein and an anti-drug antibody, wherein the protein comprises a non-naturally occurring C-terminus. In a specific embodiment, provided herein is a capping means (e.g., a histidine capping means) for reducing interaction between a protein and an anti-drug antibody, wherein the protein comprises an antigen binding region of an antibody, and wherein the antigen binding region comprises a non-naturally occurring C-terminus. In some embodiments, the anti-drug antibody is pre-existing in the serum and/or plasma of a subject (e.g., a human subject).

In another specific embodiment, provided herein is a capping means (e.g., a histidine capping means) for reducing interaction between a protein and an anti-drug antibody, wherein the protein comprises an antibody variable domain, and wherein the antibody variable domain comprises a non-naturally occurring C-terminus. In a specific embodiment, provided herein is a capping means (e.g., a histidine capping means) for reducing interaction between a protein and an anti-drug antibody, wherein the protein comprises an antibody fragment, and wherein the antibody fragment comprises a non-naturally occurring C-terminus. In certain embodiments, the protein is an antibody or an antibody fragment. In some embodiments, the antibody or antibody fragment comprises is a scFv or single domain antibody. In certain embodiments, the antibody variable domain is a light chain variable domain. In other embodiments, the antibody variable domain is heavy chain variable domain. In some embodiments, the anti-drug antibody is pre-existing in the serum and/or plasma of a subject (e.g., a human subject).

In a specific embodiment, provided herein is a capping means (e.g., a histidine capping means) for reducing interaction between a bispecific antibody and an anti-drug antibody, wherein the bispecific antibody comprises an antigen binding region of an antibody, and wherein the antigen binding region comprises a non-naturally occurring C-terminus. In another specific embodiment, provided herein is a capping means (e.g., a histidine capping means) for reducing interaction between a bispecific antibody and an anti-drug antibody, wherein the bispecific antibody comprises an antibody variable domain, and wherein the antibody variable domain comprises a non-naturally occurring C-terminus. In a specific embodiment, provided herein is a capping means (e.g., a histidine capping means) for reducing interaction between a bispecific antibody and an anti-drug antibody, wherein the bispecific antibody comprises an antibody fragment, and wherein the antibody fragment comprises a non-naturally occurring C-terminus. In some embodiments, the antibody fragment comprises is a scFv or single domain antibody. In certain embodiments, the antibody variable domain is a light chain variable domain. In other embodiments, the antibody variable domain is heavy chain variable domain. In some embodiments, the anti-drug antibody is pre-existing in the serum and/or plasma of a subject (e.g., a human subject).

In a specific embodiment, provided herein is a capping means (e.g., a histidine capping means) for reducing interaction between a multi-specific antibody and an anti-drug antibody, wherein the multi-specific antibody comprises an antigen binding region of an antibody, and wherein the antigen binding region comprises a non-naturally occurring C-terminus. In another specific embodiment, provided herein is a capping means (e.g., a histidine capping means) for reducing interaction between a multi-specific antibody and an anti-drug antibody, wherein the multi-specific antibody comprises an antibody variable domain, and wherein the antibody variable domain comprises a non-naturally occurring C-terminus. In a specific embodiment, provided herein is a capping means (e.g., a histidine capping means) for reducing interaction between a multi-specific antibody and an anti-drug antibody, wherein the multi-specific antibody comprises an antibody fragment, and wherein the antibody fragment comprises a non-naturally occurring C-terminus. In some embodiments, the antibody fragment comprises is a scFv or single domain antibody. In certain embodiments, the antibody variable domain is a light chain variable domain. In other embodiments, the antibody variable domain is heavy chain variable domain. In some embodiments, the anti-drug antibody is pre-existing in the serum and/or plasma of a subject (e.g., a human subject).

In another aspect, provided herein is a capping means (e.g., a histidine capping means) for reducing interaction between a protein and an anti-drug antibody, wherein the protein comprises a C-terminal anti-drug antibody epitope. In a specific embodiment, provided herein is a capping means (e.g., a histidine capping means) for reducing interaction between a protein and an anti-drug antibody, wherein the protein comprises an antigen binding region of an antibody, and wherein the antigen binding region comprises a C-terminal anti-drug antibody epitope. In some embodiments, the anti-drug antibody is pre-existing in the serum and/or plasma of a subject (e.g., a human subject).

In another specific embodiment, provided herein is a capping means (e.g., a histidine capping means) for reducing interaction between a protein and an anti-drug antibody, wherein the protein comprises an antibody variable domain, and wherein the antibody variable domain comprises a C-terminal anti-drug antibody epitope. In a specific embodiment, provided herein is a capping means (e.g., a histidine capping means) for reducing interaction between a protein and an anti-drug antibody, wherein the protein comprises an antibody fragment, and wherein the antibody fragment comprises a C-terminal anti-drug antibody epitope. In certain embodiments, the protein is an antibody or an antibody fragment. In some embodiments, the antibody or antibody fragment comprises is a scFv or single domain antibody. In certain embodiments, the antibody variable domain is a light chain variable domain. In other embodiments, the antibody variable domain is heavy chain variable domain. In some embodiments, the anti-drug antibody is pre-existing in the serum and/or plasma of a subject (e.g., a human subject).

In a specific embodiment, provided herein is a capping means (e.g., a histidine capping means) for reducing interaction between a bispecific antibody and an anti-drug antibody, wherein the bispecific antibody comprises an antigen binding region of an antibody, and wherein the antigen binding region comprises a C-terminal anti-drug antibody epitope. In another specific embodiment, provided herein is a capping means (e.g., a histidine capping means) for reducing interaction between a bispecific antibody and an anti-drug antibody, wherein the bispecific antibody comprises an antibody variable domain, and wherein the antibody variable domain comprises a C-terminal anti-drug antibody epitope. In a specific embodiment, provided herein is a capping means (e.g., a histidine capping means) for reducing interaction between a bispecific antibody and an anti-drug antibody, wherein the bispecific antibody comprises an antibody fragment, and wherein the antibody fragment comprises a C-terminal anti-drug antibody epitope. In some embodiments, the antibody fragment comprises is a scFv or single domain antibody. In certain embodiments, the antibody variable domain is a light chain variable domain. In other embodiments, the antibody variable domain is heavy chain variable domain. In some embodiments, the anti-drug antibody is pre-existing in the serum and/or plasma of a subject (e.g., a human subject).

In a specific embodiment, provided herein is a capping means (e.g., a histidine capping means) for reducing interaction between a multi-specific antibody and an anti-drug antibody, wherein the multi-specific antibody comprises an antigen binding region of an antibody, and wherein the antigen binding region comprises a C-terminal anti-drug antibody epitope. In another specific embodiment, provided herein is a capping means (e.g., a histidine capping means) for reducing interaction between a multi-specific antibody and an anti-drug antibody, wherein the multi-specific antibody comprises an antibody variable domain, and wherein the antibody variable domain comprises a C-terminal anti-drug antibody epitope. In a specific embodiment, provided herein is a capping means (e.g., a histidine capping means) for reducing interaction between a multi-specific antibody and an anti-drug antibody, wherein the multi-specific antibody comprises an antibody fragment, and wherein the antibody fragment comprises a C-terminal anti-drug antibody epitope. In some embodiments, the antibody fragment comprises is a scFv or single domain antibody. In certain embodiments, the antibody variable domain is a light chain variable domain. In other embodiments, the antibody variable domain is heavy chain variable domain. In some embodiments, the anti-drug antibody is pre-existing in the serum and/or plasma of a subject (e.g., a human subject).

In another aspect, provided herein is a composition comprising a protein and a capping means (e.g., a histidine capping means) for reducing interaction between the protein and an anti-drug antibody, wherein the protein comprises an exposed C-terminus. In a specific embodiment, provided herein is a composition comprising a protein and a capping means (e.g., a histidine capping means) for reducing interaction between the protein and an anti-drug antibody, wherein the protein comprises an antibody variable domain, and wherein the antibody variable domain comprises an exposed C-terminus. In another specific embodiment, provided herein is a composition comprising a protein and a capping means (e.g., a histidine capping means) for reducing interaction between the protein and an anti-drug antibody, wherein the protein comprises an antibody variable domain, and wherein the antibody variable domain comprises an exposed C-terminus. In another specific embodiment, provided herein is a composition comprising a protein and a capping means (e.g., a histidine capping means) for reducing interaction between the protein and an anti-drug antibody, wherein the protein comprises an antibody fragment, and wherein the antibody fragment comprises an exposed C-terminus. In certain embodiments, the protein is an antibody or an antibody fragment. In some embodiments, the antibody or antibody fragment comprises is a scFv or single domain antibody. In certain embodiments, the protein is a bispecific or multi-specific antibody. In certain embodiments, the antibody variable domain is a light chain variable domain. In other embodiments, the antibody variable domain is heavy chain variable domain. In some embodiments, the anti-drug antibody is pre-existing in the serum and/or plasma of a subject (e.g., a human subject).

In another aspect, provided herein is a composition comprising a protein and a capping means (e.g., a histidine capping means) for reducing interaction between the protein and an anti-drug antibody, wherein the protein comprises a non-naturally occurring C-terminus. In a specific embodiment, provided herein is a composition comprising a protein and a capping means (e.g., a histidine capping means) for reducing interaction between the protein and an anti-drug antibody, wherein the protein comprises an antigen binding region of an antibody, and wherein the antigen binding region comprises a non-naturally occurring C-terminus. In another specific embodiment, provided herein is a composition comprising a protein and a capping means (e.g., a histidine capping means) for reducing interaction between the protein and an anti-drug antibody, wherein the protein comprises an antibody variable domain, and wherein the antibody variable domain comprises a non-naturally occurring C-terminus. In another specific embodiment, provided herein is a composition comprising a protein and a capping means (e.g., a histidine capping means) for reducing interaction between the protein and an anti-drug antibody, wherein the protein comprises an antibody fragment, and wherein the antibody fragment comprises a non-naturally occurring C-terminus. In certain embodiments, the protein is an antibody or an antibody fragment. In some embodiments, the antibody or antibody fragment comprises is a scFv or single domain antibody. In certain embodiments, the protein is a bispecific or multi-specific antibody. In certain embodiments, the antibody variable domain is a light chain variable domain. In other embodiments, the antibody variable domain is heavy chain variable domain. In some embodiments, the anti-drug antibody is pre-existing in the serum and/or plasma of a subject (e.g., a human subject).

In another aspect, provided herein is a composition comprising a protein and a capping means (e.g., a histidine capping means) for reducing interaction between the protein and an anti-drug antibody, wherein the protein comprises a C-terminal anti-drug antibody epitope. In a specific embodiment, provided herein is a composition comprising a protein and a capping means (e.g., a histidine capping means) for reducing interaction between the protein and an anti-drug antibody, wherein the protein comprises an antigen binding region of an antibody, and wherein the antigen binding region comprises a C-terminal anti-drug antibody epitope. In another specific embodiment, provided herein is a composition comprising a protein and a capping means (e.g., a histidine capping means) for reducing interaction between the protein and an anti-drug antibody, wherein the protein comprises an antibody variable domain, and wherein the antibody variable domain comprises a non-naturally occurring C-terminus. In another specific embodiment, provided herein is a composition comprising a protein and a capping means (e.g., a histidine capping means) for reducing interaction between the protein and an anti-drug antibody, wherein the protein comprises an antibody fragment, and wherein the antibody fragment comprises a C-terminal anti-drug antibody epitope. In certain embodiments, the protein is an antibody or an antibody fragment. In some embodiments, the antibody or antibody fragment comprises is a scFv or single domain antibody. In certain embodiments, the protein is a bispecific or multi-specific antibody. In certain embodiments, the antibody variable domain is a light chain variable domain. In other embodiments, the antibody variable domain is heavy chain variable domain. In some embodiments, the anti-drug antibody is pre-existing in the serum and/or plasma of a subject (e.g., a human subject).

### 7.7. Use of Protein

In certain aspects, the polypeptide or protein (e.g., an antibody or another antigen binding protein) provided herein (such as a protein described in Section 7.1), polynucleotide encoding the protein provided herein (such as a polynucleotide described in Section 7.2) or a composition (such as a composition described in Section 7.4) can be used for treating and/or preventing a disease or disorder. In one embodiment, provided herein is a method for treating a disease or disorder comprising administering a capped polypeptide or a capped protein described herein (e.g., a histidine capped polypeptide or histidine capped protein described herein) or a composition thereof. In another embodiment, provided herein is a method for preventing a disease or disorder comprising administering a capped polypeptide or a capped protein described herein (e.g., a histidine capped polypeptide or histidine capped protein described herein) or a composition thereof. Non-limiting examples of diseases or disorders include a cancer (e.g., a hematopoietic, or solid tumor), sickle cell, psoriasis, osteoporosis, a viral disease or disorder (e.g., HIV), a bacterial disease or disorder, asthma, hemophilia, an autoimmune disease or disorder, ulcerative colitis, Crohn's disease, and graft-versus-host disease.

Various commercially available antibodies are provided in Table 2 above, and are suitable for use in the present disclosure, either as an intact antibody, as an antibody fragment, or as a portion of a multi-specific antibody (e.g., a fusion protein). The commercial antibodies may be used for the treatment of the indication noted in Table 2. In addition, an antibody that binds to the same target as provided in Table 2 above is suitable for use in the present disclosure and may be used for the treatment of the indication noted in Table 2.

An antibody that binds to an antigen of an organism provided in Table 3 is suitable for use in the present disclosure and may be used for the prevention and/or treatment of an infectious disease caused by the indicated organism.

In some embodiments, the prevention of a disease, disorder or condition prevents the onset of one or more symptoms of the disease, disorder or condition. In some embodiments, the treatment of a disease, disorder or condition reduces the number of symptoms and/or severity of one or more symptoms of the disease, disorder or condition.

In certain aspects, a polypeptide or protein described herein (e.g., an antibody or another antigen binding protein) provided herein (such as a protein described in Section 7.1), polynucleotide encoding the protein provided herein (such as a polynucleotide described in Section 7.2) or a composition (such as a composition described in Section 7.4) can be used in an assay to detect a molecule (e.g., an antigen). The assay may be conducted in vitro or in vivo. In specific embodiments, the assay is conducted in vitro in a biological substance. In some embodiments, the biological substance comprises serum and/or plasma (e.g., human serum and/or human plasma). For example, a blood sample, serum sample, or plasma sample may be contacted with a capped protein or capped polypeptide described herein (e.g., a histidine capped protein or histidine capped polypeptide described herein) and the binding of the capped protein or capped polypeptide to a molecule, such as, e.g., an antigen, is detected. In some embodiments, the assay is conducted in vivo. For example, a capped protein or capped polypeptide described herein (e.g., a histidine capped protein or histidine capped polypeptide described herein) is administered to a subject and the subject is imaged to detect the binding of the capped protein or capped polypeptide to a molecule, such as, e.g., an antigen.

In certain aspects, a polypeptide or protein described herein (e.g., an antibody or another antigen binding protein) provided herein (such as a protein described in Section 7.1), polynucleotide encoding the protein provided herein (such as a polynucleotide described in Section 7.2) or a composition (such as a composition described in Section 7.4) can be used in a diagnostic assay. The diagnostic assay may be conducted in vitro or in vivo. In specific embodiments, the diagnostic assay is conducted in vitro in a biological substance. In some embodiments, the biological substance comprises serum and/or plasma (e.g., human serum and/or human plasma). For example, a blood sample, serum sample, or plasma sample may be contacted with a capped protein or capped polypeptide described herein (e.g., a histidine capped protein or histidine capped polypeptide described herein) and the binding of the capped protein or capped polypeptide to a molecule, such as, e.g., an antigen, is detected. In some embodiments, the diagnostic assay is conducted in vivo. For example, a capped protein or capped polypeptide described herein (e.g., a histidine capped protein or histidine capped polypeptide described herein) is administered to a subject and the subject is imaged to detect the binding of the capped protein or capped polypeptide to a molecule, such as, e.g., an antigen. The detection of the molecule may result in the diagnosis of a disease, disorder, or condition (e.g., cancer or an infectious disease).

In certain aspects, a polypeptide or protein described herein (e.g., an antibody or another antigen binding protein) provided herein (such as a protein described in Section 7.1), or a composition (such as a composition described in Section 7.4) can be stored in a biological substance. In some embodiments, the biological substance comprises serum and/or plasma (e.g., human serum and/or human plasma). Without being bound by any theory, the capping of the protein or polypeptide reduces interaction with anti-drug antibodies in a biological substance and/or inhibits aggregation.

### 7.8. Kit

Also provided herein are kits comprising a protein (e.g., an antibody or another antigen binding protein) provided herein, or a composition (e.g., a pharmaceutical composition) thereof, packaged into suitable packaging material. In some embodiments, the kit comprises a capping means. In some embodiments, the kit comprises a histidine capping means that consists of (His)ₙ (SEQ ID NO:25), wherein n is 1, 2, 3, 4, or 5. In a specific embodiment, the histidine capping means consists of a single His. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain therapeutic proteins provided herein, or cells that contain nucleic acids encoding the therapeutic proteins provided herein. The cells in the kit can be maintained under appropriate storage conditions until ready to use.

### 7.9. Assays

7.9.1. Assays for Detecting Interaction with a Reference

Any assay known in the art for detecting interaction of a polypeptide or protein with a reference can be used. In some embodiments, the assay detects interaction of a polypeptide or protein (e.g., antibodies and/or other antigen binding proteins) with an ADA. Non-limiting examples include immunoassays (e.g., enzyme-linked immunosorbent assay (ELISA), radioimmunoprecipitation assay (RIP), electrochemiluminescence immunoassay (ECL or ECLIA), surface plasmon resonance immunoassay (SPRIA)), cell-based in vitro bioassay or competitive ligand binding assay, and may depend on the type of ADA seeking detection. For example, the protein of interest can be immobilized to a microtiter plate, and a test sample (e.g., a biological sample from a subject, such as serum and/or plasma) can be added to the wells of the plate. If the test sample contains a detectable amount of ADA that binds to the protein, detection can be achieved using a detection antibody (e.g., anti-human/mouse/rabbit IgG, IgM, IgA, or IgE HRP conjugate, anti-human/mouse/rabbit Fc HRP conjugate). Alternatively, or in addition, a subpopulation of the ADAs that neutralize and/or inhibit the functional activity of a protein (termed “neutralizing antibodies (NAbs)”) can be detected with an assay described in Section 7.9.4. Other conventional methods can also be employed as suitable.

### 7.9.2. Pharmacokinetic Assays

Any assay known in the art to assess the pharmacokinetic properties of a polypeptide or protein (e.g., antibodies and/or other antigen binding proteins) can be used. Non-limiting examples of a pharmacokinetic assay include an enzyme linked immunosorbent assay (ELISA) specific for the detection and quantification of the protein of interest, LC-MS, and/or other assay capable of measuring the absorption, distribution, metabolism, and/or excretion of the protein. For example, the protein of interest can be administered to a subject, and the rate of clearance can be measured, relative to a protein that does not include an exposed C-terminus with a cap domain (e.g., a His cap domain, SLSLSPGK (SEQ ID NO:36), or TVAPTESS (SEQ ID NO:37)) fused to the exposed C-terminus. Other conventional methods can also be employed as suitable.

### 7.9.3. Safety Assays

Any assay known in the art to assess the safety of a protein (e.g., antibodies and/or other antigen binding proteins) can be used. Non-limiting examples of safety assays include in vivo (e.g., animal models), ex vivo (isolated organs, organoids, or other test systems not involving intact animals), and in vitro (e.g., cell-based) assays that measure toxicity and/or immunogenicity of the protein. For example, in-vivo based safety assays can involve evaluation of acute infusion-related reaction (e.g., anaphylactic/anaphylactoid reactions), delayed (e.g., T-cell mediated) hypersensitivity, and/or autoimmunity/protein cross-reactivity. Alternatively, or in addition, safety assays can involve assessment of immunogenicity by detecting ADA, such as by using an assay described in Section 7.9.1. Other conventional methods can also be employed as suitable.

### 7.9.4. Neutralization Assays

Any assay known in the art to assess the neutralization of a protein (e.g., antibodies and/or other antigen binding proteins) can be used. Non-limiting examples of a neutralization assay include a cell-based bioassay or a non-cell based assay (e.g., a competitive ligand binding assay or an enzyme activity assay). A bioassay can measure cellular responses (e.g., phosphorylation of intracellular substrates, calcium mobilization, proliferation, and cell death). For example, the direct effect of neutralization on reducing and/or inhibiting bioactivity in the bioassay can be measured for a protein that directly stimulates a cellular response. Alternatively, when a protein indirectly impacts cellular activity (e.g., by blocking a receptor-ligand interaction), the indirect effect of the neutralization on restoring bioactivity in a bioassay can be measured. Other conventional methods can also be employed as suitable.

### 7.9.5. Structural Assays

Any assay known in the art to assess the structure of a protein (e.g., antibodies and/or other antigen binding proteins) can be used. Non-limiting examples of a structural assay include crystallography, such as X-ray crystallography. Structure can also be evaluated by measuring protein aggregates, and/or modifications to the protein (e.g., glycosylation, glycation). Other conventional methods can also be employed as suitable.

### 7.9.6. Protein Stability Assays

Any assay known in the art to assess the protein stability of a polypeptide or protein (e.g., antibodies and/or other antigen binding proteins) can be used. Non-limiting examples include circular dichroism (CD), dynamic light scattering (DLS), static light scattering (SLS), conventional differential scanning fluorimetry (DSF), nanoDSF, differential scanning calorimetry (DSC), size-exclusion chromatography-multi-angle light scattering (SEC-MALS), Fourier transform infrared spectroscopy (FTIR), analytical ultracentrifugation (AUC), size exclusion chromatography (SEC), intrinsic fluorescence (IF), or a combination thereof. In specific embodiments, protein stability is measured using thermal or chemical denaturation experiments. Thermal denaturation can be used to measure protein stability by employing increasing temperature at a constant rate, whereas chemical denaturation can be used to measure protein stability by employing chemical denaturants in increasing concentrations. Other conventional methods can also be employed as suitable.

### 7.9.7. Aggregation Assays

Any assay known in the art to assess the aggregation of a polypeptide or protein (e.g., antibodies and/or other antigen binding proteins), including self-aggregation can be used. Non-limiting examples include analytical ultracentrifugation, size-exclusion chromatography (SEC) (e.g., analytical SEC), hydrophobic interaction chromatography (HIC), dynamic light scattering (DLS), gel electrophoresis (e.g., capillary isoelectric focusing), electron microscopy, or a combination thereof.

Aggregation can be measured under various conditions. In specific embodiments, aggregation is measured in a high concentration liquid formulation (HCLF). In some embodiments, aggregation is measured after subjecting the polypeptide or protein (e.g., antibodies and/or other antigen binding proteins) to stress conditions, such as free-thaw, physiological stress (e.g., 37° C.), or thermal stress (e.g., 40° C.). In some embodiments, aggregation is measured following incubation for about five to about fourteen days at about 37° C. In some embodiments, aggregation is measured following incubation for about two to about seven days at about 37° C. In some embodiments, aggregation is measured following incubation for about two to about five days at about 37° C. In some embodiments, aggregation is measured following incubation for about seven to about fourteen days at about 40° C. In some embodiments, aggregation is measured following incubation for about two to about seven days at about 40° C. In some embodiments, aggregation is measured following incubation for about two to about five days at about 40° C. In some embodiments, aggregation is measured following incubation for about seven to about fourteen days at about 4° C. In some embodiments, aggregation is measured following incubation for about two to about seven days at about 4° C. In some embodiments, aggregation is measured following incubation for about two to about five days at about 4° C. In some embodiments, aggregation is measured following incubation for about seven to about fourteen days at about 25° C. In some embodiments, aggregation is measured following incubation for about two to about seven days at about 25° C. In some embodiments, aggregation is measured following incubation for about two to about five days at about 25° C. In specific embodiments, the incubation involves a biological substance (e.g., human serum and/or plasma). Other conventional methods can also be employed as suitable.

### 7.9.8. Immunoassays

Any assay known in the art to assess the immunogenicity of a polypeptide or protein (e.g., antibodies and/or other antigen binding proteins) can be used to detect interaction with a reference. Non-limiting examples of an immunoassay include an electrochemiluminescent (ECL) immunoassay, an ELISA, a radioimmunoassay, and a homogeneous mobility shift assay. Other conventional methods can also be employed as suitable.

### 8. EXAMPLE

### 8.1. Example 1: Stability of Trispecific Antibody with Cap Domain

A tri-specific antibody with the antibody structure depicted in FIG. 9B and a C-terminal cap consisting of the amino acid sequence of SLSLSPGK (SEQ ID NO:36), AS, HHHHHH (SEQ ID NO:26), H, or TVAPTESS (SEQ ID NO:37) maintains the isoelectric point of the antibody as assessed by isoelectric focusing (see, e.g., Isoelectric Focusing Maryvonne D. R. Brasher, Robin Thorpe, in Encyclopedia of Immunology (Second Edition), 1998) and the hydrophobicity of the antibody as assessed by hydrophobic interaction chromatography (see, e.g., Hofstee B H and Otillio N F (1978). Non-ionic adsorption chromatography of proteins. J Chromatogr 159, 57-69. PMID: 649758; see also Jennissen HP (1978) Multivalent interaction chromatography as exemplified by the adsorption and desorption of skeletal muscle enzymes on hydrophobic alkyl-agaroses. J Chromatogr 159, 71-83. PMID: 418077; Jennissen H P and Heilmeyer L M, Jr. (1975). General aspects of hydrophobic chromatography. Adsorption and elution characteristics of some skeletal muscle enzymes. Biochemistry 14, 754-760. PMID: 163642; Porath J et al. (1973). Salting-out in amphiphilic gels as a new approach to hydrophobic adsorption. Nature 245, 465-466. PMID: 4356152), and does not exhibit non-specific binding as assessed by surface plasmon resonance (see, e.g., Methods Mol Biol. Author manuscript; available in PMC 2010 May 5, Methods Mol Biol. 2009; 493:323-343. doi: 10.1007/978-1-59745-523-7_20, PMCID: PMC2864718, NIHMSID: NIHMS187097, PMID: 18839357, Surface Plasmon Resonance (SPR) Analysis of Binding Interactions of Proteins in Inner-Ear Sensory Epithelia Dennis G. Drescher, Neeliyath A. Ramakrishnan, and Marian J. Drescherrelative to the tri-specific antibody without any cap. In addition, a tri-specific antibody with the antibody structure depicted in FIG. 9B and a C-terminal cap consisting of the amino acid sequence of SLSLSPGK (SEQ ID NO:36), AS, HHHHHH (SEQ ID NO:26), or H maintains the conformational stability at 63° C., 70° C., and 74° C. as assessed by differential scanning calorimetry (see, e.g., J Biomol Tech. 2010 December; 21 (4): 167-193, PMCID: PMC2977967, PMID: 21119929, Differential Scanning calorimetry Techniques: Applications in Biology and Nanoscience, Pooria Gill, 1 Tahereh Tohidi Moghadam, 1 and Bijan Ranjbar), and stability in human serum after about 7 days as assessed by size exclusion chromatography (see, e.g., J Liq Chromatogr Relat Technol. 2012 November; 35 (20): 2923-2950. Published online 2012 Nov. 30. doi: 10.1080/10826076.2012.743724. PMCID: PMC3556795, PMID: 23378719, Size-Exclusion Chromatography for the Analysis of Protein Biotherapeutics and their Aggregates, Paula Hong, Stephan Koza, and Edouard S. P. Bouvier.) The C-terminal cap was added at the C-terminus of the scFv region of the tri-specific antibody as depicted in FIG. 9B. This data demonstrates the monohistidine cap does not interfere with the biophysical and chemical properties of the antibody.

### 8.2. Example 2: Binding of Multispecific Antibody to Human Pre-Existing Poly Anti-Drug Antibodies

The ability of a tri-specific antibody depicted in FIG. 9A with or with a C-terminal cap consisting of amino acid sequence SLSLSPGK (SEQ ID NO:36), AS, HHHHHH (SEQ ID NO: 26), H, or TVAPTESS (SEQ ID NO:37) to bind to human pre-existing poly anti-drug antibodies in a serum sample is assessed in an immunoassay (see, e.g., AAPS J. 2016 March; 18 (2): 311-320. Published online 2016 Jan. 28. doi: 10.1208/s12248-016-9878-1, PMCID: PMC4779092, PMID: 26821802, Pre-existing Antibody: Biotherapeutic Modality-Based Review, Boris Gorovits, corresponding author Adrienne Clements-Egan, Mary Birchler, Meina Liang, Heather Myler, Kun Peng, Shobha Purushothama, Manoj Rajadhyaksha, Laura Salazar-Fontana, Crystal Sung, and Li Xue and Recommendations for the Assessment and Management of Pre-existing Drug-Reactive Antibodies During Biotherapeutic Development Li Xue, Adrienne Clements-Egan, Lakshmi Amaravadi, Mary Birchler, Boris Gorovits, Meina Liang, Heather Myler, Shobha Purushothama, Marta Starcevic Manning & Crystal Sung, The AAPS Journal volume 19, pages 1576-1586 (2017)). For example, the C-terminal cap was added at the C-terminus of the scFv region of the tri-specific antibody as depicted in FIG. 9B. Different concentrations of serum from patients with multiple myeloma or not were incubated with the tri-specific antibody of interest with or without one of the cap domains for one to six hours at about 15° C. to 25° C. The amount of poly-anti-drug antibodies present in the serum that bound to the tri-specific antibody was determined using electrochemiluminescence. A reduction in the amount of poly-anti-drug antibodies binding to the tri-specific antibody with each of the cap domains relative to the amount of poly-anti-drug antibodies binding to the tri-specific antibody without a cap domain was observed (see, Table 4). These results indicate that a cap domain consisting of amino acid sequence SLSLSPGK (SEQ ID NO:36), AS, HHHHHH (SEQ ID NO:26), H, or TVAPTESS (SEQ ID NO:37) prevents poly-anti-drug antibodies in patient serum from binding to the tri-specific antibody.

TABLE 4

| | # Low | # Medium | # High | Percen |
|---|---|---|---|---|
| | Level | Level | Level | Pre- |
| | ADA | ADA | ADA | existing |
| | Serum | Serum | Serum | ADA |
| | (ECL < | (ECL | (ECL > | (MMS, |
| Cap | 200) | 201-1000) | 1001) | n = 80) |
| No cap | 33 | 17 | 30 | 58.8 |
| SLSLSPGK | 69 | 11 | 0 | 13.8 |
| AS | 67 | 12 | 1 | 16.3 |
| HHHHHH | 70 | 10 | 0 | 12.5 |
| H | 68 | 11 | 1 | 15 |
| TVAPTESS | 73 | 7 | 0 | 8.8 |

### 9. EMBODIMENTS

This invention provides the following non-limiting embodiments.

In one set of embodiments, provided are:

A1. An isolated polypeptide comprising one or more target binding regions and an exposed C-terminus fused to a cap domain (e.g., a His cap domain, SLSLSPGK (SEQ ID NO: 36), or TVAPTESS (SEQ ID NO:37)),

wherein the isolated polypeptide exhibits reduced interaction with a reference relative to the interaction of the isolated polypeptide lacking the cap domain with the reference.

A2. The isolated polypeptide of embodiment A1, wherein the reference is a molecule present in a biological substance.

A3. The isolated polypeptide of embodiment A2, wherein the biological substance comprises serum and/or plasma

A4. The isolated polypeptide of any one of embodiments A1 to A3, wherein the reference comprises an anti-drug antibody, optionally the anti-drug antibody is pre-existing in the serum and/or plasma of a subject.

A5. The isolated polypeptide of any one of embodiments A1 to A4, wherein the exposed C-terminus comprises an anti-drug antibody epitope.

A6. The isolated polypeptide of any one of embodiments A1 to A5, wherein the one or more target binding regions comprise an antibody variable domain or an antigen-binding fragment thereof.

A7. The isolated polypeptide of any one of embodiments A1 to A5, wherein the one or more target binding regions comprise a light chain antibody variable domain or an antigen-binding fragment thereof.

A8. The isolated polypeptide of any one of embodiments A1 to A5, wherein the one or more target binding regions comprise a heavy antibody variable domain or an antigen-binding fragment thereof.

A9. The isolated polypeptide of any one of embodiments A1 to A8, wherein the cap domain is a His cap domain and the His cap domain consists of a single histidine residue.

A10. The isolated polypeptide of any one of embodiments A1 to A8, wherein the cap domain is a His cap domain and the His cap domain consists of two histidine residues.

A11. The isolated polypeptide of any one of embodiments A1 to A8, wherein the cap domain is a His cap domain and the His cap domain consists of three histidine residues.

A12. The isolated polypeptide of any one of embodiments A1 to A8, wherein the cap domain is a His cap domain and the His cap domain consists of four histidine residues (SEQ ID NO: 27).

A13. The isolated polypeptide of any one of embodiments A1 to A8, wherein the cap domain is a His cap domain and the His cap domain consists of five histidine residues (SEQ ID NO: 28).

A14. The isolated polypeptide of any one of embodiments A1 to A6, wherein the cap domain is a His cap domain and the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), VTVSSHHHHH (SEQ ID NO:5), TKVTVL (His)n (SEQ ID NO:46), TKLTVL (His)n (SEQ ID NO: 47), TQLIIL (His)n (SEQ ID NO:48), TELTVL (His)n (SEQ ID NO:49), and TQLTVL (His)n (SEQ ID NO:50), wherein n is 1, 2, 3, 4, or 5.

A15. The isolated polypeptide of any one of embodiments A1 to A6, wherein the cap domain is a His cap domain and the exposed C-terminus fused to the His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO:9), and VEIKHHHHH (SEQ ID NO:10).

A16. The isolated polypeptide of any one of embodiments A1 to A6, wherein the cap domain is a His cap domain and the exposed C-terminus fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15).

A17. The isolated polypeptide of any one of embodiments A1 to A6, wherein the cap domain is a His cap domain and the exposed C-terminus fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO: 19), and VEIKRTHHHHH (SEQ ID NO:20).

A18. The isolated polypeptide of any one of embodiments A1 to A17, wherein interaction with the reference is determined by an immunoassay.

A19. The isolated polypeptide of any one of embodiments A1 to A17, wherein the interaction with the reference is assessed in vitro.

A20. The isolated polypeptide of any one of embodiments A1 to A17, wherein the interaction with the reference is assessed in vivo.

A21. An isolated polypeptide comprising one or more target binding regions and an exposed C-terminus fused to a cap domain (e.g., a His cap domain, SLSLSPGK (SEQ ID NO: 36), or TVAPTESS (SEQ ID NO:37)),

wherein the isolated polypeptide exhibits reduced aggregation upon incubation with a biological substance relative to the aggregation exhibited upon incubation of the isolated polypeptide lacking the cap domain with the biological substance.

A22. The isolated polypeptide of embodiment A21, wherein the biological substance is human serum and/or human plasma.

A23. The isolated polypeptide of embodiment A22, wherein incubation with the biological substance comprises incubation for about two to about five days, about two to about seven days, or about five to about fourteen days at about 37° C.

A24. The isolated polypeptide of embodiment A22, wherein incubation with the biological substance comprises incubation for about two to about five days, about two to about seven days, or about seven to about fourteen days at about 40° C.

A25. The isolated polypeptide of embodiment A22, wherein incubation with the biological substance comprises incubation for about two to about five days, about two to about seven days, or about seven to about fourteen days at about 4° C.

A26. The isolated polypeptide of embodiment A22, wherein incubation with the biological substance comprises incubation for about two to about five days, about two to about seven days, or about seven to about fourteen days at about 25° C.

A27. The isolated polypeptide of any one of embodiments A21 to A26, wherein the biological substance comprises an anti-drug antibody, optionally the anti-drug antibody is pre-existing.

A28. The isolated polypeptide of any one of embodiments A21 to A27, wherein the exposed C-terminus comprises an anti-drug antibody epitope.

A29. The isolated polypeptide of any one of embodiments A21 to A28, wherein the one or more target binding regions comprise an antibody variable domain or an antigen-binding fragment thereof.

A30. The isolated polypeptide of any one of embodiments A21 to A28, wherein the one or more target binding regions comprise a light chain antibody variable domain or an antigen-binding fragment thereof.

A31. The isolated polypeptide of any one of embodiments A21 to A28, wherein the one or more target binding regions comprise a heavy antibody variable domain or an antigen-binding fragment thereof.

A32. The isolated polypeptide of any one of embodiments A21 to A31, wherein the cap domain is a His cap domain and the His cap domain consists of a single histidine residue.

A33. The isolated polypeptide of any one of embodiments A21 to A31, wherein the cap domain is a His cap domain and the His cap domain consists of two histidine residues.

A34. The isolated polypeptide of any one of embodiments A21 to A31, wherein the cap domain is a His cap domain and the His cap domain consists of three histidine residues.

A35. The isolated polypeptide of any one of embodiments A21 to A31, wherein the cap domain is a His cap domain and the His cap domain consists of four histidine residues (SEQ ID NO: 27).

A36. The isolated polypeptide of any one of embodiments A21 to A31, wherein the cap domain is a His cap domain and the His cap domain consists of five histidine residues (SEQ ID NO: 28).

A37. The isolated polypeptide of any one of embodiments A21 to A29, wherein the cap domain is a His cap domain and the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), VTVSSHHHHH (SEQ ID NO:5), TKVTVL (His)n (SEQ ID NO:46), TKLTVL (His)n (SEQ ID NO: 47), TQLIIL (His)n (SEQ ID NO:48), TELTVL (His)n (SEQ ID NO:49), and TQLTVL (His)n (SEQ ID NO:50), wherein n is 1, 2, 3, 4, or 5.

A38. The isolated polypeptide of any one of embodiments A21 to A29, wherein the cap domain is a His cap domain and the exposed C-terminus fused to the His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO:9), and VEIKHHHHH (SEQ ID NO:10).

A39. The isolated polypeptide of any one of embodiments A21 to A29, wherein the cap domain is a His cap domain and the exposed C-terminus fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15).

A40. The isolated polypeptide of any one of embodiments A21 to A29, wherein the cap domain is a His cap domain and the exposed C-terminus fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO: 19), and VEIKRTHHHHH (SEQ ID NO:20).

A41. The isolated polypeptide of any one of embodiments A21 to A40, wherein the interaction with the biological substance is assessed by size exclusion chromatography.

A42. An isolated polypeptide comprising one or more target binding regions and an exposed C-terminus fused to a cap domain (e.g., a His cap domain, SLSLSPGK (SEQ ID NO: 36), or TVAPTESS (SEQ ID NO:37,

wherein the isolated polypeptide exhibits reduced self-aggregation relative to the self-aggregation exhibited by the isolated polypeptide lacking the cap domain under the same conditions.

A43. The isolated polypeptide of embodiment A42, wherein the exposed C-terminus comprises an anti-drug antibody epitope.

A44. The isolated polypeptide of embodiment A42 or A43, wherein the one or more target binding regions comprise an antibody variable domain or an antigen-binding fragment thereof.

A45. The isolated polypeptide of embodiment A42 or A43, wherein the one or more target binding regions comprise a light chain antibody variable domain or an antigen-binding fragment thereof.

A46. The isolated polypeptide of embodiment A42 or A43, wherein the one or more target binding regions comprise a heavy antibody variable domain or an antigen-binding fragment thereof.

A47. The isolated polypeptide of any one of embodiments A42 to A46, wherein the cap domain is a His cap domain and the His cap domain consists of a single histidine residue.

A48. The isolated polypeptide of any one of embodiments A42 to A46, wherein the cap domain is a His cap domain and the His cap domain consists of two histidine residues.

A49. The isolated polypeptide of any one of embodiments A42 to A46, wherein the cap domain is a His cap domain and the His cap domain consists of three histidine residues.

A50. The isolated polypeptide of any one of embodiments A42 to A46, wherein the cap domain is a His cap domain and the His cap domain consists of four histidine residues (SEQ ID NO: 27).

A51. The isolated polypeptide of any one of embodiments A42 to A46, wherein the cap domain is a His cap domain and the His cap domain consists of five histidine residues (SEQ ID NO: 28).

A52. The isolated polypeptide of any one of embodiments A42 to A44, wherein the cap domain is a His cap domain and the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), VTVSSHHHHH (SEQ ID NO:5), TKVTVL (His)n (SEQ ID NO:46), TKLTVL (His)n (SEQ ID NO: 47), TQLIIL (His)n (SEQ ID NO:48), TELTVL (His)n (SEQ ID NO:49), and TQLTVL (His)n (SEQ ID NO:50), wherein n is 1, 2, 3, 4, or 5.

A53. The isolated polypeptide of any one of embodiments A42 to A44, wherein the cap domain is a His cap domain and the exposed C-terminus fused to the His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO:9), and VEIKHHHHH (SEQ ID NO:10).

A54. The isolated polypeptide of any one of embodiments A42 to A44, wherein the cap domain is a His cap domain and the exposed C-terminus fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15).

A55. The isolated polypeptide of any one of embodiments A42 to A44, wherein the cap domain is a His cap domain and the exposed C-terminus fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO: 19), and VEIKRTHHHHH (SEQ ID NO:20).

A56. The isolated polypeptide of any one of embodiments A42 to A55, wherein self-aggregation is reduced in a high concentration liquid formulation (HCLF).

A57. The isolated polypeptide of embodiment A56, wherein the HCLF concentration comprises about 50 mg/mL to about 150 mg/mL of the isolated polypeptide.

A58. The isolated polypeptide of any one of embodiments A42 to A57, wherein self-aggregation is reduced under thermal stress.

A59. The isolated polypeptide of embodiment A58, wherein thermal stress comprises incubation at 40° C. for about two weeks.

A60. The isolated polypeptide of any one of embodiments A42 to A59, wherein the self-aggregation is assessed by size exclusion chromatography.

A61. An isolated protein, comprising:
(a) a first polypeptide comprising a target binding region, and an exposed C-terminus fused to a cap domain (e.g., a His cap domain, SLSLSPGK (SEQ ID NO:36), or TVAPTESS (SEQ ID NO:37)); and
(b) a second polypeptide comprising a target binding region,
wherein the isolated protein exhibits reduced interaction with a reference relative to the interaction of the isolated protein lacking the cap domain with the reference.

A62. The isolated protein of embodiment A61, wherein the exposed C-terminus of the first polypeptide comprises an anti-drug antibody epitope.

A63. The isolated protein of embodiment A61 or A62, wherein the second polypeptide comprises an exposed C-terminus fused to a cap domain (e.g., a His cap domain, SLSLSPGK (SEQ ID NO:36), or TVAPTESS (SEQ ID NO:37)).

A64. The isolated protein of embodiment A63, wherein the exposed C-terminus of the second polypeptide comprises an anti-drug antibody epitope.

A65. The isolated protein of embodiment A61 to A64, wherein the target binding region of the first and second polypeptides are capable of binding to different targets.

A66. The isolated protein of any one of embodiments A61 to A65, wherein the target binding region of the first polypeptide comprises an antibody variable domain or an antigen-binding fragment thereof.

A67. The isolated protein of any one of embodiments A64 to A66, wherein the target binding region of the second polypeptide comprises an antibody variable domain or an antigen-binding fragment thereof.

A68. The isolated protein of any one of embodiments A61 to A65, wherein the target binding region of the first polypeptide comprises a light chain antibody variable domain or an antigen-binding fragment thereof.

A69. The isolated protein of embodiment A64, A65, or A68, wherein the target binding region of the second polypeptide comprises a light chain antibody variable domain or an antigen-binding fragment thereof.

A70. The isolated protein of any one of embodiments A61 to A65, wherein the target binding region of the first polypeptide comprises a heavy chain antibody variable domain or an antigen-binding fragment thereof.

A71. The isolated protein of embodiment A64, A65, or A70, wherein the target binding region of the second polypeptide comprises a heavy chain antibody variable domain or an antigen-binding fragment thereof.

A72. The isolated protein of any one of embodiments A61 to A71, wherein the cap domain of the first polypeptide is a His cap domain and the His cap domain consists of a single histidine residue.

A73. The isolated protein of embodiment A64, A65, A67, A69, A71, or A72, wherein the cap domain of the second polypeptide is a His cap domain and the His cap domain consists of a single histidine residue.

A74. The isolated protein of any one of embodiments A61 to A71, wherein the cap domain of the first polypeptide is a His cap domain and the His cap domain consists of two histidine residues.

A75. The isolated protein of embodiment A64, A65, A67, A69, A71, A72, or A74, wherein the cap domain of the second polypeptide is a His cap domain and the His cap domain consists of two histidine residues.

A76. The isolated protein of any one of embodiments A61 to A71, wherein the cap domain of the first polypeptide is a His cap domain and the His cap domain consists of three histidine residues.

A77. The isolated protein of embodiment A64, A65, A67, A69, A71, A72, A74, or A76, wherein the cap domain of the second polypeptide is a His cap domain and the His cap domain consists of three histidine residues.

A78. The isolated protein of any one of embodiments A61 to A71, wherein the cap domain of the first polypeptide is a His cap domain and the His cap domain consists of four histidine residues (SEQ ID NO:27).

A79. The isolated protein of embodiment A64, A65, A67, A69, A71, A72, A74, A76, or A78, wherein the cap domain of the second polypeptide is a His cap domain and the His cap domain consists of four histidine residues (SEQ ID NO:27).

A80. The isolated protein of any one of embodiments A61 to A71, wherein the cap domain of the first polypeptide is a His cap domain and the His cap domain consists of five histidine residues (SEQ ID NO:28).

A81. The isolated protein of embodiment A64, A65, A67, A69, A71, A72, A74, A76, A78, or A80, wherein the cap domain of the second polypeptide is a His cap domain and the His cap domain consists of five histidine residues (SEQ ID NO:28).

A82. The isolated protein of any one of embodiments A61 to A67, wherein the cap domain is a His cap domain and the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), VTVSSHHHHH (SEQ ID NO:5), TKVTVL (His)n (SEQ ID NO:46), TKLTVL (His)n (SEQ ID NO: 47), TQLIIL (His)n (SEQ ID NO:48), TELTVL (His)n (SEQ ID NO:49), and TQLTVL (His)n (SEQ ID NO:50), wherein n is 1, 2, 3, 4, or 5.

A83. The isolated protein of any one of embodiments A61 to A67, wherein the cap domain is a His cap domain and the exposed C-terminus fused to the His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO:9), and VEIKHHHHH (SEQ ID NO:10).

A84. The isolated protein of any one of embodiments A61 to A67, wherein the cap domain is a His cap domain and the exposed C-terminus fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15).

A85. The isolated protein of any one of embodiments A61 to A67, wherein the cap domain is a His cap domain and the exposed C-terminus fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO: 19), and VEIKRTHHHHH (SEQ ID NO:20).

A86. The isolated protein of any one of embodiment A61 to A85, wherein the isolated protein further comprises a third polypeptide comprising a target binding region.

A87. The isolated protein of embodiment A86, wherein the third polypeptide comprises an exposed C-terminus fused to a cap domain (e.g., a His cap domain, SLSLSPGK (SEQ ID NO:36), or TVAPTESS (SEQ ID NO:37)).

A88. The isolated protein of embodiment A86 or A87, wherein the third polypeptide is capable of binding to a different target than the first polypeptide, the second polypeptide, or both.

A89. The isolated protein of any one of embodiments A86 to A88, wherein the exposed C-terminus of the third polypeptide comprises an anti-drug antibody epitope.

A90. The isolated protein of any one of embodiments A86 to A89, wherein the target binding region of the third polypeptide comprises an antibody variable domain or an antigen-binding fragment thereof.

A91. The isolated protein of any one of embodiments A86 to A89, wherein the target binding region of the third polypeptide comprises a light chain antibody variable domain or an antigen-binding fragment thereof.

A92. The isolated protein of any one of embodiments A86 to A89, wherein the target binding region of the third polypeptide comprises a heavy chain antibody variable domain or an antigen-binding fragment thereof.

A93. The isolated protein of any one of embodiments A87 to A92, wherein the cap domain is a His cap domain and the His cap domain of the third polypeptide consists of a single histidine residue.

A94. The isolated protein of any one of embodiments A87 to A92, wherein the cap domain is a His cap domain and the His cap domain of the third polypeptide consists of two histidine residues.

A95. The isolated protein of any one of embodiments A87 to A92, wherein the cap domain is a His cap domain and the His cap domain of the third polypeptide consists of three histidine residues.

A96. The isolated protein of any one of embodiments A87 to A92, wherein the cap domain is a His cap domain and the His cap domain of the third polypeptide consists of four histidine residues (SEQ ID NO:27).

A97. The isolated protein of any one of embodiments A87 to A92, wherein the cap domain is a His cap domain and the His cap domain of the third polypeptide consists of five histidine residues (SEQ ID NO:28).

A98. The isolated protein of any one of embodiments A87 to A89, wherein the cap domain is a His cap domain and the exposed C-terminus of the third polypeptide fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), VTVSSHHHHH (SEQ ID NO:5), TKVTVL (His)n (SEQ ID NO:46), TKLTVL (His)n (SEQ ID NO:47), TQLIIL (His)n (SEQ ID NO:48), TELTVL (His)n (SEQ ID NO: 49), and TQLTVL (His)n (SEQ ID NO:50), wherein n is 1, 2, 3, 4, or 5.

A99. The isolated protein of any one of embodiments A87 to A89, wherein the cap domain is a His cap domain and the exposed C-terminus of the third polypeptide fused to the His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO: 9), and VEIKHHHHH (SEQ ID NO:10).

A100. The isolated protein of any one of embodiments A87 to A89, wherein the cap domain is a His cap domain and the exposed C-terminus of the third polypeptide fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15).

A101. The isolated protein of any one of embodiments A87 to A89, wherein the cap domain is a His cap domain and the exposed C-terminus of the third polypeptide fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO:20).

A102. The isolated protein of any one of embodiments A87 to A85, wherein the isolated protein is bispecific.

A103. The isolated protein of any one of embodiments A61 to A101, wherein the isolated protein is multi-specific.

A104. The isolated protein of any one of embodiments A61 to A101, wherein the isolated protein is trispecific.

A105. The isolated protein of any one of embodiments A61 to A104, wherein the isolated protein is heterodimeric.

A106. An isolated nucleic acid sequence comprising a nucleotide sequence encoding the polypeptide of any one of embodiments A1 to A60, or the protein of any one of embodiments A61 to A105.

A107. An isolated cell expressing the polypeptide of any one of embodiments A1 to A60, or the protein of any one of embodiments A61 to A105.

A108. An expression vector comprising the nucleic acid sequence of embodiment A106.

A109. An isolated cell comprising the nucleic acid sequence of embodiment A106, or the vector of embodiment A108.

A110. A method for producing the polypeptide of any one of embodiments A1 to A60, or the protein of any one of embodiments A61 to A105, comprising culturing the cell of embodiment A107 or A109.

A111. The method of embodiment A110, further comprising isolating the polypeptide or protein.

A112. A bispecific or a multi-specific antibody comprising one or more target binding regions and an exposed C-terminus fused to a cap domain (e.g., a His cap domain, SLSLSPGK (SEQ ID NO:36), or TVAPTESS (SEQ ID NO:37)), wherein the antibody exhibits reduced interaction with an anti-drug antibody relative to the interaction of the antibody lacking the cap domain with the anti-drug antibody.

A113. The antibody of embodiment A112, wherein the exposed C-terminus comprises an anti-drug antibody epitope.

A114. The antibody of embodiment A112 or A113, wherein the antibody exhibits reduced interaction with an anti-drug antibody relative to the interaction of the antibody lacking the cap domain (e.g., a His cap domain, SLSLSPGK (SEQ ID NO:36), or TVAPTESS (SEQ ID NO: 37)) with the anti-drug antibody in an immunoassay.

A115. The antibody of any one of embodiments A112 to A114, wherein the one or more target binding regions comprises at least one antibody variable domain.

A116. The antibody of embodiment A115, wherein the at least one antibody variable domain comprises a heavy chain variable domain.

A117. The antibody of embodiment A115, wherein the at least one antibody variable domain comprises a light chain variable domain.

A118. The antibody of any one of embodiments A112 to A117, wherein the cap domain is a His cap domain and the His cap domain consists of a single histidine residue.

A119. The antibody of any one of embodiments A112 to A117, wherein the cap domain is a His cap domain and the His cap domain consists of two histidine residues.

A120. The antibody of any one of embodiments A112 to A117, wherein the cap domain is a His cap domain and the His cap domain consists of three histidine residues.

A121. The antibody of any one of embodiments A112 to A117, wherein the cap domain is a His cap domain and the His cap domain consists of four histidine residues (SEQ ID NO: 27).

A122. The antibody of any one of embodiments A112 to A117, wherein the cap domain is a His cap domain and the His cap domain consists of five histidine residues (SEQ ID NO: 28).

A123. The antibody of any one of embodiments A112 to A115, wherein the cap domain is a His cap domain and the exposed C-terminus fused to a His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), VTVSSHHHHH (SEQ ID NO:5), TKVTVL (His)n (SEQ ID NO:46), TKLTVL (His)n (SEQ ID NO: 47), TQLIIL (His)n (SEQ ID NO:48), TELTVL (His)n (SEQ ID NO:49), and TQLTVL (His)n (SEQ ID NO:50), wherein n is 1, 2, 3, 4, or 5.

A124. The antibody of any one of embodiments A112 to A115, wherein the cap domain is a His cap domain and the exposed C-terminus fused to a His cap domain consists of the amino acid sequence selected from the group consisting of: VEIKH (SEQ ID NO:6), VEIKHH (SEQ ID NO:7), VEIKHHH (SEQ ID NO:8), VEIKHHHH (SEQ ID NO:9), and VEIKHHHHH (SEQ ID NO:10).

A125. The antibody of any one of embodiments A112 to A115, wherein the cap domain is a His cap domain and the exposed C-terminus fused to a His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15).

A126. The antibody of any one of embodiments A112 to A115, wherein the cap domain is a His cap domain and the exposed C-terminus fused to a His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO: 19), and VEIKRTHHHHH (SEQ ID NO:20).

A127. The antibody of any one of embodiments A112 to A126, wherein the multi-specific antibody comprises a trispecific antibody.

A128. A nucleic acid sequence comprising a nucleotide sequence encoding the antibody of any one of embodiments A112 to A127.

A129. An expression vector comprising the nucleic acid sequence of embodiment A128.

A130. An isolated cell comprising the nucleic acid sequence of embodiment A128, or the vector of embodiment A129.

A131. An isolated cell expressing the antibody of any one embodiments A112 to A127.

A132. A method for producing the antibody of any one of embodiments A112 to A127, comprising culturing the cell of embodiment A130 or A131.

A133. The method of embodiment A132, further comprising isolating the antibody.

In another set of embodiments, provided are:

B1. A method for reducing the interaction between an isolated polypeptide and a reference, wherein the isolated polypeptide comprises one or more target binding regions and an exposed C-terminus, the method comprising fusing a cap domain (e.g., a His cap domain, SLSLSPGK (SEQ ID NO:36), or TVAPTESS (SEQ ID NO:37)) at the end of the exposed C-terminus.

B2. The method of embodiment B1, wherein the reference is a molecule present in a biological substance.

B3. The method of embodiment B2, wherein the biological substance comprises serum and/or plasma.

B4. The method of any one of embodiments B1 to B3, wherein the reference comprises an anti-drug antibody, optionally the anti-drug antibody is pre-existing in serum and/or plasma of a subject.

B5. The method of any one of embodiments B1 to B4, wherein the exposed C-terminus comprises an anti-drug antibody epitope.

B6. The method of any one of embodiments B1 to B5, wherein the one or more target binding regions comprise an antibody variable domain or an antigen-binding fragment thereof.

B7. The method of any one of embodiments B1 to B5, wherein the one or more target binding regions comprise a light chain antibody variable domain or an antigen-binding fragment thereof.

B8. The method of any one of embodiments B1 to B5, wherein the one or more target binding regions comprise a heavy antibody variable domain or an antigen-binding fragment thereof.

B9. The method of any one of embodiments B1 to B8, wherein the cap domain is a His cap domain and the His cap domain consists of a single histidine residue.

B10. The method of any one of embodiments B1 to B8, wherein the cap domain is a His cap domain and the His cap domain consists of two histidine residues.

B11. The method of any one of embodiments B1 to B8, wherein the cap domain is a His cap domain and the His cap domain consists of three histidine residues.

B12. The method of any one of embodiments B1 to B8, wherein the cap domain is a His cap domain and the His cap domain consists of four histidine residues (SEQ ID NO:27).

B13. The method of any one of embodiments B1 to B8, wherein the cap domain is a His cap domain and the His cap domain consists of five histidine residues (SEQ ID NO:28).

B14. The method of any one of embodiments B1 to B6, wherein the cap domain is a His cap domain and the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), VTVSSHHHHH (SEQ ID NO:5), TKVTVL (His)n (SEQ ID NO:46), TKLTVL (His)n (SEQ ID NO: 47), TQLIIL (His)n (SEQ ID NO:48), TELTVL (His)n (SEQ ID NO:49), and TQLTVL (His)n (SEQ ID NO:50), wherein n is 1, 2, 3, 4, or 5.

B15. The method of any one of embodiments B1 to B6, wherein the cap domain is a His cap domain and the exposed C-terminus fused to the His cap domain consists of the amino acid sequence selected from the group consisting of: VEIKH (SEQ ID NO:6), VEIKHH (SEQ ID NO: 7), VEIKHHH (SEQ ID NO:8), VEIKHHHH (SEQ ID NO:9), and VEIKHHHHH (SEQ ID NO: 10).

B16. The method of any one of embodiments B1 to B6, wherein the cap domain is a His cap domain and the exposed C-terminus fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15).

B17. The method of any one of embodiments B1 to B6, wherein the cap domain is a His cap domain and the exposed C-terminus fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO:20).

B18. The method of any one of embodiments B1 to B17, wherein the interaction with the reference is determined by an immunoassay.

B19. The method of any one of embodiments B1 to B17, wherein the interaction with the reference is assessed in vitro.

B20. The method of any one of embodiments B1 to B17, wherein the interaction with the reference is assessed in vivo.

B21. A method for reducing the aggregation of an isolated polypeptide in a biological substance, wherein the isolated polypeptide comprises one or more target binding regions and an exposed C-terminus, the method comprising fusing a cap domain (e.g., a His cap domain, SLSLSPGK (SEQ ID NO:36), or TVAPTESS (SEQ ID NO:37)) at the end of the exposed C-terminus.

B22. The method of embodiment B21, wherein the biological substance is human serum.

B23. The method of embodiment B22, wherein incubation with the biological substance comprises incubation for about two to about five days, about two to about seven days, or about five to about fourteen days at about 37° C.

B24. The method of embodiment B22, wherein incubation with the biological substance comprises incubation for about two to about five days, about two to about seven days, or about seven to about fourteen days at about 40° C.

B25. The method of embodiment B22, wherein incubation with the biological substance comprises incubation for about two to about five days, about two to about seven days, or about seven to about fourteen days at about 4° C.

B26. The method of embodiment B22, wherein incubation with the biological substance comprises incubation for about two to about five days, about two to about seven days, or about seven to about fourteen days at about 25° C.

B27. The method of any one of embodiments B21 to B26, wherein the biological substance comprises an anti-drug antibody, optionally the anti-drug antibody is pre-existing in serum and/or plasma of a subject.

B28. The method of any one of embodiments B21 to B27, wherein the exposed C-terminus comprises an anti-drug antibody epitope.

B29. The method of any one of embodiments B21 to B28, wherein the one or more target binding regions comprise an antibody variable domain or an antigen-binding fragment thereof.

B30. The method of any one of embodiments B21 to B28, wherein the one or more target binding regions comprise a light chain antibody variable domain or an antigen-binding fragment thereof.

B31. The method of any one of embodiments B21 to B28, wherein the one or more target binding regions comprise a heavy antibody variable domain or an antigen-binding fragment thereof.

B32. The method of any one of embodiments B21 to B31, wherein the cap domain is a His cap domain and the His cap domain consists of a single histidine residue.

B33. The method of any one of embodiments B21 to B31, wherein the cap domain is a His cap domain and the His cap domain consists of two histidine residues.

B34. The method of any one of embodiments B21 to B31, wherein the cap domain is a His cap domain and the His cap domain consists of three histidine residues.

B35. The method of any one of embodiments B21 to B31, wherein the cap domain is a His cap domain and the His cap domain consists of four histidine residues (SEQ ID NO:27).

B36. The method of any one of embodiments B21 to B31, wherein the cap domain is a His cap domain and the His cap domain consists of five histidine residues (SEQ ID NO:28).

B37. The method of any one of embodiments B21 to B29, wherein the cap domain is a His cap domain and the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), VTVSSHHHHH (SEQ ID NO:5), TKVTVL(His)n (SEQ ID NO:46), TKLTVL(His)n (SEQ ID NO: 47), TQLIIL(His)n (SEQ ID NO:48), TELTVL(His)n (SEQ ID NO:49), and TQLTVL(His)n (SEQ ID NO:50), wherein n is 1, 2, 3, 4, or 5.

B38. The method of any one of embodiments B21 to B29, wherein the cap domain is a His cap domain and the exposed C-terminus fused to the His cap domain consists of the amino acid sequence selected from the group consisting of: VEIKH (SEQ ID NO:6), VEIKHH (SEQ ID NO: 7), VEIKHHH (SEQ ID NO:8), VEIKHHHH (SEQ ID NO:9), and VEIKHHHHH (SEQ ID NO: 10).

B39. The method of any one of embodiments B21 to B29, wherein the cap domain is a His cap domain and the exposed C-terminus fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15).

B40. The method of any one of embodiments B21 to B29, wherein the cap domain is a His cap domain and the exposed C-terminus fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO:20).

B41. The method of any one of embodiments B21 to B40, wherein the interaction with the biological substance is assessed by size exclusion chromatography.

B42. A method for reducing self-aggregation of an isolated polypeptide, wherein the isolated polypeptide comprises one or more target binding regions and an exposed C-terminus, the method comprising fusing a cap domain (e.g., a His cap domain, SLSLSPGK (SEQ ID NO: 36), or TVAPTESS (SEQ ID NO:37)) at the end of the exposed C-terminus.

B43. The method of embodiment B42, wherein the exposed C-terminus comprises an anti-drug antibody epitope.

B44. The method of embodiment B42 or B43, wherein the one or more target binding regions comprise an antibody variable domain or an antigen-binding fragment thereof.

B45. The method of embodiment B42 or B43, wherein the one or more target binding regions comprise a light chain antibody variable domain or an antigen-binding fragment thereof.

B46. The method of embodiment B42 or B43, wherein the one or more target binding regions comprise a heavy antibody variable domain or an antigen-binding fragment thereof.

B47. The method of any one of embodiments B42 to B46, wherein the cap domain is a His cap domain and the His cap domain consists of a single histidine residue.

B48. The method of any one of embodiments B42 to B46, wherein the cap domain is a His cap domain and the His cap domain consists of two histidine residues.

B49. The method of any one of embodiments B42 to B46, wherein the cap domain is a His cap domain and the His cap domain consists of three histidine residues.

B50. The method of any one of embodiments B42 to B46, wherein the cap domain is a His cap domain and the His cap domain consists of four histidine residues (SEQ ID NO:27).

B51. The method of any one of embodiments B42 to B46, wherein the cap domain is a His cap domain and the His cap domain consists of five histidine residues (SEQ ID NO:28).

B52. The method of any one of embodiments B42 to B44, wherein the cap domain is a His cap domain and the exposed C-terminus fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), VTVSSHHHHH (SEQ ID NO:5), TKVTVL (His)n (SEQ ID NO:46), TKLTVL (His)n (SEQ ID NO: 47), TQLIIL (His)n (SEQ ID NO:48), TELTVL (His)n (SEQ ID NO:49), and TQLTVL (His)n (SEQ ID NO:50), wherein n is 1, 2, 3, 4, or 5.

B53. The method of any one of embodiments B42 to B44, wherein the cap domain is a His cap domain and the exposed C-terminus fused to the His cap domain consists of the amino acid sequence selected from the group consisting of: VEIKH (SEQ ID NO:6), VEIKHH (SEQ ID NO: 7), VEIKHHH (SEQ ID NO:8), VEIKHHHH (SEQ ID NO:9), and VEIKHHHHH (SEQ ID NO: 10).

B54. The method of any one of embodiments B42 to B44, wherein the cap domain is a His cap domain and the exposed C-terminus fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15).

B55. The method of any one of embodiments B42 to B44, wherein the cap domain is a His cap domain and the exposed C-terminus fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO:20).

B56. The method of any one of embodiments B42 to B55, wherein self-aggregation is reduced in a high concentration liquid formulation (HCLF).

B57. The method of embodiment B56, wherein the HCLF concentration comprises about 50 mg/mL to about 150 mg/mL of the isolated polypeptide.

B58. The method of any one of embodiments B42 to B57, wherein self-aggregation is reduced under thermal stress.

B59. The method of embodiment B58, wherein thermal stress comprises incubation at 40° C. for about two weeks.

B60. The method of any one of embodiments B42 to B59, wherein the self-aggregation is assessed by size exclusion chromatography.

B61. A method for reducing the interaction between an isolated protein and a reference, wherein the isolated protein comprises:
(a) a first polypeptide comprising a target binding region, and an exposed C-terminus; and
(b) a second polypeptide comprising a target binding region,
the method comprising fusing a cap domain (e.g., a His cap domain, SLSLSPGK (SEQ ID NO: 36), or TVAPTESS (SEQ ID NO:37)) at the end of the exposed C-terminus.

B62. The method of embodiment B61, wherein the exposed C-terminus of the first polypeptide comprises an anti-drug antibody epitope.

B63. The method of embodiment B61 or B62, wherein the exposed C-terminus of the second polypeptide comprises an anti-drug antibody epitope.

B64. The method of any one of embodiments B61 to B63, wherein the second polypeptide comprises an exposed C-terminus and the method further comprises fusing a cap domain (e.g., a His cap domain, SLSLSPGK (SEQ ID NO:36), or TVAPTESS (SEQ ID NO:37)) to the end of the exposed C-terminus of the second polypeptide.

B65. The method of embodiment B61 to B64, wherein the target binding region of the first and second polypeptides are capable of binding to different targets.

B66. The method of any one of embodiments B61 to B65, wherein the target binding region of the first polypeptide comprises an antibody variable domain or an antigen-binding fragment thereof.

B67. The method of any one of embodiments B64 to B66, wherein the target binding region of the second polypeptide comprises an antibody variable domain or an antigen-binding fragment thereof.

B68. The method of any one of embodiments B61 to B65, wherein the target binding region of the first polypeptide comprises a light chain antibody variable domain or an antigen-binding fragment thereof.

B69. The method of embodiment B64, B65, or B68, wherein the target binding region of the second polypeptide comprises a light chain antibody variable domain or an antigen-binding fragment thereof.

B70. The method of any one of embodiments B61 to B65, wherein the target binding region of the first polypeptide comprises a heavy chain antibody variable domain or an antigen-binding fragment thereof.

B71. The method of embodiment B64, B65, or B70, wherein the target binding region of the second polypeptide comprises a heavy chain antibody variable domain or an antigen-binding fragment thereof.

B72. The method of any one of embodiments B61 to B71, wherein the cap domain of the first polypeptide is a His cap domain and the His cap domain consists of a single histidine residue.

B73. The method of embodiment B64, B65, B67, B69, B71, or B72, wherein the cap domain of the second polypeptide is a His cap domain and the His cap domain consists of a single histidine residue.

B74. The method of any one of embodiments B61 to B71, wherein the cap domain of the first polypeptide is a His cap domain and the His cap domain consists of two histidine residues.

B75. The method of embodiment B64, B65, B67, B69, B71, B72, or B74, wherein the cap domain of the second polypeptide is a His cap domain and the His cap domain consists of two histidine residues.

B76. The method of any one of embodiments B61 to B71, wherein the cap domain of the first polypeptide is a His cap domain and the His cap domain consists of three histidine residues.

B77. The method of embodiment B64, B65, B67, B69, B71, B72, B74, or B76, wherein the cap domain of the second polypeptide is a His cap domain and the His cap domain consists of three histidine residues.

B78. The method of any one of embodiments B61 to B71, wherein the cap domain of the first polypeptide is a His cap domain and the His cap domain consists of four histidine residues (SEQ ID NO:27).

B79. The method of embodiment B64, B65, B67, B69, B71, B72, B74, B76, or B78, wherein the cap domain of the second polypeptide is a His cap domain and the His cap domain consists of four histidine residues (SEQ ID NO:27).

B80. The method of any one of embodiments B61 to B71, wherein the cap domain is a His cap domain of the first polypeptide and the His cap domain consists of five histidine residues (SEQ ID NO:28).

B81. The method of embodiment B64, B65, B67, B69, B71, B72, B74, B76, B78, or B80, wherein the cap domain of the second polypeptide is a His cap domain and the His cap domain consists of five histidine residues (SEQ ID NO:28).

B82. The method of any one of embodiments B61 to B67, wherein the cap domain of the first polypeptide is a His cap domain and the exposed C-terminus fused to the His cap domain consists of the amino acid sequence selected from the group consisting of: VTVSSH (SEQ ID NO: 1), VTVSSHH (SEQ ID NO:2), VTVSSHHH (SEQ ID NO:3), VTVSSHHHH (SEQ ID NO: 4), VTVSSHHHHH (SEQ ID NO:5), TKVTVL (His)n (SEQ ID NO:46), TKLTVL (His)n (SEQ ID NO:47), TQLIIL (His)n (SEQ ID NO:48), TELTVL (His)n (SEQ ID NO:49), or TQLTVL (His)n (SEQ ID NO:50), wherein n is 1, 2, 3, 4, or 5.

B83. The method of any one of embodiments B61 to B67, wherein the cap domain of the first polypeptide is a His cap domain and the exposed C-terminus fused to the His cap domain consists of the amino acid sequence selected from the group consisting of: VEIKH (SEQ ID NO: 6), VEIKHH (SEQ ID NO:7), VEIKHHH (SEQ ID NO:8), VEIKHHHH (SEQ ID NO:9), and VEIKHHHHH (SEQ ID NO:10).

B84. The method of any one of embodiments B61 to B67, the cap domain of the first polypeptide is a His cap domain and the exposed C-terminus fused to the His cap domain consists of the amino acid sequence selected from the group consisting of: VEIKRH (SEQ ID NO: 11), VEIKRHH (SEQ ID NO:12), VEIKRHHH (SEQ ID NO:13), VEIKRHHHH (SEQ ID NO: 14), and VEIKRHHHHH (SEQ ID NO:15).

B85. The method of any one of embodiments B61 to B67, wherein the cap domain of the first polypeptide is a His cap domain and the exposed C-terminus fused to the His cap domain consists of the amino acid sequence selected from the group consisting of: VEIKRTH (SEQ ID NO: 16), VEIKRTHH (SEQ ID NO:17), VEIKRTHHH (SEQ ID NO:18), VEIKRTHHHH (SEQ ID NO: 19), and VEIKRTHHHHH (SEQ ID NO:20).

B86. The method of any one of embodiment B61 to B85, wherein the isolated protein further comprises a third polypeptide comprising a target binding region.

B87. The method of embodiment B86, wherein the third polypeptide comprises an exposed C-terminus and the method further comprises fusing a cap domain (e.g., a His cap domain, SLSLSPGK (SEQ ID NO:36), or TVAPTESS (SEQ ID NO:37)) to the end of the exposed C-terminus of the third polypeptide.

B88. The method of embodiment B86 or B87, wherein the third polypeptide is capable of binding to a different target than the first polypeptide, the second polypeptide, or both.

B89. The method of any one of embodiments B86 to B88, wherein the exposed C-terminus of the third polypeptide comprises an anti-drug antibody epitope.

B90. The method of any one of embodiments B86 to B89, wherein the target binding region of the third polypeptide comprises an antibody variable domain or an antigen-binding fragment thereof.

B91. The method of any one of embodiments B86 to B89, wherein the target binding region of the third polypeptide comprises a light chain antibody variable domain or an antigen-binding fragment thereof.

B92. The method of any one of embodiments B86 to B89, wherein the target binding region of the third polypeptide comprises a heavy chain antibody variable domain or an antigen-binding fragment thereof.

B93. The method of any one of embodiments B86 to B92, wherein the cap domain of the third polypeptide is a His cap domain and the His cap domain consists of a single histidine residue.

B94. The method of any one of embodiments B86 to B92, wherein the cap domain of the third polypeptide is a His cap domain and the His cap domain consists of two histidine residues.

B95. The method of any one of embodiments B86 to B92, wherein the cap domain of the third polypeptide is a His cap domain and the His cap domain consists of three histidine residues.

B96. The method of any one of embodiments B86 to B92, wherein the cap domain of the third polypeptide is a His cap domain and the His cap domain consists of four histidine residues (SEQ ID NO:27).

B97. The method of any one of embodiments B86 to B92, wherein the cap domain of the third polypeptide is a His cap domain and the His cap domain consists of five histidine residues (SEQ ID NO:28).

B98. The method of any one of embodiments B86 to B89, wherein the cap domain is a His cap domain and the exposed C-terminus of the third polypeptide fused to the His cap domain consists of the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), VTVSSHHHHH (SEQ ID NO:5), TKVTVL (His)n (SEQ ID NO:46), TKLTVL (His)n (SEQ ID NO:47), TQLIIL (His)n (SEQ ID NO:48), TELTVL (His)n (SEQ ID NO: 49), and TQLTVL (His)n (SEQ ID NO:50), wherein n is 1, 2, 3, 4, or 5.

B99. The method of any one of embodiments B86 to B89, wherein the cap domain is a His cap domain and the exposed C-terminus of the third polypeptide fused to the His cap domain consists of the amino acid sequence selected from the group consisting of: VEIKH (SEQ ID NO: 6), VEIKHH (SEQ ID NO:7), VEIKHHH (SEQ ID NO:8), VEIKHHHH (SEQ ID NO:9), and VEIKHHHHH (SEQ ID NO:10).

B100. The method of any one of embodiments B86 to B89, wherein the cap domain is a His cap domain and the exposed C-terminus of the third polypeptide fused to the His cap domain consists of the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15).

B101. The method of any one of embodiments B86 to B89, wherein the cap domain is a His cap domain and the exposed C-terminus of the third polypeptide fused to the His cap domain consists of the amino acid sequence selected from the group consisting of: VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO:20).

B102. The method of any one of embodiments B61 to B85, wherein the isolated protein is bispecific.

B103. The method of any one of embodiments B61 to B101, wherein the isolated protein is multi-specific.

B104. The method of any one of embodiments B61 to B101, wherein the isolated protein is trispecific.

B105. The method of any one of embodiments B61 to B104, wherein the isolated protein is heterodimeric.

In another set of embodiments, provided are:

C1. A capping means (e.g., a histidine capping means) for reducing interaction between an isolated polypeptide and a reference, wherein the isolated polypeptide comprises one or more target binding regions and an exposed C-terminus.

C2. The capping means (e.g., a histidine capping means) of embodiment C1 wherein the one or more target binding regions comprise an antibody variable domain or antigen binding fragment thereof.

C3. The capping means (e.g., a histidine capping means) of embodiment C1 or C2, wherein the reference is a molecule present in a biological substance.

C4. The capping means (e.g., a histidine capping means) of embodiment C3, wherein the biological substance comprises serum and/or plasma.

C5. The capping means (e.g., a histidine capping means) of any one of embodiments C1 to C4, wherein the reference comprises an anti-drug antibody, optionally the anti-drug antibody is pre-existing in serum and/or plasma of a subject.

C6. The capping means (e.g., histidine capping means) of any one of embodiments C1 to C5, wherein the exposed C-terminus comprises an anti-drug antibody epitope.

C7. The capping means (e.g., histidine capping means) of any one of embodiments C1 to C6, wherein the exposed C-terminus consists of the amino acid sequence selected from the group consisting of: VTVSS (SEQ ID NO:21), VEIK (SEQ ID NO:22), VEIKR (SEQ ID NO: 23), and VEIKRT (SEQ ID NO:24).

C8. A capping means (e.g., histidine capping means) for reducing interaction between an isolated protein and a reference, wherein the isolated protein comprises:
(a) a first polypeptide comprising a target binding region, and an exposed C-terminus; and
(b) a second polypeptide comprising a target binding region.

C9. The capping means (e.g., histidine capping means) of embodiment C8, wherein the second polypeptide comprises an exposed C-terminus.

C10. The capping means (e.g., histidine capping means) of embodiment C8 or C9, wherein the target binding region of the first and second polypeptides are capable of binding to different targets.

C11. The capping means (e.g., histidine capping means) of any one of embodiment C8 to C10, wherein the isolated protein further comprises a third polypeptide comprising a target binding region.

C12. The capping means (e.g., histidine capping means) of embodiment C11, wherein the third polypeptide comprises an exposed C-terminus.

C13. The capping means (e.g., histidine capping means) of embodiment C11 or C12, wherein the third polypeptide is capable of binding to a different target than the first polypeptide, the second polypeptide, or both.

C14. The capping means (e.g., histidine capping means) of any one of embodiments C8 to C13, wherein the isolated protein is heterodimeric.

C15. The capping means (e.g., histidine capping means) of any one of embodiments C8 to C14, wherein the isolated protein is bispecific or multi-specific.

C16. The capping means (e.g., histidine capping means) of embodiment C15, wherein the multi-specific protein is trispecific.

C17. The capping means (e.g., histidine capping means) of any one of embodiments C8 to C16, wherein the exposed C-terminus comprises an anti-drug antibody epitope.

C18. The capping means (e.g., histidine capping means) of any one of embodiments C8 to C17, wherein the exposed C-terminus consists of the amino acid sequence selected from the group consisting of: VTVSS (SEQ ID NO:21), VEIK (SEQ ID NO:22), VEIKR (SEQ ID NO: 23), and VEIKRT (SEQ ID NO:24).

C19. A capping means (e.g., histidine capping means) for reducing interaction between a bispecific or a multi-specific antibody and a reference, wherein the bispecific or the multi-specific antibody comprises one or more target binding regions and an exposed C-terminus.

C20. The capping means (e.g., histidine capping means) of embodiment C19, wherein the one or more target binding regions comprises at least one antibody variable domain.

C21. The capping means (e.g., histidine capping means) of embodiment C20, wherein the at least one antibody variable domain comprises a heavy chain variable domain.

C22. The capping means (e.g., histidine capping means) of embodiment C20, wherein the at least one antibody variable domain comprises a light chain variable domain.

C23. The capping means (e.g., histidine capping means) of any one of embodiments C19 to C22, wherein the exposed C-terminus comprises an anti-drug antibody epitope.

C24. The capping means (e.g., histidine capping means) of any one of embodiments C19 to C23, wherein the multi-specific antibody comprises a trispecific antibody.

C25. An isolated polypeptide and a capping means (e.g., histidine capping means) for reducing interaction between the isolated polypeptide and a reference, wherein the isolated polypeptide comprises one or more target binding regions and an exposed C-terminus.

C26. The isolated polypeptide of embodiment C25, wherein the one or more target binding regions comprise an antibody variable domain or antigen binding fragment thereof.

C27. The isolated polypeptide of embodiments C25 or C26, wherein the reference is a molecule present in a biological substance.

C28. The isolated polypeptide of embodiment C27, wherein the biological substance comprises serum and/or plasma.

C29. The isolated polypeptide of any one of embodiments C25 to C28, wherein the reference comprises an anti-drug antibody, optionally the anti-drug antibody is pre-existing in serum and/or plasma of a subject.

C30. The isolated polypeptide of any one of embodiments C25 to C29, wherein the exposed C-terminus comprises an anti-drug antibody epitope.

C31. The isolated polypeptide of any one of embodiments C25 to C30, wherein the exposed C-terminus consists of the amino acid sequence selected from the group consisting of: VTVSS (SEQ ID NO:21), VEIK (SEQ ID NO:22), VEIKR (SEQ ID NO:23), and VEIKRT (SEQ ID NO:24).

C32. An isolated protein and a capping means (e.g., histidine capping means) for reducing interaction between the isolated protein and a reference, wherein the isolated protein comprises:
(a) a first polypeptide comprising a target binding region, and an exposed C-terminus; and
(b) a second polypeptide comprising a target binding region.

C33. The isolated protein of embodiment C32, wherein the second polypeptide comprises an exposed C-terminus.

C34. The isolated protein of embodiment C32 or C33, wherein the target binding region of the first and second polypeptides are capable of binding to different targets.

C35. The isolated protein of any one of embodiment C32 to C34, wherein the isolated protein further comprises a third polypeptide comprising a target binding region.

C36. The isolated protein of embodiment C35, wherein the third polypeptide comprises an exposed C-terminus.

C37. The isolated protein of embodiment C35 or C36, wherein the third polypeptide is capable of binding to a different target than the first polypeptide, the second polypeptide, or both.

C38. The isolated protein of any one of embodiments C32 to C37, wherein the isolated protein is heterodimeric.

C39. The isolated protein of any one of embodiments C32 to C38, wherein the isolated protein is bispecific or multi-specific.

C40. The isolated protein of embodiment C39, wherein the multi-specific protein is trispecific.

C41. The isolated protein of any one of embodiments C32 to C40, wherein the exposed C-terminus comprises an anti-drug antibody epitope.

C42. A bispecific or a multi-specific antibody and a capping means (e.g., histidine capping means) for reducing interaction between the bispecific or a multi-specific antibody and a reference, wherein the bispecific or the multi-specific antibody comprises one or more target binding regions and an exposed C-terminus.

C43. The antibody of embodiment C42, wherein the one or more target binding regions comprises at least one antibody variable domain.

C44. The antibody of embodiment C43, wherein the at least one antibody variable domain comprises a heavy chain variable domain.

C45. The antibody of embodiment C43, wherein the at least one antibody variable domain comprises a light chain variable domain.

C46. The antibody of any one of embodiments C42 to C45, wherein bispecific or a multi-specific antibody is heterodimeric.

C47. The antibody of any one of embodiments C42 to C46, wherein the multi-specific antibody comprises a trispecific antibody.

C48. The antibody of any one of embodiments C42 to C47, wherein the exposed C-terminus comprises an anti-drug antibody epitope.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations can be within the scope of the following claims.

1. An isolated polypeptide comprising one or more target binding regions and an exposed C-terminus fused to a cap domain, wherein the cap domain is a His cap domain, or consists of the amino acid sequence of SLSLSPGK (SEQ ID NO:36) or TVAPTESS (SEQ ID NO:37), and
wherein the isolated polypeptide exhibits (a) reduced interaction with a reference relative to the interaction of the isolated polypeptide lacking the cap domain with the reference; (b) exhibits reduced self-aggregation relative to the self-aggregation exhibited by the isolated polypeptide lacking the cap domain under the same conditions; or (c) reduced aggregation upon incubation with a biological substance relative to the aggregation exhibited upon incubation of the isolated polypeptide lacking the cap domain with the biological substance.

2. The isolated polypeptide of claim 1, wherein;
(a) the reference is a molecule present in a biological substance, optionally wherein (i) the biological substance comprises serum and/or plasma; (ii) the reference comprises an anti-drug antibody; and/or (iii) the reference comprises an anti-drug antibody pre-existing in serum and/or plasma of a subject;
(b) interaction with the reference is determined by an immunoassay or wherein the interaction with the reference is assessed in vitro or in vivo;
(c) self-aggregation is reduced in a high concentration liquid formulation (HCLF) optionally wherein the HCLF concentration comprises about 50 mg/mL to about 150 mg/mL of the isolated polypeptide;
(d) self-aggregation is reduced under thermal stress, optionally wherein thermal stress comprises incubation at 40° C. for about two weeks;
(e) self-aggregation is assessed by size exclusion chromatography; and/or
(f) wherein the biological substance is human serum and/or human plasma, optionally wherein incubation with the biological substance comprises incubation for: (i) about two to about five days, about two to about seven days, or about five to about fourteen days at about 37° C.; (ii) about two to about five days, about two to about seven days, or about seven to about fourteen days at about 40° C.; (iii) about two to about five days, about two to about seven days, or about seven to about fourteen days at about 4° C.; or (iv) about seven to about fourteen days at about 25° C.

3-16. (canceled)

17. The isolated polypeptide of claim 1, wherein;
(a) the exposed C-terminus comprises an anti-drug antibody epitope;
(b) the exposed C-terminus consists of the amino acid sequence selected from the group consisting of: VTVSS (SEQ ID NO:21), VEIK (SEQ ID NO:22), VEIKR (SEQ ID NO:23), VEIKRT (SEQ ID NO:24), TKVTVL (SEQ ID NO:38), TKLTVL (SEQ ID NO:39), TOLIIL (SEQ ID NO:40), TELTVL (SEQ ID NO:41), and TQLTVL (SEQ ID NO:42);
(c) the one or more target binding regions comprise (a) an antibody variable domain or an antigen-binding fragment thereof; or (b) a light chain antibody variable domain or an antigen-binding fragment thereof and/or a heavy antibody variable domain or an antigen-binding fragment thereof;
(d) the His cap domain consists of a single histidine residue, two histidine residues, three histidine residues, four histidine residues (SEQ ID NO:27), or five histidine residues (SEQ ID NO: 28); and/or
(e) wherein the exposed C-terminus fused to the His cap domain consists of:
(i) the amino acid sequence selected from the group consisting of: VTVSSH (SEQ ID NO:1), VTVSSHH (SEQ ID NO:2), VTVSSHHH (SEQ ID NO:3), VTVSSHHHH (SEQ ID NO:4), and VTVSSHHHHH (SEQ ID NO:5);
(ii) the amino acid sequence selected from the group consisting of: VEIKH (SEQ ID NO:6), VEIKHH (SEQ ID NO:7), VEIKHHH (SEQ ID NO:8), VEIKHHHH (SEQ ID NO: 9), and VEIKHHHHH (SEQ ID NO:10);
(iii) the amino acid sequence selected from the group consisting of: VEIKRH (SEQ ID NO:11), VEIKRHH (SEQ ID NO:12), VEIKRHHH (SEQ ID NO:13), VEIKRHHHH (SEQ ID NO:14), and VEIKRHHHHH (SEQ ID NO:15);
(iv) the amino acid sequence selected from the group consisting of: VEIKRTH (SEQ ID NO:16), VEIKRTHH (SEQ ID NO:17), VEIKRTHHH (SEQ ID NO: 18) VEIKR THHHH (SEQ ID NO:19), and VEIKRTHHHHH (SEQ ID NO:20); or
(v) the amino acid sequence of TKVTVL (His)ₙ (SEQ ID NO:46), TKLTVL (His)ₙ (SEQ ID NO:47), TQLIIL (His)ₙ (SEQ ID NO:48), TELTVL (His)ₙ (SEQ ID NO: 49), or TOLTVL (His)ₙ (SEQ ID NO:50), wherein n is 1, 2, 3, 4, or 5.

18-21. (canceled)

22. An isolated protein, comprising:
(a) a first polypeptide comprising the isolated polypeptide claim 1,
(b) a second polypeptide comprising a target binding region, and optionally
(c) a third polypeptide comprising a target binding region,
wherein the isolated protein exhibits reduced interaction with a reference relative to the interaction of the isolated protein lacking the cap domain with the reference.

23. The isolated protein of claim 22, wherein:
(a) the target binding region of the first and second polypeptides are capable of binding to different targets;
(b) the third polypeptide is capable of binding to a different target than the first polypeptide, the second polypeptide, or both;
(c) the exposed C-terminus of the first polypeptide comprises an anti-drug antibody epitope; and/or
(d) the target binding region of the first polypeptide, second polypeptide, and/or third polypeptide comprises (i) an antibody variable domain or an antigen-binding fragment thereof or (ii) comprises a light chain antibody variable domain or an antigen-binding fragment thereof and/or a heavy chain antibody variable domain or an antigen-binding fragment thereof.

24-26. (canceled)

27. The isolated protein of claim 22, wherein:
(a) the second polypeptide and/or third polypeptide comprises an exposed C-terminus fused to a cap domain, wherein the cap domain is a His cap domain, or consists of the amino acid sequence of SLSLSPGK (SEQ ID NO:36), TVAPTESS (SEQ ID NO:37), HHHHHH (SEQ ID NO: 26), or AS, optionally wherein the exposed C-terminus of the second polypeptide and/or third polypeptide comprises an anti-drug antibody epitope;
(b) the His cap domain of the second polypeptide and/or third polypeptide consists of a single histidine residue, two histidine residues, three histidine residues, four histidine residues (SEQ ID NO:27), or five histidine residues (SEQ ID NO:28); and/or
(c) wherein the exposed C-terminus fused to the His cap domain of the second polypeptide and/or third polypeptide consists of the amino acid sequence selected from the group consisting of:
(i) VTVSSH (SEQ ID NO:1), VTVSSHH (SEQ ID NO:2), VTVSSHHH (SEQ ID NO: 3), VTVSSHHHH (SEQ ID NO:4), and VTVSSHHHHH (SEQ ID NO:5);
(ii) VEIKH (SEQ ID NO:6), VEIKHH (SEQ ID NO:7), VEIKHHH (SEQ ID NO: 8), VEIKHHHH (SEQ ID NO:9), and VEIKHHHHH (SEQ ID NO:10);
(iii) VEIKRH (SEQ ID NO:11), VEIKRHH (SEQ ID NO:12), VEIKRHHH (SEQ ID NO: 13), VEIKRHHHH (SEQ ID NO: 14), and VEIKRHHHHH (SEQ ID NO: 15);
(iv) VEIKRTH (SEQ ID NO:16), VEIKRTHH (SEQ ID NO:17), VEIKRTHHH (SEQ ID NO:18), VEIKRTHHHH (SEQ ID NO:19), and VEIKRTHHHHH (SEQ ID NO:20); or
(v) the amino acid sequence of TKVTVL (His)ₙ (SEQ ID NO:46), TKLTVL (His)ₙ (SEQ ID NO:47), TQLIIL (His)ₙ (SEQ ID NO:48), TELTVL (His)ₙ (SEQ ID NO: 49), or TQLTVL (His)ₙ (SEQ ID NO:50), wherein n is 1, 2, 3, 4, or 5.

28-42. (canceled)

43. The isolated protein of claim 22, wherein the isolated protein is (a) bispecific, trispecific, or multi-specific; and/or (b) heterodimeric.

44. (canceled)

45. An isolated nucleic acid sequence comprising a nucleotide sequence encoding the polypeptide of claim 1.

46. An expression vector comprising the nucleic acid sequence of claim 45.

47. An isolated cell (a) expressing the polypeptide of claim 1, (b) comprising a nucleic acid sequence encoding the polypeptide; and/or (c) comprising a vector comprising the nucleic acid sequence.

48. (canceled)

49. A method for producing the polypeptide of claim 1, comprising culturing a cell expressing the polypeptide; (b) comprising a nucleic acid sequence encoding the polypeptide; and/or (c) comprising a vector comprising the nucleic acid; optionally comprising isolating the polypeptide.

50. A bispecific, trispecific, or a multi-specific antibody comprising the isolated polypeptide of claim 1, and wherein the antibody exhibits reduced interaction with an anti-drug antibody relative to the interaction of the antibody lacking the cap domain with the anti-drug antibody.

51-63. (canceled)

64. A method for:
(a) reducing the interaction between an isolated polypeptide and a reference, wherein the isolated polypeptide comprises one or more target binding regions and an exposed C-terminus, the method comprising producing the isolated polypeptide of claim 1 by fusing a cap domain at the end of the exposed C-terminus, wherein the cap domain is a His cap domain, or consists of the amino acid sequence of SLSLSPGK (SEQ ID NO:36) or TVAPTESS (SEQ ID NO:37);
(b) reducing the aggregation of an isolated polypeptide in a biological substance, wherein the isolated polypeptide comprises one or more target binding regions and an exposed C-terminus, the method comprising producing the isolated polypeptide of claim 1 by fusing a cap domain at the end of the exposed C-terminus, wherein the cap domain is a His cap domain, or consists of the amino acid sequence of SLSLSPGK (SEQ ID NO:36) or TVAPTESS (SEQ ID NO: 37); or
(c) reducing self-aggregation of an isolated polypeptide, wherein the isolated polypeptide comprises one or more target binding regions and an exposed C-terminus, the method comprising producing the isolated polypeptide of claim 1 by fusing a cap domain at the end of the exposed C-terminus, wherein the cap domain is a His cap domain, or consists of the amino acid sequence of SLSLSPGK (SEQ ID NO:36) or TVAPTESS (SEQ ID NO:37).

65-85. (canceled)

86. A method for reducing the interaction between an isolated protein and a reference, wherein the isolated protein comprises:
(a) a first polypeptide comprising a target binding region, and an exposed C-terminus; and
(b) a second polypeptide comprising a target binding region; and optionally
(c) a third polypeptide comprising a target binding domain,
the method comprising producing the isolated protein of claim 22 by fusing a cap domain at the end of the exposed C-terminus of the first polypeptide, wherein the cap domain is a His cap domain, or consists of the amino acid sequence of SLSLSPGK (SEQ ID NO:36) or TVAPTESS (SEQ ID NO:37).

87-108. (canceled)

109. A capping means for reducing interaction between:
(a) an isolated polypeptide and a reference, wherein the isolated polypeptide comprises one or more target binding regions and an exposed C-terminus, optionally wherein the capping means is a histidine capping means, and optionally wherein the exposed C-terminus consists of the amino acid sequence selected from the group consisting of: VTVSS (SEQ ID NO:21), VEIK (SEQ ID NO:22), VEIKR (SEQ ID NO:23), VEIKRT (SEQ ID NO:24), TKVTVL (SEQ ID NO: 38), TKLTVL (SEQ ID NO:39), TOLIIL (SEQ ID NO:40), TELTVL (SEQ ID NO:41), and TOLTVL (SEQ ID NO:42);
(b) an isolated protein and a reference, wherein the isolated protein comprises:
(i) a first polypeptide comprising a target binding region, and an exposed C-terminus; and
(ii) a second polypeptide comprising a target binding region;
optionally wherein the capping means is a histidine capping means, and optionally wherein the exposed C-terminus consists of the amino acid sequence selected from the group consisting of: VTVSS (SEQ ID NO:21), VEIK (SEQ ID NO:22), VEIKR (SEQ ID NO:23), VEIKRT (SEQ ID NO: 24), TKVTVL (SEQ ID NO:38), TKLTVL (SEQ ID NO:39), TOLIIL (SEQ ID NO:40), TELTVL (SEQ ID NO:41), and TOLTVL (SEQ ID NO:42); and/or
(c) a bispecific or a multi-specific antibody and a reference, wherein the bispecific or the multi-specific antibody comprises one or more target binding regions and an exposed C-terminus optionally wherein the capping means is a histidine capping means, and optionally wherein the exposed C-terminus consists of the amino acid sequence selected from the group consisting of: VTVSS (SEQ ID NO:21), VEIK (SEQ ID NO:22), VEIKR (SEQ ID NO:23), VEIKRT (SEQ ID NO: 24), TKVTVL (SEQ ID NO:38), TKLTVL (SEQ ID NO:39), TOLIIL (SEQ ID NO:40), TELTVL (SEQ ID NO:41), and TOLTVL (SEQ ID NO:42).

110-131. (canceled)

132. A composition comprising the capping means of claim 109 and:
(a) the isolated polypeptide;
(b) the isolated protein; or
(c) the bispecific or multispecific antibody.

133-155. (canceled)

156. A method for conducting a diagnostic assay comprising using an isolated protein comprising the isolated polypeptide of claim 1, optionally wherein:
(a) the protein is an antibody;
(b) the protein is an antibody that binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin;
(c) the protein is an antibody that binds to an antigen of a pathogen, optionally wherein the pathogen is a virus, a bacteria, a fungus, or a parasite;
(d) the protein is used in a diagnostic assay to detect the presence of a molecule in a biological substance, optionally wherein (a) the molecule is an antigen and/or (b) wherein the biological substance is serum and/or plasma; and/or
(e) the diagnostic assay is conducted in vitro or in vivo.

157-166. (canceled)

167. A method comprising administering to a subject an isolated therapeutic protein comprising the isolated polypeptide of claim 1, optionally wherein:
(a) the protein is a cytokine, optionally wherein the cytokine is IL-12, IL-23, IL-1β, IL-6, IL-15, IL-2, IL-5, TNF-alpha, IL-9, or IL-17;
(b) the protein is an antibody;
(c) the protein is an antibody that binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin; and/or
(d) the protein is an antibody that binds to an antigen of a pathogen, optionally wherein the pathogen is a virus, a bacteria, a fungus, or a parasite.

168-174. (canceled)

175. An isolated protein comprising the isolated polypeptide of claim 1, wherein the exposed C-terminus consists of the amino acid sequence VTVSS (SEQ ID NO:21), VEIK (SEQ ID NO:22), VEIKR (SEQ ID NO:23), VEIKRT (SEQ ID NO:24), TKVTVL (SEQ ID NO:38), TKLTVL (SEQ ID NO:39), TQLIIL (SEQ ID NO:40), TELTVL (SEQ ID NO:41), or TQLTVL (SEQ ID NO: 42), optionally wherein:
(a) the protein is an antibody;
(b) the protein is an antibody that binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin; an/or
(c) the protein is an antibody that binds to an antigen of a pathogen, optionally wherein the pathogen is a virus, a bacteria, a fungus, or a parasite.

176-179. (canceled)

180. The method of claim 167 wherein the isolated therapeutic protein is a therapeutic bispecific or multi-specific antibody comprising two or more target binding regions, optionally wherein:
(a) the two or more target binding regions bind to two or more antigens;
(b) the two or more target binding regions bind to two or more antigens and at least one antigen is selected from the group consisting of a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, and mesothelin; and/or
(c) the antibody binds to an antigen of a pathogen, optionally wherein the pathogen is a virus, a bacteria, a fungus, or a parasite.

181-186. (canceled)

187. A pharmaceutical composition comprising the protein of claim 175, and (a) a pharmaceutically acceptable excipient for use in therapy; or (b) a pharmaceutically acceptable excipient for use in a diagnostic assay.

188. (canceled)

189. Use of a cap domain to:
(a) reduce interaction between an anti-drug antibody and a protein, wherein the protein comprises one or more target binding regions and an exposed C-terminus, and wherein the cap domain is a His cap domain, or consists of the amino acid sequence of SLSLSPGK (SEQ ID NO: 36) or TVAPTESS (SEQ ID NO:37);
(b) reduce aggregation in a biological substance, wherein the protein comprises one or more target binding regions and an exposed C-terminus, and wherein the cap domain is a His cap domain, or consists of the amino acid sequence of SLSLSPGK (SEQ ID NO:36) or TVAPTESS (SEQ ID NO:37); and/or
(c) reduce self-aggregation, wherein the protein comprises one or more target binding regions and an exposed C-terminus, and wherein the cap domain consists is a His cap domain, or consists of the amino acid sequence of SLSLSPGK (SEQ ID NO:36) or TVAPTESS (SEQ ID NO: 37).

190-194. (canceled)

195. An isolated protein comprising a target binding region and an exposed C-terminus fused to a cap domain, wherein the cap domain consists of His cap domain, or the amino acid sequences of HHHHHH (SEQ ID NO:26)), SLSLSPGK (SEQ ID NO: 36), or AS, TVAPTESS (SEQ ID NO:37), optionally wherein:
(a) the protein exhibits reduced interaction with an anti-drug antibody in a biological substance, optionally wherein the biological substance comprises serum and/or plasma;
(b) the exposed C-terminus comprises an anti-drug antibody epitope;
(c) the exposed C-terminus consists of the amino acid sequence selected from the group consisting of: VTVSS (SEQ ID NO:21), VEIK (SEQ ID NO:22), VEIKR (SEQ ID NO:23), VEIKRT (SEQ ID NO:24), TKVTVL (SEQ ID NO:38), TKLTVL (SEQ ID NO:39), TOLIIL (SEQ ID NO:40), TELTVL (SEQ ID NO:41), or TQLTVL (SEQ ID NO:42); and/or
(d) the protein is for use in therapy or a diagnostic assay.

196-200. (canceled)
